(19) United States
(12) Patent Application Publication
(10) Pub. No.: US 2022/0325289 A1
(43) Pub. Date: Oct. 13, 2022

(54) TOOLS AND METHODS FOR USING CELL DIVISION LOCI TO CONTROL PROLIFERATION OF CELLS

(71) Applicant: Sinai Health System, Toronto (CA)

(72) Inventors: Andras NAGY, Toronto (CA); Claudio MONETTI, Toronto (CA); Qin LIANG, Toronto (CA)

(21) Appl. No.: 17/825,784

(22) Filed: May 26, 2022

Related U.S. Application Data
(63) Continuation of application No. 15/556,146, filed on Sep. 6, 2017.
(60) Provisional application No. 62/130,270, filed on Mar. 9, 2015.
(60) Provisional application No. 62/130,258, filed on Mar. 9, 2015.

(51) Int. Cl. C12N 15/65; C12N 15/85; C12N 15/90; C12N 5/0735; C12N 5/10; A61K 48/00; C12N 15/63

(52) U.S. Cl. CPC C12N 15/65; C12N 15/85; C12N 15/907; C12N 5/0606; C12N 5/10; A61K 48/005; C12N 15/63; C12N 2830/001

(57) ABSTRACT

The present disclosure provides molecular tools, methods and kits for using cell division loci (CDLs) to control cell proliferation in animal cells. CDLs, as provided herein, are loci whose transcription product(s) are expressed during cell division. CDLs may be genetically modified, as described herein, to comprise a negative selectable marker and/or an inducible activator-based gene expression system, which allows a user to permit, ablate, and/or inhibit proliferation of the genetically modified cell(s) by adding or removing an appropriate inducer.

### SEQUENCE LISTING

The instant application contains a Sequence Listing which has been submitted electronically in ASCII format and is hereby incorporated by reference in its entirety. The ASCII copy, created on May 19, 2022, is named 51276-002004_Sequence_Listing_5_19_22_ST25 and is 409,964 bytes in size.

### FIELD OF THE DISCLOSURE

The present description relates generally to the fields of cell and molecular biology. More particularly, the description relates to molecular tools, methods and kits for controlling division of animal cells and genetically modified cells related to same.

### BACKGROUND OF THE DISCLOSURE

Human pluripotent stem (hPS) cells, may be used as tools for understanding normal cellular development, disease development and for use in cellular therapeutics for treating currently incurable disorders, such as, for example, genetic disorders, degenerative diseases and/or various injuries. The pluripotent nature of these cells renders them able to differentiate into any cell type after a period of self-renewal in the stem cell state (Rossant and Nagy, 1999). The gold standard of hPS cells are the human embryonic stem (hES) cells reported in 1998 (Thomson et al., 1998). In 2006 and 2007 a method for reprogramming differentiated somatic cells, such as skin fibroblasts, into ES cell-like “induced pluripotent stem” (iPS) cells was reported and expanded the types of pluripotent cells (Takahashi and Yamanaka, 2006; Takahashi et al., 2007). The methods of generation of iPS cells and their applications toward many directions including cell-based therapies for treating diseases and aberrant physiological conditions have been developed further in the years since.

One concern regarding pluripotent cell-based therapies is safety. For example, malignant growth originating from a cell graft is of concern. The process of reprogramming differentiated cells into iPS cells is also relevant to safety, as it has been reported that reprogramming methods can cause genome damage and aberrant epigenetic changes (Hussein et al., 2011; Laurent et al., 2011; Lister et al., 2011), which may pose a risk for malignant transformation of iPS cell-derived cells.

One challenge with cell-based therapies involving pluripotent cells expanded in vitro is the pluripotent nature of the cells themselves. For example, if pluripotent cells remain among differentiated therapeutic cells, the pluripotent cells may develop into teratomas (Yoshida and Yamanaka, 2010). Attempts to increase the safety of pluripotent cell-derived products and therapies have included efforts to eliminate pluripotent cells from cell cultures after in vitro differentiation. For example: cytotoxic antibodies have been used to eliminate cells having pluripotent-specific antigens (Choo et al., 2008; Tan et al., 2009); cells have been sorted based on pluripotency cell surface markers (Ben-David et al., 2013a; Fong et al., 2009; Tang et al., 2011); tumour progression genes have been genetically altered in cells (Blum et al., 2009; Menendez et al., 2012); transgenes for assisting with separation of differentiated cells have been introduced into cells (Chung et al., 2006; Eiges et al., 2001; Huber et al., 2007); suicide genes have been introduced into cells and used to eliminate residual pluripotent stem cells after differentiation (Rong et al., 2012; Schuldiner et al., 2003); and undesired pluripotent cells have been ablated using chemicals (Ben-David et al., 2013b; Dabir et al., 2013; Tohyama et al., 2013). It is possible that even if residual pluripotent cells are eliminated from differentiated cultures, the differentiated derivatives of pluripotent cells may have oncogenic properties (Ghosh et al., 2011). Related oncogenic events could occur in therapeutic cells i) during in vitro preparation of cells; or ii) following grafting of cells into a host.

Most current strategies for eliminating or preventing unwanted cell growth and/or differentiation are based on the herpes simplex virus—thymidine kinase (HSV-TK)/ganciclovir (GCV) negatively selectable system, which may be used to eliminate a graft entirely, if malignancy develops (Schuldiner et al., 2003) or to eliminate only the pluripotent cells ‘contaminating’ the intended differentiated derivatives (Ben-David and Benvenisty, 2014; Lim et al., 2013). The mechanism of GCV-induced cell killing and apoptosis is well understood. It creates a replication-dependent formation of DNA double-strand breaks (Halloran and Fenton, 1998), which leads to apoptosis (Tomicic et al., 2002). However, many HSV-TK/GCV-based systems are unreliably expressed, at least because they rely on random integration or transient expression of HSV-TK. Strategies involving negative selectable markers with different killing mechanisms, such as, for example, Caspase 9 (Di Stasi et al., 2011) have been tested, but reliable expression of the negative selectable marker has not been shown. Cell-based therapies may require millions or billions of cells, which may amplify any issues caused by unwanted cell growth and/or differentiation.

It is an object of the present disclosure to mitigate and/or obviate one or more of the above deficiencies.

### SUMMARY OF THE DISCLOSURE

In an aspect, a method of controlling proliferation of an animal cell is provided. The method comprises: providing an animal cell; genetically modifying in the animal cell a cell division locus (CDL), the CDL being one or more loci whose transcription product(s) is expressed by dividing cells; the genetic modification of the CDL comprising one or more of: a) an ablation link (ALINK) system, the ALINK system comprising a DNA sequence encoding a negative selectable marker that is transcriptionally linked to a DNA sequence encoding the CDL; and b) an inducible exogenous activator of regulation of a CDL (EARC) system, the EARC system comprising an inducible activator-based gene expression system that is operably linked to the CDL; controlling proliferation of the genetically modified animal cell comprising the ALINK system with an inducer of the negative selectable marker; and/or controlling proliferation of the genetically modified animal cell comprising the EARC system with an inducer of the inducible activator-based gene expression system.

In an embodiment of the method of controlling proliferation of an animal cell provided herein, the controlling of the ALINK-modified animal cell comprises one or more of: permitting proliferation of the genetically modified animal cell comprising the ALINK system by maintaining the genetically modified animal cell comprising the ALINK system in the absence of an inducer of the negative selectable marker; and ablating or inhibiting proliferation of the genetically modified animal cell comprising the ALINK system by exposing the animal cell comprising the ALINK system to the inducer of the negative selectable marker.

In an embodiment of the method of controlling proliferation of an animal cell provided herein, the controlling of the EARC-modified animal cell comprises one or more of: permitting proliferation of the genetically modified animal cell comprising the EARC system by exposing the genetically modified animal cell comprising the EARC system to an inducer of the inducible activator-based gene expression system; and preventing or inhibiting proliferation of the genetically modified animal cell comprising the EARC system by maintaining the animal cell comprising the EARC system in the absence of the inducer of the inducible activator-based gene expression system.

In various embodiments of the method of controlling proliferation of an animal cell provided herein, the genetic modification of the CDL comprises preforming targeted replacement of the CDL with one or more of: a) a DNA vector comprising the ALINK system; b) a DNA vector comprising the EARC system; and c) a DNA vector comprising the ALINK system and the EARC system.

In various embodiments of the method of controlling proliferation of an animal cell provided herein, the ALINK genetic modification of the CDL is homozygous, heterozygous, hemizygous or compound heterozygous and/or wherein the EARC genetic modification ensures that functional CDL modification can only be generated through EARC-modified alleles.

In various embodiments of the method of controlling proliferation of an animal cell provided herein, the CDL is one or more loci recited in Table 2. In various embodiments, the CDL encodes a gene product whose function is involved with one or more of: cell cycle, DNA replication, RNA transcription, protein translation, and metabolism. In various embodiments the CDL is one or more of Cdk1/CDK1, Top2A/TOP2A, Cenpa/CENPA, Birc5/BIRC5, and Eef2/EEF2, preferably the CDL is Cdk1 or CDK1.

In various embodiments of the method of controlling proliferation of an animal cell provided herein, the ALINK system comprises a herpes simplex virus-thymidine kinase/ganciclovir system, a cytosine deaminase/5-fluorocytosine system, a carboxyl esterase/irinotecan system or an iCasp9/AP1903 system, preferably the ALINK system is a herpes simplex virus-thymidine kinase/ganciclovir system.

In various embodiments of the method of controlling proliferation of an animal cell provided herein, the EARC system is a dox-bridge system, a cumate switch inducible system, an ecdysone inducible system, a radio wave inducible system, or a ligand-reversible dimerization system, preferably the EARC system is a dox-bridge system.

In various embodiments of the method of controlling proliferation of an animal cell provided herein, the animal cell is a mammalian cell or an avian cell. In various embodiment, the mammalian cell is a human, mouse, rat, hamster, guinea pig, cat, dog, cow, horse, deer, elk, bison, oxen, camel, llama, rabbit, pig, goat, sheep, or non-human primate cell, preferably the mammalian cell is a human cell.

In various embodiments of the method of controlling proliferation of an animal cell provided herein, the animal cell is a pluripotent stem cell a multipotent cell, a monopotent progenitor cell, or a terminally differentiated cell.

In various embodiments of the method of controlling proliferation of an animal cell provided herein, the animal cell is derived from a pluripotent stem cell, a multipotent cell, a monopotent progenitor cell, or a terminally differentiated cell.

In an aspect, an animal cell genetically modified to comprise at least one mechanism for controlling cell proliferation is provided. The genetically modified animal cell comprises: a genetic modification of one or more cell division locus (CDL), the CDL being one or more loci whose transcription product(s) is expressed by dividing cells. The genetic modification being one or more of: a) an ablation link (ALINK) system, the ALINK system comprising a DNA sequence encoding a negative selectable marker that is transcriptionally linked to a DNA sequence encoding the CDL; and b) an exogenous activator of regulation of a CEDL (EARC) system, the EARC system comprising an inducible activator-based gene expression system that is operably linked to the CDL.

In an embodiment of the animal cell genetically modified to comprise at least one mechanism for controlling cell proliferation provided herein, the genetic modification of the CDL comprises preforming targeted replacement of the CDL with one or more of: a) a DNA vector comprising the ALINK system; b) a DNA vector comprising the EARC system; and c) a DNA vector comprising the ALINK system and the EARC system.

In various embodiments of the animal cell genetically modified to comprise at least one mechanism for controlling cell proliferation provided herein, the ALINK genetic modification of the CDL is homozygous, heterozygous, hemizygous or compound heterozygous and/or wherein the EARC genetic modification ensures that functional CDL modification can only be generated through EARC-modified alleles.

In various embodiments of the animal cell genetically modified to comprise at least one mechanism for controlling cell proliferation provided herein, the CDL is one or more loci recited in Table 2. In various embodiments, the CDL encodes a gene product whose function is involved with one or more of: cell cycle, DNA replication, RNA transcription, protein translation, and metabolism. In various embodiments, the CDL is one or more of Cdk1/CDK1, Top2A/TOP2A, Cenpa/CENPA, Birc5/BIRC5, and Eef2/EEF2, preferably the CDL is Cdk1 or CDK1.

In various embodiments of the animal cell genetically modified to comprise at least one mechanism for controlling cell proliferation provided herein, the ALINK system comprises a herpes simplex virus-thymidine kinase/ganciclovir system, a cytosine deaminase/5-fluorocytosine system, a carboxyl esterase/irinotecan system or an iCasp9/AP1903 system, preferably the ALINK system is a herpes simplex virus-thymidine kinase/ganciclovir system.

In various embodiments of the animal cell genetically modified to comprise at least one mechanism for controlling cell proliferation provided herein, the EARC system is a dox-bridge system, a cumate switch inducible system, an ecdysone inducible system, a radio wave inducible system, or a ligand-reversible dimerization system, preferably the EARC system is a dox-bridge system.

In various embodiments of the animal cell genetically modified to comprise at least one mechanism for controlling cell proliferation provided herein, the animal cell is a mammalian cell or an avian cell. In various embodiments, the mammalian cell is a human, mouse, rat, hamster, guinea pig, cat, dog, cow, horse, deer, elk, bison, oxen, camel, llama, rabbit, pig, goat, sheep, or non-human primate cell, preferably the mammalian cell is a human cell.

In various embodiments of the animal cell genetically modified to comprise at least one mechanism for controlling cell proliferation provided herein, the animal cell is a pluripotent stem cell a multipotent cell, a monopotent progenitor cell, or a terminally differentiated cell.

In various embodiments of the animal cell genetically modified to comprise at least one mechanism for controlling cell proliferation provided herein, the animal cell is derived from a pluripotent stem cell, a multipotent cell, a monopotent progenitor cell, or a terminally differentiated cell.

In an aspect, a DNA vector for modifying expression of a cell division locus (CDL), the CDL being one or more loci whose transcription product(s) is expressed by dividing cells is provided. The DNA vector comprises: an ablation link (ALINK) system, the ALINK system comprising a DNA sequence encoding a negative selectable marker that is transcriptionally linked to the CDL, wherein if the DNA vector is inserted into one or more host cells, proliferating host cells comprising the DNA vector will be killed if the proliferating host cells comprising the DNA vector are exposed to an inducer of the negative selectable marker.

In an aspect, DNA vector for modifying expression of a cell division essential locus (CDL), the CDL being one or more loci whose transcription product(s) is expressed by dividing cells is provided. The DNA vector comprises: an exogenous activator of regulation of a CDL (EARC) system, the EARC system comprising an inducible activator-based gene expression system that is operably linked to the CDL, wherein if the DNA vector is inserted into one or more host cells, proliferating host cells comprising the DNA vector will be killed if the proliferating host cells comprising the DNA vector are not exposed to an inducer of the inducible activator-based gene expression system.

In an aspect, a DNA vector for modifying expression of a cell division essential locus (CDL), the CDL being one or more loci whose transcription product(s) is expressed by dividing cells is provided. The DNA vector comprises: an ablation link (ALINK) system, the ALINK system being a DNA sequence encoding a negative selectable marker that is transcriptionally linked to the CDL; and an exogenous activator of regulation of CDL (EARC) system, the EARC system comprising an inducible activator-based gene expression system that is operably linked to the CDL, wherein if the DNA vector is inserted into one or more host cells, proliferating host cells comprising the DNA vector will be killed if the proliferating host cells comprising the DNA vector are exposed to an inducer of the negative selectable marker and if the proliferating host cells comprising the DNA vector are not exposed to an inducer of the inducible activator-based gene expression system.

In various embodiments of the DNA vectors provided herein, the CDL is one or more loci recited in Table 2. In various embodiments, the CDL encodes a gene product whose function is involved with one or more of: cell cycle, DNA replication, RNA transcription, protein translation, and metabolism. In various embodiments, the CDL is one or more of Cdk1/CDK1, Top2A/TOP2A, Cenpa/CENPA, Birc5/BIRC5, and Eef2/EEF2, preferably the CDL is Cdk1 or CDK1.

In various embodiments of the DNA vectors provided herein, the ALINK system comprises a herpes simplex virus-thymidine kinase/ganciclovir system, a cytosine deaminase/5-fluorocytosine system, a carboxyl esterase/irinotecan system or an iCasp9/AP1903 system, preferably the ALINK system is a herpes simplex virus-thymidine kinase/ganciclovir system.

In various embodiments of the DNA vectors provided herein, the EARC system is a dox-bridge system, a cumate switch inducible system, an ecdysone inducible system, a radio wave inducible system, or a ligand-reversible dimerization system, preferably the EARC system is a dox-bridge system.

In an aspect, a kit for controlling proliferation of an animal cell by genetically modifying one or more cell division essential locus/loci (CDL), the CDL being one or more loci whose transcription product(s) is expressed by dividing cells is provided. The kit comprises: a DNA vector comprising an ablation link (ALINK) system, the ALINK system comprising a DNA sequence encoding a negative selectable marker that is transcriptionally linked to a DNA sequence encoding the CDL; and/or a DNA vector comprising an exogenous activator of regulation of a CDL (EARC) system, the EARC system comprising an inducible activator-based gene expression system that is operably linked to the CDL; and/or a DNA vector comprising an ALINK system and an EARC system, the ALINK and EARC systems each being operably linked to the CDL; and instructions for targeted replacement of the CDL in an animal cell using one or more of the DNA vectors.

In an embodiment of the kit provided herein, the CDL is one or more loci recited in Table 2. In various embodiments, the CDL encodes a gene product whose function is involved with one or more of: cell cycle, DNA replication, RNA transcription, protein translation, and metabolism. In various embodiments, the CDL is one or more of Cdk1/CDK1, Top2A/TOP2A, Cenpa/CENPA, Birc5/BIRC5, and Eef2/EEF2, preferably the CDL is Cdk1 or CDK1.

In various embodiments of the kit provided herein, the ALINK system comprises a herpes simplex virus-thymidine kinase/ganciclovir system, a cytosine deaminase/5-fluorocytosine system, a carboxyl esterase/irinotecan system or an iCasp9/AP1903 system, preferably the ALINK system is a herpes simplex virus-thymidine kinase/ganciclovir system.

In various embodiments of the kit provided herein, the EARC system is a dox-bridge system, a cumate switch inducible system, an ecdysone inducible system, a radio wave inducible system, or a ligand-reversible dimerization system, preferably the EARC system is a dox-bridge system.

### DESCRIPTION OF THE DRAWINGS

The patent or application file contains at least one drawing executed in color. Copies of this patent or patent application publication with color drawing(s) will be provided by the Office upon request and payment of the necessary fee.

These and other features of the disclosure will become more apparent in the following detailed description in which reference is made to the appended drawings wherein:

FIGS. 1A-1G depict schematics illustrating the concept of induced negative effectors of proliferation (iNEPs) and examples of iNEP systems contemplated for use in the methods and tools provided herein. FIG. 1A depicts a schematic representing different examples of iNEP-modified CDLs, including a homozygous modification in CDL1, homozygous insertions in CDL1 and CDL2, CDL comprising two separate loci that together are essential for cell division (CDL3). FIG. 1B depicts schematics representing examples of iNEP comprising an ablation link (ALINK) and an exogenous activator of regulation of a CDL (EARC) in different configurations. FIG. 1C depicts a schematic illustrating transcription activator-like effector (TALE) technology combined with dimerizer-regulated expression induction. FIG. 1D depicts a schematic illustrating a reverse-cumate-Trans-Activator (rcTA) system. FIG. 1E depicts a schematic illustrating a retinoid X receptor (RXR) and an N-terminal truncation of ecdysone receptor (EcR) fused to the activation domain of Vp16 (VpEcR). FIG. 1F depicts a schematic illustrating a transient receptor potential vanilloid-1 (TRPV1), together with ferritin, which is one example of an iNEP system, as set forth herein. FIG. 1G depicts a schematic illustrating how an IRES and a dimerization agent may be used as an iNEP.

FIGS. 2A-2F depict schematics illustrating targeting HSV-TK into the 3′UTR of the Cdk1 locus to generate an ALINK, which enables elimination of dividing modified CDK1-expressing cells. FIG. 2A shows a schematic of the mouse Cdk1 locus. FIG. 2B shows a schematic of mouse target vector I. FIG. 2C shows a schematic of a Cdk1TC allele. FIG. 2D shows a schematic of mouse target vector II. FIG. 2E shows a schematic of a Cdk1TClox allele. FIG. 2F depicts the position of the CRISPR guide RNA (SEQ ID NO: 155); the sequence in the yellow box is the 8th exon of Cdk1 (sense strand: SEQ ID NO: 153; anti-sense strand: SEQ ID NO: 154).

FIGS. 3A-3G depict generation of ALINK example, HSV-TK-mCherry into the 3′UTR of the CDK1 locus to generate ALINK in mouse ES cell lines. FIG. 3A shows the overall steps of generating ALINK in mouse C2 ES cells. FIG. 3B shows southern blotting result of correct genotyping of Cdk1(TK/+), Cdk1(TK, loxP-TK), and Cdk1(TK/TK). FIG. 3C shows the locations of the primers used in ALINK genotyping in mouse cells. FIG. 3D includes PCR results illustrating targeting of Targeting Vector I into the 3′UTR of the CDK1 locus. FIG. 3E shows PCR results illustrating the excision event of selection marker in a mouse ES cell line already correctly targeted with Targeting Vector I to activate the expression of HSV-TK-mCherry. FIG. 3F shows PCR results illustrating targeting of Targeting Vector II into Cdk1(TK/+) cells. FIG. 3G shows PCR results illustrating the excision event of selection marker in Cdk1(TK, loxP-TK) to activate the 2nd allele expression of HSV-TK-mCherry, thus generating Cdk1(TK/TK).

FIGS. 4A-4K depict generation of an ALINK modification, HSV-TK-mCherry into the 3′UTR of the CDK1 locus, in human ES cell lines. FIG. 4A shows the overall steps of generating ALINK in human CA1 ES cells. FIG. 4B shows the locations of the primers used in ALINK genotyping in human CA1 cells. FIG. 4C shows PCR results illustrating targeting of Targeting Vector I into the 3′UTR of the CDK1 locus. FIG. 4D shows flow cytometry illustrating the excision event of selection marker in human Cdk1(PB-TK/+) ES cell line to activate the expression of HSV-TK-mCherry; the Y-axis shows the mCherry expression level, while the X-axis is an autofluorescence channel. FIG. 4E shows PCR results illustrating targeting of Targeting Vector II (puro-version) into Cdk1(TK/+) cells; the upper panel is PCR using primers flanking the 5′homology arm; the lower panel is PCR using primers inside 5′ and 3′ homology arm, so absence of 0.7 kb band and presence of 2.8 kb band means that the clone is homozygous in ALINK, and presence of 0.7 kb band means that the clone is heterozygous in ALINK or the population is not clonal. FIG. 4F shows flow cytometry analysis illustrating the excision event of selection marker in Cdk1(TK, loxP-TK) to activate the 2nd allele expression of HSV-TK-mCherry; the Y-axis shows the mCherry expression level, while the X-axis is an autofluorescence channel. FIG. 4G shows the overall steps of generating ALINK in human H1 ES cells. FIG. 4H shows the locations of the primers used in ALINK genotyping in human H1 cells. FIG. 4I shows PCR results illustrating targeting of Targeting Vector II into the 3′UTR of the CDK1 locus. FIG. 4J shows PCR results illustrating the excision event of selection marker in human H1 Cdk1(loxP-TK/+) to activate the expression of HSV-TK-mCherry; the Y-axis shows the mCherry expression level, while the X-axis is an autofluorescence channel. FIG. 4K shows fluorescence-activated cell sorting (FACS) of targeting of Targeting Vector III (GFP-version) into Cdk1(TK/+) cells. After FACS sorting, clones picked from sparse plating were genotyped with mCherry-allele-specific primers, eGFP-allele-specific primers and primers in 5′ and 3′ homology arms; clones labeled with orange star sign are homozygous ALINK with one allele of mCherry and one allele of eGFP; the one clone labeled with green star sign is homozygous ALINK with two alleles of eGFP.

FIGS. 5A-5C depict teratoma histology (endoderm, mesoderm and ectoderm portions of the teratoma are shown from left to right, respectively). FIG. 5A depicts photomicrographs of a teratoma derived from a mouse ES Cdk1+/+, alink/alink cell. FIG. 5B depicts photomicrographs of a teratoma derived from a mouse ES Cdk1earc/earc, alink/alink cell. FIG. 5C depicts photomicrographs of a teratoma derived from a human ES Cdk1+/+, alink/alink cell.

FIGS. 6A-6B depict in vitro functional analysis of mouse ES cells with an HSV-TK—mCherry knock-in into the 3′UTR of the CDK1 locus. FIG. 6A illustrates killing efficiency provided by the TK.007 gene after cells were exposed to different concentrations of GCV for 3 days. Colony size and number are directly proportional to GCV concentration. The second lowest concentration of 0.01 μM did not affect the colony number but slowed down cell growth as evidenced by the reduced colony size (n=5). FIG. 6B illustrates expression of mCherry before (Cdk1⋅HSV-TKNeoIN) and after (Cdk1⋅HSV-TK) PB-mediated removal of the neo-cassette.

FIGS. 7A-7F depict results of cellular experiments using ALINK-modified cells. FIG. 7A graphically depicts results of GCV treatment of subcutaneous teratomas comprising ALINK-modified mouse C2 cells. FIG. 7B graphically depicts results of GCV treatment of subcutaneous teratomas comprising ALINK-modified H1 ES cells. FIG. 7C graphically depicts results of GCV treatment of mammary gland tumors comprising ALINK-modified cells. FIG. 7D schematically depicts experimental design of neural assay. FIG. 7E is a microscopic image of Neural Epithelial Progenitor (NEP) cells derived from Cdk1+/+, +/alink human CA1 ES cells. FIG. 7F depicts microscopic images illustrating GCV-induced killing of dividing ALINK-modified NEPs and non-killing of non-dividing neurons.

FIG. 8 depicts a graph showing the expected number of cells comprising spontaneous mutations in the HSV-TK gene as a population is expanded from heterozygous (blue line) and homozygous (red line) ALINK cells.

FIGS. 9A-9B depict targeting of a dox-bridge into the 5′UTR of the mouse Cdk1 locus to generate EARC and behavior of the bridge after insertion into Cdk1. FIG. 9A is a schematic illustrating the structure of the mouseCdk1 locus, the target vector, and the position of the primers used for genotyping for homologous recombination events. FIG. 9B depicts PCR results showing the genotyping of the puromycin resistant colonies to identify those that integrated the dox-bridge to the Cdk1 5′UTR.

FIG. 10 depicts a flow chart illustrating that ES cells having a homozygous dox-bridge knock-in survive and divide only in the presence of doxycycline (or drug with doxycycline overlapping function).

FIG. 11 depicts representative photomicrographs illustrating that homozygous dox-bridge knock-in ES cells show doxycycline concentration dependent survival and growth.

FIG. 12 depicts dox-bridge removal with Cre recombinase-mediated excision, which rescues the doxycycline dependent survival of the ES cells.

FIGS. 13A-13B depict the effect of doxycycline withdrawal on the growth of dox-bridged ES cells. FIG. 13A depicts a graph showing that in the presence of doxycycline the cells grew exponentially (red line with circle), indicating their normal growth. Upon doxycycline withdrawal on Day 1, the cells grew only for two days and then they started disappearing from the plates until no cell left on Day 9 on (dark blue line with square). The 20× lower doxycycline concentration (50 ng/ml) after an initial 3 days of growth kept a constant number of cells on the plate for at least five days (FIG. 13A, light blue line with triangle). On Day 10 the normal concentration of doxycycline was added back to the plates and the cells started growing again as normal ES cells. FIG. 13B depicts a bar graph showing the level of Cdk1 mRNA (as measured by quantitative-PCR) after 0, 1 and 2 days of Dox removal. Expression levels are normalized to beta-actin.

FIG. 14 depicts the process of growing dox-bridged ES cells and illustrates that no escaper cells were found among 100,000,000 dox-bridged ES cells when doxycycline was withdrawn from the media, but the sentinel (wild type, GFP positive) cells survived with high efficiency.

FIG. 15 depicts a graph showing the effect of high doxycycline concentration (10 μg/ml) on dox-bridged ES cells: in the presence of high doxycycline, the cells slow down their growth rate similarly to when in low-doxycycline (high dox was 10 μg/ml, normal dox was 1 μg/ml, low dox was 0.05 μg/ml), indicating that there is a window for Dox concentration defining optimal level of CDK1 expression for cell proliferation.

FIGS. 16A-16B depict targeting of dox-bridge into the 5′UTR of the Cdk1 locus of mouse cells comprising ALINK modifications (i.e., Cdk1(TK/TK) cells; the cell product described in FIGS. 3A-3G). FIG. 16A is a schematic illustrating the structure of the Cdk1 locus in Cdk1(TK/TK) cells, the bridge target vector, and the location of genotyping primers. FIG. 16B depicts PCR results showing the genotyping of the puromycin resistant colonies to identify those that integrated the dox-bridge to the Cdk1 5′UTR in mouse Cdk1(TK/TK) cells, thus generating mouse cell product Cdk1earc/earc, alink/alink.

FIGS. 17A-17B depict targeting of dox-bridge into the 5′UTR of the Cdk1 locus of human cells comprising ALINK modifications (i.e., Cdk1(TK/TK) cells; the cell product described in FIGS. 4A-4F). FIG. 17A is a schematic illustrating the structure of the Cdk1 locus in Cdk1(TK/TK) cells, the bridge target vector, and the location of genotyping primers. FIG. 17B depicts PCR results showing the genotyping of the puromycin resistant colonies to identify those that integrated the dox-bridge to the Cdk1 5′UTR in human Cdk1(TK/TK) cells, thus generating human cell product Cdk1earc/earc, alink/alink.

FIGS. 18A-18B depict targeting of a dox-bridge into the 5′UTR of the Top2a locus to generate EARC insertion into Top2a. FIG. 18A is a schematic illustrating the structure of the Top2a locus and the target vector. TOP2a_5scrF, rttaRev, CMVforw and TOP2a_3scrR indicate the position of the primers used for genotyping for homologous recombination events. FIG. 18B depicts PCR results showing the genotyping of the puro resistant colonies to identify those that integrated the dox-bridge to the Top2a 5′UTR. Nine of these cell lines were found to be homozygous targeted comprising a dox-bridge inserted by homologous recombination into the 5′UTR of both alleles of Top2a.

FIGS. 19A-19B depict the effect of doxycycline withdrawal on the growth of Top2a-EARC ES cells. FIG. 19A shows that withdrawal of doxycycline results in complete elimination of mitotically active ES cells within 4 days. FIG. 19B depicts how different concentrations of doxycycline affected proliferation of the dox-bridge ES cells by measuring cell growth for 4 days. ES cells in the presence of doxycycline grew exponentially, indicating their normal growth. In contrast, two days after doxycycline removal, cells growth was completely arrested.

FIGS. 20A-20B depict targeting of a dox-bridge into the 5′UTR of the Cenpa locus to generate EARC insertion into Cenpa. FIG. 20A is a schematic illustrating the structure of the Cenpa locus and the target vector. Cenpa_5scrF, rttaRev, CMVforw and Cenpa_3scrR indicate the position of the primers used for genotyping for homologous recombination events. FIG. 20B depicts PCR results showing the genotyping of the puro resistant colonies to identify those that integrated the dox-bridge to the Cenpa 5′UTR. Six of these cells were found to have a correct insertion at the 5′ and 3′, and at least one clone (Cenpa #4) was found to have homozygous targeting comprising a dox-bridge inserted by homologous recombination into the 5′UTR of both alleles of Cenpa.

FIGS. 21A-21B depict the effect of doxycycline withdrawal on the growth of Cenpa-EARC ES cells. FIG. 21A depicts that withdrawal of doxycycline results in complete elimination of mitotically active ES cells within 4 days. FIG. 21B is the Cenpa gene expression level (determined by q-PCR) in Cenpa-EARC cells with Dox and after 2 days of Dox removal, and compared it to the expression level in wild type mouse ES cells (C2). As expected Cenpa expression level is greatly reduced in Cenpa-EARC cells without Dox for 2 days.

FIG. 22 depicts how different concentrations of doxycycline affected proliferation of the Cenpa-EARC ES cells by measuring cell growth for 4 days. ES cells in the presence of doxycycline grew exponentially, indicating their normal growth. In contrast, 80 hours after doxycycline removal, cells growth was completely arrested.

FIGS. 23A-23B depict targeting of a dox-bridge into the 5′UTR of the Birc5 locus to generate EARC insertion into Birc5. FIG. 23A is a schematic illustrating the structure of the Birc5 locus and the target vector. Birc_5scrF and rttaRev indicate the position of the primers used for genotyping for homologous recombination events. FIG. 23B depicts PCR results showing the genotyping of the puro resistant colonies to identify those that integrated the dox-bridge to the Birc5 5′UTR. Five clones were found to be correctly targeted comprising a dox-bridge inserted by recombination into the 5′UTR of both alleles of Birc5. One of these clones, Birc #3, was found to stop growing or die in the absence of Dox.

FIGS. 24A-24B depict the effect of doxycycline withdrawal on the growth of Birc5-EARC ES cells. FIG. 24A depicts that withdrawal of doxycycline results in complete elimination of mitotically active ES cells within 4 days. FIG. 24B is the Birc5 gene expression level (determined by q-PCR) in Birc5-EARC cells with Dox and after 2 days of Dox removal, and compared it to the expression level in wild type mouse ES cells (C2). As expected Birc5 expression level is greatly reduced in Birc5-EARC cells without Dox for 2 days.

FIG. 25 depicts how different concentrations of doxycycline affected proliferation of the Birc5-EARC ES cells by measuring cell growth for 4 days. ES cells in the presence of doxycycline grew exponentially, indicating their normal growth. In contrast, 50 hours after doxycycline removal, cells growth was completely arrested. Interestingly, it appears that lower Dox concentrations (0.5 and 0.05 μg/ml) promote better cell growth than a higher concentration (1 μg/ml).

FIGS. 26A-26B depict targeting of a dox-bridge into the 5′UTR of the Eef2 locus to generate EARC insertion into Eef2. FIG. 26A is a schematic illustrating the structure of the Eef2 locus and the target vector. Eef2_5scrF and rttaRev indicate the position of the primers used for genotyping for homologous recombination events. FIG. 26B depicts PCR results showing the genotyping of the puro resistant colonies to identify those that integrated the dox-bridge to the Eef2 5′UTR. Nine of these cell lines were found to be correctly targeted with at least one clone growing only in Dox-media.

FIG. 27 depicts the effect of doxycycline withdrawal on the growth of Eef2-EARC ES cells. Withdrawal of doxycycline results in complete elimination of mitotically active ES cells within 4 days.

FIG. 28 depicts how different concentrations of doxycycline affected proliferation of the Eef2-EARC ES cells by measuring cell growth for 4 days. ES cells in the presence of doxycycline grew exponentially, indicating their normal growth. In contrast, without doxycycline cells completely fail to grow.

### DETAILED DESCRIPTION OF THE DISCLOSURE

Unless defined otherwise, all technical and scientific terms used herein have the same meaning as commonly understood by one of ordinary skill in the art to which this disclosure belongs.

### Definitions

The terms “cell division locus”, “cell division loci”, and “CDL” as used herein, refer to a genomic locus (or loci) whose transcription product(s) is expressed by dividing cells. When a CDL comprises a single locus, absence of CDL expression in a cell (or its derivatives) means that tumour initiation and/or formation is prohibited either because the cell(s) will be ablated in the absence of CDL expression or because proliferation of the cell(s) will be blocked or compromised in the absence of CDL expression. When a CDL comprises multiple loci, absence of expression by all or subsets of the loci in a cell (or its derivatives) means that tumour initiation and/or formation is prohibited either because the cell(s) will be ablated in the absence of CDL expression or because proliferation of the cell(s) will be blocked or compromised in the absence of CDL expression. A CDL may or may not be expressed in non-dividing and/or non-proliferating cells. A CDL may be endogenous to a host cell or it may be a transgene. If a CDL is a transgene, it may be from the same or different species as a host cell or it may be of synthetic origin. In an embodiment, a CDL is a single locus that is transcribed during cell division. For example, in an embodiment, a single locus CDL is CDK1. In an embodiment, a CDL comprises two or more loci that are transcribed during cell division. For example, in an embodiment, a multi-locus CDL comprises two MYC genes (c-Myc and N-myc) (Scognamiglio et al., 2016). In an embodiment, a multi-locus CDL comprises AURORA B and C kinases, which may have overlapping functions (Fernandez-Miranda et al., 2011). Cell division and cell proliferation are terms that may be used interchangeably herein.

The terms “normal rate of cell division”, “normal cell division rate”, “normal rate of cell proliferation”, and “normal cell proliferation rate” as used herein, refer to a rate of cell division and/or proliferation that is typical of a non-cancerous healthy cell. A normal rate of cell division and/or proliferation may be specific to cell type. For example, it is widely accepted that the number of cells in the epidermis, intestine, lung, blood, bone marrow, thymus, testis, uterus and mammary gland is maintained by a high rate of cell division and a high rate of cell death. In contrast, the number of cells in the pancreas, kidney, cornea, prostate, bone, heart and brain is maintained by a low rate of cell division and a low rate of cell death (Pellettieri and Sanchez Alvarado, 2007).

The terms “inducible negative effector of proliferation” and “iNEP” as used herein, refer to a genetic modification that facilitates use of CDL expression to control cell division and/or proliferation by: i) inducibly stopping or blocking CDL expression, thereby prohibiting cell division and proliferation; ii) inducibly ablating at least a portion of CDL-expressing cells (i.e., killing at least a portion of proliferating cells); or iii) inducibly slowing the rate of cell division relative to a cell's normal cell division rate, such that the rate of cell division would not be fast enough to contribute to tumor formation.

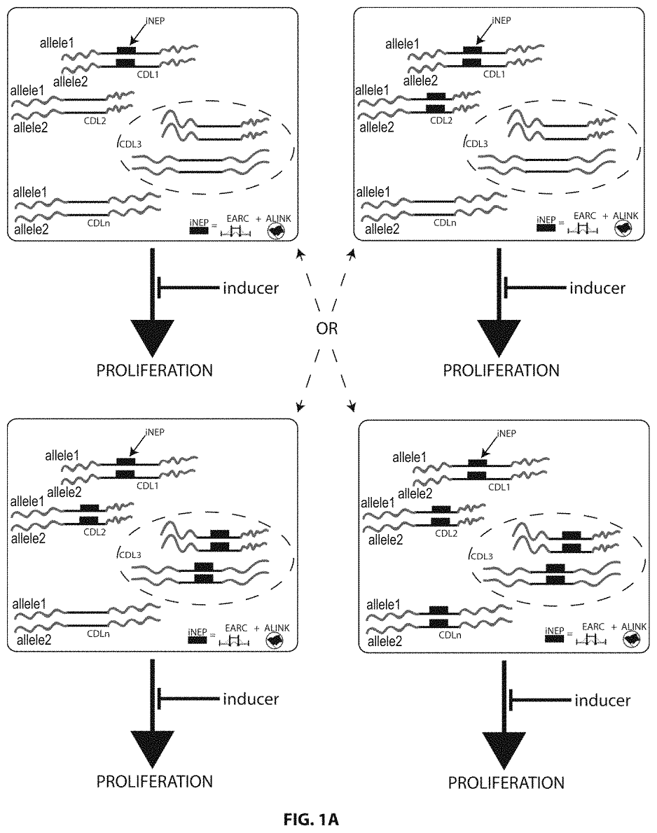

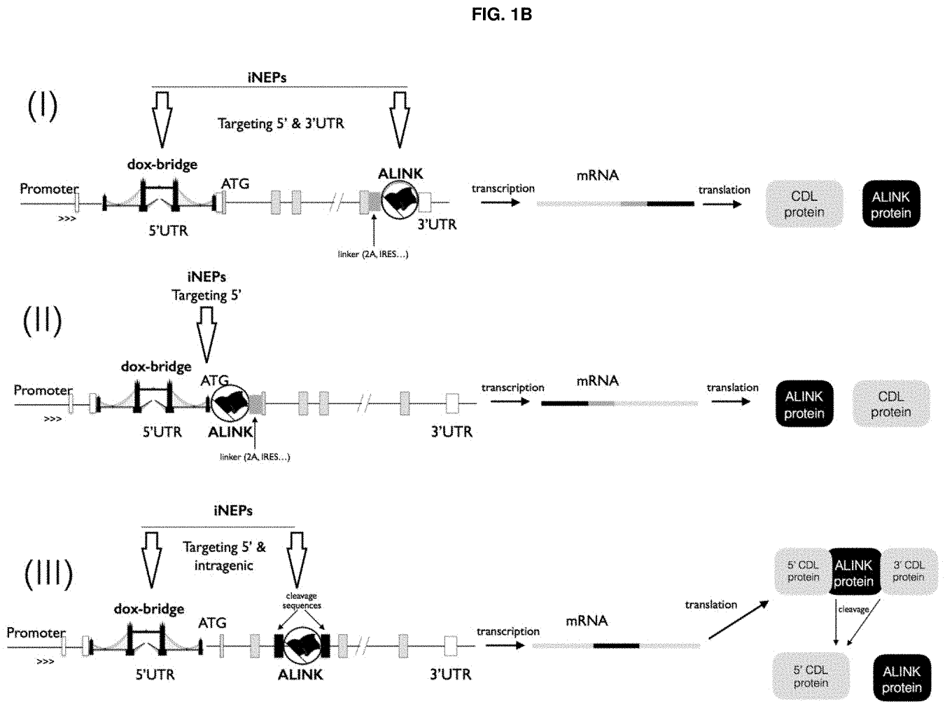

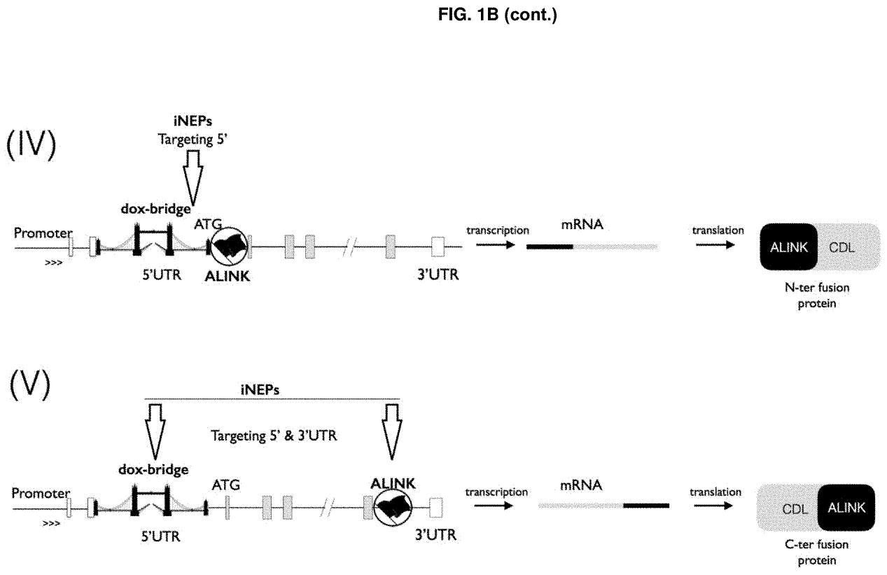

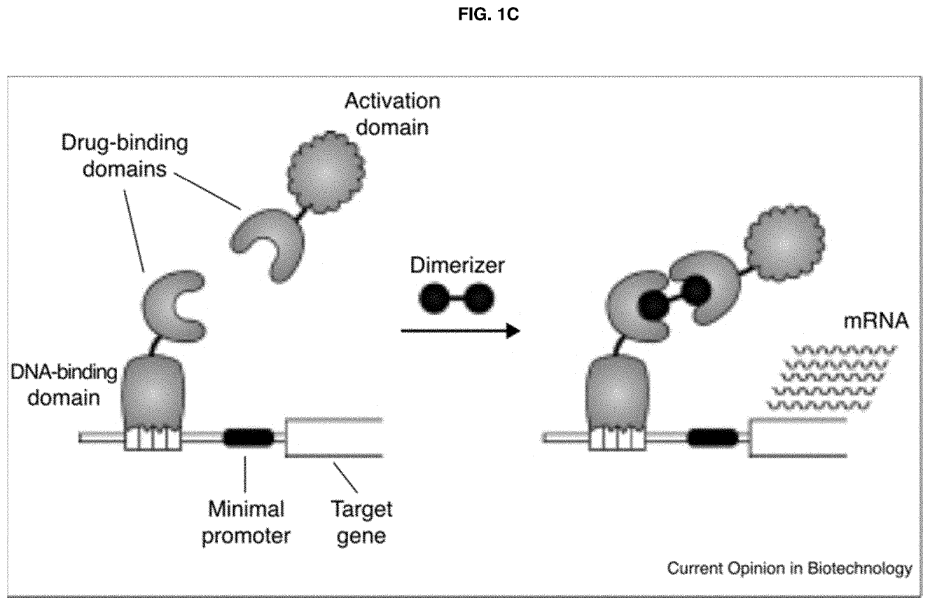

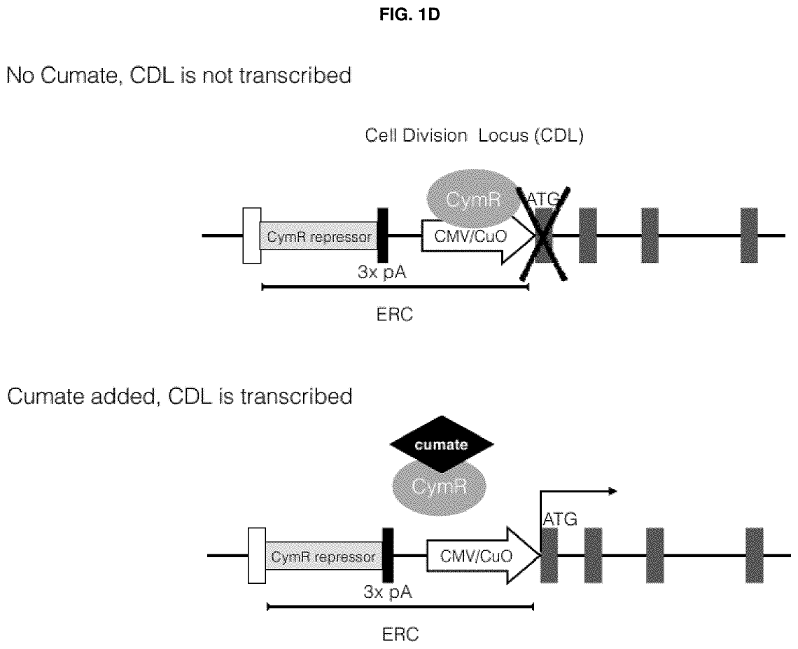

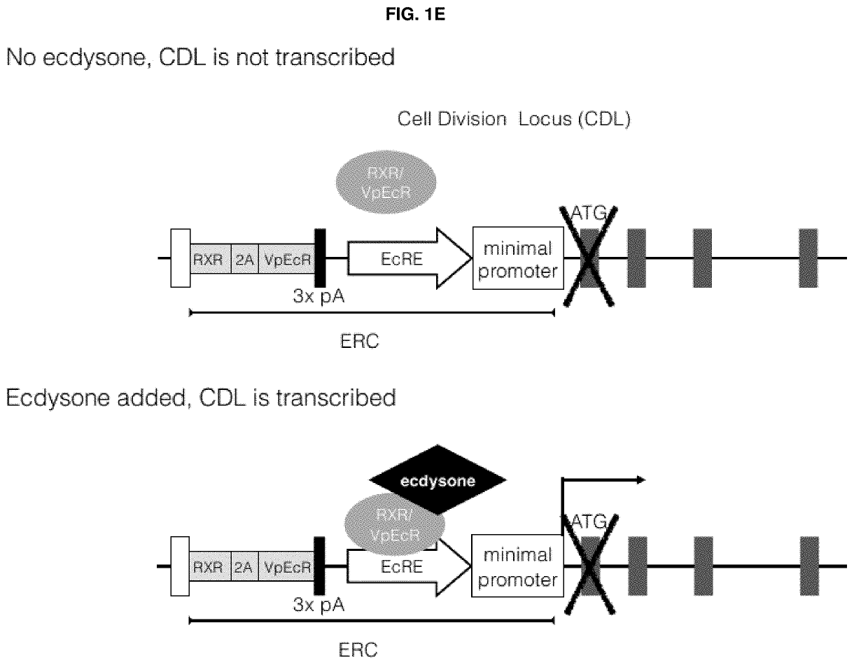

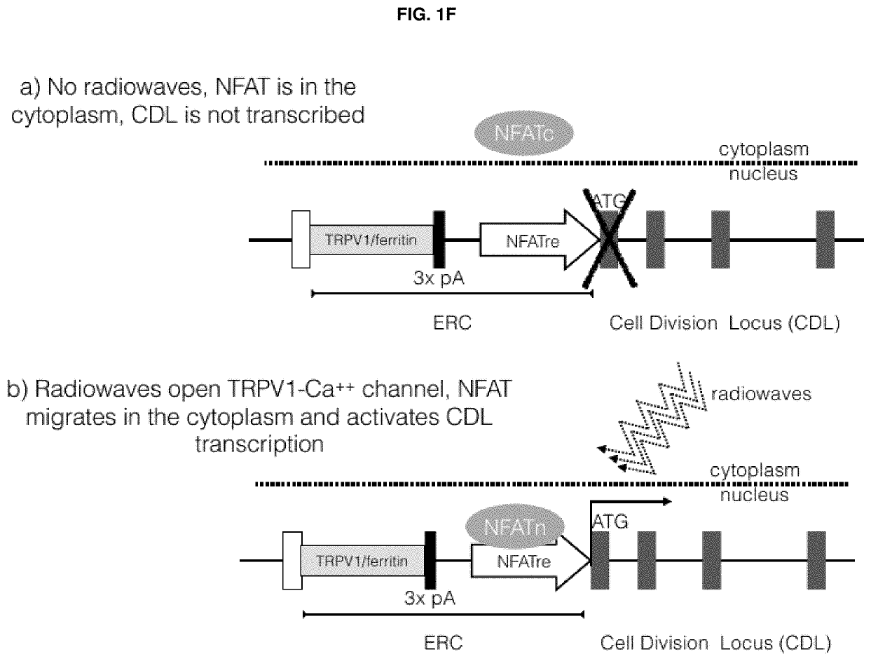

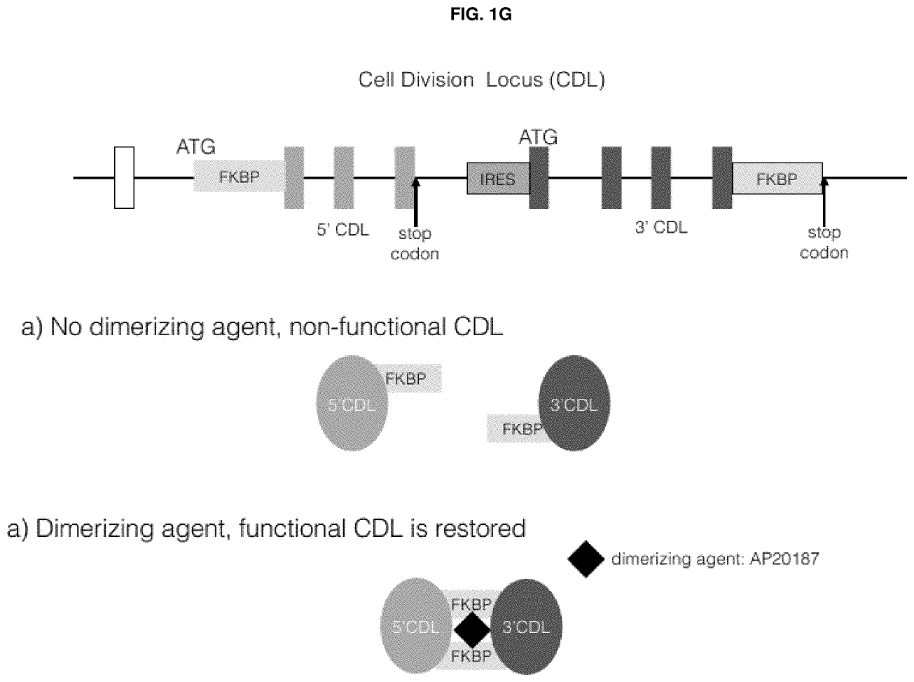

The terms “ablation link” and “ALINK” as used herein, refer to an example of an iNEP, which comprises a transcriptional link between a CDL and a sequence encoding a negative selectable marker. The ALINK modification allows a user to inducibly kill proliferating host cells comprising the ALINK or inhibit the host cell's proliferation by killing at least a portion of proliferating cells by exposing the ALINK-modified cells to an inducer of the negative selectable marker. For example, a cell modified to comprise an ALINK at a CDL may be treated with an inducer (e.g., a prodrug) of the negative selectable marker in order to ablate proliferating cells or to inhibit cell proliferation by killing at least a portion of proliferating cells (FIG. 1B).

The terms “exogenous activator of regulation of CDL” and “EARC” as used herein, refer to an example of an iNEP, which comprises a mechanism or system that facilitates exogenous alteration of non-coding or coding DNA transcription or corresponding translation via an activator. An EARC modification allows a user to inducibly stop or inhibit division of cells comprising the EARC by removing from the EARC-modified cells an inducer that permits transcription and/or translation of the EARC-modified CDL. For example, an inducible activator-based gene expression system may be operably linked to a CDL and used to exogenously control expression of a CDL or CDL translation, such that the presence of a drug inducible activator and corresponding inducer drug are required for CDL transcription and/or translation. In the absence of the inducer drug, cell division and/or proliferation would be stopped or inhibited (e.g., slowed to a normal cell division rate). For example, the CDL Cdk1/CDK1 may be modified to comprise a dox-bridge (FIG. 1B), such that expression of Cdk1/CDK1 and cell division and proliferation are only possible in the presence of an inducer (e.g., doxycycline).

The term “proliferation antagonist system” as used herein, refers to a natural or engineered compound(s) whose presence inhibits (completely or partially) proliferation of a cell.

General Description of Tools and Methods

As described herein, the inventors have provided molecular tools, methods and kits for using one or more cell division loci (CDL) in an animal cell to generate genetically modified cells in which cell division and/or proliferation can be controlled by a user through one or more iNEPs (FIG. 1A). For example, division of cells generated using one or more tools and/or methods provided herein could be stopped, blocked or inhibited by a user such that a cell's division rate would not be fast enough to contribute to tumor formation. For example, proliferation of cells generated using one or more tools and/or methods provided herein could be stopped, blocked or inhibited by a user, by killing or stopping at least a portion of proliferating cells, such that a cell's proliferation rate or volume may be maintained at a rate or size, respectively, desired by the user.

Tools and methods for controlling cell division and/or proliferation are desirable, for example, in instances wherein faster cell division rates (relative to normal cell division rates) are undesirable. For example, cells that divide at faster than normal rates may form tumors in situ, which may be harmful to a host. In an embodiment, the genetically modified animal cells provided herein comprise one or more mechanisms for allowing normal cell division and/or proliferation and for stopping, ablating, blocking and/or slowing cell division and/or proliferation, such that undesirable cell division and/or proliferation may be controlled by a user (FIG. 1B). Referring to FIG. 1B, in example (I) EARC is inserted at the 5′ UTR of the CDL and ALINK is inserted at the 3′ UTR, the product of transcription is a bi-cistronic mRNA that get processed in two proteins. In example (II) both EARC and ALINK are inserted at the 5′ UTR of the CDL, the product of transcription is a bi-cistronic mRNA that get processed in two proteins. In example (III) EARC is inserted at the 5′ UTR of the CDL and ALINK is inserted within the CDL coding sequence, the product of transcription is a mRNA that get processed in a precursor protein that will generate two separate protein upon cleavage of specifically designed cleavage sequences. In example (IV) both EARC and ALINK are inserted at the 5′ UTR of the CDL, the product of transcription is a mRNA that get processed into a fusion protein that maintains both CDL and ALINK functions. In example (V) EARC is inserted at the 5′ UTR of the CDL and ALINK is inserted at the 3′ UTR, the product of transcription is a mRNA that get processed into a fusion protein that maintains both CDL and ALINK functions.

For example, the genetically modified animal cells provided herein may be used in a cell therapeutic treatment applied to a subject. If one or more of the genetically modified animal cells provided to the subject were to begin dividing at an undesirable rate (e.g., faster than normal), then a user could stop or slow division of cells dividing at the undesirable rate or block, slow or stop cells proliferating at the undesirable rate by i) applying to the cells dividing at the undesirable rate an inducer corresponding to the genetic modification in the cells; or ii) restricting access of the cells dividing at the undesirable rate to an inducer corresponding to the genetic modification in the cells, i) or ii) being determined based on the type of iNEP(s) provided in the genetically modified animal cells.

In an embodiment, the genetically modified animal cells provided herein may be referred to as “fail-safe cells”. A fail-safe cell contains one or more homozygous, heterozygous, hemizygous or compound heterozygous ALINKs in one or more CDLs. In an embodiment, a fail-safe cell further comprises one or more EARCs in one or more CDL. In an embodiment, a fail-safe cell comprises a CDL comprising both ALINK and EARC modifications.

As used herein, the term “fail-safe”, refers to the probability (designated as pFS) defining a cell number. For example, the number of cells that can be grown from a single fail-safe cell (clone volume) where the probability of obtaining a clone containing cells, which have lost all ALINKs is less than an arbitrary value (pFS). For example, a pFS=0.01 refers to a scenario wherein if clones were grown from a single cell comprising an ALINK-modified CDL 100 times, only one clone expected to have cells, which lost ALINK function (the expression of the negative selectable marker) while still capable of cell division. The fail-safe volume will depend on the number of ALINKs and the number of ALINK-targeted CDLs. The fail-safe property is further described in Table 1.

TABLE 1
Fail-safe cell volumes and their relationship to a human body were calculated
using mathematical modelling. The model did not take into a count the events
when CDL expression was co-lost with the loss of negative selectable marker
activity, compromising cell proliferation. Therefore the values are underestimates
and were calculated assuming 10-6 forward mutation rate for the negative selectable
marker. The estimated number of cells in a human body as 3.72 × 10¹³
was taken from (Bianconi et al., 2013).

| | | | Fail-safe | Relative (x) to | Estimated | |
|---|---|---|---|---|---|---|
| | | Genotype | volume | a human body = | weight of | |
| CDL # | ALINK # | in CDLs | (#cells) | 3.72 × 10¹³ cells | clones | |
| 1 | 1 | het | 512 | 0.0000000000137 | 1 | μg |
| 1 | 2 | horn | 16777216 | 0.000000451 | 31 | mg |
| 2 | 3 | het, horn | 1.374E+11 | 0.004 | 0.26 | kg |
| 2 | 4 | horn, horn | 1.13E+15 | 30 | 2100 | kg |

It is contemplated herein that fail-safe cells may be of use in cell-based therapies wherein it may be desirable to eliminate cells exhibiting undesirable growth rates, irrespective of whether such cells are generated before or after grafting the cells into a host.

Cell Division Loci (CDLs)

The systems, methods and compositions provided herein are based on the identification of one or more CDLs, such as, for example, the CDLs set forth in Table 2. It is contemplated herein that various CDLs could be targeted using the methods provided herein.

In various embodiments, a CDL is a locus identified as an “essential gene” as set forth in Wang et al., 2015, which is incorporated herein by reference as if set forth in its entirety. Essential genes in Wang et al., 2015, were identified by computing a score (i.e., a CRISPR score) for each gene that reflects the fitness cost imposed by inactivation of the gene. In an embodiment, a CDL has a CRISPR score of less than about—1.0 (Table 2, column 5).

In various embodiments, a CDL is a locus/loci that encodes a gene product that is relevant to cell division and/or replication (Table 2, column 6). For example, in various embodiments, a CDL is a locus/loci that encodes a gene product that is relevant to one or more of: i) cell cycle; ii) DNA replication; iii) RNA transcription and/or protein translation; and iv) metabolism (Table 2, column 7).

In an embodiment, a CDL is one or more cyclin-dependent kinases that are involved with regulating progression of the cell cycle (e.g., control of G1/S G2/M and metaphase-to-anaphase transition), such as CDK1, CDK2, CDK3, CDK4, CDK5, CDK6, CDK7, CDK8, CDK9 and/or CDK11 (Morgan, 2007). In an embodiment, a CDL is one or more cyclins that are involved with controlling progression of the cell cycle by activating one or more CDK, such as, for example, cyclinB, cyclinE, cyclinA, cyclinC, cyclinD, cyclinH, cyclinC, cyclinT, cyclinL and/or cyclinF (FUNG and POON, 2005). In an embodiment, a CDL is one or more loci involved in the anaphase-promoting complex that controls the progression of metaphase to anaphase transition in the M phase of the cell cycle (Peters, 2002). In an embodiment, a CDL is one or more loci involved with kinetochore components that control the progression of metaphase to anaphase transition in the M phase of the cell cycle (Fukagawa, 2007). In an embodiment, a CDL is one or more loci involved with microtuble components that control microtubule dynamics required for the cell cycle (Cassimeris, 1999).

In various embodiments, a CDL is a locus/loci involved with housekeeping. As used herein, the term “housekeeping gene” or “housekeeping locus” refers to one or more genes that are required for the maintenance of basic cellular function. Housekeeping genes are expressed in all cells of an organism under normal and patho-physiological conditions.

In various embodiments, a CDL is a locus/loci that encodes a gene product that is relevant to cell division and/or proliferation and has a CRISPR score of less than about −1.0. For example, in an embodiment, a CDL is a locus/loci that encodes a gene product that is relevant to one or more of: i) cell cycle; ii) DNA replication; iii) RNA transcription and/or protein translation; and iv) metabolism, and has a CRISPR score of less than about −1.0. In an embodiment, the CDL may also be a housekeeping gene.

In an embodiment, to identify potential CDLs, the inventors examined early mouse embryonic lethal phenotypes of gene knockouts (KOs; Table 2, column 8). For example, the inventors found that mouse embryos homozygous null for Cdk1 (cyclin-dependent kinase 1, also referred to as cell division cycle protein 2 homolog (CDC2)) null mutation die at the 2-cell stage (E1.5) (Santamaría et al., 2007). Cdk1 (referred to as CDK1 in humans) is a highly conserved serine/threonine kinase whose function is critical in regulating the cell cycle. Protein complexes of Cdk1 phosphorylate a large number of target substrates, which leads to cell cycle progression. In the absence of Cdk1 expression, a cell cannot transition through the G2 to M phase of the cell cycle.

Cdk1/CDK1 is one example of a single locus CDL. Genetic modifications of Cdk1/CDK1, in which transcription of the locus is ablated by insertion of an ALINK modification and/or exogenously controlled by insertion of an EARC modification, are examined herein as set forth in Examples 1, 2 and 3. Top2A/TOP2A is one example of a CDL. Cenpa/CEPNA is one example of a CDL. Birc5/BIRC5 is one example of a CDL. Eef2/EEF2 is one example of a CDL. Genetic modifications of Top2a, Cenpa, Birc5, and Eef2 in which transcription of the locus can be exogenously controlled by insertion of an EARC modification are examined herein as set forth in Examples 4-7, respectively.

It an embodiment, is contemplated herein that alternative and/or additional loci are CDLs that could be targeted using the method provided herein.

For example, RNAi screening of human cell lines identified a plurality of genes essential for cell proliferation (Harborth et al., 2001; Kittler et al., 2004). The inventors predicted that a subset of these loci were CDLs after confirming the loci's early embryonic lethal phenotype of mouse deficient of the orthologues and/or analyzing the Loci's GO term and/or genecards (Table 2, column 8).

Targeting a CDL with an Ablation Link (ALINK) Genetic Modification

In one aspect, the disclosure provides molecular tools, methods and kits for modifying a CDL by linking the expression of a CDL with that of a DNA sequence encoding a negative selectable marker, thereby allowing drug-induced ablation of mitotically active cells consequently expressing the CDL and the negative selectable marker. Ablation of proliferating cells may be desirable, for example, when cell proliferation is uncontrolled and/or accelerated relative to a cell's normal division rate (e.g., uncontrolled cell division exhibited by cancerous cells). Ablation of proliferating cells may be achieved via a genetic modification to the cell, referred to herein as an “ablation link” (ALINK), which links the expression of a DNA sequence encoding a negative selectable marker to that of a CDL, thereby allowing elimination or sufficient inhibition of ALINK-modified proliferating cells consequently expressing the CDL locus (sufficient inhibition being inhibition of cell expansion rate to a rate that is too low to contribute to tumour formation). In the presence of a pro-drug or other inducer of the negatively selectable system, cells expressing the negative selectable marker will stop proliferating or die, depending on the mechanism of action of the selectable marker. Cells may be modified to comprise homozygous, heterozygous, hemizygous or compound heterozygous ALINKS. In one embodiment, to improve fidelity of ablation, a negative selectable marker may be introduced into all alleles functional of a CDL. In one preferred embodiment, a negative selectable marker may be introduced into all functional alleles of a CDL.

An ALINK may be inserted in any position of CDL, which allows co-expression of the CDL and the negative selectable marker.

As discussed further below in Example 1, DNA encoding a negatively selectable marker (e.g., HSV-TK), may be inserted into a CDL (e.g., CDK1) in a host cell, such that expression of the negative selectable marker causes host cells expressing the negative selectable marker and, necessarily, the CDL, to be killed in the presence of an inducer (e.g., prodrug) of the negative selectable marker (e.g., ganciclovir (GCV)). In this example, host cells modified with the ALINK will produce thymidine kinase (TK) and the TK protein will convert GCV into GCV monophosphate, which is then converted into GCV triphosphate by cellular kinases. GCV triphosphate incorporates into the replicating DNA during S phase, which leads to the termination of DNA elongation and cell apoptosis (Halloran and Fenton, 1998).

A modified HSV-TK gene (Preuf3 et al., 2010) is disclosed herein as one example of DNA encoding a negative selectable marker that may be used in an ALINK genetic modification to selectively ablate cells comprising undesirable cell division rate.

It is contemplated herein that alternative and/or additional negative selectable systems could be used in the tools and/or methods provided herein. Various negative selectable marker systems are known in the art (e.g., dCK.DM (Neschadim et al., 2012)).

For example, various negative selectable system having clinical relevance have been under active development in the field of “gene-direct enzyme/prodrug therapy” (GEPT), which aims to improve therapeutic efficacy of conventional cancer therapy with no or minimal side-effects (Hedley et al., 2007; Nawa et al., 2008). Frequently, GEPT involves the use of viral vectors to deliver a gene into cancer cells or into the vicinity of cancer cells in an area of the cancer cells that is not found in mammalian cells and that produces enzymes, which can convert a relatively non-toxic prodrug into a toxic agent.

HSV-TK/GCV, cytosine deaminase/5-fluorocytosine (CD/5-FC), and carboxyl esterase/irinotecan (CE/CPT-11) are examples of negative selectable marker systems being evaluated in GEPT pre- and clinical trials (Danks et al., 2007; Shah, 2012).

To overcome the potential immunogenicity issue of Herpes Simplex Virus type 1 thymidine kinase/ganciclovir (TK/GCV) system, a “humanized” suicide system has been developed by engineering the human deoxycytidine kinase enzyme to become thymidine-active and to work as a negative selectable (suicide) system with non-toxic prodrugs: bromovinyl-deoxyuridine (BVdU), L-deoxythymidine (LdT) or L-deoxyuridine (LdU) (Neschadim et al., 2012).

The CD/5-FC negative selectable marker system is a widely used “suicide gene” system. Cytosine deaminase (CD) is a non-mammalian enzyme that may be obtained from bacteria or yeast (e.g., from Escherichia coli or Saccharomyces cerevisiae, respectively) (Ramnaraine et al., 2003). CD catalyzes conversion of cytosine into uracil and is an important member of the pyrimidine salvage pathway in prokaryotes and fungi, but it does not exist in mammalian cells. 5-fluorocytosine (5-FC) is an antifungal prodrug that causes a low level of cytotoxicity in humans (Denny, 2003). CD catalyzes conversion of 5-FC into the genotoxic agent 5-FU, which has a high level of toxicity in humans (Ireton et al., 2002).

The CE/CPT-11 system is based on the carboxyl esterase enzyme, which is a serine esterase found in a different tissues of mammalian species (Humerickhouse et al., 2000). The anti-cancer agent CPT-11 is a prodrug that is activated by CE to generate an active referred to as 7-ethyl-10-hydroxycamptothecin (SN-38), which is a strong mammalian topoisomerase I inhibitor (Wierdl et al., 2001). SN-38 induces accumulation of double-strand DNA breaks in dividing cells (Kojima et al., 1998).

Another example of a negative selectable marker system is the iCasp9/AP1903 suicide system, which is based on a modified human caspase 9 fused to a human FK506 binding protein (FKBP) to allow chemical dimerization using a small molecule AP1903, which has tested safely in humans. Administration of the dimerizing drug induces apoptosis of cells expressing the engineered caspase 9 components. This system has several advantages, such as, for example, including low potential immunogenicity, since it consists of human gene products, the dimerizer drug only effects the cells expressing the engineered caspase 9 components (Straathof et al., 2005). The iCasp/AP1903 suicide system is being tested in clinical settings (Di Stasi et al., 2011).

It is contemplated herein that the negative selectable marker system of the ALINK system could be replaced with a proliferation antagonist system. The term “proliferation antagonist” as used herein, refers to a natural or engineered compound(s) whose presence inhibits (completely or partially) division of a cell. For example, Omomyc^{ER} is the fusion protein of MYC dominant negative Omomyc with mutant murine estrogen receptor (ER) domain. When induced with tamoxifen (TAM), the fusion protein Omomyc^{ER} localizes to the nucleus, where the dominant negative Omomyc dimerizes with C-Myc, L-Myc and N-Myc, sequestering them in complexes that are unable to bind the Myc DNA binding consensus sequences (Soucek et al., 2002). As a consequence of the lack of Myc activity, cells are unable to divide (Oricchio et al., 2014). Another example of a proliferation antagonist is A-Fos, a dominant negative to activation protein-1 (AP1) (a heterodimer of the oncogenes Fos and Jun) that inhibits DNA binding in an equimolar competition (Olive et al., 1997). A-Fos can also be fused to ER domain, rendering its nuclear localization to be induced by TAM. Omomyc^{ER}/tamoxifen or A-Fos^{ER}/tamoxifen could be a replacement for TK/GCV to be an ALINK.

Targeting a CDL with an EARC Genetic Modification

In an aspect, the disclosure provides molecular tools, methods and kits for exogenously controlling a CDL by operably linking the CDL with an EARC, such as an inducible activator-based gene expression system. Under these conditions, the CDL will only be expressed (and the cell can only divide) in the presence of the inducer of the inducible activator-based gene expression system. Under these conditions, EARC-modified cells stop dividing, significantly slow down, or die in the absence of the inducer, depending on the mechanism of action of the inducible activator-based gene expression system and CDL function. Cells may be modified to comprise homozygous or compound heterozygous EARCs or may be altered such that only EARC-modified alleles could produce functional CDLs. In an embodiment, an EARC modification may be introduced into all alleles of a CDL, for example, to provide a mechanism for cell division control.

An EARC may be inserted in any position of CDL that permits co-expression of the CDL and the activator component of the inducible system in the presence of the inducer.

In an embodiment, an “activator” based gene expression system is preferable to a “repressor” based gene expression system. For example, if a repressor is used to suppress a CDL a loss of function mutation of the repressor could release CDL expression, thereby allowing cell proliferation. In a case of an activation-based suppression of cell division, the loss of activator function (mutation) would shut down CDL expression, thereby disallowing cell proliferation.

As discussed further below in Examples 2-6, a dox-bridge may be inserted into a CDL (e.g., CDK1) in a host cell, such that in the presence of an inducer (e.g., doxycycline or “DOX”) the dox-bridge permits CDL expression, thereby allowing cell division and proliferation. Host cells modified with a dox-bridge EARC may comprise a reverse tetracycline Trans-Activator (rtTA) gene (Urlinger et al., 2000) under the transcriptional control of a promoter, which is active in dividing cells (e.g., in the CDL). This targeted insertion makes the CDL promoter no longer available for CDL transcription. To regain CDL transcription, a tetracycline responder element promoter (for example TRE (Agha-Mohammadi et al., 2004)) is inserted in front of the CDL transcript, which will express the CDL gene only in a situation when rtTA is expressed and doxycycline is present. When the only source of CDL expression is dox-bridged alleles, there is no CDL gene expression in the absence of doxycycline. The lack of CDL expression causes the EARC-modified cells to be compromised in their proliferation, either by death, stopping cell division, or by rendering the cell mitotic rate so slow that the EARC-modified cell could not contribute to tumor formation.

The term “dox-bridge” as used herein, refers to a mechanism for separating activity of a promoter from a target transcribed region by expressing rtTA (Gossen et al., 1995) by the endogenous or exogenous promoter and rendering the transcription of target region under the control of TRE. As used herein, “rtTA” refers to the reverse tetracycline transactivator elements of the tetracycline inducible system (Gossen et al., 1995) and “TRE” refers to a promoter consisting of TetO operator sequences upstream of a minimal promoter. Upon binding of rtTA to the TRE promoter in the presence of doxycycline, transcription of loci downstream of the TRE promoter increases. The rtTA sequence may be inserted in the same transcriptional unit as the CDL or in a different location of the genome, so long as the transcriptional expression's permissive or non-permissive status of the target region is controlled by doxycycline. A dox-bridge is an example of an EARC.

Introduction of an EARC system into the 5′ regulatory region of a CDL is also contemplated herein.

It is contemplated herein that alternative and/or additional inducible activator-based gene expression systems could be used in the tools and or methods provided herein to produce EARC modifications. Various inducible activator-based gene expression systems are known in the art.

For example, destabilizing protein domains (Banaszynski et al., 2006) fused with an acting protein product of a coding CDL could be used in conjunction with a small molecule synthetic ligand to stabilize a CDL fusion protein when cell division and/or proliferation is desirable. In the absence of a stabilizer, destabilized-CDL-protein will be degraded by the cell, which in turn would stop proliferation. When the stabilizer compound is added, it would bind to the destabilized-CDL-protein, which would not be degraded, thereby allowing the cell to proliferate.

For example, transcription activator-like effector (TALE) technology (Maeder et al., 2013) could be combined with dimerizer-regulated expression induction (Pollock and Clackson, 2002). The TALE technology could be used to generate a DNA binding domain designed to be specific to a sequence, placed together with a minimal promoter replacing the promoter of a CDL. The TALE DNA binding domain also extended with a drug dimerizing domain. The latter can bind to another engineered protein having corresponding dimerizing domain and a transcriptional activation domain. (FIG. 1C)

For example, referring to FIG. 1D, a reverse-cumate-Trans-Activator (rcTA) may be inserted in the 5′ untranslated region of the CDL, such that it will be expressed by the endogenous CDL promoter. A 6-times repeat of a Cumate Operator (6×CuO) may be inserted just before the translational start (ATG) of CDL. In the absence of cumate in the system, rcTA cannot bind to the 6×CuO, so the CDL will not be transcribed because the 6×CuO is not active. When cumate is added, it will form a complex with rcTA, enabling binding to 6×CuO and enabling CDL transcription (Mullick et al., 2006).

For example, referring to FIG. 1E, a retinoid X receptor (RXR) and an N-terminal truncation of ecdysone receptor (EcR) fused to the activation domain of Vp16 (VpEcR) may be inserted in the 5′ untranslated region of a CDL such that they are co-expressed by an endogenous CDL promoter. Ecdysone responsive element (EcRE), with a downstream minimal promoter, may also be inserted in the CDL, just upstream of the starting codon. Co-expressed RXR and VpEcR can heterodimerize with each other. In the absence of ecdysone or a synthetic drug analog muristerone A, dimerized RXR/VpEcR cannot bind to EcRE, so the CDL is not transcribed. In the presence of ecdysone or muristerone A, dimerized RXR/VpEcR can bind to EcRE, such that the CDL is transcribed (No et al., 1996).

For example, referring to FIG. 1F, a transient receptor potential vanilloid-1 (TRPV1), together with ferritin, may be inserted in the 5′ untranslated region of a CDL and co-expressed by an endogenous CDL promoter. A promoter inducible by NFAT (NFATre) may also be inserted in the CDL, just upstream of the starting codon. In a normal environment, the NFAT promoter is not active. However, upon exposure to low-frequency radio waves, TRPV1 and ferritin create a wave of Ca⁺⁺ entering the cell, which in turn converts cytoplasmatic-NFAT (NFATc) to nuclear-NFAT (NFATn), that ultimately will activate the NFATre and transcribe the CDL (Stanley et al., 2015).

For example, referring to FIG. 1G, a CDL may be functionally divided in to parts/domains: 5′-CDL and 3′CDL, and a FKBP peptide sequence may be inserted into each domain. An IRES (internal ribosomal entry site) sequence may be placed between the two domains, which will be transcribed simultaneously by a CDL promoter but will generate two separate proteins. Without the presence of an inducer, the two separate CDL domains will be functionally inactive. Upon introduction of a dimerization agent, such as rapamycin or AP20187, the FKBP peptides will dimerize, bringing together the 5′ and 3′ CDL parts and reconstituting an active protein (Rollins et al., 2000).

Methods of Controlling Division of an Animal Cell

In an aspect, a method of controlling division of an animal cell is provided herein.

The method comprises providing an animal cell. For example, the animal cell may be an avian or mammalian cell. For example, the mammalian cell may be an isolated human or non-human cell that is pluripotent (e.g., embryonic stem cell or iPS cell), multipotent, monopotent progenitor, or terminally differentiated. The mammalian cell may be derived from a pluripotent, multipotent, monopotent progenitor, or terminally differentiated cell. The mammalian cell may be a somatic stem cell, a multipotent or monopotent progenitor cell, a multipotent somatic cell or a cell derived from a somatic stem cell, a multipotent progenitor cell or a somatic cell. Preferably, the animal cell is amenable to genetic modification. Preferably, the animal cell is deemed by a user to have therapeutic value, meaning that the cell may be used to treat a disease, disorder, defect or injury in a subject in need of treatment for same. In various embodiments, the non-human mammalian cell may be a mouse, rat, hamster, guinea pig, cat, dog, cow, horse, deer, elk, bison, oxen, camel, llama, rabbit, pig, goat, sheep, or non-human primate cell. In a preferred embodiment, the animal cell is a human cell.

The method further comprises genetically modifying in the animal cell a CDL. The step of genetically modifying the CDL comprises introducing into the host animal cell an iNEP, such as one or more ALINK systems or one or more of an ALINK system and an EARC system. Techniques for introducing into animal cells various genetic modifications, such as negative selectable marker systems and inducible activator-based gene expression systems, are known in the art, including techniques for targeted (i.e., non-random), compound heterozygous and homozygous introduction of same. In cases involving use of EARC modifications, the modification should ensure that functional CDL expression can only be generated through EARC-modified alleles. For example, targeted replacement of a CDL or a CDL with a DNA vector comprising one or more of an ALINKalone or together with one or more EARC systems may be carried out to genetically modify the host animal cell.

The method further comprises permitting division of the genetically modified animal cell(s) comprising the iNEP system.

For example, permitting division of ALINK-modified cells by maintaining the genetically modified animal cells comprising the ALINK system in the absence of an inducer of the corresponding ALINK negative selectable marker. Cell division and proliferation may be carried out in vitro and/or in vivo. For example, genetically modified cells may be allowed to proliferate and expand in vitro until a population of cells that is large enough for therapeutic use has been generated. For example, one or more of the genetically modified animal cell(s) cells that have been proliferated and expanded may be introduced into a host (e.g., by grafting) and allowed to proliferate further in vivo. In various embodiment, ablating and/or inhibiting division of the genetically modified animal cell(s) comprising an ALINK system, may be done, in vitro and/or in vivo, by exposing the genetically modified animal cell(s) comprising the ALINK system to the inducer of the corresponding negative selectable marker. Such exposure will ablate proliferating cells and/or inhibit the genetically modified animal cell's rate of proliferation by killing at least a portion of proliferating cells. Ablation of genetically modified cells and/or inhibition of cell proliferation of the genetically modified animal cells may be desirable if, for example, the cells begin dividing at a rate that is faster than normal in vitro or in vivo, which could lead to tumor formation and/or undesirable cell growth.

For example, permitting division of EARC-modified cells by maintaining the genetically modified animal cell comprising the EARC system in the presence of an inducer of the inducible activator-based gene expression system. Cell division and proliferation may be carried out in vitro and/or in vivo. For example, genetically modified cells may be allowed to proliferate and expand in vitro until a population of cells that is large enough for therapeutic use has been generated. For example, one or more of the genetically modified animal cell(s) cells that have been proliferated and expanded may be introduced into a host (e.g., by grafting) and allowed to proliferate further in vivo. In various embodiment, ablating and/or inhibiting division of the genetically modified animal cell(s) comprising the EARC system, may be done, in vitro and/or in vivo, by preventing or inhibiting exposure the genetically modified animal cell(s) comprising the EARC system to the inducer of the inducible activator-based gene expression system. The absence of the inducer will ablate proliferating cells and/or inhibit the genetically modified animal cell's expansion by proliferation such that it is too slow to contribute to tumor formation. Ablation and/or inhibition of cell division of the genetically modified animal cells may be desirable if, for example, the cells begin dividing at a rate that is faster than normal in vitro or in vivo, which could lead to tumor formation and/or undesirable cell growth.

For example, in various embodiments of the method provided herein, set forth in various Examples below, the inducers are doxycycline and ganciclovir.

In an embodiment, doxycycline may be delivered to cells in vitro by adding to cell growth media a concentrated solution of the inducer, such as, for example, about 1 mg/ml of Dox dissolved in H₂O to a final concentration in growth media of about 1 μg/ml. In vivo, doxycycline may be administered to a subject orally, for example through drinking water (e.g., at a dosage of about 5-10 mg/kg) or eating food (e.g., at a dosage of about 100 mg/kg), by injection (e.g., I.V. or I.P. at a dosage of about 50 mg/kg) or by way of tablets (e.g., at a dosage of about 1-4 mg/kg).

In an embodiment, ganciclovir may be delivered to cells in vitro by adding to cell growth media a concentrated solution of the inducer, such as, for example, about 10 mg/ml of GCV dissolved in H₂O to a final concentration in growth media of about 0.25-25 μg/ml. In vivo, GCV may be administered to a subject orally, for example through drinking water (e.g., at a dosage of about 4-20 mg/kg) or eating food (e.g., at a dosage of about 4-20 mg/kg), by injection (e.g., at a dosage of about I.V. or I.P. 50 mg/kg) or by way of tablets (e.g., at a dosage of about 4-20 mg/kg).

In an embodiment, to assess whether the inducers are working in vitro, cell growth and cell death may be measured (e.g., by cell counting and viability assay), for example every 24 hours after treatment begins. To assess whether the inducers are working in vivo, the size of teratomas generated from genetically modified pluripotent cells may be measured, for example, every 1-2 days after treatment begins.

In a particularly preferred embodiment of the method provided herein, an animal cell may be genetically modified to comprise both ALINK and EARC systems. The ALINK and EARC systems may target the same or different CDLs. Such cells may be desirable for certain applications, for example, because they provide a user with at least two mechanisms for ablating and/or inhibiting cell division and/or ablating and/or inhibiting proliferation by killing at least a portion of proliferating cells.

It is contemplated herein that the method provided herein may be used to control division and/or proliferation of an avian cell, such as, for example, a chicken cell.

Cells Engineered to Comprise at Least One Mechanism for Controlling Cell Division

In an aspect, an animal cell genetically modified to comprise at least one mechanism for controlling cell division and/or proliferation, and populations of same, are provided herein. For example, the mammalian cell may be an isolated human or non-human cell that is pluripotent (e.g., embryonic stem cell or iPS cell), multipotent, monopotent progenitor, or terminally differentiated. The mammalian cell may be derived from a pluripotent, multipotent, monopotent progenitor, or terminally differentiated cell. The mammalian cell may be a somatic stem cell, a multipotent, monopotent progenitor, progenitor cell or a somatic cell or a cell derived from a somatic stem cell, a multipotent or monopotent progenitor cell or a somatic cell. Preferably, the animal cell is amenable to genetic modification. Preferably, the animal cell is deemed by a user to have therapeutic value, meaning that the cell may be used to treat a disease, disorder, defect or injury in a subject in need of treatment for same. In some embodiments, the non-human mammalian cell may be a mouse, rat, hamster, guinea pig, cat, dog, cow, horse, deer, elk, bison, oxen, camel, llama, rabbit, pig, goat, sheep, or non-human primate cell.

The genetically modified cells provided herein comprise one or more genetic modification of one or more CDL. The genetic modification of a CDL being an ALINK system and, in the case of CDLs, one or more of an ALINK system and an EARC system, such as, for example, one or more of the ALINK and/or EARC systems described herein. For example, a genetically modified animal cell provided herein may comprise: an ALINK system in one or more CDLs; an EARC system in one or more CDLs; or ALINK and EARC systems in one or more CDLS, wherein the ALINK and EARC systems correspond to the same or different CDLs. The genetically modified cells may comprise homozygous, heterozygous, hemizygous or compound heterozygous ALINK genetic modifications. In the case of EARC modifications, the modification should ensure that functional CDL expression can only be generated through EARC-modified alleles.

It is contemplated that the genetically modified cells provided herein may be useful in cellular therapies directed to treat a disease, disorder or injury and/or in cellular therapeutics that comprise controlled cellular delivery of compounds and/or compositions (e.g., natural or engineered biologics). As indicated above, patient safety is a concern in cellular therapeutics, particularly with respect to the possibility of malignant growth arising from therapeutic cell grafts. For cell-based therapies where intensive proliferation of the therapeutic cell graft is not required, it is contemplated that the genetically modified cells comprising one or more iNEP modifications, as described herein, would be suitable for addressing therapeutic and safety needs. For cell-based therapies where intensive proliferation of the therapeutic cell graft is required, it is contemplated that the genetically modified cells comprising two or more iNEP modifications, as described herein, would be suitable for addressing therapeutic and safety needs.

It is contemplated herein that avian cells, such as chicken cells, may be provided, wherein the avian cells comprise the above genetic modifications.

Molecular Tools for Targeting CDLs

In an aspect, various DNA vectors for modifying expression of a CDL are provided herein.

In one embodiment, the DNA vector comprises an ALINK system, the ALINK system comprising a DNA sequence encoding a negative selectable marker. The expression of the negative selectable marker is linked to that of a CDL.

In one embodiment, the DNA vector comprises an EARC system, the EARC system comprising an inducible activator-based gene expression system that is operably linked to a CDL, wherein expression of the CDL is inducible by an inducer of the inducible activator-based gene expression system.

In one embodiment, the DNA vector comprises an ALINK system, as described herein, and an EARC system, as described herein. When such a cassette is inserted into a host cell, CDL transcription product expression may be prevented and/or inhibited by an inducer of the negative selectable marker of the ALINK system and expression of the CDL is inducible by an inducer of the inducible activator-based gene expression system of the EARC system.

In various embodiments, the CDL in the DNA vector is a CDL listed in Table 2.

In various embodiments, the ALINK system in the DNA vector is a herpes simplex virus-thymidine kinase/ganciclovir system, a cytosine deaminase/5-fluorocytosine system, a carboxyl esterase/irinotecan system or an iCasp9/AP1903 system.

In various embodiments, the EARC system in the DNA vector is a dox-bridge system, a cumate switch inducible system, an ecdysone inducible system, a radio wave inducible system, or a ligand-reversible dimerization system.

Kits

The present disclosure contemplates kits for carrying out the methods disclosed herein. Such kits typically comprise two or more components required for using CDLs and/or CDLs to control cell proliferation. Components of the kit include, but are not limited to, one or more of compounds, reagents, containers, equipment and instructions for using the kit. Accordingly, the methods described herein may be performed by utilizing pre-packaged kits provided herein. In one embodiment, the kit comprises one or more DNA vectors and instructions. In some embodiments, the instructions comprise one or more protocols for introducing the one or more DNA vectors into host cells. In some embodiments, the kit comprises one or more controls.

In one embodiment, the kit comprises one or more DNA vector for modifying expression of a CDL, as described herein. By way of example, the kit may contain a DNA vector comprising an ALINK system; and/or a DNA vector comprising an EARC system; and/or a DNA vector comprising an ALINK system and an EARC system; and instructions for targeted replacement of a CDL and/or CDL in an animal cell using one or more of the DNA vectors. In preferred embodiments, the kit may further comprise one or more inducers (e.g., drug inducer) that correspond with the ALINK and/or EARC systems provided in the DNA vector(s) of the kit.

The following non-limiting examples illustrative of the disclosure are provided.

### Example 1: Generation of ALINK-Modified Cells (Mouse and Human)

In Example 1, construction of ALINK (HSV-TK) vectors targeting Cdk1/CDK1 and use of same to control cell proliferation in mouse and human ES cells, by way of killing at least a portion of proliferating cells, is described. In this example, Cdk1/CDK1 is the CDL and HSV-TK is the negative selectable marker.

Cdk1/CDK1 is expressed in all mitotically active (i.e., dividing) cells. In cells modified to comprise a homozygous ALINK between the CDK1 locus and HSV-TK, all mitotically active cells express CDK1 and HSV-TK. Thus, the ALINK-modified mitotically active cells can be eliminated by treatment with GCV (the pro-drug of HSV-TK). If all the functional CDK1 expressing allele is ALINK modified and the cells were to silence HSV-TK expression then likely CDK1 expression would also be silenced and the cells would no longer be able to divide. Quiescent (i.e., non-dividing) cells do not express Cdk1/CDK1. Thus, ALINK-modified quiescent cells would not express the Cdk1/CDK1-HSV-TK link.

In Example 1, the transcriptional link between Cdk1/CDK1 and HSV-TK was achieved by homologous recombination-based knock-ins.

Methods

Generation of Target Vectors

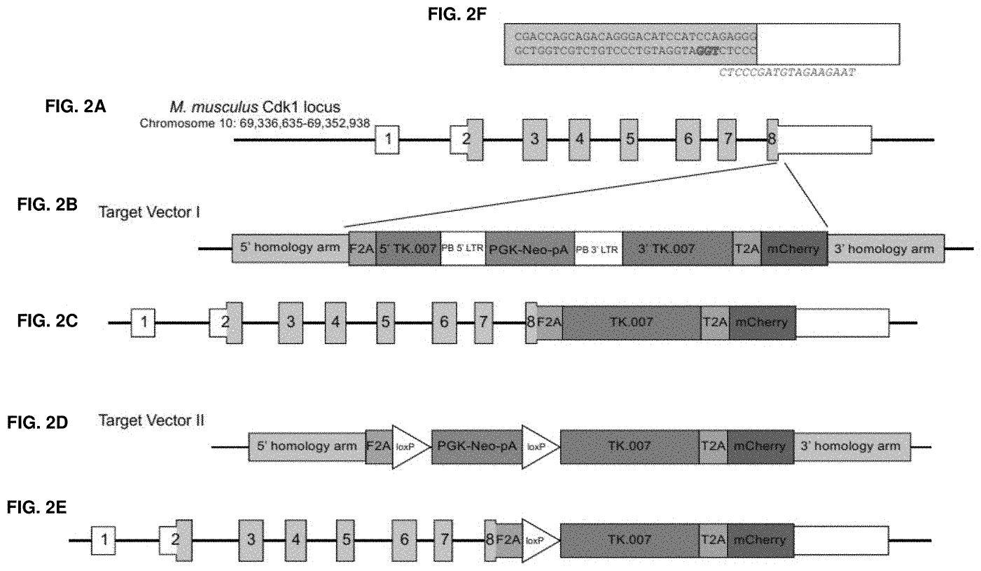

Mouse Target Vector I: The mouse Cdk1 genomic locus is shown in FIG. 2A. Referring to FIG. 2B, two DNA fragments: 5TK (SEQ ID NO: 1) and 3TK (SEQ ID NO: 2) (SalI-F2A-5′TK.007-PB 5′LTR-NotI-SacII and SalI-SacII-3′TK.007-PB 3′LTR-3′TK.007-T2A-XhoI-mCherry-NheI) were obtained by gene synthesis in a pUC57 vector (GenScript). Fragment 5TK was digested with SalI+SacII and cloned into 3TK with the same digestion to generate pUC57-5TK-3TK. A PGK-Neomycin cassette was obtained by cutting the plasmid pBluescript-M214 (SEQ ID NO: 3) with NotI+HindIII and it was ligated into the NotI+SacII site of pUC57-5TK-3TK to generate the ALINK cassette to be inserted at the 3′ end of Cdk1 (i.e., the CDL).

Homology arms for the insertion ALINK at the 3′ of the CDL: Cdk1 DNA coding sequences were cloned by recombineering: DH10B E. coli cell strain containing bacterial artificial chromosomes (BACs) with the genomic sequences of Cdk1 (SEQ ID NO: 4), which were purchased from The Center for Applied Genomics (TCAG). The recombineering process was mediated by the plasmid pSC101-BAD-γβα Red/ET (pRET) (GeneBridges, Heidelberg Germany). pRET was first electroporated into BAC-containing DH10B E. coli at 1.8 kV, 25 μF, 400 Ohms (BioRad GenePulserI/II system, BioRad, ON, CA) and then selected for choloramphenicol and tetracycline resistance. Short homology arms (50 bp) (SEQ ID NOs: 5 and 6 respectively) spanning the ALINK insertion point (5′ and 3′ of the Cdk1 stop codon) were added by PCR to the cassette, F2A-5′TK-PB-PGKneo-PB-3′TK-T2A-cherry. This PCR product was then electroporated into Bac+pRET DH10B E. coli under the conditions described above and then selected for kanamycin resistance. The final targeting cassette, consisting of 755 bp and 842 base pair (bp) homology arms (SEQ ID NOs: 7 and 8, respectively), was retrieved by PCR with primers (SEQ ID NOs: 9 and 10, respectively) and cloned into a pGemT-Easy vector to generate mouse Target Vector I. The critical junction regions of the vector were sequenced at TCAG and confirmed.

Mouse Target Vector II: referring to FIG. 2D, F2A-loxP-PGK-neo-pAdoxP-AscI (SEQ ID NO: 11) was PCR amplified from pLoxPNeo1 vector and TA cloned into a pDrive vector (Qiagen). AscI-TK-T2A-mCherry-EcoRI (SEQ ID NO: 12) was PCR amplified from excised TC allele I, and TA cloned into the pDrive vector. The latter fragment was then cloned into the former vector by BamHI+AscI restriction sites. This F2A-loxP-PGK-neo-pAdoxP-TK-T2A-mCherry cassette was inserted between mouse Cdk1 homology arms by GeneArt® Seamless Cloning and Assembly Kit (Life Technologies). To generate the puromycin (puro) version vector, PGK-puro-pA fragment (SEQ ID NO: 13) was cut from pNewDockZ with BamHI+NotI and T4 blunted. The neo version vector was cut with AscI+ClaI, T4 blunted and ligated with PGK-puro-pA.

Human Target Vector I: Similar to mouse Target Vector I, 847 bp upstream of human CDK1 stop codon (SEQ ID NO: 14)+F2A-5′TK-PB-PGKneo-PB-3′TK-T2A-cherry (SEQ ID NO: 15)+831 bp downstream of human CDK1 stop codon (SEQ ID NO: 16) was generated by recombineering technology. A different version of the vector containing a puromycin resistant cassette for selection, was generated to facilitate one-shot generation of homozygous targeting: AgeI-PGK-puro-pA-FseI (SEQ ID NO: 17) was amplified from pNewDockZ vector, digested and cloned into neo version vector cut by AgeI+FseI.

Human Target Vector II: BamHI-F2A-loxP-PGK-neo-pAdoxP-TK-T2A-mCherry (SEQ ID NO: 18) and BamHI-F2A-loxP-PGK-puro-pAdoxP-TK-T2A-mCherry (SEQ ID NO: 19) were amplified from the corresponding mouse Target Vector II, and digested with BamHI+SgrAI. The mCherry (3′ 30 bp)-hCDK13′HA-pGemEasy-hCDK15′HA-BamHI (SEQ ID NO: 20) was PCR-amplified and also digested with BamHI+SgrAI. The neo and puromycin version of human Target Vector II were generated by ligation of the homology arm backbone and the neo or puromycin version ALINK cassette.

Human Target Vector III: Target vectors with no selection cassette were made for targeting with fluorescent marker (mCherry or eGFP) by FACS and avoiding the step of excision of selection cassette. BamHI-F2A-TK-T2A-mCherry-SgrAI (SEQ ID NO: 58) was PCR amplified from excised TC allele I, digested with BamHI+SgrAI, and ligated with digested mCherry (3′ 30 bp)-hCDK13′HA-pGemEasy-hCDK15′HA-BamHI (SEQ ID NO: 20). The CRISPR PAM site in the target vector was mutagenized with primers PAM_fwd (SEQ ID NO: 59) and PAM_rev (SEQ ID NO: 60) using site-directed PCR-based mutagenesis protocol. The GFP version vector was generated by fusion of PCR-amplified XhoI-GFP (SEQ ID NO: 61) and pGemT-hCdk1-TK-PAMmut (SEQ ID NO: 62) with NEBuiler HiFi DNA Assembly Cloning Kit (New England Biolabs Inc.).

Generation of CRISPR/Cas9 Plasmids

CRISPR/Cas9-assisted gene targeting was used to achieve high targeting efficiency (Cong et al., 2013). Guide sequences for CRISPR/Cas9 were analyzed using the online CRISPR design tool (http://crispr.mit.edu) (Hsu et al., 2013).

CRISPR/Cas9 plasmids pX335-mCdkTK-A (SEQ ID NO: 21) and pX335-mCdkTK-B (SEQ ID NO: 22) were designed to target mouse Cdk1 at SEQ ID NO: 23.

CRISPR/Cas9 plasmids pX330-hCdkTK-A (SEQ ID NO: 24) and pX459-hCdkTK-A (SEQ ID NO: 25) were designed to target the human Cdk1 at SEQ ID NO: 26.

CRISPRs were generated according to the suggested protocol with backbone plasmids purchased from Addgene. (Ran et al., 2013).

Generation of ALINK-Modified Mouse ES Cells

Mouse ES Cell Culture: Mouse ES cells are cultured in Dulbecco's modified Eagle's medium (DMEM) (high glucose, 4500 mg/liter) (Invitrogen), supplemented with 15% Fetal Bovine Serum (Invitrogen), 1 mM Sodium pyruvate (Invitrogen), 0.1 mM MEM Non-essential Amino-acids (Invitrogen), 2 mM GlutaMAX (Invitrogen), 0.1 mM 2-mEARCaptoethanol (Sigma), 50 U/ml each Penicillin/Streptomycin (Invitrogen) and 1000 U/ml Leukemia-inhibiting factor (LIF) (Chemicon). Mouse ES cells are passed with 0.25% trypsin and 0.1% EDTA.

Targeting: 5×10⁵ mouse C57BL/6 C2 ES cells (Gertsenstein et al., 2010) were transfected with 2 ug DNA (Target Vector:0.5 μg, CRISPR vector: 1.5 μg) by JetPrime transfection (Polyplus). 48h after transfection cells were selected for G418 or/and puromycin-resistant. Resistant clones were picked independently and transferred to 96-well plates. 96-well plates were replicated for freezing and genotyping (SEQ ID NOs: 27, 28, 29 and 30). PCR-positive clones were expanded, frozen to multiple vials, and genotyped by southern blotting.

Excision of the selection cassette: correctly targeted ES clones were transfected with Episomal-hyPBase (for Target Vector I) (SEQ ID NO: 34) or pCAGGs-NLS-Cre-Ires-Puromycin (for Target Vector II) (SEQ ID NO: 35). 2-3 days following transfection, cells were trypsinized and plated clonally (1000-2000 cells per 10 cm plate). mCherry-positive clones were picked and transferred to 96-well plates independently and genotyped by PCR (SEQ ID NOs: 31 and 36) and Southern blots to confirm the excision event. The junctions of the removal region were PCR-amplified, sequenced and confirmed to be intact and seamless without frame shift.

Homozygous targeting: ES clones that had already been correctly targeted with a neo version target vector and excised of selection cassette were transfected again with a puromycin-resistant version of the target vector. Selection of puromycin was added after 48 hours of transfection, then colonies were picked and analyzed, as described above (SEQ ID NOs: 31 and 32). Independent puro-resistant clones were grown on gelatin, then DNA was extracted for PCR to confirm the absence of a wild-type allele band (SEQ ID NOs: 31, 33).

Generation of ALINK-Modified Human ES Cells

Human ES Cell Culture: Human CA1 or H1 (Adewumi et al., 2007) ES cells were cultured with mTeSR1 media (STEMCELL Technologies) plus penicillin-streptomycin (Gibco by Life Technologies) on Geltrex (Life Technologies) feeder-free condition. Cells were passed by TrypIE Express (Life Technologies) or Accutase (STEMCELL Technologies) and plated on mTeSR media plus ROCK inhibitor (STEMCELL Technologies) for the first 24 h, then changed to mTeSR media. Half of cells from a fully confluent 6-well plate were frozen in 1 ml 90% FBS (Life Technologies)+10% DMSO (Sigma).

Targeting: 6×10⁶ CA1 hES cells were transfected by Neon protocol 14 with 24 ug DNA (Target Vector: pX330-hCdkTK-A=18 ug:6 ug). After transfection, cells were plated on four 10-cm plates. G418 and/or puromycin selection was started 48h after transfection. Independent colonies were picked to 96-well plates. Each plate was duplicated for further growth and genotyping (SEQ ID NOs: 37, 38, 39 and 40). PCR-positive clones were expanded, frozen to multiple vials and genotyped with southern blotting.

Excision of the selection cassette: ALINK-targeted ES clones were transfected with hyPBase or pCAGGs-NLS-Cre-IRES-Puromycin and plated in a 6-well plate. When cells reached confluence in 6-well plates, cells were suspended in Hanks Balanced Salt Solution (HBSS) (Ca2+/Mg2+ Free) (25 mM HEPES pH7.0, 1% Fetal Calf Serum), and mCherry-positive cells were sorted to a 96-well plate using an ASTRIOS EQ cell sorter (Beckman Coulter).

Homozygous Targeting: Homozygous targeting can be achieved by the same way as in the mouse system or by transfecting mCherry and eGFP human target vector III plus pX330-hCdkTK-A or pX459-hCdkTK-A followed by FACS sorting for mCherry-and-eGFP double-positive cells.

Teratoma Assay

Matrigel Matrix High Concentration (Corning) was diluted 1:3 with cold DMEM media on ice. 5-10×10⁶ cells were suspended into 100 ul of 66% DMEM+33% Matrigel media and injected subcutaneously into either or both dorsal flanks of B6N mice (for mouse C2 ES cells) and NOD-SCID mice (for human ES cells). Teratomas formed 2-4 weeks after injection. Teratoma size was measured by caliper, and teratoma volume was calculated using the formula V=(L×W×H)π/6. GCV/PBS treatment was performed by daily injection with 50 mg/kg into the peritoneal cavity with different treatment durations. At the end of treatment, mice were sacrificed and tumors were dissected and fixed in 4% paraformaldehyde for histology analysis.

Mammary Gland Tumor Assay

Chimeras of Cdk1^{+/+, +/loxp-alink} mouse C2 ES and CD-1 backgrounds were generated through diploid aggregation, and then were bred with B6N WT mice to generate Cdk1^{+/+, +/loxp-alink} mice through germline transmission. Cdk1^{+/+, +/loxp-alink} mice were bred with Ella-Cre mice to generate Cdk1^{+/+, +/alink} mice. Cdk1^{+/+, +/alink} mice were then bred with MMTV-PyMT mice (Guy et al., 1992) to get double-positive pups with mammary gland tumors and ALINK modification. Mammary gland tumors with fail-safe modification were isolated, cut into 1 mm³ pieces, and transplanted into the 4th mammary gland of wild-type B6N females. GCV/PBS treatment was injected every other day at the dosage of 50 mg/kg into the peritoneal cavity with different treatment durations. Mammary gland tumor size was measured by calipers and calculated with the formula V=Length*Width*Height*π/6.

Neuronal Progenitor Vs. Neuron Killing Assay

Cdk1^{+/+, +/alink} human CA1 ES cells were differentiated to neural epithelial progenitor cells (NEPs). NEPs were subsequently cultured under conditions for differentiation into neurons, thereby generating a mixed culture of non-dividing neurons and dividing NEPs, which were characterized by immunostaining of DAPI, Ki67 and Sox2. GCV (10 uM) was provided to the mixed culture every other day for 20 days. Then, GCV was withdrawn from culture for 4 days before cells were fixed by 4% PFA. Fixed cells were immunostained for proliferation marker Ki67 to check whether all the leftover cells have exited cell cycle, and mature neutron marker beta-TublinIII.

Results

The mouse Cdk1 genomic locus is shown in FIG. 2a. Two vectors targeting murine Cdk1 were generated (FIGS. 2B and D), each configured to modify the 3′UTR of the Cdk1 gene (FIG. 2A) by replacing the STOP codon of the last exon with an F2A (Szymczak et al., 2004) sequence followed by an enhanced HSV-TK (TK.007 (Preuß et al., 2010)) gene connected to an mCherry reporter with a T2A (Szymczak et al., 2004) sequence.

Referring to FIG. 2B and mouse target vector I, the PGK-neo-pA selectable marker (necessary for targeting) was inserted into the TK.007 open-reading-frame with a piggyBac transposon, interrupting TK expression. The piggyBac transposon insertion was designed such that transposon removal restored the normal ORF of TK.007, resulting in expression of functional thymidine kinase (FIG. 2C).

Referring to FIG. 2D and mouse target vector II, the neo cassette was loxP-flanked and inserted between the F2A and TK.007.

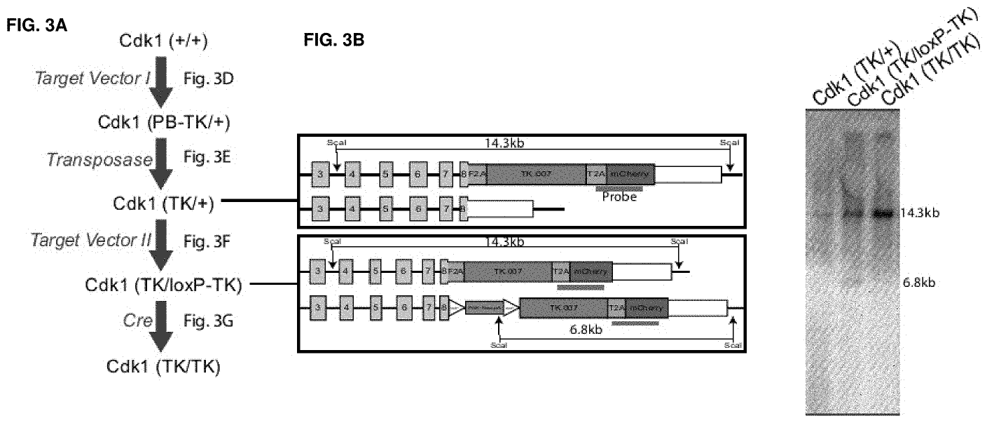

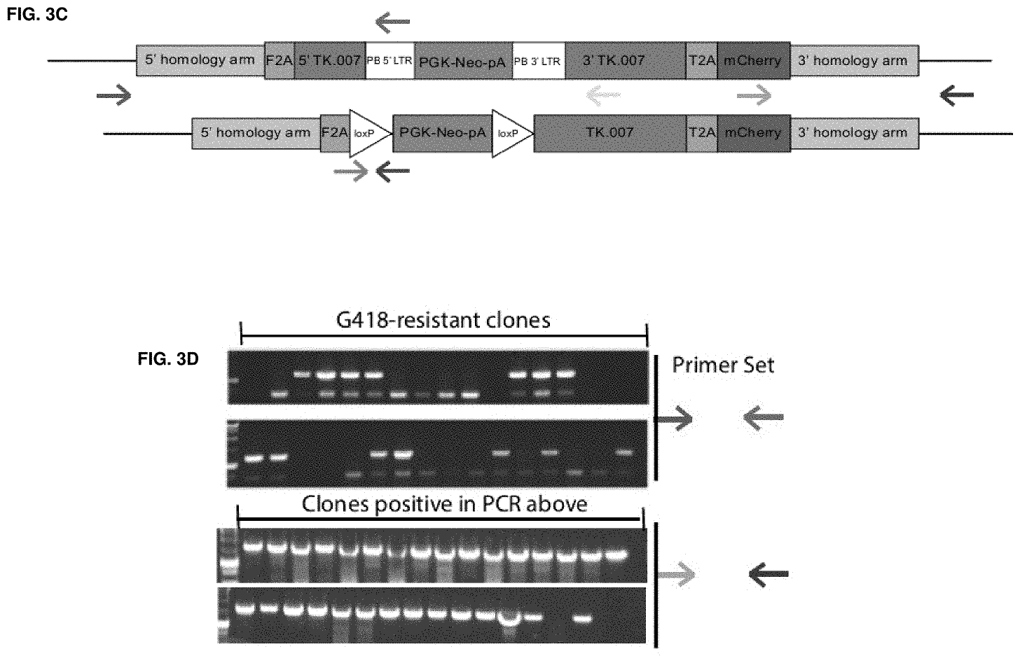

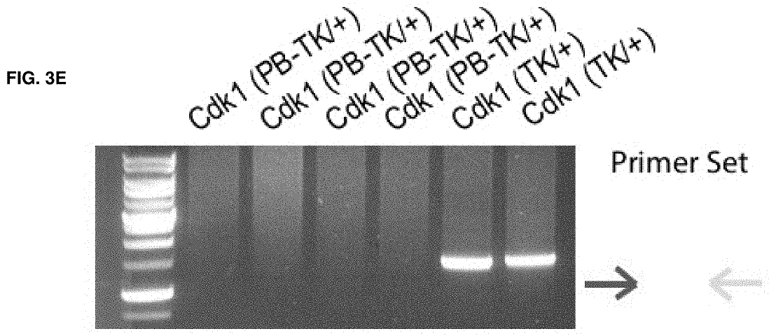

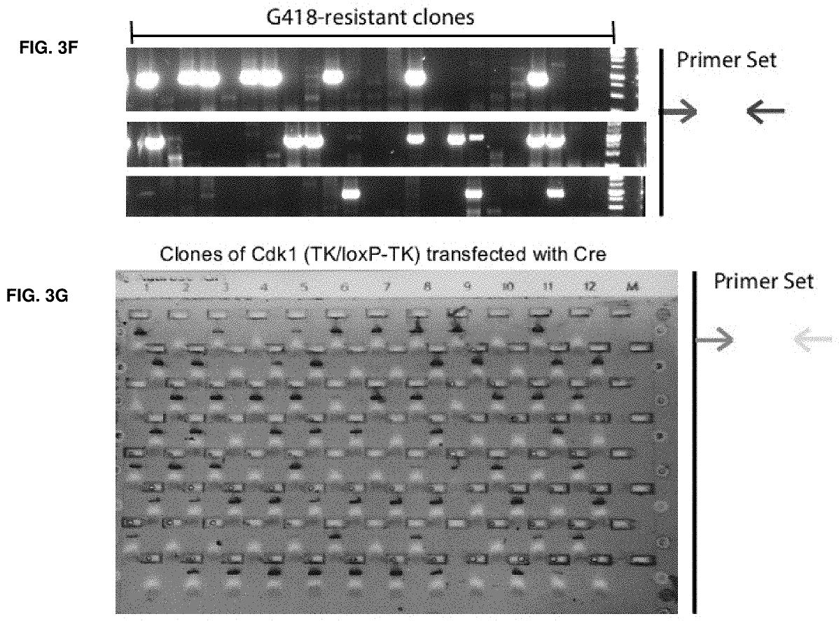

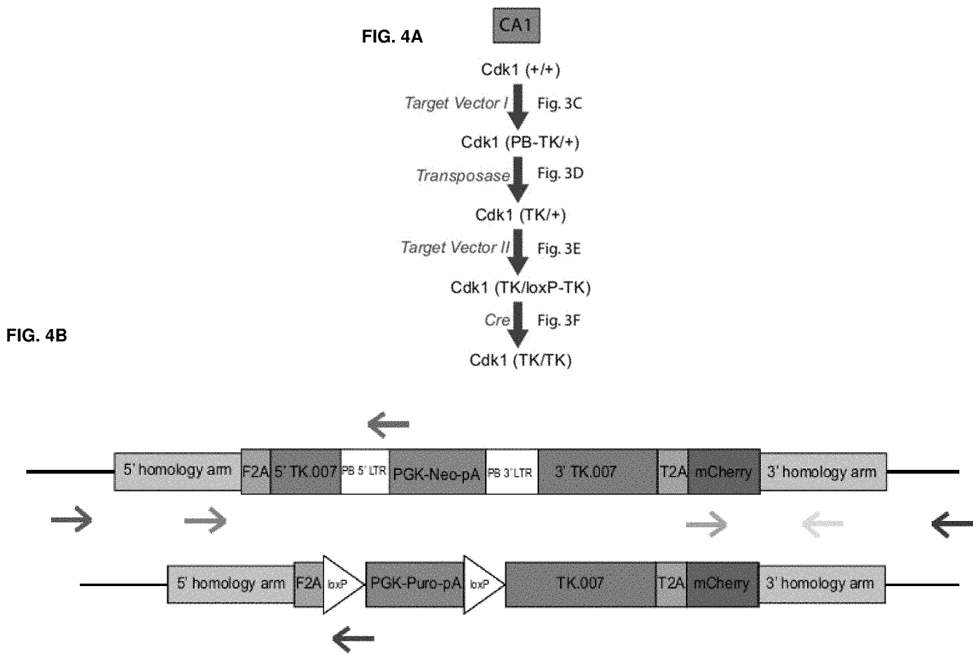

Target vectors I and II had short (˜800 bp) homology arms, which were sufficient for CRISPRs assisted homologous recombination targeting and made the PCR genotyping for identifying targeting events easy and reliable. The CRISPRs facilitated high targeting frequency at 40% PCR-positive of drug-resistant clones (FIG. 3D).

Both the piggyBac-inserted and the loxP-flanked neo cassettes were removed by transient expression of the piggyBac transposase and Cre recombinase, respectively, resulting in cell lines comprising alleles shown in FIGS. 2C and 2E, respectively. Referring to FIG. 2E, the remaining loxP site was in frame with TK and added 13 amino acids to the N-terminus of TK. The TK functionality test (GCV killing) proved that this N-terminus insertion did not interfere with TK function.

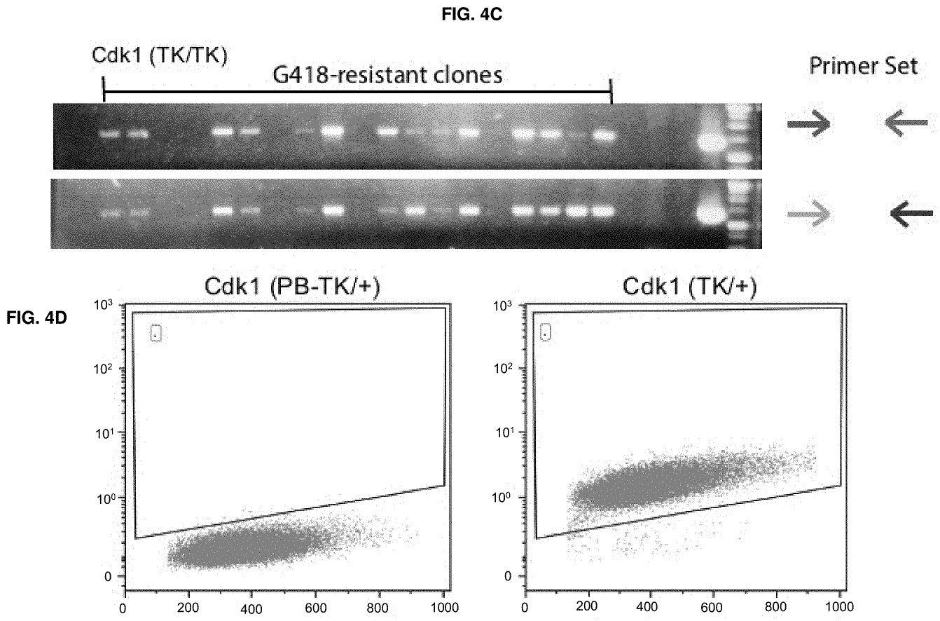

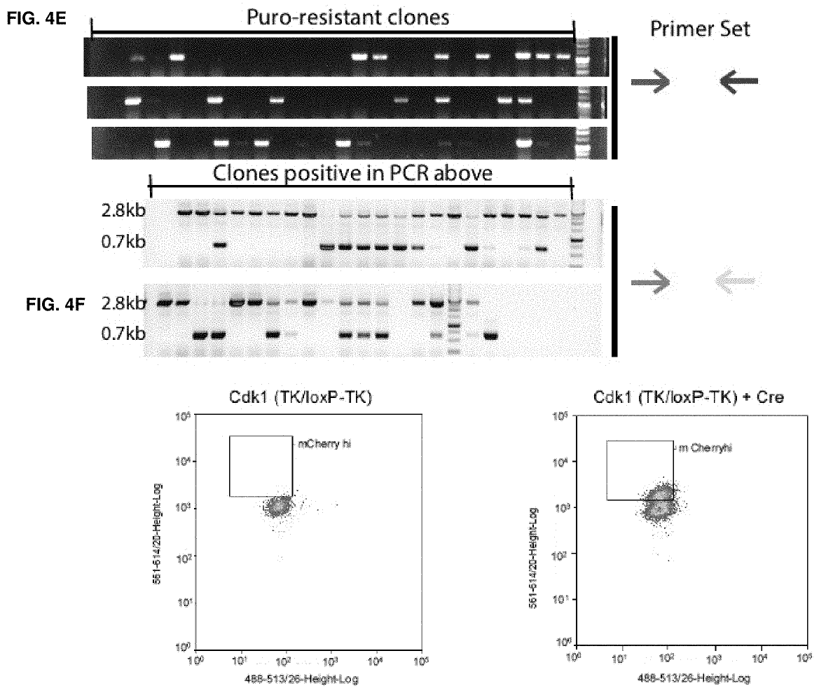

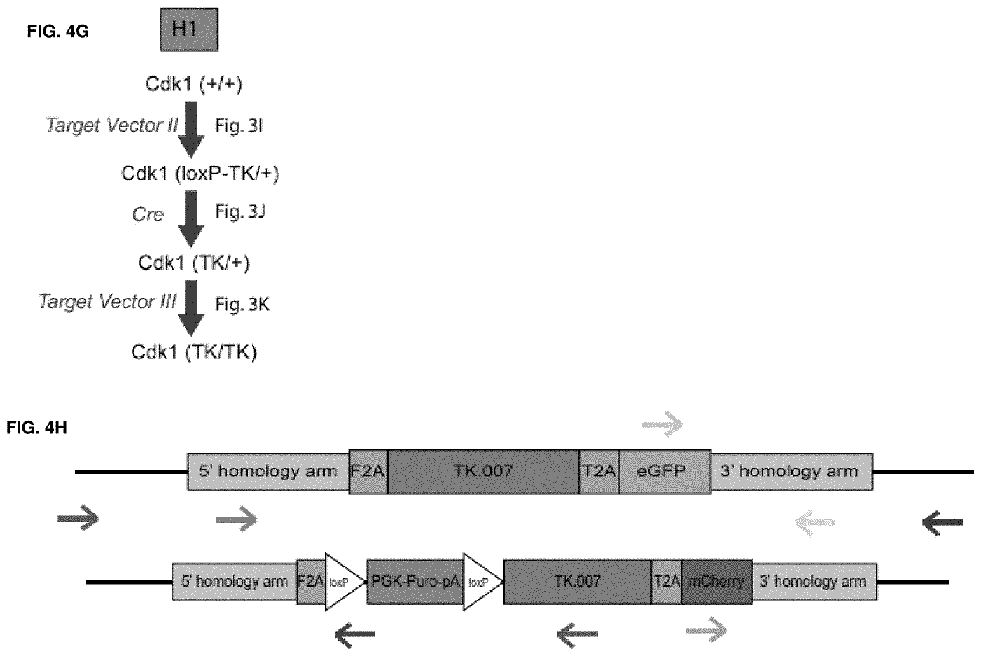

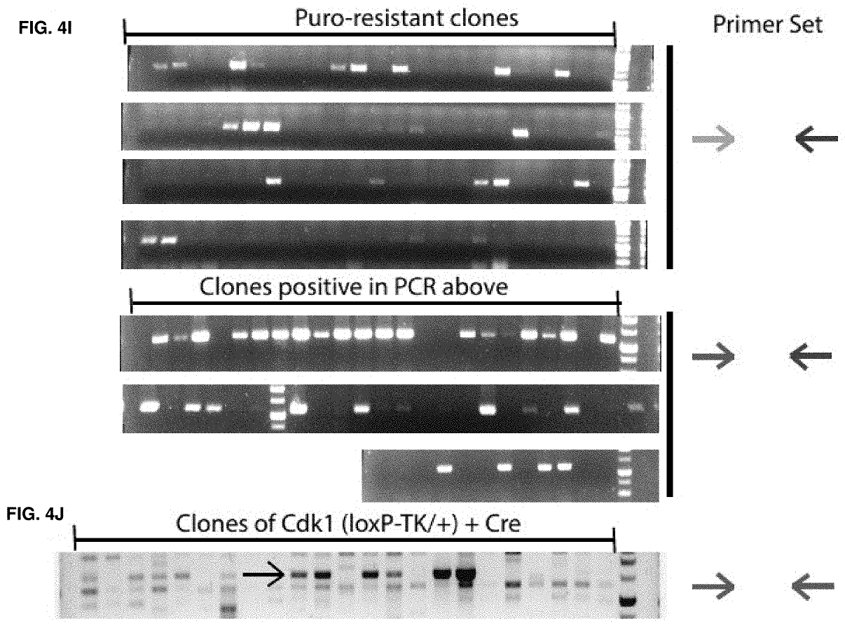

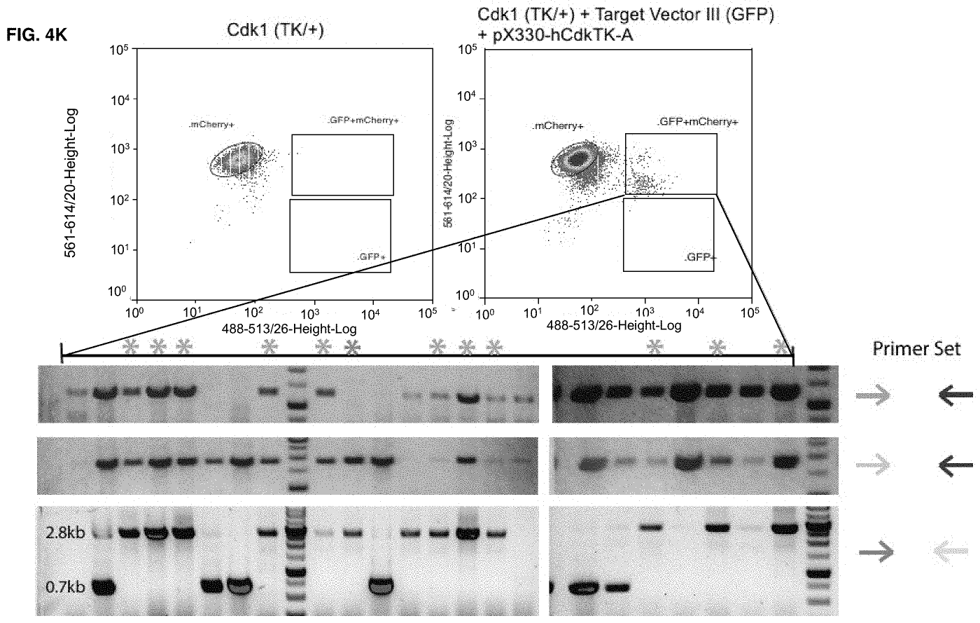

Referring to FIG. 4, assisted with CRISPR-Cas9 technology, homozygous ALINK can also be generated efficiently in two different human ES cell lines, CA1 and H1 (Adewumi et al., 2007).

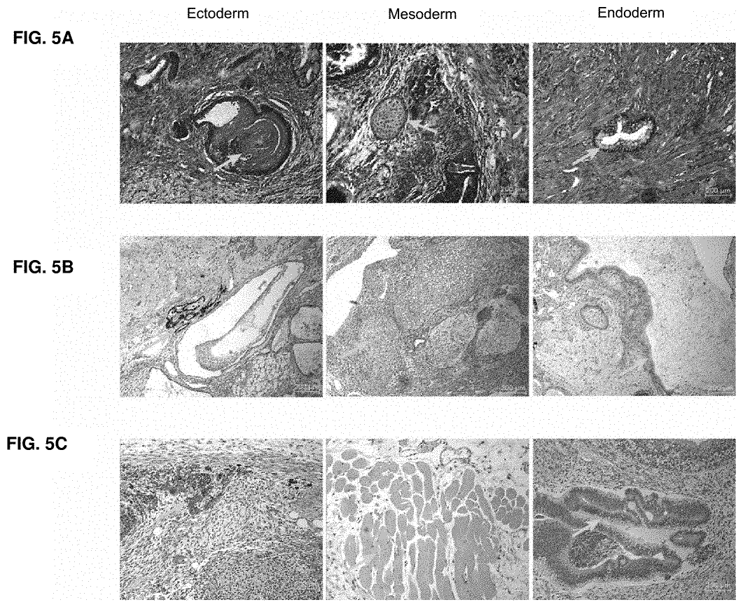

Referring to FIGS. 5A and 5C, the data indicate that: i) the TK.007 insertion into the 3′UTR of Cdk1 does not interfere with Cdk1 expression; ii) the ALINK-modified homozygous mouse C2 ES cells properly self-renew under ES cell conditions and differentiate in vivo and form complex teratomas; iii) the ALINK-modified homozygous human CA1 ES cells properly self-renew under ES cell conditions and differentiate in vivo and form complex teratomas.

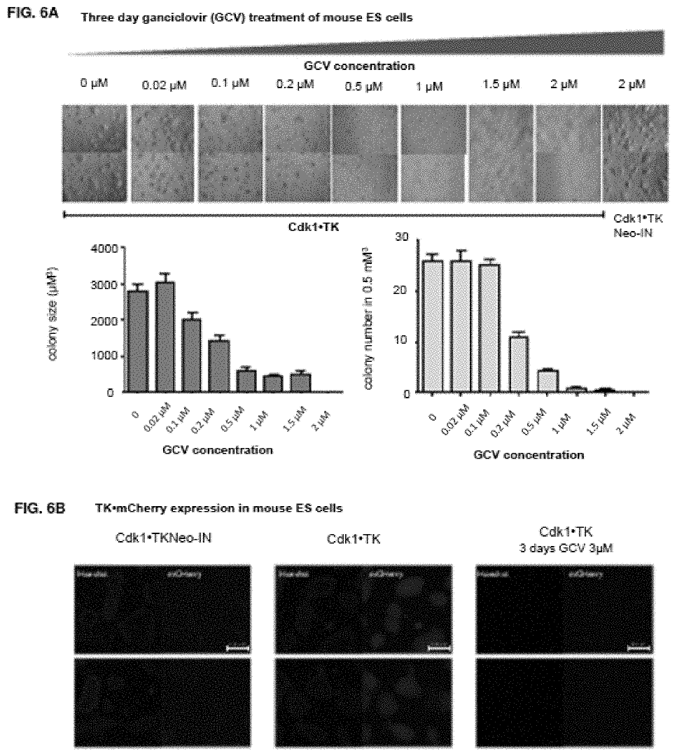

Referring to FIG. 6, the data indicate that: i) TK.007 is properly expressed; GCV treatment of undifferentiated ES cells ablates both homozygously- and heterozygously-modified cells (FIG. 6A); and ii) the T2A-linked mCherry is constitutively expressed in ES cells (FIG. 6B).

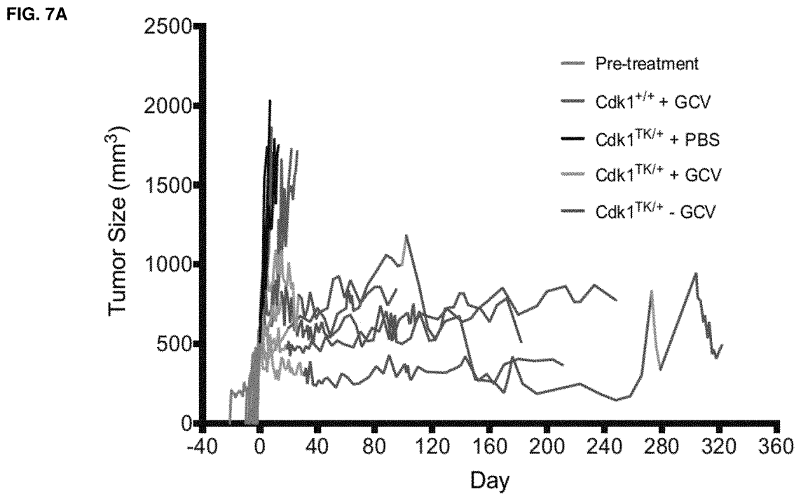

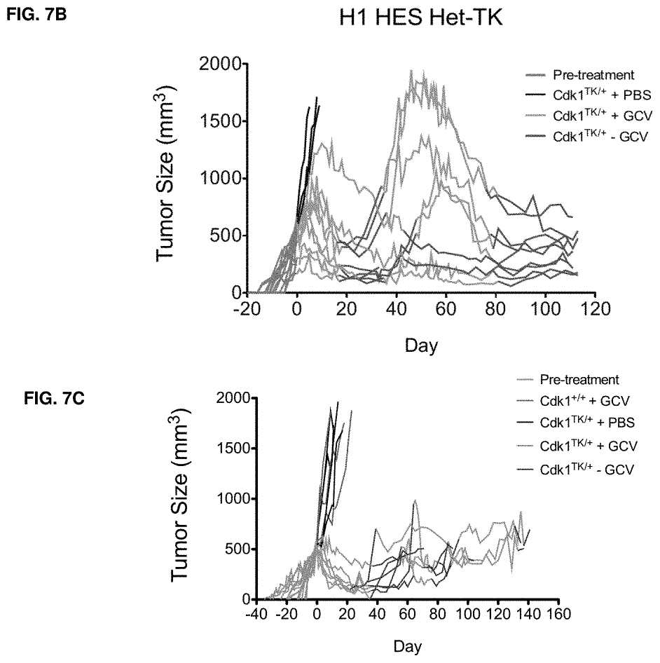

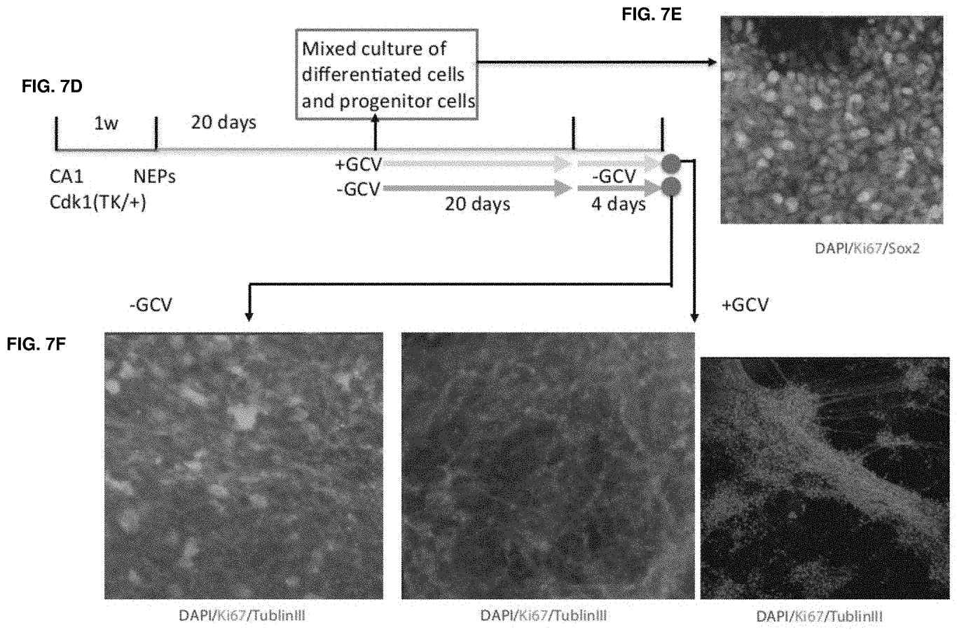

Referring to FIG. 7A, the data indicate that in hosts comprising ALINK-modified cell grafts, GCV treatment of subcutaneous teratomas comprising the ALINK-modified ES cells stops teratoma growth by ablating dividing cells. GCV treatment did not affect quiescent cells of the teratoma. A brief (3 week) GCV treatment period of the recipient was sufficient to render the teratomas dormant. Referring to FIG. 7B, in NOD scid gamma mouse hosts comprising ALINK-modified human cell grafts, two rounds of GCV treatment (1st round 15 days+2^{nd} round 40 days) rendered the teratomas to dormancy.

Referring to FIG. 7C, in B6N hosts comprising ALINK-modified MMTV-PyMT-transformed mammary epithelial tumorigenic cell grafts, GCV treatment was able to render the mammary gland tumors to dormancy.

Referring to FIGS. 7D-F, in a mixed culture of non-dividing neurons and dividing NEPs, all cells having been derived from Cdk1^{+/+, +/alink} human CA1 ES cells, GCV killed the dividing NEPs but did not kill the non-dividing neurons.

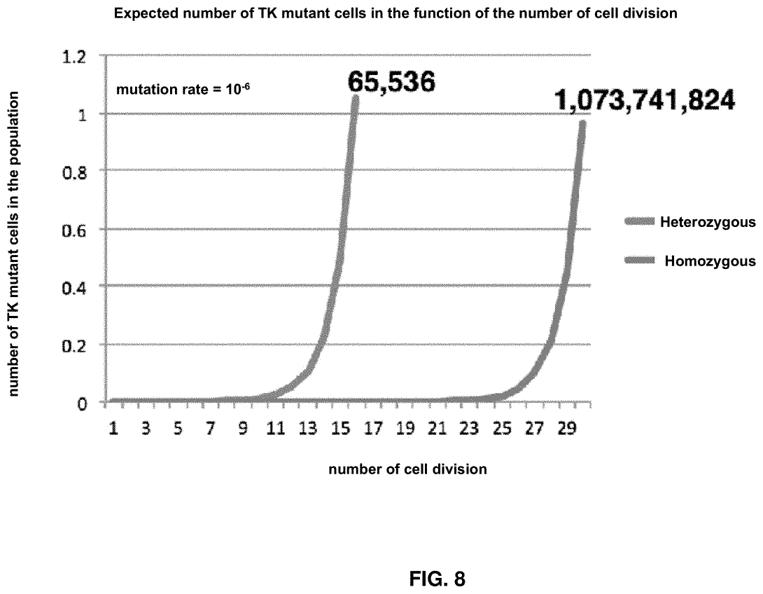

In an embodiment, it is contemplated that one or more dividing cells could escape GCV-mediated ablation if an inactivating mutation were to occur in the HSV-TK component of the CDL-HSV-TK transcriptional link. To address the probability of cell escape, the inventors considered the general mutation rate per cell division (i.e., 10⁻⁶) and determined that the expected number of cell divisions required to create 1 mutant cell would be 16 in cells comprising a heterozygous Cdk1-HSV-TK transcriptional link, and 30 cell divisions in cells comprising a homozygous Cdk1-HSV-TK transcriptional link. This means that if a single heterozygous ALINK-modified cell is expanded to 2¹⁶ (i.e., 65,000 cells) and a single homozygous ALINK-modified cell is expanded to 2³⁰ (i.e., 1 billion cells), then an average of one mutant cell comprising lost HSV-TK activity per heterozygous and homozygous cell population would be generated (FIG. 8). Accordingly, the inventors have determined that homozygous ALINK-modified cells would be very safe for use in cell-based therapies. Another way of calculating the level of safety of cell therapy was presented above.

### Example 2: Generation of EARC-Modified Mouse ES Cells in the Cdk1 Locus

In Example 2, construction of EARC (dox-bridge) vectors targeting Cdk1 and use of same to control cell division in mouse ES cells is described. In this example, Cdk1/CDK1 is the CDL, which is targeted with an inducible gene expression system, wherein a dox-bridge is inserted and doxycycline induces expression of the CDL.

As described above, Cdk1/CDK1 is expressed in all mitotically active (i.e., dividing) cells. In cells modified to comprise an EARC (dox-bridge) insertion at the Cdk1 locus, cell division is only possible in the presence of the inducer (doxycycline), which permits expression of Cdk1. Thus, cell division of EARC-modified mitotically active cells can be eliminated in the absence of doxycycline.

In Example 2, dox-bridge insertion into the 5′UTR of the Cdk1 gene was achieved by homologous recombination knock-in technology.

Methods

Construction of EARC Targeting Vector Comprising a Dox-Bridge

A fragment containing an rTTA coding sequence (SEQ ID NO: 41) followed by a 3×SV40 pA signal was amplified by PCR from a pPB-CAGG-rtta plasmid, using primers containing a lox71 site added at the 5′ of the rTTA (rtta3xpaFrw1 (SEQ ID NO: 63), rtta3xpaRev1(SEQ ID NO: 64)). This fragment was subcloned into a pGemT plasmid, to generate pGem-bridge-step1. Subsequently, a SacII fragment containing a TetO promoter (SEQ ID NO: 42) (derived from pPB-TetO-IRES-mCherry) was cloned into the SacII site of the pGem-bridge-step1, generating a pGem-bridge-step2. The final element of the bridge was cloned by inserting a BamHI IRES-Puromycin fragment (SEQ ID NO: 43) into the BamHI site of the pGem-bridge-step2, generating a pGem-bridge-step3. The 5′ homology arm was cloned by PCR-amplifying a 900 bp fragment (SEQ ID NO: 44) from C57/B6 genomic DNA (primers cdk5FrwPst (SEQ ID NO: 45) and cdk5RevSpe (SEQ ID NO: 46) and cloning it into SbfI and SpeI of the pGem-bridge-step3. Similarly, the 3′ homology arm (900 bp) (SEQ ID NO: 47) was amplified by PCR using primers dkex3_5′FSpe (SEQ ID NO: 48), cdkex3_3lox (SEQ ID NO: 49) and cloned into SphI and NcoI to generate a final targeting vector, referred to as pBridge (SEQ ID NO: 148).

Construction of CRISPR/Cas9 Plasmids

A double-nickase strategy was chosen to minimize the possibility of off-target mutations. Guide RNA sequences (SEQ ID NOs: 50, 51, 52 and 53) were cloned into pX335 (obtained from Addgene, according to the suggested protocol) (Ran et al., 2013).

Generation of EARC-Modified Mouse ES Cells

Mouse ES cell culture: All genetic manipulations were performed on a C57BL/6N mouse ES cell line previously characterized (C2) (Gertsenstein et al., 2010). Mouse ES cells were grown in media based on high-glucose DMEM (Invitrogen), supplemented with 15% ES cell-grade FBS (Gibco), 0.1 mM 2-mEARCaptophenol, 2 mM L-glutamine, 1 mM sodium pyruvate, 0.1 mM non-essential amino acids, and 2,000 units/ml leukemia inhibitory factor (LIF). Cells were maintained at 37° C. in 5% CO₂ on mitomycin C-treated mouse embryonic fibroblasts (MEFs).

Targeting: Plasmids containing the CRISPR/Cas9 components (pX335-cdk-ex3A (SEQ ID NO: 151) and px335-cdk-ex3B (SEQ ID NO: 152)) and the targeting plasmid (pBridge; SEQ ID NO: 148) were co-transfected in mouse ES cells using FuGENE HD (Clontech), according to the manufacturer's instructions, using a FuGENE:DNA ratio of 8:2, (2 μg total DNA: 250 ng for each pX330 and 1500 ng for pBridge). Typical transfection was performed on 3×10⁵ cells, plated on 35 mm plates. Upon transfection, doxycycline was added to the media to a final concentration of 1 μg/ml. 2 days following transfection, cells were plated on a 100 mm plate and selection was applied with 1 μg/ml of puromycin. Puromycin-resistant colonies were picked 8-10 days after start of selection and maintained in 96 well plates until PCR-screening.

Genotyping: DNA was extracted from ES cells directly in 96 well plates according to (Nagy et al., 2003). Clones positive for correct insertion by homologous recombination of pBridge in the 5′ of the Cdk1 gene were screened by PCR using primers spanning the 5′ and 3′ homology arms (primers rttaRev (SEQ ID NO: 54), ex3_5scr (SEQ ID NO: 55) for the 5′ arm, primers CMVforw (SEQ ID NO: 56), ex3_3scr (SEQ ID NO: 57) for the 3′ arm).

Targeted cell growth: F3-bridge targeted cells were trypsinized and plated on gelatinized 24 well plates at a density of 5×10⁴ cells per well. Starting one day after plating, cell counting was performed by trypsinizing 3 wells for each condition and counting live cells using a Countess automated cell counter (Life Technologies). Doxycycline was removed or reduced to 0.05 ng/ml 2 days after plating and live cells were counted every day up to 18 days in the different conditions.

Cre-excision: F3-bridge cells (grown in Dox+ media) were trypsinized and transfected with 2 μg of a plasmid expressing Cre (pCAGG-NLS-Cre). Transfection was performed using JetPrime (Polyplus) according to the manufacturer's protocol. After transfection, doxycycline was removed and colonies were trypsinized and expanded as a pool.

Quantitative PCR: Total RNA was extracted from cells treated for 2 days with 1 μg/ml and 0 μg/ml of Dox using the GeneElute total RNA miniprep kit (Sigma) according to the manufacturer's protocol. cDNA was generated by reverse transcription of 1 μg of RNA using the QuantiTect reverse transcription kit (Qiagen), according to the manufacturer's protocol. Real-time qPCR were set up in a BioRad CFX thermocycler, using SensiFast-SYBR qPCR mix (Bioline). The primers used were: qpercdk1_F (SEQ ID NO: 65), qpercdk1_R (SEQ ID NO:66) and actBf (SEQ ID NO: 67), actBr (SEQ ID NO: 68). Results were analyzed with the ΔΔCT method and normalized for beta-actin.

Results

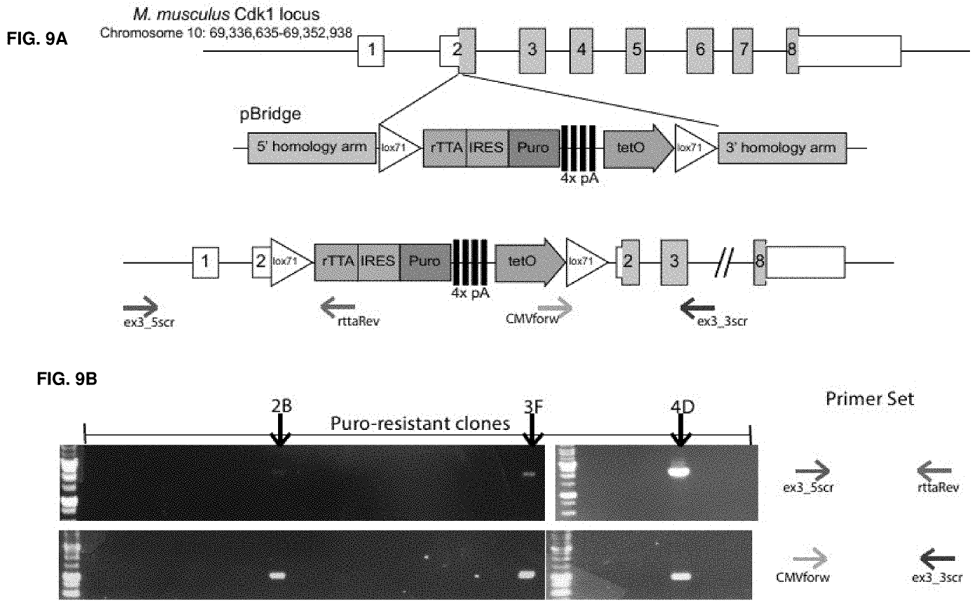

Referring to FIG. 9, the dox-bridge target vector, depicted in FIG. 9A, was used to generate three targeted C2 mouse ES cell lines (FIG. 9B). One of these cell lines was found to be a homozygous targeted line (3F in FIG. 9B) comprising a dox-bridge inserted by homologous recombination into the 5′UTR of both alleles of Cdk1.

As expected, this ES cell line grows only in the presence of doxycycline. In the presence of doxycycline, the Cdk1 promoter activity produced rtTA binds to TRE and initiates transcription of the Cdk1. Similarly to the 3′ modification, the dox-bridge may be inserted into the 5′UTR into both alleles of Cdk1, to ensure that the CDL expression could occur only through EARC. An alternative is to generate null mutations in all the remaining, non-EARC modified alleles of CDL.

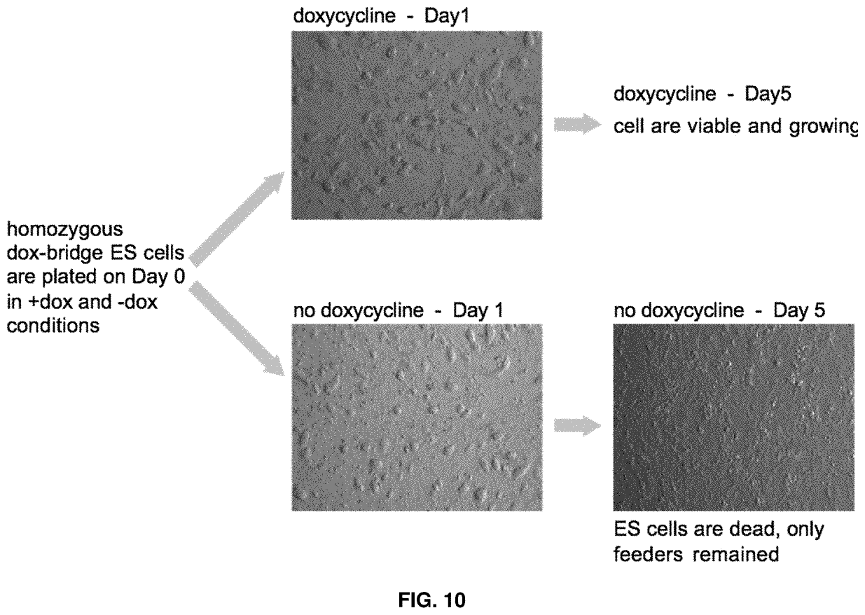

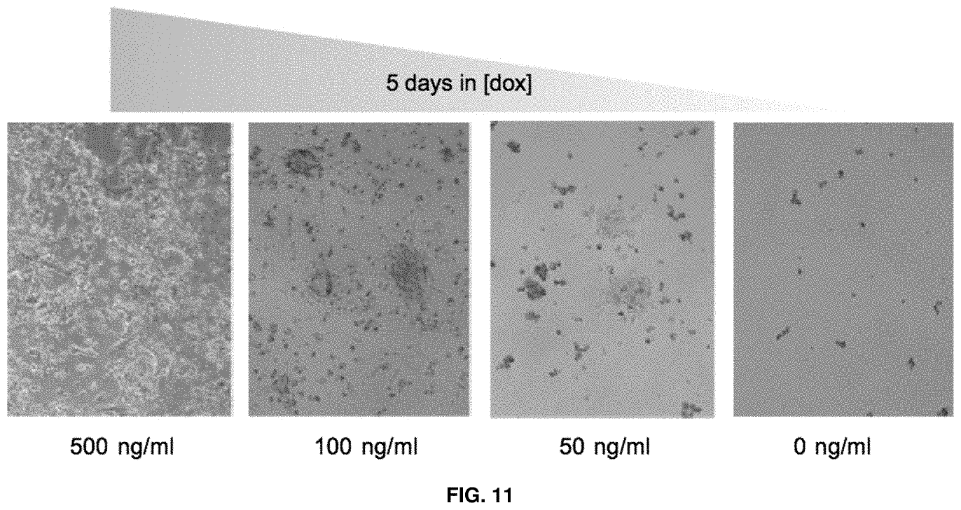

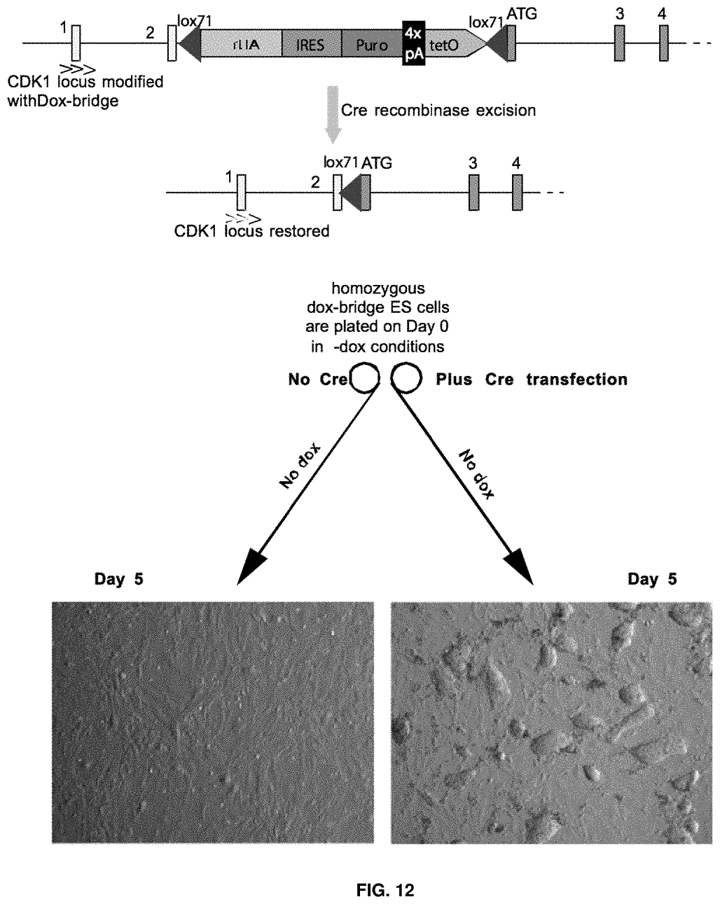

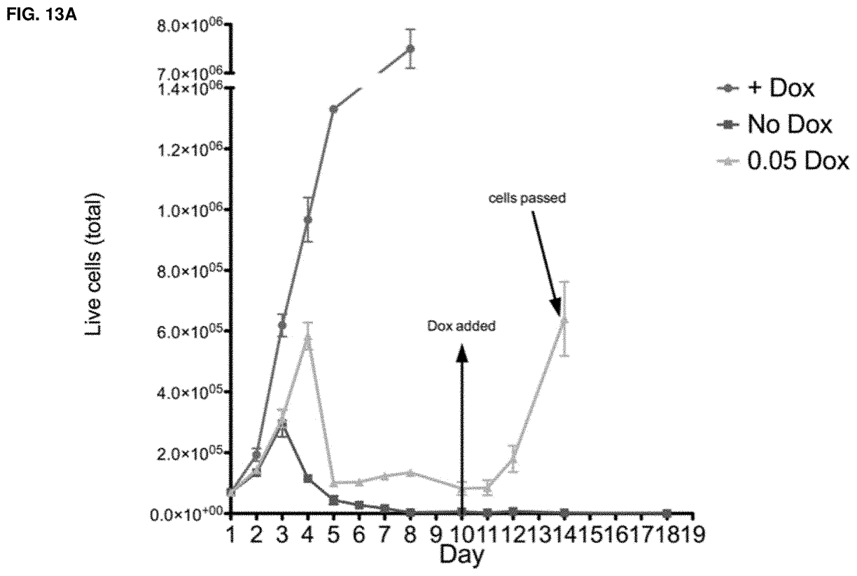

Withdrawal of doxycycline resulted in complete elimination of mitotically active ES cells within 5 days (FIG. 10). Lowering the doxycycline concentration by 20× (50 ng/ml) compared to the concentration used for derivation and maintenance of the doc-bridged cell line, allowed some cells/colonies to survive the 5 days period (FIG. 11).

Referring to FIG. 12, the dox-bridge was removable with a Cre recombinase mediated excision of the segment between the two lox71 sites, which restore the original endogenous expression regulation of the allele and rescues the cell lethality from the lack of doxycycline. These data indicate that the dox-bridge was working in the cells as predicted.

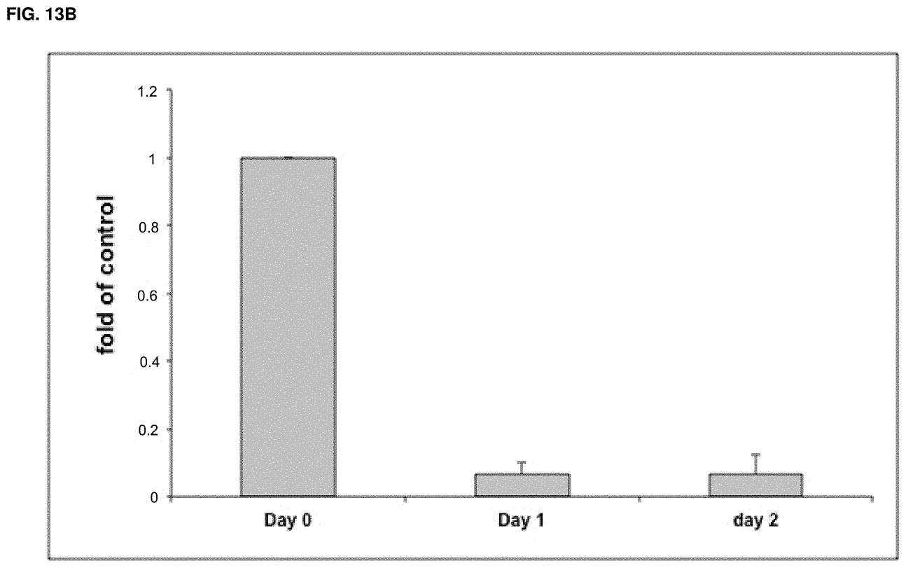

Referring to FIG. 13, the inventors determined how doxycycline withdrawal affected elimination of the dox-bridge ES cells by measuring cell growth in the presence and absent of doxycycline. ES cells in the presence of doxycycline grew exponentially, indicating their normal growth. In contrast, upon withdrawal of doxycycline (Day 1) cells grew for only two days and then cells death began until no live cells were present on Day 9. A 20× lower doxycycline concentration (50 ng/ml) provided after an initial 3 days of cell growth was sufficient to maintain a constant number of cells on the plates for at least five days (FIG. 13, light blue line). When the normal concentration of doxycycline was added back to the plate on day 10, cells started growing again as normal ES cells.

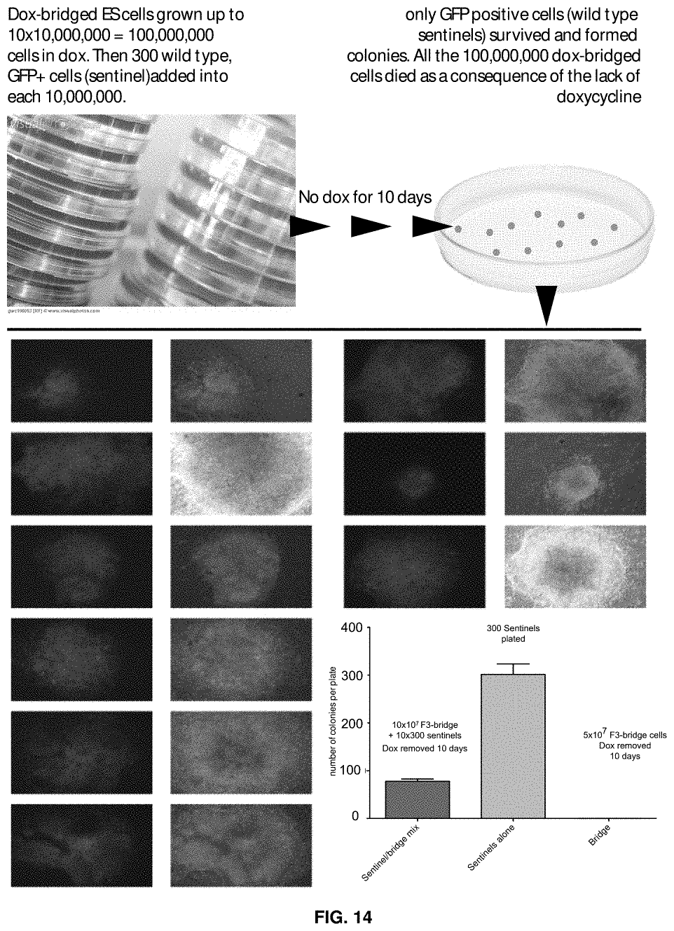

It is contemplated that dividing cells could escape EARC (dox-bridge)-modification of Cdk1 when grown in media lacking doxycycline. To address the probability of cell escape, EARC (dox-bridge)-modified mouse ES cells were grown up to 100,000,000 cells/plate on ten plates in medium containing doxycycline. 300 GFP-positive wild-type ES cells (sentinels) were then mixed into each 10 plate of modified ES cells and doxycycline was withdrawn from the culture medium. Only GFP positive colonies were recovered (FIG. 14) indicating that there were no escapee dox-bridged ES cells among the 100,000,000 cells in the culture. Accordingly, the inventors have determined that EARC (dox-bridge)-modified ES cells would add an additional level of safety to ALINK modification for certain cell therapy applications, because loss of the dox-bridge is unlikely to occur by mutation and cell division is not possible in the absence of the inducer (doxycycline) due to the block of CDL expression.

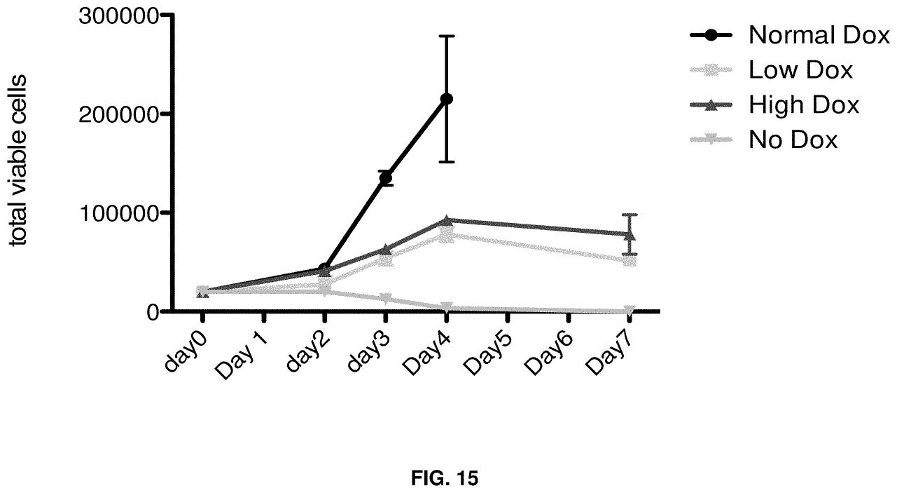

Referring to FIG. 15, the effect of high doxycycline concentration (10 μg/ml) on the growth of dox-bridged ES cells was examined. In the presence of high concentration doxycycline, the growth rate of dox-bridged ES cells slowed to a rate similar to that of cells grown in low concentration doxycycline. These data suggest that there is a range of doxycycline concentrations that may permit optimal Cdk1 expression for wild-type cell-like proliferation.

### Example 3: Generation of EARC-ALINK Modified Cells in the CDK1 Locus (Mouse and Human)

In Example 3, construction of EARC (dox-bridge) vectors targeting CDK1 and use of same to control cell division in both mouse and human ALINK-modified ES cells is described. In this example, Cdk1/CDK1 is the CDL, the dox-bridge is the EARC, and HSV-TK is the ALINK. CDL Cdk1 is modified with both EARC and ALINK systems in the homozygous form, wherein doxycycline is required to induce expression of the CDL, and wherein doxycycline and GCV together provide a way of killing the modified proliferating cells.

In Example 3, dox-bridge insertion into the 5′UTR of the CDK1 gene was achieved by homologous recombination knock-in technology.

Methods

Construction of mouse EARC targeting vector, CRISPR/Cas9 plasmids for mouse targeting are the same as in Example 2. Targeting and genotyping methods are also the same as described in Example 2 except that instead of C2 WT cells, Cdk1(TK/TK) cells generated in Example 1 (FIG. 3A-3G) were used for transfection.

Construction of EARC Targeting Vector Comprising a Dox-Bridge for Human CDK1

The 5′ homology arm (SEQ ID NO: 69) was cloned by PCR-amplifying a 981 bp fragment from CA1 genomic DNA (primers hcdk5′F (SEQ ID NO: 70) and hcdk5′R (SEQ ID NO: 71) and cloning it into SbfI of the pGem-bridge-step3. Similarly, the 3′ homology arm (943 bp; SEQ ID NO: 72) was amplified by PCR using primers hcdk3′F (SEQ ID NO: 73) and hcdk3′R (SEQ ID NO: 74) and cloned into SphI and NcoI to generate a final targeting vector, referred to as pBridge-hCdk1 (SEQ ID NO: 75).

Construction of CRISPR/Cas9 Plasmids for Human Targeting

Guide RNA (hCdk1A_up (SEQ ID NO: 76), hCdk1A_low (SEQ ID NO: 77), hCdk1B_up (SEQ ID NO: 78), hCdk1B_low (SEQ ID NO: 79)) were cloned in to pX335 (SEQ ID NO: 149) and pX330 (SEQ ID NO: 150) to generate pX335-1A (SEQ ID NO: 80), pX335-1B (SEQ ID NO: 81) and pX330-1B (SEQ ID NO: 82).

Generation of EARC-Modified Human ES Cells

Targeting: 2×10⁶ CA1 Cdk1(TK/TK) (i.e., the cell product described in FIGS. 4A-4F) hES cells were transfected by Neon protocol 14 with 8 ug DNA (Target Vector: pX330-hCdkTK-A=6 ug:2 ug). After transfection, cells were plated on four 10-cm plates. Upon transfection, doxycycline was added to the media to a final concentration of 1 μg/ml. 2 days following transfection, selection was applied with 0.75 μg/ml of puromycin. Puromycin-resistant colonies were picked to 96-well plates, duplicated for further growth and genotyping with primers (hCdk1Br-5HAgen_F1 (SEQ ID NO: 83), rtTA_rev_1 (SEQ ID NO: 84), mCMV_F (SEQ ID NO:85), hCdk1Br-3HAgen_R1 (SEQ ID NO: 86)).

Results

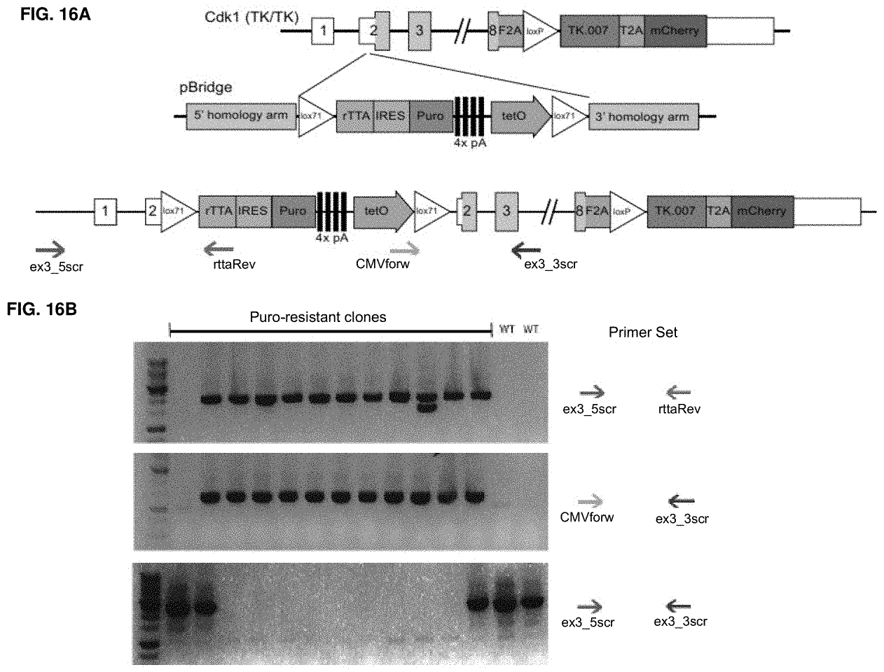

Referring to FIG. 16A, the mouse dox-bridge target vector, pBridge was used to target mouse cell products generated in Example 1, Cdk1(TK/TK), generating mouse Cdk1^{earc/earc,alink/alink} cells. Nine Cdk1^{earc/earc,alink/alink} clones were generated by one-shot transfection (FIG. 16B).

Referring to FIG. 5B, the data indicate that the EARC-and-ALINK-modified homozygous mouse C2 ES Cdk1^{earc/earc,alink/alink} cells properly self-renewed under ES cell conditions, differentiated in vivo, and formed complex teratomas.

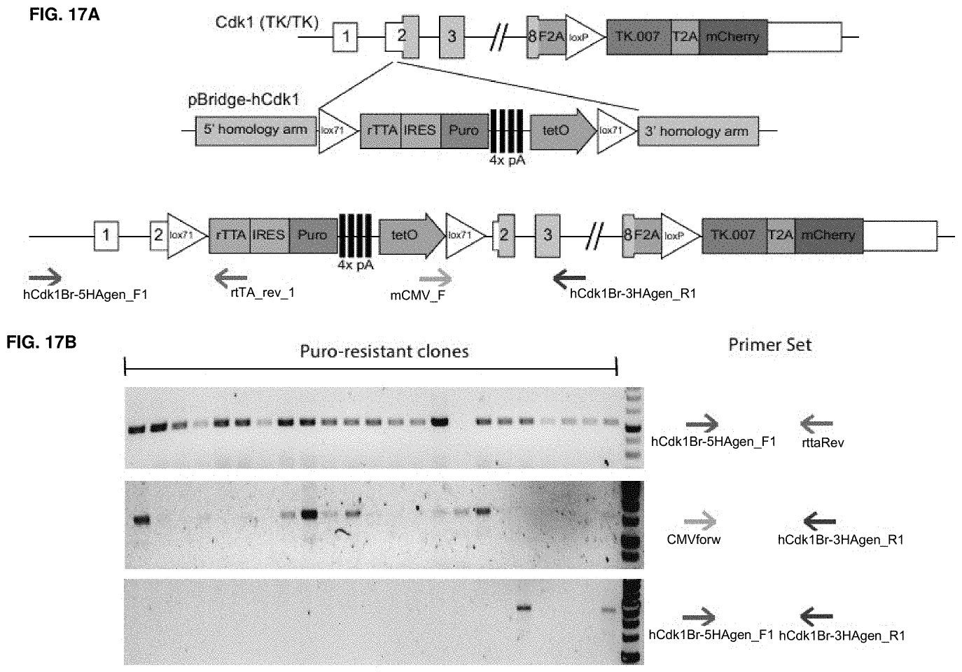

Referring to FIG. 17A, the human dox-bridge target vector, pBridge-hCdk1 was used to target human CA1 cell products generated in Example 1, Cdk1(TK/TK), generating human Cdk1^{earc/earc,alink/alink} cells. At least Cdk1^{earc/earc,alink/alink} CA1 clones were generated by one-shot transfection (FIG. 17B).

### Example 4: Generation of EARC-Modified Mouse ES Cells in the Top2a Locus

In Example 4, construction of EARC (dox-bridge) vectors targeting Top2a and use of same to control cell division in mouse ES cells is described. In this example, Top2a/TOP2A is the CDL, which is targeted with an inducible gene expression system, wherein a dox-bridge is inserted and doxycycline induces expression of the CDL.

As described above, Top2a/TOP2A is expressed in all mitotically active (i.e., dividing) cells. In cells modified to comprise an EARC (dox-bridge) insertion at the Top2a locus, cell division is only possible in the presence of the inducer (doxycycline), which permits expression of Top2a. Thus, cell division of EARC-modified mitotically active cells can be eliminated in the absence of doxycycline.

In Example 4, dox-bridge insertion into the 5′UTR of the Top2a gene was achieved by homologous recombination knock-in technology.

Methods

Construction of EARC Targeting Vector Comprising a Dox-Bridge for Top2a

The 5′ homology arm (SEQ ID NO: 87) was cloned by PCR-amplifying a 870 bp fragment from C57/B6 genomic DNA (primers Top5F (SEQ ID NO: 88) and Top5R (SEQ ID NO: 89) and cloning it into SbfI and SpeI of the pGem-bridge-step3. Similarly, the 3′ homology arm (818 bp; SEQ ID NO: 90) was amplified by PCR using primers Top3F (SEQ ID NO: 91), Top3R (SEQ ID NO: 92) and cloned into SphI and NcoI to generate a final targeting vector, referred to as pBridge-Top2a (SEQ ID NO: 93).

Construction of CRISPR/Cas9 Plasmids

A double-nickase strategy was chosen to minimize the possibility of off-target mutations. Guide RNA sequences were cloned into pX335 (Addgene) using oligos: TOP2A1 BF (SEQ ID NO: 94), TOP2A1BR (SEQ ID NO: 95), TOP2A1AF (SEQ ID NO: 96), TOP2A1AR (SEQ ID NO: 97), according to the suggested protocol (Ran et al., 2013), generating the CRISPR vectors pX335-Top2aA (SEQ ID NO: 98) and px335-Top2aB (SEQ ID NO: 99).

Generation of EARC-Modified Mouse ES Cells

Mouse ES cell culture: All genetic manipulations were performed on a C57/B6 mouse ES cell line previously characterized (C2) (Gertsenstein et al., 2010). Mouse ES cells were grown in media based on high-glucose DMEM (Invitrogen), supplemented with 15% ES cell-grade FBS (Gibco), 0.1 mM 2-mEARCaptophenol, 2 mM L-glutamine, 1 mM sodium pyruvate, 0.1 mM non-essential amino acids, and 2,000 units/ml leukemia inhibitory factor (LIF). Cells were maintained at 37° C. in 5% CO₂ on mitomycin C-treated mouse embryonic fibroblasts (MEFs).

Targeting: Plasmids containing the CRISPR/Cas9 components (pX335-Top2aA (SEQ ID NO: 98) and px335-Top2aB (SEQ ID NO: 99)) and the targeting plasmid (pBridge-Top2a (SEQ ID NO: 93)) were co-transfected in mouse ES cells using FuGENE HD (Clontech), according to the manufacturer's instructions, using a FuGENE:DNA ratio of 8:2, (2 μg total DNA: 250 ng for each pX335 and 1500 ng for pBridge-Top2a). Typical transfection was performed on 3×10⁵ cells, plated on 35 mm plates. Upon transfection, doxycycline was added to the media to a final concentration of 1 μg/ml. 2 days following transfection, cells were plated on a 100 mm plate and selection was applied with 1 μg/ml of puromycin. Puromycin-resistant colonies were picked 8-10 days after start of selection and maintained in 96 well plates until PCR-screening.

Genotyping: DNA was extracted from ES cells directly in 96 well plates according to (Nagy et al., 2003). Clones positive for correct insertion by homologous recombination of pBridge-Top2a in the 5′ of the Top2a gene were screened by PCR using primers spanning the 5′ and 3′ homology arms (primers rttaRev (SEQ ID NO: 54), top2a_5scrF (SEQ ID NO: 55) for the 5′ arm, primers CMVforw (SEQ ID NO: 56), top2a_3scrR (SEQ ID NO: 57) for the 3′ arm).

Targeted cell growth: Top2a homozygously-targeted cells were trypsinized and plated on gelatinized 24 well plates at a density of 5×10⁴ cells per well. Starting one day after plating, cells were exposed to different Dox concentrations (1 μg/ml, 0.5 μg/ml, 0.05 μg/ml and 0 μg/ml), the plate was analyzed in a IncucyteZoom system (Essen Bioscience) by taking pictures every two hours for 3-4 days and measuring confluency.

Results

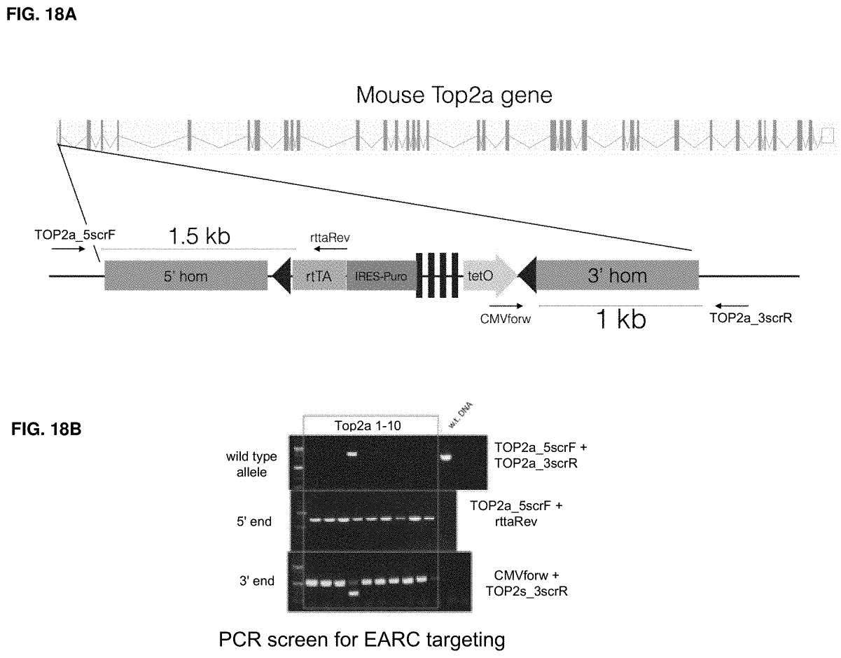

Referring to FIG. 18, the dox-bridge target vector, depicted in FIG. 18A, was used to generate several targeted C2 mouse ES cell lines (FIG. 18B). Nine of these cell lines were found to be homozygous targeted (FIG. 18B) comprising a dox-bridge inserted by homologous recombination into the 5′UTR of both alleles of Top2a.

As expected, this ES cell lines grows only in the presence of doxycycline. In the presence of doxycycline, the rtTA produced by Top2a promoter, binds to TRE and initiates transcription of the Top2a coding sequence. The dox-bridge may be inserted into the 5′UTR into both alleles of Top2a to ensure that the CDL expression could occur only through EARC. An alternative is to generate null mutations in all the remaining, non-EARC modified alleles of CDL.

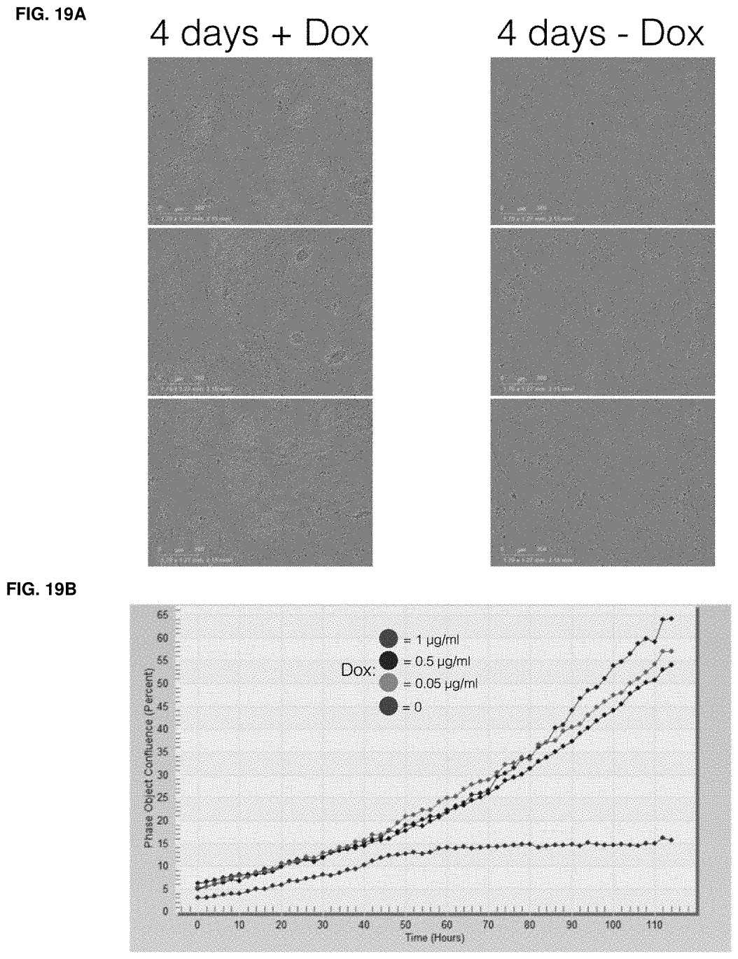

Withdrawal of doxycycline resulted in complete elimination of mitotically active ES cells within 4 days (FIG. 19A).

Referring to FIG. 19B, the inventors determined how different concentrations of doxycycline affected proliferation of the dox-bridge ES cells by measuring cell growth for 4 days. ES cells in the presence of doxycycline grew exponentially, indicating their normal growth. In contrast, two days after doxycycline removal, cells growth of EARC-modified cells was completely arrested.

### Example 5: Generation of EARC-Modified Mouse ES Cells in the Cenpa Locus

In Example 5, construction of EARC (dox-bridge) vectors targeting Cenpa and use of same to control cell division in mouse ES cells is described. In this example, Cenpa/CENPA is the CDL, which is targeted with an inducible gene expression system, wherein a dox-bridge is inserted and doxycycline induces expression of the CDL.

As described above, Cenpa/CENPA is expressed in all mitotically active (i.e., dividing) cells. In cells modified to comprise an EARC (dox-bridge) insertion at the Cenpa locus, cell division is only possible in the presence of the inducer (doxycycline), which permits expression of Cenpa. Thus, cell division of EARC-modified mitotically active cells can be eliminated in the absence of doxycycline.

In Example 5, dox-bridge insertion into the 5′UTR of the Cenpa gene was achieved by homologous recombination knock-in technology.

Methods

Construction of EARC Targeting Vector Comprising a Dox-Bridge

The 5′ homology arm (SEQ ID NO: 100) was cloned by PCR-amplifying a 874 bp fragment from C57/B6 genomic DNA (primers Cenpa5F (SEQ ID NO: 101) and Cenpa5R (SEQ ID NO: 102) and cloning it into SbfI and SpeI of the pGem-bridge-step3. Similarly, the 3′ homology arm (825 bp; SEQ ID NO: 103) was amplified by PCR using primers Cenpa3F (SEQ ID NO: 104), Cenpa3R (SEQ ID NO: 105) and cloned into SphI and NcoI to generate a final targeting vector, referred to as pBridge-Cenpa (SEQ ID NO: 106).

Construction of CRISPR/Cas9 Plasmids

A double-nickase strategy was chosen to minimize the possibility of off-target mutations. Guide RNA sequences were cloned into pX335 (Addgene) using oligos CenpaAF (SEQ ID NO: 107), CenpaAR (SEQ ID NO: 108), CenpaBF (SEQ ID NO: 109), CenpaBR (SEQ ID NO: 110), according to the suggested protocol (Ran et al., 2013), generating the CRISPR vectors pX335-CenpaA (SEQ ID NO: 111) and px335-CenpaB (SEQ ID NO: 112).

Generation of EARC-Modified Mouse ES Cells

Mouse ES Cell Culture: As in Example 4.

Targeting: Plasmids containing the CRISPR/Cas9 components (pX335-CenpaA; SEQ ID NO: 111, and px335-CenpaB; SEQ ID NO: 112) and the targeting plasmid (pBridge-Cenpa; SEQ ID NO: 106) were co-transfected in mouse ES cells using FuGENE HD (Clontech), as in Example 4.

Genotyping: DNA was extracted as in Example 4. Clones positive for correct insertion by homologous recombination of pBridge-Cenpa in the 5′ of the Cenpa gene were screened by PCR using primers spanning the 5′ and 3′ homology arms (primers rttaRev (SEQ ID NO: 54), Cenpa_5scr (SEQ ID NO: 113) for the 5′ arm, primers CMVforw (SEQ ID NO: 114), Cenpa_3scr (SEQ ID NO: 115) for the 3′ arm).

Targeted cell growth: Cenpa homozygously-targeted cells were trypsinized and plated on gelatinized 24 well plates at a density of 5×10⁴ cells per well. Starting one day after plating, cells were exposed to different Dox concentrations (1 μg/ml, 0.5 μg/ml, 0.05 μg/ml and 0 μg/ml), the plate was analyzed in a IncucyteZoom system (Essen Bioscience) by taking pictures every two hours for 3-4 days and measuring confluency.

Quantitative PCR: Total RNA was extracted from cells treated for 2 days with 1 μg/ml and 0 μg/ml of Dox using the GeneElute total RNA miniprep kit (Sigma) according to the manufacturer's protocol. cDNA was generated by reverse transcription of 1 μg of RNA using the QuantiTect reverse transcription kit (Qiagen), according to the manufacturer's protocol. Real-time qPCR were set up in a BioRad CFX thermocycler, using SensiFast-SYBR qPCR mix (Bioline). The primers used were: qpercenpa_F (SEQ ID NO: 116), qpercenpa_R (SEQ ID NO: 117) and actBf (SEQ ID NO: 67), actBr (SEQ ID NO: 68). Results were analyzed with the ΔΔCT method and normalized for beta-actin.

Results

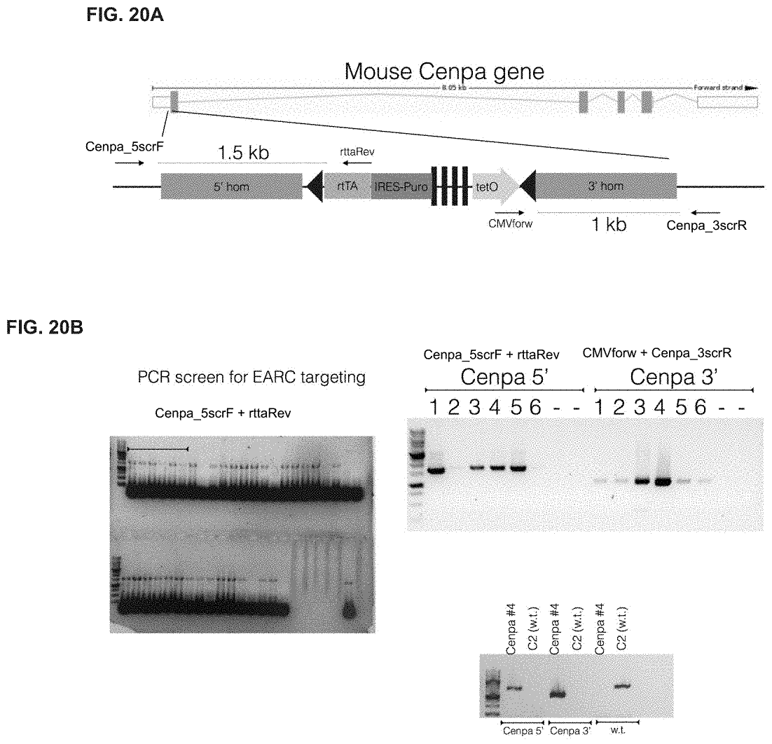

Referring to FIG. 20, the dox-bridge target vector, depicted in FIG. 20A, was used to generate several targeted C2 mouse ES cell lines (FIG. 20B). Six of these cells were found to have a correct insertion at the 5′ and 3′, and at least one clone (Cenpa #4), was found to have homozygous targeting (FIG. 20B) comprising a dox-bridge inserted by homologous recombination into the 5′UTR of both alleles of Cenpa.

As expected, this ES cell lines grows only in the presence of doxycycline. In the presence of doxycycline, the rtTA produced by Cenpa promoter, binds to TRE and initiates transcription of the Cenpa coding sequence. The dox-bridge may be inserted into the 5′UTR into both alleles of Cenpa, to ensure that the CDL expression could occur only through EARC. An alternative is to generate null mutations in all the remaining, non-EARC modified alleles of CDL.

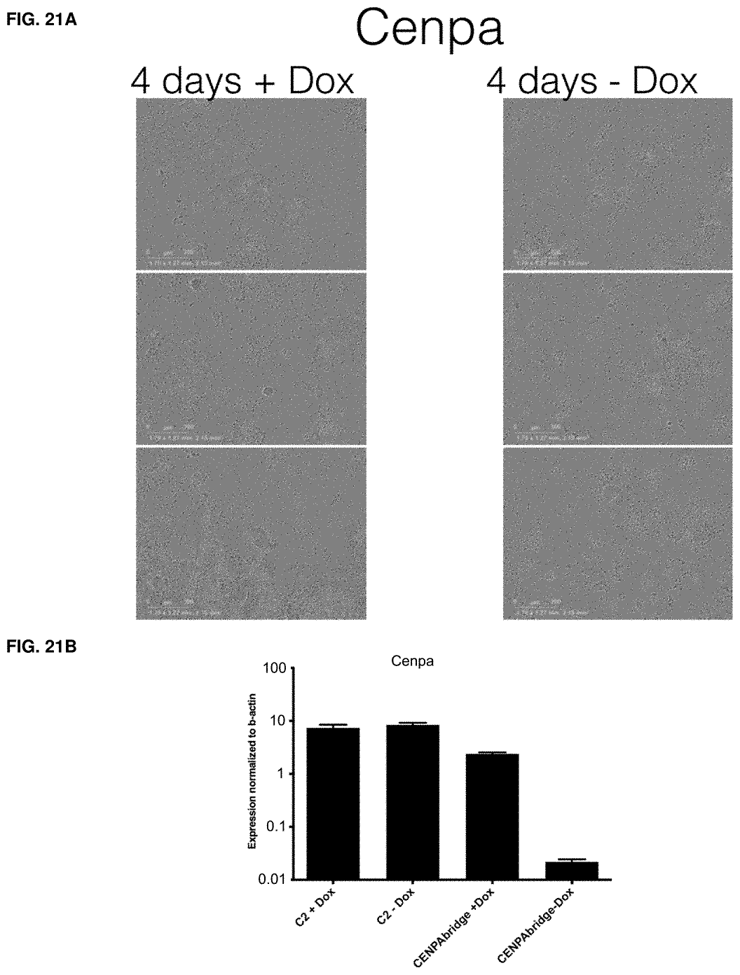

Withdrawal of doxycycline resulted in complete elimination of mitotically active ES cells within 4 days (FIG. 21A).

Referring to FIG. 21B, the inventors determined by qPCR the Cenpa gene expression level in Cenpa-EARC cells with Dox and after 2 days of Dox removal, and compared it to the expression level in wild type mouse ES cells (C2). As expected Cenpa expression level is greatly reduced in Cenpa-EARC cells without Dox for 2 days.

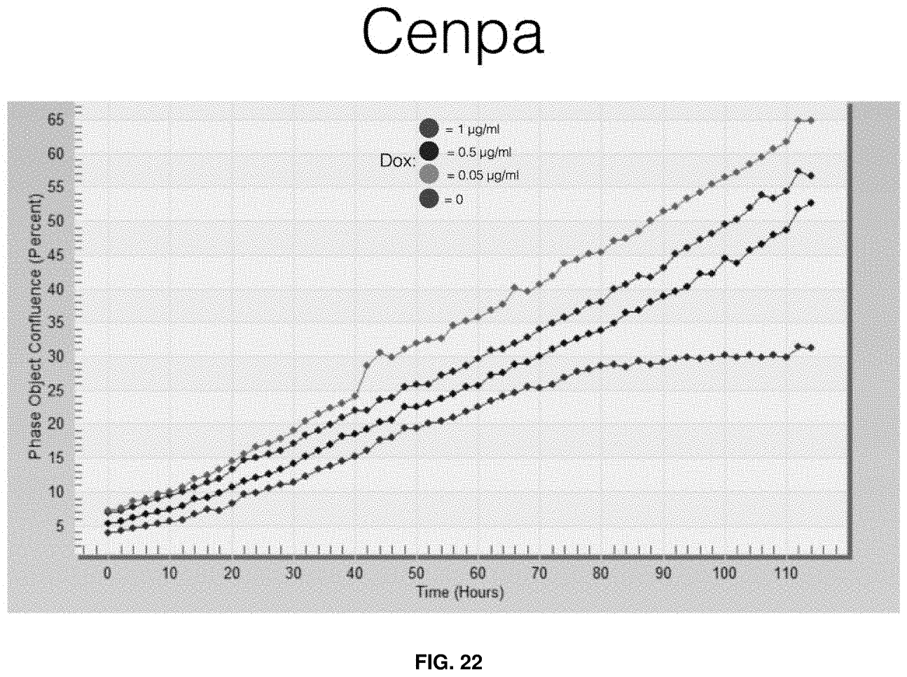

Referring to FIG. 22, the inventors determined how different concentrations of doxycycline affected proliferation of the dox-bridge ES cells by measuring cell growth for 4 days. ES cells in the presence of doxycycline grew exponentially, indicating their normal growth. In contrast, 80 hours after doxycycline removal, cells growth was completely arrested.

### Example 6: Generation of EARC-Modified Mouse ES Cells in the Birc5 Locus

In Example 6, construction of EARC (dox-bridge) vectors targeting Birc5 and use of same to control cell division in mouse ES cells is described. In this example, Birc5/BIRC5 is the CDL, which is targeted with an inducible gene expression system, wherein a dox-bridge is inserted and doxycycline induces expression of the CDL.

As described above, Birc5/BIRC5 is expressed in all mitotically active (i.e., dividing) cells. In cells modified to comprise an EARC (dox-bridge) insertion at the Birc5 locus, cell division is only possible in the presence of the inducer (doxycycline), which permits expression of Birc5. Thus, cell division of EARC-modified mitotically active cells can be eliminated in the absence of doxycycline.

In Example 6, dox-bridge insertion into the 5′UTR of the Birc5 gene was achieved by homologous recombination knock-in technology.

Methods

Construction of EARC Targeting Vector Comprising a Dox-Bridge

The 3′ homology arm (SEQ ID NO: 118) was cloned by PCR-amplifying a 775 bp fragment from C57/B6 genomic DNA (primers Birc3F (SEQ ID NO: 119), Birc3R (SEQ ID NO: 120)), and cloning it into SbfI and NcoI of the pGem-bridge-step3. Similarly, the 5′ homology arm (617 bp; SEQ ID NO: 121) was amplified by PCR using primers Birc5F (SEQ ID NO: 122) and Birc5R PstI (SEQ ID NO: 123) and SpeI and cloned into to generate a final targeting vector, referred to as pBridge-Birc5 (SEQ ID NO: 124).

Construction of CRISPR/Cas9 Plasmids

A double-nickase strategy was chosen to minimize the possibility of off-target mutations. Guide RNA sequences were cloned into pX335 (Addgene) using oligos Birc5AF (SEQ ID NO: 125), Birc5AR (SEQ ID NO: 126), Birc5BF (SEQ ID NO: 127), Birc5BR (SEQ ID NO: 128), according to the suggested protocol (Ran et al., 2013), generating the CRISPR vectors pX335-Birc5A (SEQ ID NO: 129) and px335-Birc5B (SEQ ID NO: 130).

Generation of EARC-Modified Mouse ES Cells

Mouse ES cell culture: As in Example 4.

Targeting: Plasmids containing the CRISPR/Cas9 components (pX335-Birc5A and px335-Birc5B) and the targeting plasmid (pBridge-Birc5) were co-transfected in mouse ES cells using FuGENE HD (Clontech), as in Example 4.

Genotyping: DNA was extracted as in Example 4. Clones positive for correct insertion by homologous recombination of pBridge-Birc5 in the 5′ of the Birc5 gene were screened by PCR using primers spanning the 5′ homology arm (primers rttaRev (SEQ ID NO: 54), Birc_5scrF (SEQ ID NO: 131)).

Targeted cell growth: Birc5 homozygously-targeted cells were trypsinized and plated on gelatinized 24 well plates at a density of 5×10⁴ cells per well. Starting one day after plating, cells were exposed to different Dox concentrations (1 μg/ml, 0.5 μg/ml, 0.05 μg/ml and 0 μg/ml), the plate was analyzed in a IncucyteZoom system (Essen Bioscience) by taking pictures every two hours for 3-4 days and measuring confluence.

Quantitative PCR: Total RNA was extracted from cells treated for 2 days with 1 μg/ml and 0 μg/ml of Dox using the GeneElute total RNA miniprep kit (Sigma) according to the manufacturer's protocol. cDNA was generated by reverse transcription of 1 μg of RNA using the QuantiTect reverse transcription kit (Qiagen), according to the manufacturer's protocol. Real-time qPCR were set up in a BioRad CFX thermocycler, using SensiFast-SYBR qPCR mix (Bioline). The primers used were: qperbirc_F (SEQ ID NO: 132), qperbirc_R (SEQ ID NO: 133) and actBf (SEQ ID NO: 67), actBr (SEQ ID NO: 68). Results were analyzed with the ΔΔCT method and normalized for beta-actin.

Results

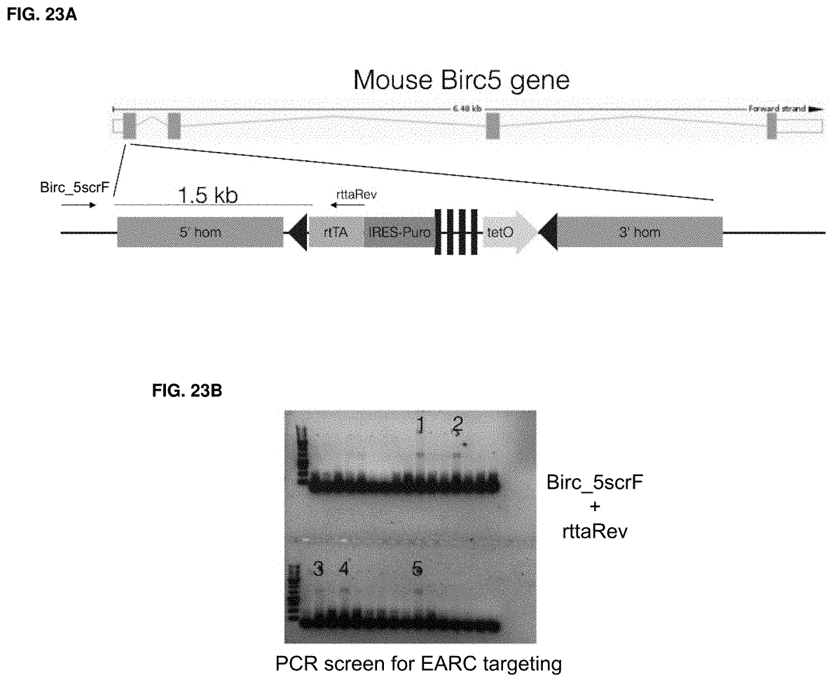

Referring to FIG. 23, the dox-bridge target vector, depicted in FIG. 23A, was used to generate targeted C2 mouse ES cell lines (FIG. 23B). Five clones were found to be correctly targeted (FIG. 23B) comprising a dox-bridge inserted by recombination into the 5′UTR of both alleles of Birc5. One of these clones was Birc #3, was found to stop growing or die in the absence of Dox.

As expected, this ES cell lines grows only in the presence of doxycycline. In the presence of doxycycline, the rtTA produced by Birc5 promoter, binds to TRE and initiates transcription of the Birc5 coding sequence. The dox-bridge may be inserted into the 5′UTR into both alleles of Birc5, to ensure that the CDL expression could occur only through EARC. An alternative is to generate null mutations in all the remaining, non-EARC modified alleles of CDL.

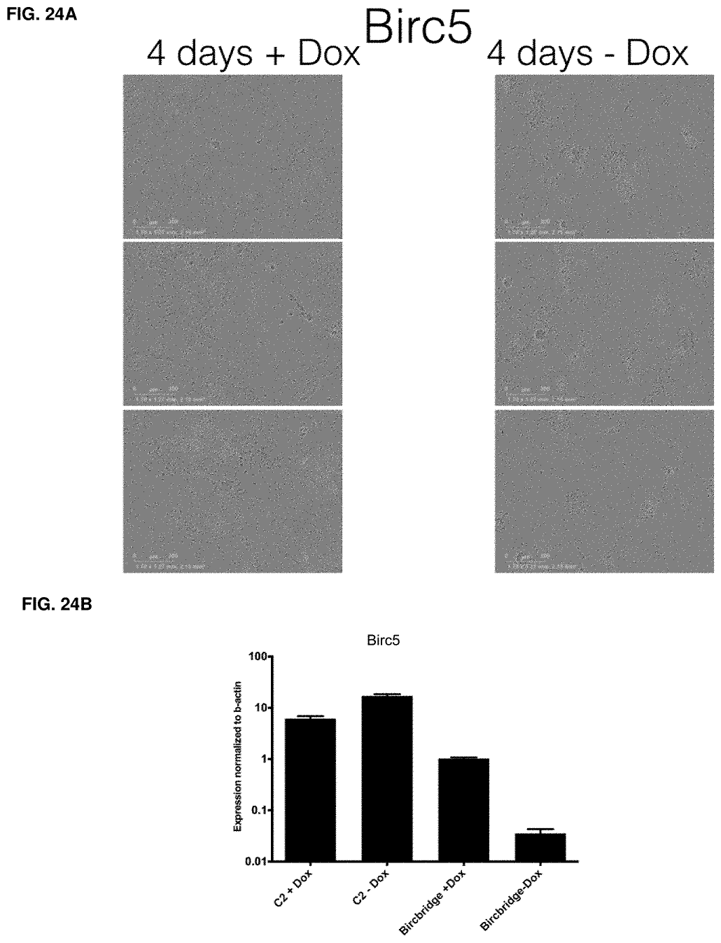

Withdrawal of doxycycline resulted in complete elimination of mitotically active ES cells within 4 days (FIG. 24A).

Referring to FIG. 24B, the inventors determined by qPCR the Birc5 gene expression level in Birc5-EARC cells with Dox and after 2 days of Dox removal, and compared it to the expression level in wild type mouse ES cells (C2). As expected Birc5 expression level is greatly reduced in Birc5-EARC cells without Dox for 2 days.

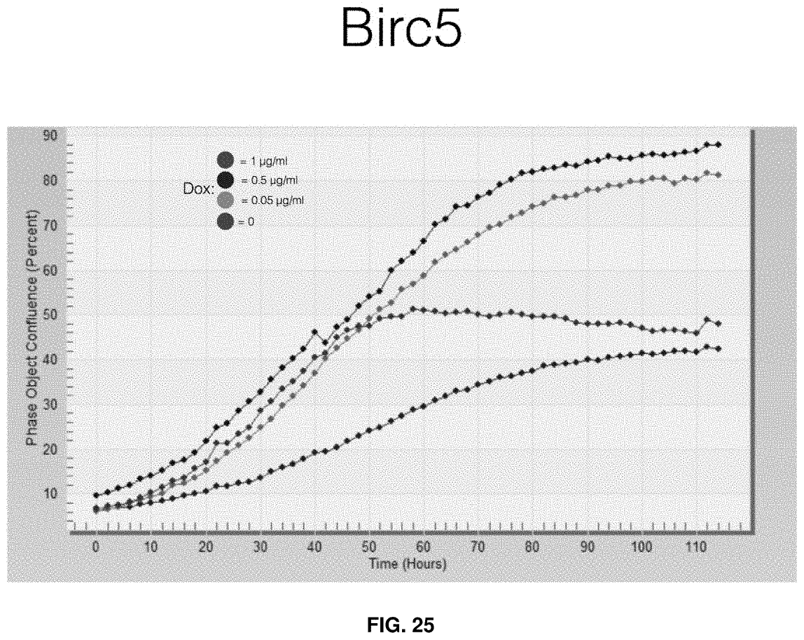

Referring to FIG. 25, the inventors determined how different concentrations of doxycycline affected proliferation of the dox-bridge ES cells by measuring cell growth for 4 days. ES cells in the presence of doxycycline grew exponentially, indicating their normal growth. In contrast, 50 hours after doxycycline removal, cells growth was completely arrested. Interestingly, it appears that lower Dox concentrations (0.5 and 0.05 μg/ml) promote better cell growth than a higher concentration (1 μg/ml).

### Example 7: Generation of EARC-Modified Mouse ES Cells in the Eef2 Locus

In Example 7, construction of EARC (dox-bridge) vectors targeting Eef2 and use of same to control cell division in mouse ES cells is described. In this example, Eef2/EEF2 is the CDL, which is targeted with an inducible gene expression system, wherein a dox-bridge is inserted and doxycycline induces expression of the CDL.

As described above, Eef2/EEF2 is expressed in all mitotically active (i.e., dividing) cells. In cells modified to comprise an EARC (dox-bridge) insertion at the Eef2 locus, cell division is only possible in the presence of the inducer (doxycycline), which permits expression of Eef2. Thus, cell division of EARC-modified mitotically active cells can be eliminated in the absence of doxycycline.

In Example 7, dox-bridge insertion into the 5′UTR of the Eef2 gene was achieved by homologous recombination knock-in technology.

Methods

Construction of EARC Targeting Vector Comprising a Dox-Bridge

The 5′ homology arm was cloned by PCR-amplifying a 817 bp fragment (SEQ ID NO: 134) from C57/B6 genomic DNA (primers Eef2_5F (SEQ ID NO: 135) and Eef2_5R (SEQ ID NO: 136) and cloning it into SbfI and SpeI of the pGem-bridge-step3. Similarly, the 3′ homology arm (826 bp; SEQ ID NO: 137) was amplified by PCR using primers Eef2_3F (SEQ ID NO: 138), Eef2_3R (SEQ ID NO: 139) and cloned into SphI to generate a final targeting vector, referred to as pBridge-Eef2 (SEQ ID NO: 140).

Construction of CRISPR/Cas9 Plasmids

A double-nickase strategy was chosen to minimize the possibility of off-target mutations. Guide RNA sequences were cloned into pX335 (Addgene) using oligos Eef2aFWD (SEQ ID NO: 141), Eef2aREV (SEQ ID NO: 142), Eef2bFWD (SEQ ID NO: 143), Eef2bREV (SEQ ID NO: 144), according to the suggested protocol (Ran et al., 2013), generating the CRISPR vectors pX335-Eef2A (SEQ ID NO: 145) and px335-Eef2B (SEQ ID NO: 146).

Generation of EARC-Modified Mouse ES Cells

Mouse ES cell culture: As in Example 4.

Targeting: Plasmids containing the CRISPR/Cas9 components (pX335-Eef2A and px335-Eef2B) and the targeting plasmid (pBridge-Eef2) were co-transfected in mouse ES cells using FuGENE HD (Clontech), as in Example 4.

Genotyping: DNA was extracted as in Example 4. Clones positive for correct insertion by homologous recombination of pBridge-Eef2 in the 5′ of the Eef2 gene were screened by PCR using primers spanning the 5′ homology arm (primers rttaRev (SEQ ID NO: 54), Eef2_5scrF (SEQ ID NO: 147)).

Targeted cell growth: Eef2 homozygously-targeted cells were trypsinized and plated on gelatinized 24 well plates at a density of 5×10⁴ cells per well. Starting one day after plating, cells were exposed to different Dox concentrations (1 μg/ml, 0.5 μg/ml, 0.05 μg/ml and 0 μg/ml), the plate was analyzed in a IncucyteZoom system (Essen Bioscience) by taking pictures every two hours for 3-4 days and measuring confluence.

Results

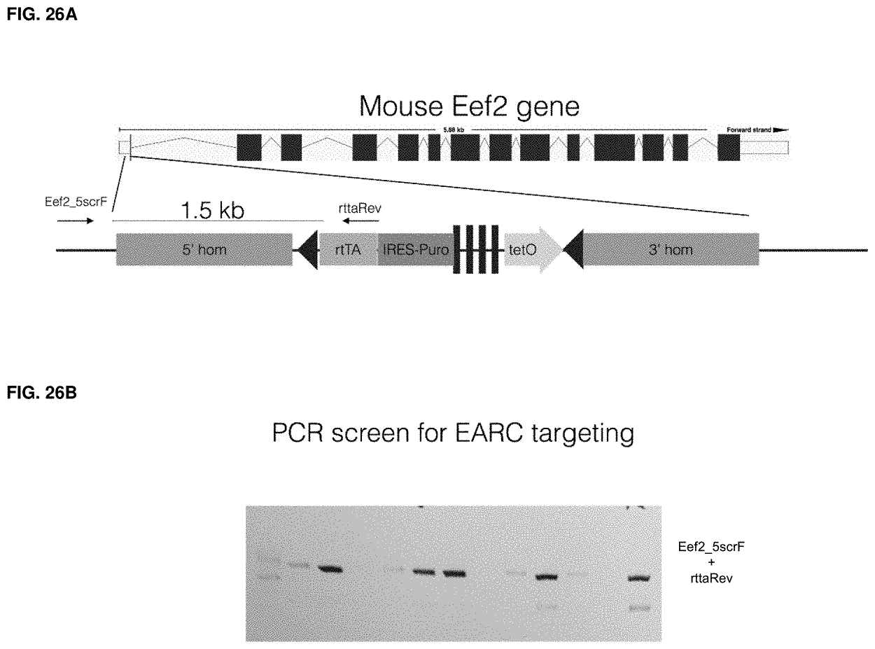

Referring to FIG. 26, the dox-bridge target vector, depicted in FIG. 26A, was used to generate several targeted C2 mouse ES cell lines (FIG. 26B). Nine of these cell lines was found to be correctly targeted (FIG. 26B) with at least one clone growing only in Dox-media.

As expected, this ES cell lines grows only in the presence of doxycycline. In the presence of doxycycline, the rtTA produced by Eef2 promoter, binds to TRE and initiates transcription of the Eef2 coding sequence. The dox-bridge may be inserted into the 5′UTR into both alleles of Eef2, to ensure that the CDL expression could occur only through EARC. An alternative is to generate null mutations in all the remaining, non-EARC modified alleles of CDL.

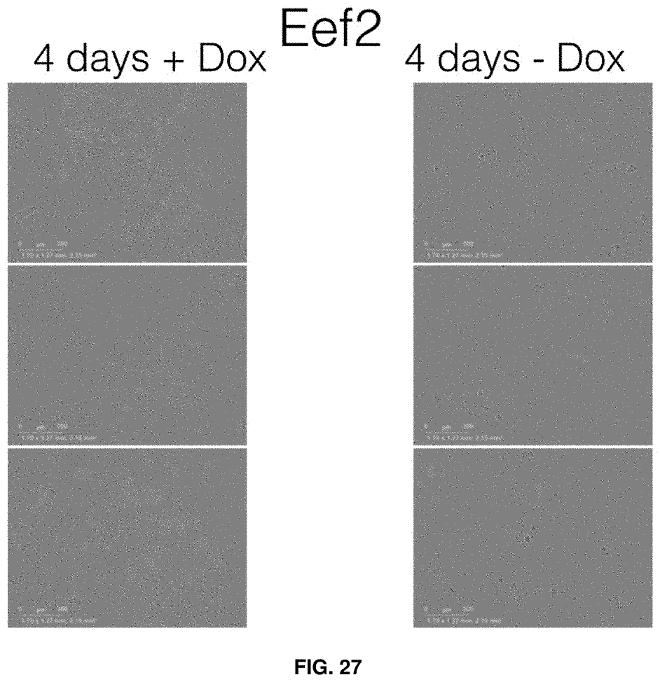

Withdrawal of doxycycline resulted in complete elimination of mitotically active ES cells within 4 days (FIG. 27).

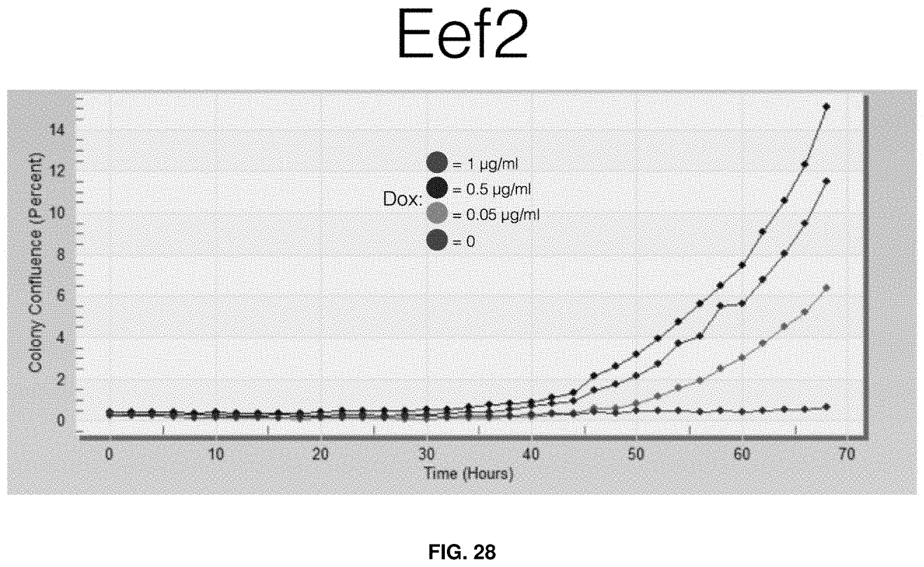

Referring to FIG. 28, the inventors determined how different concentrations of doxycycline affected proliferation of the dox-bridge ES cells by measuring cell growth for 4 days. ES cells in the presence of doxycycline grew exponentially, indicating their normal growth. In contrast, without doxycycline cells completely failed to grow.

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

TABLE 2
Predicted CDLs (ID refers to EntrezGene identification number; CS score refers to the CRISPR score average provided in Wang et al.,
2015; function refers to the known or predicted function the locus, of predictions being based on GO terms, as set forth in the
Gene Ontology Consortium website http://geneontology.org/; functional category refers to 4 categories of cell functions based on
the GO term-predicted function; CDL (basis) refers to information that the inventors used to predict that a gene is a CDL, predictions
being based on CS score, available gene knockout (KO) data, gene function, and experimental data provided herein).

| Name | ID | Name | ID | CS | Function | Functional | CDL | |
|---|---|---|---|---|---|---|---|---|
| (mouse) | (mouse) | (human) | (human) | score | (GO term) | category | (basis) | Citation |
| Actr8 | 56249 | ACTR8 | 93973 | −1.88 | chromatin | Cell cycle | CS score, | |
| | | | | | remodeling | | function | |
| Alg11 | 207958 | ALG11 | 440138 | −1.27 | dolichol- | Cell cycle | CS score, | |
| | | | | | linked | | | |
| | | | | | oligosaccharide | | function | |
| | | | | | biosynthetic process | | | |
| Anapc11 | 66156 | ANAPC11 | 51529 | −2.68 | protein ubiquitination | Cell cycle | CS score, | |
| | | | | | involved in ubiquitin- | | function | |
| | | | | | dependent protein | | | |
| | | | | | catabolic process | | | |
| Anapc2 | 99152 | ANAPC2 | 29882 | −2.88 | mitotic cell cycle | Cell cycle | CS score, | Wirth K G, et al. |
| | | | | | | | mouse | Genes Dev. 2004 |
| | | | | | | | K.O., | Jan. 1; 18(1): 88-98 |
| | | | | | | | function | |
| Anapc4 | 52206 | ANAPC4 | 29945 | −1.79 | regulation of mitotic | Cell cycle | CS score, | |
| | | | | | metaphase/anaphase | | function | |
| | | | | | transition | | | |
| Anapc5 | 59008 | ANAPC5 | 51433 | −1.66 | mitotic cell cycle | Cell cycle | CS score, | |
| | | | | | | | function | |
| Aurka | 20878 | AURKA | 6790 | −2.26 | meiotic spindle | Cell cycle | CS score, | Sasai K, et al. |
| | | | | | organization | | mouse | Oncogene. 2008 Jul. |
| | | | | | | | K.O., | 3; 27(29): 4122-7 |
| | | | | | | | function | |
| Banf1 | 23825 | BANF1 | 8815 | −2.14 | mitotic cell cycle | Cell cycle | CS score, | |
| | | | | | | | function | |
| Birc5 | 11799 | BIRC5 | 332 | −2.24 | regulation of signal | Cell cycle | CS score, | Uren A G et al. Curr |
| | | | | | transduction | | mouse | Biol. 2000 Nov. |
| | | | | | | | K.O., | 2; 10(21): 1319-28 |
| | | | | | | | function | |
| Bub3 | 12237 | BUB3 | 9184 | −3.15 | mitotic sister | Cell cycle | CS score, | Kalitsis F, et al. |
| | | | | | chromatid | | mouse | Genes Dev. 2000 |
| | | | | | segregation | | K.O., | Sep. |
| | | | | | | | function | 15; 14(18): 2277-82 |
| Casc5 | 76464 | CASC5 | 57082 | −1.16 | mitotic cell cycle | Cell cycle | CS score, | Overbeek P A, et al. |
| | | | | | | | mouse | MGI Direct Data |
| | | | | | | | K.O., | Submission. 2011 |
| | | | | | | | function | |
| Ccna2 | 12428 | CCNA2 | 890 | −1.59 | regulation of cyclin- | Cell cycle | CS score, | Kalaszczynska I, et |
| | | | | | dependent protein | | mouse | al. Cell. 2009 Jul. |
| | | | | | serine/threonine | | K.O., | 23; 138(2): 352-65 |
| | | | | | kinase activity | | function | |
| Ccnh | 66671 | CCNH | 902 | −2.01 | regulation of cyclin- | Cell cycle | CS score. | |
| | | | | | dependent protein | | function | |
| | | | | | serine/threonine | | | |
| | | | | | kinase activity | | | |
| Cdc123 | 98828 | CDC123 | 8872 | −2.45 | cell cycle | Cell cycle | CS score, | |
| | | | | | | | function | |
| Cdc16 | 69957 | CDC16 | 8881 | −3.58 | cell division | Cell cycle | CS score. | |
| | | | | | | | function | |
| Cdc20 | 107995 | CDC20 | 99 | −2.97 | mitotic cell cycle | Cell cycle | CS score, | Li M, et al. Mol Cell |
| | | | | | | | mouse | Biol. 2007 |
| | | | | | | | K.O., | May; 27(9): 3481-8 |
| | | | | | | | function | |
| Cdc23 | 52563 | CDC23 | 8697 | −2.28 | mitotic cell cycle | Cell cycle | CS score, | |
| | | | | | | | function | |
| Cdk1 | 12534 | CDK1 | 983 | −2.44 | cell cycle | Cell cycle | CS score, | Diril M K, et al. Proc |
| | | | | | | | mouse | Natl Acad Sci USA. |
| | | | | | | | K.O., | 2012 Mar. |
| | | | | | | | function | 6; 109(10): 3826-31 |
| Cenpa | 12615 | CENPA | 1058 | −1.87 | cell cycle | Cell cycle | CS score, | Howman E V, et al. |
| | | | | | | | mouse | Proc Natl Acad Sci |
| | | | | | | | K.O., | USA. 2000 Feb. |
| | | | | | | | function | 1; 97(3): 1148-53 |
| Cenpm | 66570 | CENPM | 79019 | −2.53 | mitotic cell cycle | Cell cycle | CS score, | |
| | | | | | | | function | |
| Chek1 | 12649 | CHEK1 | 1111 | −1.67 | protein | Cell cycle | CS score, | Takai H, et al. |
| | | | | | phosphorylation | | mouse | Genes Dev. 2000 |
| | | | | | | | K.O., | Jun. 15; 14(12): 1439- |
| | | | | | | | function | 47 |
| Chmp2a | 68953 | CHMP2A | 27243 | −2.40 | vacuolar transport | Cell cycle | CS score, | |
| | | | | | | | function | |
| Ckap5 | 75786 | CKAP5 | 9793 | −2.94 | G2/M transition of | Cell cycle | CS score, | Barbarese E, et al. |
| | | | | | mitotic cell cycle | | mouse | PLoS One. |
| | | | | | | | K.O., | 2013; 8(8): e69989 |
| | | | | | | | function | |
| Cltc | 67300 | CLTC | 1213 | −1.75 | intracellular protein | Cell cycle | CS score, | |
| | | | | | transport | | function | |
| Cops5 | 26754 | COPS5 | 10987 | −1.75 | protein deneddylation | Cell cycle | CS score, | Tian L, et al. |
| | | | | | | | mouse | Oncogene. 2010 |
| | | | | | | | K.O., | Nov. |
| | | | | | | | function | 18; 29(46): 6125-37 |
| Dctn2 | 69654 | DCTN2 | 10540 | −1.48 | G2/M transition of | Cell cycle | CS score, | |
| | | | | | mitotic cell cycle | | function | |
| Dctn3 | 53598 | DCTN3 | 11258 | −1.77 | G2/M transition of | Cell cycle | CS score, | |
| | | | | | mitotic cell cycle | | function | |
| Dhfr | 13361 | DHFR | 1719 | −2.84 | G1/S transition of | Cell cycle | CS score, | |
| | | | | | mitotic cell cycle | | function | |
| Dtl | 76843 | DTL | 51514 | 2.69 | protein | Cell cycle | CS score, | Liu C L, et al. J Biol |
| | | | | | polyubiquitination | | mouse | Chem. 2007 Jan. |
| | | | | | | | K.O., | 12; 282(2): 1109-18 |
| | | | | | | | function | |
| Dync1h1 | 13424 | DYNC1H1 | 1778 | −3.44 | G2/M transition of | Cell cycle | CS score, | Harada A, et al. J |
| | | | | | mitotic cell cycle | | mouse | Cell Biol. 1998 Apr. |
| | | | | | | | K.O., | 6; 141(1): 51-9 |
| | | | | | | | function | |
| Ecd | 70601 | ECD | 11319 | −3.18 | regulation of | Cell cycle | CS score, | |
| | | | | | glycolytic process | | function | |
| Ect2 | 13605 | ECT2 | 1894 | −1.80 | cell morphogenesis | Cell cycle | CS score, | Hansen J, et al. |
| | | | | | | | mouse | Proc Natl Acad Sci |
| | | | | | | | K.O., | USA. 2003 Aug. |
| | | | | | | | function | 19; 100(17): 9918-22 |
| Ep300 | 328572 | EP300 | 2033 | −2.04 | G2/M transition of | Cell cycle | CS score, | Yao T P, et al. Cell. |
| | | | | | mitotic cell cycle | | mouse | 1998 May |
| | | | | | | | K.O., | 1; 93(3): 361-72 |
| | | | | | | | function | |
| Ercc3 | 13872 | ERCC3 | 2071 | −2.10 | nucleotide- | Cell cycle | CS score, | Andressoo J O, et |
| | | | | | excision repair | | mouse | al. Mol Cell Biol. |
| | | | | | | | K.O., | 2009 |
| | | | | | | | function | March; 29(5): 1276-90 |
| Espl1 | 105988 | ESPL1 | 9700 | −3.24 | proteolysis | Cell cycle | CS score, | Wirth K G et al. J |
| | | | | | | | mouse | Cell Biol. 2006 Mar. |
| | | | | | | | K.O., | 13; 172(6): 847-60 |
| | | | | | | | function | |
| Fntb | 110606 | FNTB | 2342 | −2.42 | phototransduction, | Cell cycle | CS score, | Mijimolle N, et al. |
| | | | | | visible light | | mouse | Cancer Cell. 2005 |
| | | | | | | | K.O., | April; 7(4): 313-24 |
| | | | | | | | function | |
| Gadd45gip1 | 102060 | GADD45GIP1 | 90480 | −1.81 | organelle | Cell cycle | CS score, | Kwon M C, et al. |
| | | | | | organization | | mouse | EMBO J. 2008 Feb. |
| | | | | | | | K.O., | 20; 27(4): 642-53 |
| | | | | | | | function | |
| Gins1 | 69270 | GINS1 | 9837 | −1.84 | mitotic cell cycle | Cell cycle | CS score, | Ueno M, et al. Mol |
| | | | | | | | mouse | Cell Biol. 2005 |
| | | | | | | | K.O., | December; 25(23): 10528- |
| | | | | | | | function | 32 |
| Gnb2l1 | 14694 | GNB2L1 | 10399 | −2.84 | osteoblast | Cell cycle | CS score, | |
| | | | | | differentiation | | function | |
| Gspt1 | 14852 | GSPT1 | 2935 | −1.77 | G1/S transition of | Cell cycle | CS score, | |
| | | | | | mitotic cell cycle | | function | |
| Haus1 | 225745 | HAUS1 | 115106 | −1.92 | spindle assembly | Cell cycle | CS score, | |
| | | | | | | | function | |
| Haus3 | 231123 | HAUS3 | 79441 | −1.38 | mitotic nuclear | Cell cycle | CS score, | |
| | | | | | division | | function | |
| Haus5 | 71909 | HAUS5 | 23354 | −2.55 | spindle assembly | Cell cycle | CS score, | |
| | | | | | | | function | |
| Haus8 | 76478 | HAUS8 | 93323 | −1.73 | mitotic nuclear | Cell cycle | CS score, | |
| | | | | | division | | function | |
| Hdac3 | 15183 | HDAC3 | 8841 | −2.12 | histone deacetylation | Cell cycle | CS score, | Bhaskara S, et al. |
| | | | | | | | mouse | Mol Cell. 2008 Apr. |
| | | | | | | | K.O., | 11; 30(1): 61-72 |
| | | | | | | | function | |
| Kif11 | 16551 | KIF11 | 3832 | −3.23 | microtubule- | Cell cycle | CS score, | Castillo A, et al. |
| | | | | | based movement | | mouse | Biochem Biophys |
| | | | | | | | K.O., | Res Commun. 2007 |
| | | | | | | | function | Jun. 8; 357(3): 694-9 |
| Kif23 | 71819 | KIF23 | 9493 | −1.59 | microtubule- | Cell cycle | CS score, | |
| | | | | | based movement | | function | |
| Kpnb1 | 16211 | KPNB1 | 3837 | −3.19 | nucleocytoplasmic | Cell cycle | CS score, | Miura K, et al. |
| | | | | | transport | | mouse | Biochem Biophys |
| | | | | | | | K.O., | Res Commun. 2006 |
| | | | | | | | function | Mar. 3; 341(1): 132-8 |
| Mastl | 67121 | MASTL | 84930 | −2.36 | protein | Cell cycle | CS score, | Alvarez-Fernandez |
| | | | | | phosphorylation | | mouse | M, et al. Proc Natl |
| | | | | | | | K.O., | Acad Sci USA. |
| | | | | | | | function | 2013 Oct. |
| | | | | | | | | 22; 110(43): 17374-9 |
| Mau2 | 74549 | MAU2 | 23383 | −2.71 | mitotic cell cycle | Cell cycle | CS score, | Smith T G, et al. |
| | | | | | | | mouse | Genesis. 2014 |
| | | | | | | | K.O., | July; 52(7): 687-94 |
| | | | | | | | function | |
| Mcm3 | 17215 | MCM3 | 4172 | −2.52 | G1/S transition of | Cell cycle | CS score, | |
| | | | | | mitotic cell cycle | | function | |
| Mcm4 | 17217 | MCM4 | 4173 | −1.87 | G1/S transition of | Cell cycle | CS score, | Shima N, et al. Nat |
| | | | | | mitotic cell cycle | | mouse | Genet. 2007 |
| | | | | | | | K.O., | January; 39(1): 93-8 |
| | | | | | | | function | |
| Mcm7 | 17220 | MCM7 | 4176 | −2.39 | G1/S transition of | Cell cycle | CS score, | |
| | | | | | mitotic cell cycle | | function | |
| Mnat1 | 17420 | MNAT1 | 4331 | −1.22 | regulation of cyclin- | Cell cycle | CS score, | Rossi D J. et al. |
| | | | | | dependent protein | | mouse | EMBO J. 2001 Jun. |
| | | | | | serine/threonine | | K.O., | 1; 20(11): 2844-56 |
| | | | | | kinase activity | | function | |
| Mybbp1a | 18432 | MYBBP1A | 10514 | −2.17 | osteoblast | Cell cycle | CS score, | Mori S, et al. PLoS |
| | | | | | differentiation | | mouse | One. |
| | | | | | | | K.O., | 2012; 7(10): e39723 |
| | | | | | | | function | |
| Ncapd2 | 68298 | NCAPD2 | 9918 | −2.03 | mitotic chromosome | Cell cycle | CS score, | |
| | | | | | condensation | | function | |
| Ncaph | 215387 | NCAPH | 23397 | −2.33 | mitotic chromosome | Cell cycle | CS score, | Nishide K, et al. |
| | | | | | condensation | | mouse | PLoS Genet. 2014 |
| | | | | | | | K.O., | December; 10(12): |
| | | | | | | | function | e1004847 |
| Ndc80 | 67052 | NDC80 | 10403 | −2.98 | attachment of mitotic | Cell cycle | CS score, | |
| | | | | | spindle microtubules | | function | |
| | | | | | to kinetochore | | | |
| Nle1 | 217011 | NLE1 | 54475 | −1.88 | somitogenesis | Cell cycle | CS score, | Hentges K E, et al. |
| | | | | | | | mouse | Gene Exor |
| | | | | | | | K.O., | Patterns. 2006 |
| | | | | | | | function | August; 6(6): 653-65 |
| Nsl1 | 381318 | NSL1 | 25936 | −1.90 | mitotic cell cycle | Cell cycle | CS score, | |
| | | | | | | | function | |
| Nudc | 18221 | NUDC | 10726 | −1.93 | mitotic cell cycle | Cell cycle | CS score, | |
| | | | | | | | function | |
| Nuf2 | 66977 | NUF2 | 83540 | −1.78 | mitotic nuclear | Cell cycle | CS score, | |
| | | | | | division | | function | |
| Nup133 | 234865 | NUP133 | 55746 | −2.26 | mitotic cell cycle | Cell cycle | CS score, | Garcia-Garcia M J, |
| | | | | | | | mouse | et al. Proc Natl |
| | | | | | | | K.O., | Acad Sci USA. |
| | | | | | | | function | 2005 Apr. |
| | | | | | | | | 26; 102(17): 5913-9 |
| Nup160 | 59015 | NUP160 | 23279 | −2.64 | mitotic cell cycle | Cell cycle | CS score, | |
| | | | | | | | function | |
| Nup188 | 227699 | NUP188 | 23511 | −1.16 | mitotic cell cycle | Cell cycle | CS score, | |
| | | | | | | | function | |
| Nup214 | 227720 | NUP214 | 8021 | −2.70 | mitotic cell cycle | Cell cycle | CS score, | van Deursen J, et |
| | | | | | | | mouse | al. EMBO J. 1996 |
| | | | | | | | K.O., | Oct. 15; 15(20): 5574- |
| | | | | | | | function | 83 |
| n/a | n/a | NUP62 | 23636 | −2.35 | mitotic cell cycle | Cell cycle | CS score, | |
| | | | | | | | function | |
| Nup85 | 445007 | NUP85 | 79902 | −2.47 | mitotic cell cycle | Cell cycle | CS score, | |
| | | | | | | | function | |
| Orc3 | 50793 | ORC3 | 23595 | −1.67 | G1/S transition of | Cell cycle | CS score, | |
| | | | | | mitotic cell cycle | | function | |
| Pafah1b1 | 18472 | PAFAH1B1 | 5048 | −2.34 | G2/M transition of | Cell cycle | CS score, | Cahana A, et al. |
| | | | | | mitotic cell cycle | | mouse | Proc Natl Acad Sci |
| | | | | | | | K.O., | USA. 2001 May |
| | | | | | | | function | 22; 98(11): 6429-34 |
| Pcid2 | 234069 | PCID2 | 55795 | −1.98 | negative regulation of | Cell cycle | CS score, | |
| | | | | | apoptotic process | | function | |
| Pfas | 237823 | PFAS | 5198 | −2.58 | purine nucleotide | Cell cycle | CS score, | |
| | | | | | biosynthetic process | | function | |
| Phb2 | 12034 | PHB2 | 11331 | −2.98 | protein import into | Cell cycle | CS score, | Park S E, et al. Mol |
| | | | | | nucleus, | | mouse | Cell Biol. 2005 |
| | | | | | translocation | | K.O., | March; 25(5): 1989-99 |
| | | | | | | | function | |
| Pkmyt1 | 268930 | PKMYT1 | 9088 | −1.93 | regulation of cyclin- | Cell cycle | CS score, | |
| | | | | | dependent protein | | function | |
| | | | | | serine/threonine | | | |
| | | | | | kinase activity | | | |
| Plk1 | 18817 | PLK1 | 5347 | −2.83 | protein | Cell cycle | CS score, | Lu L Y, et al. Mol |
| | | | | | phosphorylation | | mouse | Cell Biol. 2008 |
| | | | | | | | K.O., | November; 28(22): 6870- |
| | | | | | | | function | 6 |
| Pmf1 | 67037 | PMF1 | 11243 | −2.15 | mitotic cell cycle | Cell cycle | CS score, | |
| | | | | | | | function | |
| Pole2 | 18974 | POLE2 | 5427 | −3.08 | G1/S transition of | Cell cycle | CS score, | |
| | | | | | mitotic cell cycle | | function | |
| Ppat | 231327 | PPAT | 5471 | −2.15 | G1/S transition of | Cell cycle | CS score, | |
| | | | | | mitotic cell cycle | | function | |
| Psma6 | 26443 | PSMA6 | 5687 | −3.51 | G1/S transition of | Cell cycle | CS score, | |
| | | | | | mitotic cell cycle | | function | |
| Psma7 | 26444 | PSMA7 | 5688 | −2.91 | G1/S transition of | Cell cycle | CS score, | |
| | | | | | mitotic cell cycle | | function | |
| Psmb1 | 19170 | PSMB1 | 5689 | −1.63 | G1/S transition of | Cell cycle | CS score, | |
| | | | | | mitotic cell cycle | | function | |
| Psmb4 | 19172 | PSMB4 | 5692 | −2.91 | G1/S transition of | Cell cycle | CS score, | |
| | | | | | mitotic cell cycle | | function | |
| Psmd12 | 66997 | PSMD12 | 5718 | −1.69 | G1/S transition of | Cell cycle | CS score, | |
| | | | | | mitotic cell cycle | | function | |
| Psmd13 | 23997 | PSMD13 | 5719 | −1.57 | G1/S transition of | Cell cycle | CS score, | |
| | | | | | mitotic cell cycle | | function | |
| Psmd14 | 59029 | PSMD14 | 10213 | −3.01 | G1/S transition of | Cell cycle | CS score, | |
| | | | | | mitotic cell cycle | | function | |
| Psmd7 | 17463 | PSMD7 | 5713 | −2.18 | G1/S transition of | Cell cycle | CS score, | Soriano P, et al. |
| | | | | | mitotic cell cycle | | mouse | Genes Dev. 1987 |
| | | | | | | | K.O., | June; 1(4): 366-75 |
| | | | | | | | function | |
| Racgap1 | 26934 | RACGAP1 | 29127 | −1.94 | mitotic spindle | Cell cycle | CS score, | Van de Putte T, et |
| | | | | | assembly | | mouse | al. Mech Dev. 2001 |
| | | | | | | | K.O., | April; 102(1-2): 33-44 |
| | | | | | | | function | |
| Rad21 | 19357 | RAD21 | 5885 | −2.12 | mitotic cell cycle | Cell cycle | CS score, | |
| | | | | | | | function | |
| Rae1 | 66679 | RAE1 | 8480 | −2.15 | mitotic cell cycle | Cell cycle | CS score, | Babu J R. et al. J |
| | | | | | | | mouse | Cell Biol. 2003 Feb. |
| | | | | | | | K.O., | 3; 160(3): 341-53 |
| | | | | | | | function | |
| Rcc1 | 100088 | RCC1 | 1104 | −2.91 | G1/S transition of | Cell cycle | CS score, | |
| | | | | | mitotic cell cycle | | function | |
| Rfc3 | 69263 | RFC3 | 5983 | −2.74 | mitotic cell cycle | Cell cycle | CS score, | |
| | | | | | | | function | |
| Rps27a | 78294 | RPS27A | 6233 | −2.74 | G1/S transition of | Cell cycle | CS score, | |
| | | | | | mitotic cell cycle | | function | |
| Rrm2 | 20135 | RRM2 | 6241 | −3.09 | G1/S transition of | Cell cycle | CS score, | |
| | | | | | mitotic cell cycle | | function | |
| Sae1 | 56459 | SAE1 | 10055 | −2.08 | cellular protein | Cell cycle | CS score, | |
| | | | | | modification process | | function | |
| Sec13 | 110379 | SEC13 | 6396 | −2.96 | mitotic cell cycle | Cell cycle | CS score, | |
| | | | | | | | function | |
| Smarcb1 | 20587 | SMARCB1 | 6598 | −1.98 | chromatin | Cell cycle | CS score, | Guidi C J, et al. Mol |
| | | | | | remodeling | | mouse | Cell Biol. 2001 May |
| | | | | | | | K.O., | 15; 21(10): 3598-603 |
| | | | | | | | function | |
| Smc2 | 14211 | SMC2 | 10592 | −2.13 | mitotic chromosome | Cell cycle | CS score, | Nishide K, et al. |
| | | | | | condensation | | mouse | PLoS Genet. 2014 |
| | | | | | | | K.O., | December; 10(12): e10048 |
| | | | | | | | function | 47 |
| Smc4 | 70099 | SMC2 | 10051 | −1.47 | chromosome | Cell cycle | CS score, | |
| | | | | | organization | | function | |
| Son | 20658 | SON | 6651 | −1.99 | microtubule | Cell cycle | CS score, | |
| | | | | | cytoskeleton | | function | |
| | | | | | organization | | | |
| Spc24 | 67629 | SPC24 | 147841 | −2.83 | mitotic cell cycle | Cell cycle | CS score, | |
| | | | | | | | function | |
| Spc25 | 66442 | SPC25 | 57405 | −1.63 | mitotic cell cycle | Cell cycle | CS score, | |
| | | | | | | | function | |
| Terf2 | 21750 | TERF2 | 7014 | −2.17 | telomere | Cell cycle | CS score, | Celli G B, et al. Nat |
| | | | | | maintenance | | mouse | Cell Biol. 2005 |
| | | | | | | | K.O., | July; 7(7): 712-8 |
| | | | | | | | function | |
| Tpx2 | 72119 | TPX2 | 22974 | −2.08 | apoptotic process | Cell cycle | CS score, | Aguirre-Portoles C, |
| | | | | | | | mouse | et al. Cancer Res. |
| | | | | | | | K.O., | 2012 Mar. |
| | | | | | | | function | 15; 72(6): 1518-28 |
| Tubg1 | 103733 | TUBG1 | 7283 | −2.08 | microtubule | Cell cycle | CS score, | Yuba-Kubo A, et al. |
| | | | | | nucleation | | mouse | Dev Biol. 2005 Jun. |
| | | | | | | | K.O., | 15; 282(2): 361-73 |
| | | | | | | | function | |
| Tubgcp2 | 74237 | TUBGCP2 | 10844 | −2.78 | microtubule | Cell cycle | CS score, | |
| | | | | | cytoskeleton | | function | |
| | | | | | organization | | | |
| Tubgcp5 | 233276 | TUBGCP5 | 114791 | −1.76 | microtubule | Cell cycle | CS score, | |
| | | | | | cytoskeleton | | function | |
| | | | | | organization | | | |
| Tubgcp6 | 328580 | TUBGCP6 | 85378 | −1.52 | microtubule | Cell cycle | CS score, | |
| | | | | | cytoskeleton | | function | |
| | | | | | organization | | | |
| Txnl4a | 27366 | TXNL4A | 10907 | −3.89 | mitotic nuclear | Cell cycle | CS score, | |
| | | | | | division | | function | |
| Usp39 | 28035 | USP39 | 10713 | −2.85 | spliceosomal | Cell cycle | CS score, | |
| | | | | | complex assembly | | function | |
| Wdr43 | 72515 | WDR43 | 23160 | −3.02 | reproduction | Cell cycle | CS score, | |
| | | | | | | | function | |
| Zfp830 | 66983 | ZNF830 | 91603 | −1.52 | blastocyst growth | Cell cycle | CS score, | Houlard M, et al. |
| | | | | | | | mouse | Cell Cycle. 2011 |
| | | | | | | | K.O., | Jan. 1; 10(1): 108-17 |
| | | | | | | | function | |
| Aatf | 56321 | AATF | 26574 | −1.46 | cellular response to | DNA | CS score, | Thomas T, et al. |
| | | | | | DNA damage | replication, | mouse | Dev Biol. 2000 Nov. |
| | | | | | stimulus | DNA repair | K.O., | 15; 227(2): 324-42 |
| | | | | | | | function | |
| Alyref | 21681 | ALYREF | 10189 | −1.92 | regulation of DNA | DNA | CS score, | |
| | | | | | recombination | replication, | function | |
| | | | | | | DNA repair | | |
| Brf2 | 66653 | BRF2 | 55290 | −2.30 | DNA- | DNA | CS score, | |
| | | | | | templated transcription, | replication, | function | |
| | | | | | initiation | DNA repair | | |
| Cdc45 | 12544 | CDC45 | 8318 | −3.69 | DNA replication | DNA | CS score, | Yoshida K, et al. |
| | | | | | checkpoint | replication, | mouse | Mol Cell Biol. 2001 |
| | | | | | | DNA repair | K.O., | July; 21(14): 4598-603 |
| | | | | | | | function | |
| Cdc6 | 23834 | CDC6 | 990 | −1.87 | DNA replication | DNA | CS score, | |
| | | | | | initiation | replication, | function | |
| | | | | | | DNA repair | | |
| Cdt1 | 67177 | CDT1 | 81620 | −2.74 | DNA replication | DNA | CS score, | |
| | | | | | initiation | replication, | function | |
| | | | | | | DNA repair | | |
| Cinp | 67236 | CINP | 51550 | −1.64 | DNA replication | DNA | CS score, | |
| | | | | | | replication, | function | |
| | | | | | | DNA repair | | |
| Cirh1a | 21771 | CIRH1A | 84916 | −2.62 | transcription, DNA- | DNA | CS score, | |
| | | | | | templated | replication, | function | |
| | | | | | | DNA repair | | |
| Ddb1 | 13194 | DDB1 | 1642 | −2.14 | nucleotide- | DNA | CS score, | Cang Y, et al. Cell. |
| | | | | | excision repair, DNA | replication, | mouse | 2006 Dec. |
| | | | | | damage removal | DNA repair | K.O., | 1; 127(5): 929-40 |
| | | | | | | | function | |
| Ercc2 | 13871 | ERCC2 | 2068 | −2.80 | DNA duplex | DNA | CS score, | de Boer J, et al. |
| | | | | | unwinding | replication, | mouse | Cancer Res. 1998 |
| | | | | | | DNA repair | K.O., | Jan. 1; 58(1): 89-94 |
| | | | | | | | function | |
| Gabpb1 | 14391 | GABPB1 | 2553 | −1.74 | transcription, DNA- | DNA | CS score, | Xue H H, et al. Mol |
| | | | | | templated | replication, | mouse | Cell Biol. 2008 |
| | | | | | | DNA repair | K.O., | July; 28(13): 4300-9 |
| | | | | | | | function | |
| Gtf2b | 229906 | GTF2B | 2959 | −2.76 | regulation of | DNA | CS score, | |
| | | | | | transcription, DNA- | replication, | function | |
| | | | | | templated | DNA repair | | |
| Gtf2h4 | 14885 | GTF2H4 | 2968 | −1.93 | nucleotide- | DNA | CS score, | |
| | | | | | excision repair, DNA | replication, | function | |
| | | | | | damage removal | DNA repair | | |
| Gtf3a | 66596 | GTF3A | 2971 | −2.25 | regulation of | DNA | CS score, | |
| | | | | | transcription, DNA- | replication, | function | |
| | | | | | templated | DNA repair | | |
| Gtf3c1 | 233863 | GTF3C1 | 2975 | −2.45 | transcription, DNA- | DNA | CS score, | |
| | | | | | templated | replication, | function | |
| | | | | | | DNA repair | | |
| Gtf3c2 | 71752 | GTF3C2 | 2976 | −2.09 | transcription, DNA- | DNA | CS score, | |
| | | | | | templated | replication, | function | |
| | | | | | | DNA repair | | |
| Hinfp | 102423 | HINFP | 25988 | −2.35 | DNA damage | DNA | CS score, | Xie R, et al. Proc |
| | | | | | checkpoint | replication, | mouse | Natl Acad Sci USA. |
| | | | | | | DNA repair | K.O., | 2009 Jul. 9 |
| | | | | | | | function | |
| n/a | n/a | HIST2H2AA3 | 8337 | −1.71 | DNA repair | DNA | CS score, | |
| | | | | | | replication, | function | |
| | | | | | | DNA repair | | |
| Ints3 | 229543 | INTS3 | 65123 | −3.14 | DNA repair | DNA | CS score, | |
| | | | | | | replication, | function | |
| | | | | | | DNA repair | | |
| Kin | 16588 | KIN | 22944 | −1.99 | DNA replication | DNA | CS score, | |
| | | | | | | replication, | function | |
| | | | | | | DNA repair | | |
| Mcm2 | 17216 | MCM2 | 4171 | −2.86 | DNA replication | DNA | CS score, | |
| | | | | | initiation | replication, | function | |
| | | | | | | DNA repair | | |
| Mcm6 | 17219 | MCM6 | 4175 | −1.55 | DNA replication | DNA | CS score, | |
| | | | | | | replication, | function | |
| | | | | | | DNA repair | | |
| Mcrs1 | 51812 | MCRS1 | 10445 | −1.23 | DNA repair | DNA | CS score, | |
| | | | | | | replication, | function | |
| | | | | | | DNA repair | | |
| Med11 | 66172 | MED11 | 400569 | −2.39 | transcription, DNA- | DNA | CS score, | |
| | | | | | templated | replication, | function | |
| | | | | | | DNA repair | | |
| Mtpap | 67440 | MTPAP | 55149 | −1.86 | transcription, DNA- | DNA | CS score, | |
| | | | | | templated | replication, | function | |
| | | | | | | DNA repair | | |
| Myc | 17869 | MYC | 4609 | −2.49 | regulation of | DNA | CS score, | Trumpp A, et al. |
| | | | | | transcription, DNA- | replication, | mouse | Nature. 2001 Dec. |
| | | | | | templated | DNA repair | K.O., | 13; 414(6865): 768- |
| | | | | | | | function | 73 |
| Ndnl2 | 66647 | NDNL2 | 56160 | −2.03 | DNA repair | DNA | CS score, | |
| | | | | | | replication, | function | |
| | | | | | | DNA repair | | |
| Nol11 | 68979 | NOL11 | 25926 | −1.59 | transcription, DNA- | DNA | CS score, | |
| | | | | | templated | replication, | function | |
| | | | | | | DNA repair | | |
| Nol8 | 70930 | NOL8 | 55035 | −1.35 | DNA replication | DNA | CS score, | |
| | | | | | | replication, | function | |
| | | | | | | DNA repair | | |
| Pcna | 18538 | PCNA | 5111 | −3.60 | DNA replication | DNA | CS score, | Roa S, et al. Proc |
| | | | | | | replication, | mouse | Natl Acad Sci USA. |
| | | | | | | DNA repair | K.O., | 2008 Oct. 21; |
| | | | | | | | function | 105(42): 16248-53 |
| Pola1 | 18968 | POLA1 | 5422 | −2.28 | DNA- | DNA | CS score, | |
| | | | | | dependent DNA | replication, | function | |
| | | | | | replication | DNA repair | | |
| Pold2 | 18972 | POLD2 | 5425 | −2.51 | DNA replication | DNA | CS score, | |
| | | | | | | replication, | function | |
| | | | | | | DNA repair | | |
| Pole | 18973 | POLE | 5426 | −2.90 | DNA replication | DNA | CS score, | |
| | | | | | | replication, | function | |
| | | | | | | DNA repair | | |
| Polr1a | 20019 | POLR1A | 25885 | −2.62 | transcription, DNA- | DNA | CS score, | |
| | | | | | templated | replication, | function | |
| | | | | | | DNA repair | | |
| n/a | n/a | POLR2J2 | 246721 | −3.08 | transcription, DNA- | DNA | CS score, | |
| | | | | | templated | replication, | function | |
| | | | | | | DNA repair | | |
| Polr3a | 218832 | POLR3A | 11128 | −2.43 | transcription, DNA- | DNA | CS score, | |
| | | | | | templated | replication, | function | |
| | | | | | | DNA repair | | |
| Polr3c | 74414 | POLR3C | 10623 | −2.02 | transcription, DNA- | DNA | CS score, | |
| | | | | | templated | replication, | function | |
| | | | | | | DNA repair | | |
| Polr3h | 78929 | POLR3H | 171568 | −2.66 | transcription, DNA- | DNA | CS score, | |
| | | | | | templated | replication, | function | |
| | | | | | | DNA repair | | |
| Prmt1 | 15469 | PRMT1 | 3276 | −2.40 | regulation of | DNA | CS score, | Pawlak M R, et al. |
| | | | | | transcription, DNA- | replication, | mouse | Mol Cell Biol. 2000 |
| | | | | | templated | DNA repair | K.O., | July; 20(13): 14859-69 |
| | | | | | | | function | |
| Prmt5 | 27374 | PRMT5 | 10419 | −2.69 | regulation of | DNA | CS score, | Tee W W, et al. |
| | | | | | transcription, DNA- | replication, | mouse | Genes Dev. 2010 |
| | | | | | templated | DNA repair | K.O., | Dec. 15; 24(24): 2772-7 |
| | | | | | | | function | |
| Puf60 | 67959 | PUF60 | 22827 | −2.69 | transcription, DNA- | DNA | CS score, | |
| | | | | | templated | replication, | function | |
| | | | | | | DNA repair | | |
| Rad51 | 19361 | RAD51 | 5888 | −2.29 | DNA repair | DNA | CS score, | Tsuzuki T, et al. |
| | | | | | | replication, | mouse | Proc Natl Acad Sci |
| | | | | | | DNA repair | K.O., | USA. 1996 Jun. |
| | | | | | | | function | 25; 93(13): 6236-40 |
| Rad51c | 114714 | RAD51C | 5889 | −1.62 | DNA repair | DNA | CS score, | Smeenk G, et al. |
| | | | | | | replication, | mouse | Mutat Res. 2010 Jul. |
| | | | | | | DNA repair | K.O., | 7; 689(1-2): 50-58 |
| | | | | | | | function | |
| Rbx1 | 56438 | RBX1 | 9978 | −2.19 | DNA repair | DNA | CS score, | Tan M, et al. Proc |
| | | | | | | replication, | mouse | Natl Acad Sci USA. |
| | | | | | | DNA repair | K.O., | 2009 Apr. |
| | | | | | | | function | 14; 106(15): 6203-8 |
| Rfc2 | 19718 | RFC2 | 5982 | −2.88 | DNA- | DNA | CS score, | |
| | | | | | dependent DNA | replication, | function | |
| | | | | | replication | DNA repair | | |
| Rfc4 | 106344 | RFC4 | 5984 | −1.92 | DNA- | DNA | CS score, | |
| | | | | | dependent DNA | replication, | function | |
| | | | | | replication | DNA repair | | |
| Rfc5 | 72151 | RFC5 | 5985 | −2.78 | DNA- | DNA | CS score, | |
| | | | | | dependent DNA | replication, | function | |
| | | | | | replication | DNA repair | | |
| Rpa1 | 68275 | RPA1 | 6117 | −2.61 | DNA replication | DNA | CS score, | Wang Y, et al. Nat |
| | | | | | | replication, | mouse | Genet. 2005 |
| | | | | | | DNA repair | K.O., | July; 37(7): 750-5 |
| | | | | | | | function | |
| Rps3 | 27050 | RPS3 | 6188 | −2.75 | DNA repair | DNA | CS score, | |
| | | | | | | replication, | function | |
| | | | | | | DNA repair | | |
| Rrm1 | 20133 | RRM1 | 6240 | −4.16 | DNA replication | DNA | CS score, | |
| | | | | | | replication, | function | |
| | | | | | | DNA repair | | |
| Ruvbl1 | 56505 | RUVBL1 | 8607 | −3.26 | DNA duplex | DNA | CS score, | |
| | | | | | unwinding | replication, | function | |
| | | | | | | DNA repair | | |
| Ruvbl2 | 20174 | RUVBL2 | 10856 | −3.91 | DNA repair | DNA | CS score, | |
| | | | | | | replication, | function | |
| | | | | | | DNA repair | | |
| Sap30bp | 57230 | SAP30BP | 29115 | −2.18 | regulation of | DNA | CS score, | |
| | | | | | transcription, DNA- | replication, | function | |
| | | | | | templated | DNA repair | | |
| Smc1a | 24061 | SMC1A | 8243 | −2.76 | DNA repair | DNA | CS score, | |
| | | | | | | replication, | function | |
| | | | | | | DNA repair | | |
| Smc3 | 13006 | SMC3 | 9126 | −3.22 | DNA repair | DNA | CS score, | White J K, et al. Cell. |
| | | | | | | replication, | mouse | 2013 Jul. |
| | | | | | | DNA repair | K.O., | 18; 154(2): 452-64 |
| | | | | | | | function | |
| Snapc4 | 227644 | SNAPC4 | 6621 | −2.78 | regulation of | DNA | CS score, | |
| | | | | | transcription, DNA- | replication, | function | |
| | | | | | templated | DNA repair | | |
| Snapc5 | 330959 | SNAPC5 | 10302 | −2.24 | regulation of | DNA | CS score, | |
| | | | | | transcription, DNA- | replication, | function | |
| | | | | | templated | DNA repair | | |
| Snip1 | 76793 | SNIP1 | 79753 | −1.78 | regulation of | DNA | CS score, | |
| | | | | | transcription, DNA- | replication, | function | |
| | | | | | templated | DNA repair | | |
| Srrt | 83701 | SRRT | 51593 | −2.18 | transcription, DNA- | DNA | CS score, | Wilson M D, et al. |
| | | | | | templated | replication, | mouse | Mol Cell Biol. 2008 |
| | | | | | | DNA repair | K.O., | March; 28(5): 1503-14 |
| | | | | | | | function | |
| Ssrp1 | 20833 | SSRP1 | 6749 | −1.45 | DNA replication | DNA | CS score, | Cao S, et al. 5 |
| | | | | | | replication, | mouse | mouse embryos |
| | | | | | | DNA repair | K.O., | Mol Cell Biol. 2003 |
| | | | | | | | function | August; 23(15): 5301-7 |
| Taf10 | 24075 | TAF10 | 6881 | −1.38 | DNA-templated | DNA | CS score, | Mohan W S Jr, et al. |
| | | | | | transcription, | replication, | mouse | Mol Cell Biol. 2003 |
| | | | | | initiation | DNA repair | K.O., | Jun. 23; (12): 4307-18 |
| | | | | | | | function | |
| Taf1c | 21341 | TAF1C | 9013 | −1.80 | chromatin silencing | DNA | CS score, | |
| | | | | | at rDNA | replication, | function | |
| | | | | | | DNA repair | | |
| Taf6 | 21343 | TAF6 | 6878 | −1.84 | DNA- | DNA | CS score, | |
| | | | | | templated | replication, | function | |
| | | | | | transcription, | DNA repair | | |
| | | | | | initiation | | | |
| Taf6l | 67706 | TAF6L | 10629 | −1.53 | DNA- | DNA | CS score, | |
| | | | | | templated | replication, | function | |
| | | | | | transcription, | DNA repair | | |
| | | | | | initiation | | | |
| Ticrr | 77011 | TICRR | 90381 | −2.03 | DNA replication | DNA | CS score, | |
| | | | | | | replication, | function | |
| | | | | | | DNA repair | | |
| Top1 | 21969 | TOP1 | 7150 | −2.02 | DNA topical | DNA | CS score, | Morham S G, et al. |
| | | | | | change | replication, | mouse | Mol Cell Biol. 1996 |
| | | | | | | DNA repair | K.O., | December; 16(12): 6804-9 |
| | | | | | | | function | |
| Top2a | 21973 | TOP2A | 7153 | −1.50 | DNA replication | DNA | CS score, | |
| | | | | | | replication, | function | |
| | | | | | | DNA repair | | |
| Trrap | 100683 | TRRAP | 8295 | −2.36 | DNA repair | DNA | CS score, | Herceg Z et al. Nat |
| | | | | | | replication, | mouse | Genet. 2001 |
| | | | | | | DNA repair | K.O., | October; 29(2): 206-11 |
| | | | | | | | function | |
| Zbtb11 | 271377 | ZBTB11 | 27107 | −2.34 | transcription, DNA- | DNA | CS score, | |
| | | | | | templated | replication, | function | |
| | | | | | | DNA repair | | |
| Actl6a | 56456 | ACTL6A | 86 | −2.33 | neural retina | DNA | CS score, | Krasteva V, et al. |
| | | | | | development | replication, | mouse | Blood. 2012 Dec. |
| | | | | | | DNA repair | K.O., | 6; 120(24): 4720-32 |
| | | | | | | | function | |
| Atr | 245000 | ATR | 545 | −2.01 | double-strand break | DNA | CS score, | de Klein A, et al. |
| | | | | | repair via | replication, | mouse | Curr Biol. 2000 Apr. |
| | | | | | homologous | DNA repair | K.O., | 20; 10(8): 479-82 |
| | | | | | recombination | | function | |
| Chd4 | 107932 | CHD4 | 1108 | −1.71 | chromatin | DNA | CS score, | |
| | | | | | organization | replication, | function | |
| | | | | | | DNA repair | | |
| Ciao1 | 26371 | CIAO1 | 9391 | −1.94 | chromosome | DNA | CS score, | |
| | | | | | segregation | replication, | function | |
| | | | | | | DNA repair | | |
| Ddx21 | 56200 | DDX21 | 9188 | −2.84 | osteoblast | DNA | CS score, | |
| | | | | | differentiation | replication, | function | |
| | | | | | | DNA repair | | |
| Dnaja3 | 83945 | DNAJA3 | 9093 | −2.19 | mitochondrion | DNA | CS score, | Lo J F, et al. Mol |
| | | | | | organization | replication, | mouse | Cell Biol. 2004 |
| | | | | | | DNA repair | K.O., | March; 24(6): 2226-36 |
| | | | | | | | function | |
| Dnmt1 | 13433 | DNMT1 | 1786 | −1.97 | methylation | DNA | CS score, | Lei H, et al. |
| | | | | | | replication, | mouse | Development. 1996 |
| | | | | | | DNA repair | K.O., | October; 122(10): 3195- |
| | | | | | | | function | 205 |
| Gins2 | 272551 | GINS2 | 51659 | −3.32 | double-strand break | DNA | CS score, | |
| | | | | | repair via break- | replication, | function | |
| | | | | | induced replication | DNA repair | | |
| Gtf2h3 | 209357 | GTF2H3 | 2967 | −1.84 | nucleotide- | DNA | CS score, | |
| | | | | | excision repair | replication, | function | |
| | | | | | | DNA repair | | |
| n/a | n/a | HIST2H2BF | 440689 | −1.70 | chromatin | DNA | CS score, | |
| | | | | | organization | replication, | function | |
| | | | | | | DNA repair | | |
| Mms22l | 212377 | MMS22L | 253714 | −1.38 | double-strand break | DNA | CS score, | |
| | | | | | repair via | replication, | function | |
| | | | | | homologous | DNA repair | | |
| | | | | | recombination | | | |
| Mtor | 56717 | MTOR | 2475 | −1.98 | double-strand break | DNA | CS score, | Murakami M, et al. |
| | | | | | repair via | replication, | mouse | Mol Cell Biol. 2004 |
| | | | | | homologous | DNA repair | K.O., | August; 24(15): 6710-8 |
| | | | | | recombination | | function | |
| Narfl | 67563 | NARFL | 64428 | −2.13 | response hypoxia | DNA | CS score, | Song D, et al. J Biol |
| | | | | | | replication, | mouse | Chem. 2011 Mar. 2 |
| | | | | | | DNA repair | K.O., | |
| | | | | | | | function | |
| Ndufa13 | 67184 | NDUFA13 | 51079 | −1.31 | positive regulation of | DNA | CS score, | Huang G, et al. Mol |
| | | | | | peptidase activity | replication, | mouse | Cell Biol. 2004 |
| | | | | | | DNA repair | K.O., | October; 24(19): 8447-56 |
| | | | | | | | function | |
| Nol12 | 97961 | NOL12 | 79159 | −1.61 | poly(A) RNA binding | DNA | CS score, | |
| | | | | | | replication, | function | |
| | | | | | | DNA repair | | |
| Nup107 | 103468 | NUP107 | 57122 | −1.30 | transport | DNA | CS score, | |
| | | | | | | replication, | function | |
| | | | | | | DNA repair | | |
| Oraov1 | 72284 | ORAOV1 | 220064 | −2.26 | biological_process | DNA | CS score, | |
| | | | | | | replication, | function | |
| | | | | | | DNA repair | | |
| Pam16 | 66449 | PAM16 | 51025 | −2.13 | protein import into | DNA | CS score, | |
| | | | | | mitochondrial matrix | replication, | function | |
| | | | | | | DNA repair | | |
| Pola2 | 18969 | POLA2 | 23649 | −2.84 | protein import into | DNA | CS score, | |
| | | | | | nucleus, | replication, | function | |
| | | | | | translocation | DNA repair | | |
| Ppie | 56031 | PPIE | 10450 | −1.63 | protein peptidyl-prolyl | DNA | CS score, | |
| | | | | | isomerization | replication, | function | |
| | | | | | | DNA repair | | |
| Prpf19 | 28000 | PRPF19 | 27339 | −3.96 | generation of | DNA | CS score, | Fortschegger K, et |
| | | | | | catalytic spliceosome | replication, | mouse | al. Mol Cell Biol. |
| | | | | | for first | DNA repair | K.O., | 2007 |
| | | | | | transesterification | | function | April; 27(8): 3123-30 |
| | | | | | step | | | |
| Psmc5 | 19184 | PSMC5 | 5705 | −2.57 | ER- | DNA | CS score, | |
| | | | | | associated ubiquitin- | replication, | function | |
| | | | | | dependent protein | DNA repair | | |
| | | | | | catabolic process | | | |
| Rbbp5 | 213464 | RBBP5 | 5929 | −1.70 | chromatin | DNA | CS score, | |
| | | | | | organization | replication, | function | |
| | | | | | | DNA repair | | |
| Rbbp6 | 19647 | RBBP6 | 5930 | −1.78 | in utero embryonic | DNA | CS score, | Li L, et al. Proc Natl |
| | | | | | development | replication, | mouse | Acad Sci USA. |
| | | | | | | DNA repair | K.O., | 2007 May |
| | | | | | | | function | 8; 104(19): 7951-6 |
| Rptor | 74370 | RPTOR | 57521 | −2.43 | TOR signalling | DNA | CS score, | Guertin D A, et al. |
| | | | | | | replication, | mouse | Dev Cell. 2006 |
| | | | | | | DNA repair | K.O., | December; 11(6): 859-71 |
| | | | | | | | function | |
| Rrn3 | 106298 | RRN3 | 54700 | −1.85 | in utero embryonic | DNA | CS score, | Yuan X, et al. Mol |
| | | | | | development | replication, | mouse | Cell. 2005 Jul. |
| | | | | | | DNA repair | K.O., | 1; 19(1): 77-87 |
| | | | | | | | function | |
| Smg1 | 233789 | SMG1 | 23049 | −1.94 | double-strand break | DNA | CS score, | Roberts T L, et al. |
| | | | | | repair via | replication, | mouse | Proc Natl Acad Sci |
| | | | | | homologous | DNA repair | K.O., | USA. 2013 Jan. |
| | | | | | recombination | | function | 22; 110(4): E285-94 |
| Supt6 | 20926 | SUPT6H | 6830 | −1.78 | chromatin | DNA | CS score, | Dietrich J E, et al. |
| | | | | | remodeling | replication, | mouse | EMBO Rep. 2015 |
| | | | | | | DNA repair | K.O., | August; 16(8): 1005-21 |
| | | | | | | | function | |
| Tada2b | 231151 | TADA2B | 93624 | −1.23 | chromatin | DNA | CS score, | |
| | | | | | organization | replication, | function | |
| | | | | | | DNA repair | | |
| Tfip11 | 54723 | TFIP11 | 24144 | −2.19 | spliceosomal | DNA | CS score, | |
| | | | | | complex disassembly | replication, | function | |
| | | | | | | DNA repair | | |
| Tonsl | 66914 | TONSL | 4796 | −3.03 | double-strand break | DNA | CS score, | |
| | | | | | repair via | replication, | function | |
| | | | | | homologous | DNA repair | | |
| | | | | | recombination | | | |
| Tpt1 | 22070 | TPT1 | 7178 | −2.05 | calcium ion transport | DNA | CS score, | Susini L, et al. Cell |
| | | | | | | replication, | mouse | Death Differ. 2008 |
| | | | | | | DNA repair | K.O., | August; 15(8): 1211-20 |
| | | | | | | | function | |
| Uba1 | 22201 | UBA1 | 7317 | −2.90 | protein ubiquitination | DNA | CS score, | |
| | | | | | | replication, | function | |
| | | | | | | DNA repair | | |
| Vps25 | 28084 | VPS25 | 84313 | −2.31 | protein targeting | DNA | CS score, | |
| | | | | | to vacuole involved | replication, | function | |
| | | | | | in ubiquitin- | DNA repair | | |
| | | | | | dependent protein | | | |
| | | | | | catabolic process via | | | |
| | | | | | the multivesicular | | | |
| | | | | | body sorting pathway | | | |
| Wbscr22 | 66138 | WBSCR22 | 114049 | −2.70 | methylation | DNA | CS score, | |
| | | | | | | replication, | function | |
| | | | | | | DNA repair | | |
| Wdr5 | 140858 | WDR5 | 11091 | −1.99 | skeletal system | DNA | CS score, | |
| | | | | | development | replication, | function | |
| | | | | | | DNA repair | | |
| Xab2 | 67439 | XAB2 | 56949 | −2.86 | generation of | DNA | CS score, | Yonemasu R, et al. |
| | | | | | catalytic spliceosome | replication, | mouse | DNA Repair (Amst). |
| | | | | | for first | DNA repair | K.O., | 2005 Apr. |
| | | | | | transesterification | | function | 4; 4(4): 473-91 |
| | | | | | step | | | |
| Zmat2 | 66492 | ZMAT2 | 153527 | −2.17 | histidine- | DNA | CS score, | |
| | | | | | tRNA ligase | replication, | function | |
| | | | | | activity | DNA repair | | |
| Zfp335 | 329559 | ZNF335 | 63925 | −1.58 | in utero embryonic | DNA | CS score, | Yang Y J, et al. Cell. |
| | | | | | development | replication, | mouse | 2012 Nov. |
| | | | | | | DNA repair | K.O., | 21; 151(5): 1097-112 |
| | | | | | | | function | |
| Acly | 104112 | ACLY | 47 | −1.54 | acetyl-CoA metabolic | Metabolism | CS score, | Beigneux A P, et al. |
| | | | | | process | | mouse | J Biol Chem. 2004 |
| | | | | | | | K.O., | Mar. |
| | | | | | | | function | 5; 279(10): 9557-64 |
| Adsl | 11564 | ADSL | 158 | −2.39 | metabolic process | Metabolism | CS score, | |
| | | | | | | | function | |
| Ahcy | 269378 | AHCY | 191 | −2.07 | sulfur amino acid | Metabolism | CS score, | |
| | | | | | metabolic process | | function | |
| Arl2 | 56327 | ARL2 | 402 | −2.29 | energy reserve | Metabolism | CS score, | |
| | | | | | metabolic process | | function | |
| Chka | 12660 | CHKA | 1119 | −1.64 | lipid metabolic | Metabolism | CS score, | Wu G, et al. J Biol |
| | | | | | process | | mouse | Chem. 2008 Jan. |
| | | | | | | | K.O., | 18; 283(3): 1456-62 |
| | | | | | | | function | |
| Coasy | 71743 | COASY | 80347 | −1.82 | vitamin metabolic | Metabolism | CS score, | |
| | | | | | process | | function | |
| Cox4i1 | 12857 | COX4I1 | 1327 | −2.00 | generation of | Metabolism | CS score, | |
| | | | | | precursor | | function | |
| | | | | | metabolites and | | | |
| | | | | | energy | | | |
| n/a | n/a | COX7C | 1350 | −1.59 | generation of | Metabolism | CS score, | |
| | | | | | precursor | | function | |
| | | | | | metabolites and | | | |
| | | | | | energy | | | |
| n/a | n/a | CTPS1 | 1503 | −2.52 | nucleobase- | Metabolism | CS score, | |
| | | | | | containing compound | | function | |
| | | | | | metabolic process | | | |
| Ddx10 | 77591 | DDX10 | 1662 | −2.02 | metabolic process | Metabolism | CS score, | |
| | | | | | | | function | |
| Ddx20 | 53975 | DDX20 | 11218 | −2.49 | metabolic process | Metabolism | CS score, | Mouillet J F, et al. |
| | | | | | | | mouse | Endocrinology. |
| | | | | | | | K.O., | 2008 |
| | | | | | | | function | May; 149(5): 2168-75 |
| Dhdds | 67422 | DHDDS | 79947 | −2.86 | metabolic process | Metabolism | CS score, | |
| | | | | | | | function | |
| Dhx30 | 72831 | DHX30 | 22907 | −1.93 | metabolic process | Metabolism | CS score, | |
| | | | | | | | function | |
| Dhx8 | 217207 | DHX8 | 1659 | −2.61 | metabolic process | Metabolism | CS score, | |
| | | | | | | | function | |
| Dhx9 | 13211 | DHX9 | 1660 | −1.73 | metabolic process | Metabolism | CS score, | Lee C G, et al. Proc |
| | | | | | | | mouse | Natl Acad Sci USA. |
| | | | | | | | K.O., | 1998 Nov. |
| | | | | | | | function | 10; 95(23): 13709-13 |
| Dlst | 78920 | DLST | 1743 | −1.93 | metabolic process | Metabolism | CS score, | |
| | | | | | | | function | |
| Dpagt1 | 13478 | DPAGT1 | 1798 | −2.80 | UDP-N- | Metabolism | CS score, | Marek K W, et al. |
| | | | | | acetylglucosamine | | mouse | Glycobiology. 1999 |
| | | | | | metabolic process | | K.O., | November; 9(11): 1263-71 |
| | | | | | | | function | |
| Gfpt1 | 14583 | GFPT1 | 2673 | −1.81 | fructose 6-phosphate | Metabolism | CS score, | |
| | | | | | metabolic process | | function | |
| Gmps | 229363 | GMPS | 8833 | −1.80 | Purine nucleobase | Metabolism | CS score, | |
| | | | | | metabolic process | | function | |
| Gpn1 | 74254 | GPN1 | 11321 | −1.79 | metabolic process | Metabolism | CS score, | |
| | | | | | | | function | |
| Gpn3 | 68080 | GPN3 | 51184 | −3.12 | metabolic process | Metabolism | CS score, | |
| | | | | | | | function | |
| Guk1 | 14923 | GUK1 | 2987 | −2.67 | purine nucleotide | Metabolism | CS score, | |
| | | | | | metabolic process | | function | |
| Hsd17b10 | 15108 | HSD17B10 | 3028 | −1.84 | lipid metabolic | Metabolism | CS score, | |
| | | | | | process | | function | |
| Lrr1 | 69706 | LRR1 | 122769 | −3.44 | metabolic process | Metabolism | CS score, | |
| | | | | | | | function | |
| Mtg2 | 52856 | MTG2 | 26164 | −2.04 | metabolic process | Metabolism | CS score, | |
| | | | | | | | function | |
| Myh9 | 17886 | MYH9 | 4627 | −1.70 | metabolic process | Metabolism | CS score, | Matsushita T, et al. |
| | | | | | | | mouse | Biochem Biophys |
| | | | | | | | K.O., | Res Commun. 2004 |
| | | | | | | | function | Dec. |
| | | | | | | | | 24; 325(4): 1163-71 |
| Nampt | 59027 | NAMPT | 10135 | −2.40 | vitamin metabolic | Metabolism | CS score, | Revollo J R, et al. |
| | | | | | process | | mouse | Cell Metab. 2007 |
| | | | | | | | K.O., | November; 6(5): 363-75 |
| | | | | | | | function | |
| Ncbp1 | 433702 | NCBP1 | 4686 | −1.62 | RNA metabolic | Metabolism | CS score, | |
| | | | | | process | | function | |
| Nfs1 | 18041 | NFS1 | 9054 | −2.40 | metabolic process | Metabolism | CS score, | |
| | | | | | | | function | |
| Ppcdc | 66812 | PPCDC | 60490 | −1.98 | metabolic process | Metabolism | CS score, | |
| | | | | | | | function | |
| Qrsl1 | 76563 | QRSL1 | 55278 | −1.67 | metabolic process | Metabolism | CS score, | |
| | | | | | | | function | |
| Rpp14 | 67053 | RPP14 | 11102 | −1.72 | fatty acid metabolic | Metabolism | CS score, | |
| | | | | | process | | function | |
| Smarca4 | 20586 | SMARCA4 | 6597 | −1.89 | metabolic process | Metabolism | CS score, | Bultman S, et al. |
| | | | | | | | mouse | Mol Cell. 2000 |
| | | | | | | | K.O., | December; 6(6): 1287-95 |
| | | | | | | | function | |
| Snrnp200 | 320632 | SNRNP200 | 23020 | −2.50 | metabolic process | Metabolism | CS score, | |
| | | | | | | | function | |
| Srbd1 | 78586 | SRBD1 | 55133 | −2.35 | nucleobase- | Metabolism | CS score, | |
| | | | | | containing compound | | function | |
| | | | | | metabolic process | | | |
| Srcap | 100043597 | SRCAP | 10847 | −1.43 | metabolic process | Metabolism | CS score, | |
| | | | | | | | function | |
| Ube2i | 22196 | UBE2I | 7329 | −2.55 | metabolic process | Metabolism | CS score, | Nacerddine K, et al. |
| | | | | | | | mouse | Dev Cell. 2005 |
| | | | | | | | K.O., | December; 9(6): 769-79 |
| | | | | | | | function | |
| Ube2m | 22192 | UBE2M | 9040 | −2.39 | metabolic process | Metabolism | CS score, | |
| | | | | | | | function | |
| Vcp | 269523 | VCP | 7415 | −2.85 | metabolic process | Metabolism | CS score, | Muller J M, et al. |
| | | | | | | | mouse | Biochem Biophys |
| | | | | | | | K.O., | Res Commun. 2007 |
| | | | | | | | function | Mar. 9; 354(2): 459- |
| | | | | | | | | 465 |
| Aamp | 227290 | AAMP | 14 | −2.37 | angiogenesis | Metabolism | CS score, | |
| | | | | | | | function | |
| Acin1 | 56215 | ACIN1 | 22985 | −1.53 | positive regulation of | Metabolism | CS score, | |
| | | | | | defense response to | | function | |
| | | | | | virus by host | | | |
| Aco2 | 11429 | ACO2 | 50 | −2.08 | tricarboxylic acid | Metabolism | CS score, | |
| | | | | | cycle | | function | |
| Adss | 11566 | ADSS | 159 | −2.46 | purine nucleotide | Metabolism | CS score, | |
| | | | | | biosynthetic process | | function | |
| Alg2 | 56737 | ALG2 | 85365 | −2.29 | biosynthetic process | Metabolism | CS score, | |
| | | | | | | | function | |
| Ap2s1 | 232910 | AP2S1 | 1175 | −2.00 | intracellular protein | Metabolism | CS score, | |
| | | | | | transport | | function | |
| Arcn1 | 213827 | ARCN1 | 372 | −1.91 | intracellular protein | Metabolism | CS score, | |
| | | | | | transport | | function | |
| Armc7 | 276905 | ARMC7 | 79637 | −2.02 | molecular_function | Metabolism | CS score, | |
| | | | | | | | function | |
| Atp2a2 | 11938 | ATP2A2 | 488 | −3.01 | calcium ion | Metabolism | CS score, | Andersson K B, et |
| | | | | | transmembrane | | mouse | al. Cell Calcium. |
| | | | | | transport | | K.O., | 2009 |
| | | | | | | | function | September; 46(3): 219-25 |
| Atp5a1 | 11946 | ATP5A1 | 498 | −1.99 | negative regulation of | Metabolism | CS score, | |
| | | | | | endothelial cell | | function | |
| | | | | | proliferation | | | |
| Atp5d | 66043 | ATP5D | 513 | −2.21 | oxidative | Metabolism | CS score, | |
| | | | | | phosphorylation | | function | |
| Atp5o | 28080 | ATP5O | 539 | −1.17 | ATP biosynthetic | Metabolism | CS score, | |
| | | | | | process | | function | |
| Atp6v0b | 114143 | ATP6V0B | 533 | −3.01 | cellular iron ion | Metabolism | CS score, | |
| | | | | | homeostasis | | function | |
| Atp6v0c | 11984 | ATP6V0C | 527 | −3.84 | cellular iron ion | Metabolism | CS score, | Sun-Wada G H, et |
| | | | | | homeostasis | | mouse | al. Dev Biol. 2000 |
| | | | | | | | K.O., | Dec. 15; 228(2): 315- |
| | | | | | | | function | 25 |
| Atp6v1a | 11964 | ATP6V1A | 523 | −3.58 | proton transport | Metabolism | CS score, | |
| | | | | | | | function | |
| Atp6v1b2 | 11966 | ATP6V1B2 | 526 | −2.94 | cellular iron ion | Metabolism | CS score, | |
| | | | | | homeostasis | | function | |
| Atp6v1d | 73834 | ATP6V1D | 51382 | −2.58 | transmembrane | Metabolism | CS score, | |
| | | | | | transport | | function | |
| Aurkaip1 | 66077 | AURKAIP1 | 54998 | −1.56 | organelle | Metabolism | CS score, | |
| | | | | | organization | | function | |
| n/a | n/a | C1orf109 | 54955 | −2.43 | molecular_function | Metabolism | CS score, | |
| | | | | | | | function | |
| n/a | n/a | C21orf59 | 56683 | −2.77 | cell projection | Metabolism | CS score, | |
| | | | | | morphogenesis | | function | |
| Ccdc84 | 382073 | CCDC84 | 338657 | −1.86 | molecular_function | Metabolism | CS score, | |
| | | | | | | | function | |
| Cct2 | 12461 | CCT2 | 10576 | −3.23 | protein folding | Metabolism | CS score, | |
| | | | | | | | function | |
| Cct3 | 12462 | CCT3 | 7203 | −3.31 | protein folding | Metabolism | CS score, | |
| | | | | | | | function | |
| Cct4 | 12464 | CCT4 | 10575 | −2.62 | protein folding | Metabolism | CS score, | |
| | | | | | | | function | |
| Cct5 | 12465 | CCT5 | 22948 | −2.84 | protein folding | Metabolism | CS score, | |
| | | | | | | | function | |
| Cct7 | 12468 | CCT7 | 10574 | −2.47 | protein folding | Metabolism | CS score, | |
| | | | | | | | function | |
| Cct8 | 12469 | CCT8 | 10694 | 2.03 | protein folding | Metabolism | CS score, | |
| | | | | | | | function | |
| Cdipt | 52858 | CDIPT | 10423 | −2.53 | phospholipid | Metabolism | CS score, | |
| | | | | | biosynthetic process | | function | |
| Cenpi | 102920 | CENPI | 249 | −1.81 | centromere complex | Metabolism | CS score, | |
| | | | | | assembly | | function | |
| Chordc1 | 66917 | CHORDC1 | 26973 | −1.52 | regulation of | Metabolism | CS score, | Ferretti R, et al. Dev |
| | | | | | centrosome | | mouse | Cell. 2010 Mar. |
| | | | | | duplication | | K.O., | 16; 18(3): 486-95 |
| | | | | | | | function | |
| Coa5 | 76178 | COA5 | 493753 | −2.33 | mitochondrion | Metabolism | CS score, | |
| | | | | | | | function | |
| Cog4 | 102339 | COG4 | 25839 | −1.39 | Golgi vesicle | Metabolism | CS score, | |
| | | | | | transport | | function | |
| Copa | 12847 | COPA | 1314 | −1.63 | intracellular protein | Metabolism | CS score, | |
| | | | | | transport | | function | |
| Copb1 | 70349 | COPB1 | 1315 | −2.30 | intracellular protein | Metabolism | CS score, | |
| | | | | | transport | | function | |
| Copb2 | 50797 | COPB2 | 9276 | −2.65 | intracellular protein | Metabolism | CS score, | |
| | | | | | transport | | function | |
| Cope | 59042 | COPE | 11316 | −2.93 | ER to Golgi vesicle- | Metabolism | CS score, | |
| | | | | | mediated transport | | function | |
| Copz1 | 56447 | COPZ1 | 22818 | −1.87 | transport | Metabolism | CS score, | |
| | | | | | | | function | |
| Coq4 | 227683 | COQ4 | 51117 | −1.29 | ubiquinone | Metabolism | CS score, | |
| | | | | | biosynthetic process | | function | |
| Cox15 | 226139 | COX15 | 1355 | −2.14 | mitochondrial | Metabolism | CS score, | Viscomi C, et al. |
| | | | | | electron transport, | | mouse | Cell Metab. 2011 |
| | | | | | cytochrome c | | K.O., | Jul. 6; 14(1): 80-90 |
| | | | | | to oxygen | | function | |
| Cox17 | 12856 | COX17 | 10063 | −1.97 | copper ion transport | Metabolism | CS score, | Takahashi Y, et al. |
| | | | | | | | mouse | Mol Cell Biol. 2002 |
| | | | | | | | K.O., | November; 22(21): 7614- |
| | | | | | | | function | 21 |
| Cse1l | 110750 | CSE1L | 1434 | −2.31 | protein export from | Metabolism | CS score, | Bera T K, et al. Mol |
| | | | | | nucleus | | mouse | Cell Biol. 2001 |
| | | | | | | | K.O., | October; 21(20): 7020-4 |
| | | | | | | | function | |
| Csnk2b | 13001 | CSNK2B | 1460 | −1.94 | regulation of protein | Metabolism | CS score, | Buchou T, et al. Mol |
| | | | | | kinase activity | | mouse | Cell Biol, 2003 |
| | | | | | | | K.O., | February; 23(3): 908-15 |
| | | | | | | | function | |
| Cycs | 13063 | CYCS | 54205 | −2.36 | response to reactive | Metabolism | CS score, | Li K, et al. Cell. |
| | | | | | oxygen species | | mouse | 2000 May |
| | | | | | | | K.O., | 12; 101(4): 389-99 |
| | | | | | | | function | |
| Dad1 | 13135 | DAD1 | 1603 | −2.21 | protein glycosylation | Metabolism | CS score, | Brewster J L, et al. |
| | | | | | | | mouse | Genesis. 2000 |
| | | | | | | | K.O., | April; 26(4): 271-8 |
| | | | | | | | function | |
| Dap3 | 65111 | DAP3 | 7818 | −1.70 | apoptotic process | Metabolism | CS score, | Kim H R, et al. |
| | | | | | | | mouse | FASEB J. 2007 |
| | | | | | | | K.O., | January; 21(1): 188-96 |
| | | | | | | | function | |
| Dctn5 | 59288 | DCTN5 | 84516 | −2.39 | antigen processing | Metabolism | CS score, | |
| | | | | | and presentation of | | function | |
| | | | | | exogenous peptide | | | |
| | | | | | antigen via MHC | | | |
| | | | | | class II | | | |
| Ddost | 13200 | DDOST | 1650 | −2.38 | protein N-linked | Metabolism | CS score, | |
| | | | | | glycosylation via | | function | |
| | | | | | asparagine | | | |
| Dgcr8 | 94223 | DGCR8 | 54487 | −2.10 | gene expression | Metabolism | CS score, | Ouchi Y, et al. J |
| | | | | | | | mouse | Neurosci. 2013 May |
| | | | | | | | K.O., | 29; 33(22): 9408-19 |
| | | | | | | | function | |
| Dhodh | 56749 | DHODH | 1723 | −2.57 | de novo' pyrimidine | Metabolism | CS score, | |
| | | | | | nucleobase | | function | |
| | | | | | biosynthetic process | | | |
| Dnlz | 52838 | DNLZ | 728489 | −1.92 | protein folding | Metabolism | CS score, | |
| | | | | | | | function | |
| Dnm1l | 74006 | DNM1L | 10059 | 3.25 | mitochondrial fission | Metabolism | CS score, | Wakabayashi J, et |
| | | | | | | | mouse | al. J Cell Biol. 2009 |
| | | | | | | | K.O., | Sep. 21; 186(6): 805- |
| | | | | | | | function | 16 |
| Dnm2 | 13430 | DNM2 | 1785 | −3.98 | endocytosis | Metabolism | CS score, | Ferguson S M, et al. |
| | | | | | | | mouse | Dev Cell. 2009 |
| | | | | | | | K.O., | December; 17(6): 811-22 |
| | | | | | | | function | |
| Dohh | 102115 | DOHH | 83475 | −1.76 | peptidyl- | Metabolism | CS score, | |
| | | | | | lysine modification | | function | |
| | | | | | to peptidyl- | | | |
| | | | | | hypusine | | | |
| Dolk | 227697 | DOLK | 22845 | −2.38 | dolichol- | Metabolism | CS score, | |
| | | | | | linked | | function | |
| | | | | | oligosaccharide | | | |
| | | | | | biosynthetic process | | | |
| Donson | 60364 | DONSON | 29980 | −2.30 | multicellular | Metabolism | CS score, | |
| | | | | | organismal | | function | |
| | | | | | development | | | |
| Dph3 | 105638 | DPH3 | 285381 | −1.62 | peptidyl- | Metabolism | CS score, | Liu S, et al. Mol Cell |
| | | | | | diphthamide | | mouse | Biol. 2006 |
| | | | | | biosynthetic process | | K.O., | May; 26(10): 3835-41 |
| | | | | | from peptidyl- | | function | |
| | | | | | histidine | | | |
| Dtymk | 21915 | DTYMK | 1841 | −3.54 | phosphorylation | Metabolism | CS score, | |
| | | | | | | | function | |
| Eif2b2 | 217715 | EIF2B2 | 8892 | −2.24 | ovarian follicle | Metabolism | CS score, | |
| | | | | | development | | function | |
| Eif2s2 | 67204 | EIF2S2 | 8894 | −2.33 | in utero embryonic | Metabolism | CS score, | Heaney J D, et al. |
| | | | | | development | | mouse | Hum Mol Genet. |
| | | | | | | | K.O., | 2009 Apr. |
| | | | | | | | function | 15; 18(8): 1395-404 |
| Emc1 | 230866 | EMC1 | 23065 | −1.34 | protein folding in | Metabolism | CS score, | |
| | | | | | endoplasmic | | function | |
| | | | | | reticulum | | | |
| Emc7 | 73024 | EMC7 | 56851 | −2.27 | biological_process | Metabolism | CS score, | |
| | | | | | | | function | |
| Eno1 | 13806 | ENO1 | 2023 | −2.03 | glycolytic process | Metabolism | CS score, | Couldrey C, et al. |
| | | | | | | | mouse | Dev Dyn. 1998 |
| | | | | | | | K.O., | June; 212(2): 284-92 |
| | | | | | | | function | |
| Fam50a | 108160 | FAM50A | 9130 | −3.16 | spermatogenesis | Metabolism | CS score, | |
| | | | | | | | function | |
| Fam96b | 68523 | FAM96B | 51647 | −1.90 | iron-sulfur cluster | Metabolism | CS score, | |
| | | | | | assembly | | function | |
| Fdps | 110196 | FDPS | 2224 | −2.41 | isoprenoid | Metabolism | CS score, | |
| | | | | | biosynthetic process | | function | |
| Gapdh | 14433 | GAPDH | 2597 | −2.40 | oxidation- | Metabolism | CS score, | |
| | | | | | reduction process | | function | |
| Gart | 14450 | GART | 2618 | −1.87 | purine nucleobase | Metabolism | CS score, | |
| | | | | | biosynthetic process | | function | |
| Gemin4 | 276919 | GEMIN4 | 50628 | −1.56 | spliceosomal snRNP | Metabolism | CS score, | |
| | | | | | assembly | | function | |
| Gemin5 | 216766 | GEMIN5 | 25929 | −2.51 | spliceosomal snRNP | Metabolism | CS score, | |
| | | | | | assembly | | function | |
| Ggps1 | 14593 | GGPS1 | 9453 | −1.62 | cholesterol | Metabolism | CS score, | |
| | | | | | biosynthetic process | | function | |
| Gmppb | 331026 | GMPPB | 29925 | −3.22 | biosynthetic process | Metabolism | CS score, | |
| | | | | | | | function | |
| Gnb1l | 13972 | GNB1L | 54584 | −1.93 | G-protein coupled | Metabolism | CS score, | |
| | | | | | receptor signaling | | function | |
| | | | | | pathway | | | |
| n/a | n/a | GOLGA6L1 | 283767 | −3.15 | | Metabolism | CS score, | |
| | | | | | | | function | |
| Gosr2 | 56494 | GOSR2 | 9570 | −1.13 | protein targeting to | Metabolism | CS score, | |
| | | | | | vacuole | | function | |
| Gpkow | 209416 | GPKOW | 27238 | −1.36 | biological_process | Metabolism | CS score, | |
| | | | | | | | function | |
| Gpn2 | 100210 | GPN2 | 54707 | −3.71 | biological_process | Metabolism | CS score, | |
| | | | | | | | function | |
| Gps1 | 209318 | GPS1 | 2873 | −2.11 | inactivation of MAPK | Metabolism | CS score, | |
| | | | | | activity | | function | |
| Grpel1 | 17713 | GRPEL1 | 80273 | −2.61 | protein folding | Metabolism | CS score, | |
| | | | | | | | function | |
| Grwd1 | 101612 | GRWD1 | 83743 | −1.90 | poly(A) RNA binding | Metabolism | CS score, | |
| | | | | | | | function | |
| Hmgcr | 15357 | HMGCR | 3156 | −2.94 | cholesterol | Metabolism | CS score, | Ohashi K. et al. J |
| | | | | | biosynthetic process | | mouse | Biol Chem. 2003 |
| | | | | | | | K.O., | Oct. |
| | | | | | | | function | 31; 278(44): 42936- |
| | | | | | | | | 41 |
| Hmgcs1 | 208715 | HMGCS1 | 3157 | −2.41 | liver development | Metabolism | CS score, | |
| | | | | | | | function | |
| Hspa5 | 14828 | HSPA5 | 3309 | −3.86 | platelet degranulation | Metabolism | CS score, | Luo S, et al. Mol |
| | | | | | | | mouse | Cell Biol. 2006 |
| | | | | | | | K.O., | August; 26(15): 5688-97 |
| | | | | | | | function | |
| Hspa9 | 15526 | HSPA9 | 3313 | −3.55 | protein folding | Metabolism | CS score, | |
| | | | | | | | function | |
| Hspd1 | 15510 | HSPD1 | 3329 | −1.95 | response to hypoxia | Metabolism | CS score, | Christensen J H, et |
| | | | | | | | mouse | al. Cell Stress |
| | | | | | | | K.O., | Chaperones, 2010 |
| | | | | | | | function | November; 15(6): 851-63 |
| Hspe1 | 15528 | HSPE1 | 3336 | −3.75 | osteoblast | Metabolism | CS score, | |
| | | | | | differentiation | | function | |
| Hyou1 | 12282 | HYOU1 | 10525 | −2.06 | response to ischemia | Metabolism | CS score, | |
| | | | | | | | function | |
| Ipo13 | 230673 | IPO13 | 9670 | −2.84 | intracellular protein | Metabolism | CS score, | |
| | | | | | transport | | function | |
| Iscu | 66383 | ISCU | 23479 | −2.40 | cellular iron ion | Metabolism | CS score, | |
| | | | | | homeostasis | | function | |
| Itpk1 | 217837 | ITPK1 | 3705 | −1.55 | phosphorylation | Metabolism | CS score, | |
| | | | | | | | function | |
| Kansl2 | 69612 | KANSL2 | 54934 | −1.19 | chromatin | Metabolism | CS score, | |
| | | | | | organization | | function | |
| Kansl3 | 226976 | KANSL3 | 55683 | −1.53 | chromatin | Metabolism | CS score, | |
| | | | | | organization | | function | |
| Kri1 | 215194 | KRI1 | 65095 | −2.49 | poly(A) RNA binding | Metabolism | CS score, | |
| | | | | | | | function | |
| Lamtor2 | 83409 | LAMTOR2 | 28956 | −1.62 | activation of MAPKK | Metabolism | CS score, | Teis D, et al. J Cell |
| | | | | | activity | | mouse | Biol. 2006 Dec. |
| | | | | | | | K.O., | 18; 175(6): 861-8 |
| | | | | | | | function | |
| Leng8 | 232798 | LENG8 | 114823 | −1.75 | biological_process | Metabolism | CS score, | |
| | | | | | | | function | |
| Ltv1 | 353258 | LTV1 | 84946 | −1.81 | nucleoplasm | Metabolism | CS score, | |
| | | | | | | | function | |
| Mak16 | 67920 | MAK16 | 84549 | −2.30 | poly(A) RNA binding | Metabolism | CS score, | |
| | | | | | | | function | |
| Mat2a | 232087 | MAT2A | 4144 | −2.34 | S-adenosylmethionine | Metabolism | CS score, | |
| | | | | | biosynthetic process | | function | |
| Mcm3ap | 54387 | MCM3AP | 8888 | −1.58 | immune system | Metabolism | CS score, | Yoshida M, et al. |
| | | | | | process | | mouse | Genes Cells. 2007 |
| | | | | | | | K.O., | October; 12(10): 1205-13 |
| | | | | | | | function | |
| Mdn1 | 100019 | MDN1 | 23195 | −1.68 | protein complex | Metabolism | CS score, | |
| | | | | | assembly | | function | |
| n/a | n/a | MFAP1 | 4236 | −1.94 | biological_process | Metabolism | CS score, | |
| | | | | | | | function | |
| Mmgt1 | 236792 | MMGT1 | 93380 | −1.55 | magnesium ion | Metabolism | CS score, | |
| | | | | | transport | | function | |
| Mrpl16 | 94063 | MRPL16 | 54948 | −1.80 | organelle | Metabolism | CS score, | |
| | | | | | organization | | function | |
| Mrpl17 | 27397 | MRPL17 | 63875 | −1.80 | mitochondrial | Metabolism | CS score, | |
| | | | | | genome | | function | |
| | | | | | maintenance | | | |
| Mrpl33 | 66845 | MRPL33 | 9553 | −1.62 | organelle | Metabolism | CS score, | |
| | | | | | organization | | function | |
| Mrpl38 | 60441 | MRPL38 | 64978 | −1.95 | organelle | Metabolism | CS score, | |
| | | | | | organization | | function | |
| Mrpl39 | 27393 | MRPL39 | 54148 | −1.71 | organelle | Metabolism | CS score, | |
| | | | | | organization | | function | |
| Mrpl45 | 67036 | MRPL45 | 84311 | −1.75 | organelle | Metabolism | CS score, | |
| | | | | | organization | | function | |
| Mrpl46 | 67308 | MRPL46 | 26589 | −1.83 | organelle | Metabolism | CS score, | |
| | | | | | organization | | function | |
| Mrpl53 | 68499 | MRPL53 | 116540 | −1.84 | organelle | Metabolism | CS score, | |
| | | | | | organization | | function | |
| Mrps22 | 64655 | MRPS22 | 56945 | −1.32 | organelle | Metabolism | CS score, | |
| | | | | | organization | | function | |
| Mrps25 | 64658 | MRPS25 | 64432 | −1.63 | organelle | Metabolism | CS score, | |
| | | | | | organization | | function | |
| Mrps35 | 232536 | MRPS35 | 60488 | −1.60 | organelle | Metabolism | CS score, | |
| | | | | | organization | | function | |
| Mrps5 | 77721 | MRPS5 | 64969 | −1.65 | organelle | Metabolism | CS score, | |
| | | | | | organization | | function | |
| Mvd | 192156 | MVD | 4597 | −3.24 | isoprenoid | Metabolism | CS score, | |
| | | | | | biosynthetic process | | function | |
| Mvk | 17855 | MVK | 4598 | −1.73 | isoprenoid | Metabolism | CS score, | |
| | | | | | biosynthetic process | | function | |
| Naa25 | 231713 | NAA25 | 80018 | −2.40 | peptide alpha-N- | Metabolism | CS score, | |
| | | | | | acetyltransferase | | function | |
| | | | | | activity | | | |
| Napa | 108124 | NAPA | 8775 | −2.31 | intracellular protein | Metabolism | CS score, | |
| | | | | | transport | | function | |
| Nat10 | 98956 | NAT10 | 55226 | −2.16 | biological_process | Metabolism | CS score, | |
| | | | | | | | function | |
| Ndor1 | 78797 | NDOR1 | 27158 | −2.10 | cell death | Metabolism | CS score, | |
| | | | | | | | function | |
| Ndufab1 | 70316 | NDUFAB1 | 4706 | −1.83 | fatty acid biosynthetic | Metabolism | CS score, | |
| | | | | | process | | function | |
| Nol10 | 217431 | NOL10 | 79954 | −1.79 | poly(A) RNA binding | Metabolism | CS score, | |
| | | | | | | | function | |
| Nop9 | 67842 | NOP9 | 161424 | −1.44 | biological_process | Metabolism | CS score, | |
| | | | | | | | function | |
| Nrde2 | 217827 | NRDE2 | 55051 | −2.69 | biological_process | Metabolism | CS score, | |
| | | | | | | | function | |
| Nsf | 18195 | NSF | 4905 | −2.76 | intra-Golgi vesicle- | Metabolism | CS score, | |
| | | | | | mediated transport | | function | |
| Nubp1 | 26425 | NUBP1 | 4682 | −2.05 | cellular iron ion | Metabolism | CS score, | |
| | | | | | homeostasis | | function | |
| Nudcd3 | 209586 | NUDCD3 | 23386 | −1.71 | molecular_function | Metabolism | CS score, | |
| | | | | | | | function | |
| Nup155 | 170762 | NUP155 | 9631 | −1.59 | nucleocytoplasmic | Metabolism | CS score, | Zhang X, et al. Cell. |
| | | | | | transport | | mouse | 2008 Dec. |
| | | | | | | | K.O., | 12; 135(6): 1017-27 |
| | | | | | | | function | |
| Nup93 | 71805 | NUP93 | 9688 | −2.11 | protein import into | Metabolism | CS score, | |
| | | | | | nucleus | | function | |
| Nus1 | 52014 | NUS1 | 116150 | −1.94 | angiogenesis | Metabolism | CS score, | Park E J, et al. Cell |
| | | | | | | | mouse | Metab. 2014 Sep. |
| | | | | | | | K.O., | 2; 20(3): 448-57 |
| | | | | | | | function | |
| Nvl | 67459 | NVL | 4931 | −2.61 | positive regulation of | Metabolism | CS score, | |
| | | | | | telomerase activity | | function | |
| Ogdh | 18293 | OGDH | 4967 | −2.98 | tricarboxylic acid | Metabolism | CS score, | |
| | | | | | cycle | | function | |
| Osbp | 76303 | OSBP | 5007 | −2.06 | lipid transport | Metabolism | CS score, | |
| | | | | | | | function | |
| Pak1ip1 | 68083 | PAK1IP1 | 55003 | −2.28 | cell proliferation | Metabolism | CS score, | |
| | | | | | | | function | |
| Pfdn2 | 18637 | PFDN2 | 5202 | −1.32 | protein folding | Metabolism | CS score, | |
| | | | | | | | function | |
| Pgam1 | 18648 | PGAM1 | 5223 | −2.37 | glycolytic process | Metabolism | CS score, | |
| | | | | | | | function | |
| Pkm | 18746 | PKM | 5315 | −1.68 | glycolytic process | Metabolism | CS score, | Lewis S E, et al. |
| | | | | | | | mouse | 1983:267-78. |
| | | | | | | | K.O., | Plenum Publ. Corp. |
| | | | | | | | function | |
| Pmpcb | 73078 | PMPCB | 9512 | −1.77 | proteolysis | Metabolism | CS score, | |
| | | | | | | | function | |
| Ppil2 | 66053 | PPIL2 | 23759 | −3.01 | protein | Metabolism | CS score, | |
| | | | | | polyubiquitination | | function | |
| Ppp4c | 56420 | PPP4C | 5531 | −2.89 | protein | Metabolism | CS score, | Toyo-oka K, et al. J |
| | | | | | dephosphorylation | | mouse | Cell Biol. 2008 Mar. |
| | | | | | | | K.O., | 24; 180(6): 1133-47 |
| | | | | | | | function | |
| Prelid1 | 66494 | PRELID1 | 27166 | −2.27 | apoptotic process | Metabolism | CS score, | |
| | | | | | | | function | |
| Prpf31 | 68988 | PRPF31 | 26121 | −3.20 | spliceosomal tri- | Metabolism | CS score, | Bujakowska K, et al. |
| | | | | | snRNP complex | | mouse | Invest Ophthalmol |
| | | | | | assembly | | K.O., | Vis Sci. 2009 |
| | | | | | | | function | December; 50(12): 5927- |
| | | | | | | | | 33 |
| Prpf6 | 68879 | PRPF6 | 24148 | −2.96 | spliceosomal tri- | Metabolism | CS score, | |
| | | | | | snRNP complex | | function | |
| | | | | | assembly | | | |
| Psma1 | 26440 | PSMA1 | 5682 | −2.39 | proteasomal | Metabolism | CS score, | |
| | | | | | ubiquitin- | | function | |
| | | | | | independent protein | | | |
| | | | | | catabolic process | | | |
| Psma2 | 19166 | PSMA2 | 5683 | −2.23 | proteasomal | Metabolism | CS score, | |
| | | | | | ubiquitin- | | function | |
| | | | | | independent protein | | | |
| | | | | | catabolic process | | | |
| Psma3 | 19167 | PSMA3 | 5684 | −2.30 | proteasomal | Metabolism | CS score, | |
| | | | | | ubiquitin- | | function | |
| | | | | | independent protein | | | |
| | | | | | catabolic process | | | |
| Psmb2 | 26445 | PSMB2 | 5690 | −2.12 | proteasomal | Metabolism | CS score, | |
| | | | | | ubiquitin- | | function | |
| | | | | | independent protein | | | |
| | | | | | catabolic process | | | |
| Psmb3 | 26446 | PSMB3 | 5691 | −2.78 | proteolysis involved | Metabolism | CS score, | |
| | | | | | in cellular protein | | function | |
| | | | | | catabolic process | | | |
| Psmb5 | 19173 | PSMB5 | 5693 | −1.67 | proteasomal | Metabolism | CS score, | |
| | | | | | ubiquitin- | | function | |
| | | | | | independent protein | | | |
| | | | | | catabolic process | | | |
| Psmb6 | 19175 | PSMB6 | 5694 | −2.42 | proteasomal | Metabolism | CS score, | |
| | | | | | ubiquitin- | | function | |
| | | | | | independent protein | | | |
| | | | | | catabolic process | | | |
| Psmb7 | 19177 | PSMB7 | 5695 | −2.69 | proteasomal | Metabolism | CS score, | |
| | | | | | ubiquitin- | | function | |
| | | | | | independent protein | | | |
| | | | | | catabolic process | | | |
| Psmc2 | 19181 | PSMC2 | 5701 | −2.35 | protein catabolic | Metabolism | CS score, | |
| | | | | | process | | function | |
| Psmc3 | 19182 | PSMC3 | 5702 | −2.76 | ER- | Metabolism | CS score, | Sakao Y, et al. |
| | | | | | associated ubiquitin- | | mouse | Genomics. 2000 Jul. |
| | | | | | dependent protein | | K.O., | 1; 67(1): 1-7 |
| | | | | | catabolic process | | function | |
| Psmc4 | 23996 | PSMC4 | 5704 | −2.36 | blastocyst | Metabolism | CS score, | Sakao Y, et al. |
| | | | | | development | | mouse | Genomics. 2000 Jul. |
| | | | | | | | K.O., | 1; 67(1): 1-7 |
| | | | | | | | function | |
| Psmd1 | 70247 | PSMD1 | 5707 | −1.88 | regulation of protein | Metabolism | CS score, | |
| | | | | | catabolic process | | function | |
| Psmd2 | 21762 | PSMD2 | 5708 | −2.16 | regulation of protein | Metabolism | CS score, | |
| | | | | | catabolic process | | function | |
| Psmd3 | 22123 | PSMD3 | 5709 | −2.10 | regulation of protein | Metabolism | CS score, | |
| | | | | | catabolic process | | function | |
| Psmd4 | 19185 | PSMD4 | 5710 | −1.77 | ubiquitin- | Metabolism | CS score, | Soriano P, et al. |
| | | | | | dependent protein | | mouse | Genes Dev. 1987 |
| | | | | | catabolic process | | K.O., | June; 1(4): 366-75 |
| | | | | | | | function | |
| Psmd6 | 66413 | PSMD6 | 9861 | −2.27 | proteasome- | Metabolism | CS score, | |
| | | | | | mediated ubiquitin- | | function | |
| | | | | | dependent protein | | | |
| | | | | | catabolic process | | | |
| Psmg3 | 66506 | PSMG3 | 84262 | −2.57 | molecular_function | Metabolism | CS score, | |
| | | | | | | | function | |
| Ptpmt1 | 66461 | PTPMT1 | 114971 | −2.89 | protein | Metabolism | CS score, | Shen J, et al. Mol |
| | | | | | dephosphorylation | | mouse | Cell Biol. 2011 |
| | | | | | | | K.O., | December; 31(24): 4902- |
| | | | | | | | function | 16 |
| Ptpn23 | 104831 | PTPN23 | 25930 | −1.59 | negative regulation of | Metabolism | CS score, | Gingras M C, et al. |
| | | | | | epithelial cell | | mouse | Int J Dev Biol. |
| | | | | | migration | | K.O., | 2009; 53(7): 1069-74 |
| | | | | | | | function | |
| Rabggta | 56187 | RABGGTA | 5875 | −3.18 | protein prenylation | Metabolism | CS score, | |
| | | | | | | | function | |
| Rabggtb | 19352 | RABGGTB | 5876 | −2.44 | protein | Metabolism | CS score, | |
| | | | | | geranylgeranylation | | function | |
| Rbm19 | 74111 | RBM19 | 9904 | −2.03 | multicellular | Metabolism | CS score, | Zhang J, et al. BMC |
| | | | | | organismal | | mouse | Dev Biol. |
| | | | | | development | | K.O., | 2008; 8:115 |
| | | | | | | | function | |
| Rfk | 54391 | RFK | 55312 | −1.56 | riboflavin biosynthetic | Metabolism | CS score, | Yazdanpanah B, et |
| | | | | | process | | mouse | al. Nature. 2009 |
| | | | | | | | K.O., | Aug. |
| | | | | | | | function | 27; 460(7259): 1159-63 |
| Rheb | 19744 | RHEB | 6009 | −1.38 | signal transduction | Metabolism | CS score, | Zou J, et al. Dev |
| | | | | | | | mouse | Cell. 2011 Jan. |
| | | | | | | | K.O., | 18; 20(1): 97-108 |
| | | | | | | | function | |
| Riok1 | 71340 | RIOK1 | 83732 | −1.27 | protein | Metabolism | CS score, | |
| | | | | | phosphorylation | | function | |
| Rpn1 | 103963 | RPN1 | 6184 | −2.13 | protein glycosylation | Metabolism | CS score, | |
| | | | | | | | function | |
| Rtfdc1 | 66404 | RTFDC1 | 51507 | −2.09 | biological_process | Metabolism | CS score, | |
| | | | | | | | function | |
| Sacm1l | 83493 | SACM1L | 22908 | −1.80 | protein | Metabolism | CS score, | |
| | | | | | dephosphorylation | | function | |
| Samm50 | 68653 | SAMM50 | 25813 | −1.62 | protein targeting to | Metabolism | CS score, | |
| | | | | | mitochondrion | | function | |
| Sco2 | 100126824 | SCO2 | 9997 | −1.60 | eye development | Metabolism | CS score, | Yang H, et al. Hum |
| | | | | | | | mouse | Mol Genet. 2010 |
| | | | | | | | K.O., | Jan. 1; 19(1): 170-80 |
| | | | | | | | function | |
| Sdha | 66945 | SDHA | 6389 | −2.20 | tricarboxylic acid | Metabolism | CS score, | |
| | | | | | cycle | | function | |
| Sdhb | 67680 | SDHB | 6390 | −2.33 | tricarboxylic acid | Metabolism | CS score, | |
| | | | | | cycle | | function | |
| Sec61a1 | 53421 | SEC61A1 | 29927 | −2.42 | protein transport | Metabolism | CS score, | |
| | | | | | | | function | |
| Slc20a1 | 20515 | SLC20A1 | 6574 | −2.38 | sodium ion transport | Metabolism | CS score, | Festing M H, et al. |
| | | | | | | | mouse | Genesis. 2009 |
| | | | | | | | K.O., | December; 47(12): 858-63 |
| | | | | | | | function | |
| Slc7a6os | 66432 | SLC7A6OS | 84138 | −2.30 | hematopoietic | Metabolism | CS score, | |
| | | | | | progenitor cell | | function | |
| | | | | | differentiation | | | |
| Smn1 | 20595 | SMN1 | 6606 | −1.58 | spliceosomal | Metabolism | CS score, | Hsieh-Li H M, et al. |
| | | | | | complex assembly | | mouse | Nat Genet. 2000 |
| | | | | | | | K.O., | Janunary; 24(1): 66-70 |
| | | | | | | | function | |
| Smu1 | 74255 | SMU1 | 55234 | −3.65 | molecular_function | Metabolism | CS score, | |
| | | | | | | | function | |
| Snrpd1 | 20641 | SNRPD1 | 6632 | −2.79 | spliceosomal | Metabolism | CS score, | |
| | | | | | complex assembly | | function | |
| Snrpd3 | 67332 | SNRPD3 | 6634 | −3.62 | spliceosomal | Metabolism | CS score, | |
| | | | | | complex assembly | | function | |
| Snrpe | 20643 | SNRPE | 6635 | −2.74 | spliceosomal | Metabolism | CS score, | |
| | | | | | complex assembly | | function | |
| Spata5 | 57815 | SPATA5 | 166378 | −1.50 | multicellular | Metabolism | CS score, | |
| | | | | | organismal | | function | |
| | | | | | development | | | |
| Spata5l1 | 214616 | SPATA5L1 | 79029 | −2.70 | molecular_function | Metabolism | CS score, | |
| | | | | | | | function | |
| Tango6 | 272538 | TANGO6 | 79613 | −2.29 | integral component of | Metabolism | CS score, | |
| | | | | | membrane | | function | |
| n/a | n/a | TBC1D3B | 414059 | −1.67 | positive regulation of | Metabolism | CS score, | |
| | | | | | GTPase activity | | function | |
| n/a | n/a | TBC1D3C | 414060 | −2.01 | positive regulation of | Metabolism | CS score, | |
| | | | | | GTPase activity | | function | |
| Tbcb | 66411 | TBCB | 1155 | −1.97 | nervous system | Metabolism | CS score, | |
| | | | | | development | | function | |
| Tbcc | 72726 | TBCC | 6903 | −3.02 | cell morphogenesis | Metabolism | CS score, | |
| | | | | | | | function | |
| Tbcd | 108903 | TBCD | 6904 | −1.82 | microtubule | Metabolism | CS score, | |
| | | | | | cytoskeleton | | function | |
| | | | | | organization | | | |
| Tcp1 | 21454 | TCP1 | 6950 | −2.34 | protein folding | Metabolism | CS score, | |
| | | | | | | | function | |
| Telo2 | 71718 | TELO2 | 9894 | −2.34 | regulation of TOR | Metabolism | CS score, | Takai H, et al. Cell. |
| | | | | | signaling | | mouse | 2007 Dec. |
| | | | | | | | K.O., | 28; 131(7): 1248-59 |
| | | | | | | | function | |
| Tex10 | 269536 | TEX10 | 54881 | −1.26 | integral component of | Metabolism | CS score, | |
| | | | | | membrane | | function | |
| | | | | | mouse | | | |
| Tfrc | 22042 | TFRC | 7037 | −3.40 | cellular iron ion | Metabolism | CS score, | Levy J E, et al. Nat |
| | | | | | homeostasis | | mouse | Genet. 1999 |
| | | | | | | | K.O., | April; 21(4): 396-9 |
| | | | | | | | function | |
| Timm10 | 30059 | TIMM10 | 26519 | −1.99 | protein targeting to | Metabolism | CS score, | |
| | | | | | mitochondrion | | function | |
| Timm13 | 30055 | TIMM13 | 26517 | −1.62 | protein targeting to | Metabolism | CS score, | |
| | | | | | mitochondrion | | function | |
| Timm23 | 53600 | TIMM23 | 100287932 | −2.00 | protein targeting to | Metabolism | CS score, | Ahting U, et al. |
| | | | | | mitochondrion | | mouse | Biochim Biophys |
| | | | | | | | K.O., | Acta. 2009 |
| | | | | | | | function | May; 1787(5): 371-6 |
| Timm44 | 21856 | TIMM44 | 10469 | −1.73 | protein import into | Metabolism | CS score, | |
| | | | | | mitochondrial matrix | | function | |
| Tmx2 | 66958 | TMX2 | 51075 | −2.29 | biological_process | Metabolism | CS score, | |
| | | | | | | | function | |
| Tnpo3 | 320938 | TNPO3 | 23534 | −1.82 | splicing factor protein | Metabolism | CS score, | |
| | | | | | import into nucleus | | function | |
| Trmt112 | 67674 | TRMT112 | 51504 | −3.70 | peptidyl-glutamine | Metabolism | CS score, | |
| | | | | | methylation | | function | |
| Trnau1ap | 71787 | TRNAU1AP | 54952 | −1.40 | selenocysteine | Metabolism | CS score, | |
| | | | | | incorporation | | function | |
| Ttc1 | 66827 | TTC1 | 7265 | −1.74 | protein folding | Metabolism | CS score, | |
| | | | | | | | function | |
| Ttc27 | 74196 | TTC27 | 55622 | −2.54 | biological_process | Metabolism | CS score, | |
| | | | | | | | function | |
| Tti1 | 75425 | TTI1 | 9675 | −2.91 | regulation of TOR | Metabolism | CS score, | |
| | | | | | signaling | | function | |
| Tti2 | 234138 | TTI2 | 80185 | −1.94 | molecular_function | Metabolism | CS score, | |
| | | | | | | | function | |
| n/a | n/a | TUBB | 203068 | −3.40 | microtubule- | Metabolism | CS score, | |
| | | | | | based process | | function | |
| Txn2 | 56551 | TXN2 | 25828 | −1.41 | sulfate assimilation | Metabolism | CS score, | Nonn L, et al. Mol |
| | | | | | | | mouse | Cell Biol. 2003 |
| | | | | | | | K.O., | February; 23(3): 916-22 |
| | | | | | | | function | |
| Uqcrc1 | 22273 | UQCRC1 | 7384 | −1.29 | oxidative | Metabolism | CS score, | |
| | | | | | phosphorylation | | function | |
| Uqcrh | 66576 | UQCRH | 7388 | −1.28 | oxidative | Metabolism | CS score, | |
| | | | | | phosphorylation | | function | |
| Urb2 | 382038 | URB2 | 9816 | −2.25 | molecular_function | Metabolism | CS score, | |
| | | | | | | | function | |
| Vmp1 | 75909 | VMP1 | 81671 | −1.75 | exocytosis | Metabolism | CS score, | |
| | | | | | | | function | |
| n/a | n/a | VPS28 | 51160 | −3.06 | protein targeting | Metabolism | CS score, | |
| | | | | | to vacuole involved | | function | |
| | | | | | in ubiquitin- | | | |
| | | | | | dependent | | | |
| | | | | | protein catabolic | | | |
| | | | | | process via the | | | |
| | | | | | multivesicular body | | | |
| | | | | | sorting pathway | | | |
| Vps29 | 56433 | VPS29 | 51699 | −2.05 | intracellular protein | Metabolism | CS score, | |
| | | | | | transport | | function | |
| Vps52 | 224705 | VPS52 | 6293 | −1.85 | ectodermal cell | Metabolism | CS score, | Sugimoto M, et al. |
| | | | | | differentiation | | mouse | Cell Rep. 2012 Nov. |
| | | | | | | | K.O., | 29; 2(5): 1363-74 |
| | | | | | | | function | |
| Wars2 | 70560 | WARS2 | 10352 | −1.16 | vasculogenesis | Metabolism | CS score, | |
| | | | | | | | function | |
| Wdr7 | 104082 | WDR7 | 23335 | −1.47 | hematopoietic | Metabolism | CS score, | |
| | | | | | progenitor cell | | function | |
| | | | | | differentiation | | | |
| Wdr70 | 545085 | WDR70 | 55100 | −1.69 | enzyme binding | Metabolism | CS score, | |
| | | | | | | | function | |
| Wdr74 | 107071 | WDR74 | 54663 | −2.84 | blastocyst formation | Metabolism | CS score, | |
| | | | | | | | function | |
| Wdr77 | 70465 | WDR77 | 79084 | −2.19 | spliceosomal snRNP | Metabolism | CS score, | Zhou L, et al. J Mol |
| | | | | | assembly | | mouse | Endocrinol. 2006 |
| | | | | | | | K.O., | October; 37(2): 283-300 |
| | | | | | | | function | |
| Yae1d1 | 67008 | YAE1D1 | 57002 | −1.71 | molecular_function | Metabolism | CS score, | |
| | | | | | | | function | |
| Yrdc | 230734 | YRDC | 79693 | −2.33 | negative regulation of | Metabolism | CS score, | |
| | | | | | transport | | function | |
| Znhit2 | 29805 | ZNHIT2 | 741 | −2.70 | metal ion binding | Metabolism | CS score, | |
| | | | | | | | function | |
| Aars | 234734 | AARS | 16 | −2.48 | alanyl-tRNA | RNA | CS score, | |
| | | | | | aminoacylation | tran- | function | |
| | | | | | | scription, | | |
| | | | | | | protein | | |
| | | | | | | translation | | |
| Bms1 | 213895 | BMS1 | 9790 | −1.36 | ribosome assembly | RNA | CS score, | |
| | | | | | | tran- | function | |
| | | | | | | scription, | | |
| | | | | | | protein | | |
| | | | | | | translation | | |
| Bud31 | 231889 | BUD31 | 8896 | −2.46 | mRNA splicing, via | RNA | CS score, | |
| | | | | | spliceosome | tran- | function | |
| | | | | | | scription, | | |
| | | | | | | protein | | |
| | | | | | | translation | | |
| Bysl | 53414 | BYSL | 705 | −2.24 | maturation of SSU- | RNA | CS score, | Aoki R, et al. FEBS |
| | | | | | rRNA from tricistronic | tran- | mouse | Lett. 2006 Nov. |
| | | | | | rRNA transcript | scription, | K.O., | 13; 580(26): 6062-8 |
| | | | | | (SSU-rRNA, 5.8S | protein | function | |
| | | | | | rRNA, LSU-rRNA) | translation | | |
| Cars | 27267 | CARS | 833 | −2.45 | tRNA aminoacylation | RNA | CS score, | |
| | | | | | for protein translation | tran- | function | |
| | | | | | | scription, | | |
| | | | | | | protein | | |
| | | | | | | translation | | |
| Cdc5l | 71702 | CDC5L | 988 | −2.09 | mRNA splicing, via | RNA | CS score, | |
| | | | | | spliceosome | tran- | function | |
| | | | | | | scription, | | |
| | | | | | | protein | | |
| | | | | | | translation | | |
| Cdc73 | 214498 | CDC73 | 79577 | −2.58 | negative regulation of | RNA | CS score, | Wang P, et al. Mol |
| | | | | | transcription from | tran- | mouse | Cell Biol. 2008 |
| | | | | | RNA polymerase II | scription, | K.O., | May; 28(9): 2930-40 |
| | | | | | promoter | protein | function | |
| | | | | | | translation | | |
| Cebpz | 12607 | CEBPZ | 10153 | −2.11 | transcription from | RNA | CS score, | |
| | | | | | RNA polymerase II | tran- | function | |
| | | | | | promoter | scription, | | |
| | | | | | | protein | | |
| | | | | | | translation | | |
| Clasrp | 53609 | CLASRP | 11129 | −1.30 | mRNA processing | RNA | CS score, | |
| | | | | | | tran- | function | |
| | | | | | | scription, | | |
| | | | | | | protein | | |
| | | | | | | translation | | |
| Clp1 | 98985 | CLP1 | 10978 | −3.47 | mRNA splicing, via | RNA | CS score, | Hanada T, et al. |
| | | | | | spliceosome | tran- | mouse | Nature. 2013 Mar. |
| | | | | | | scription, | K.O., | 28; 495(7442): 474- |
| | | | | | | protein | function | 80 |
| | | | | | | translation | | |
| Cox5b | 12859 | COX5B | 1329 | −1.50 | transcription initiation | RNA | CS score, | |
| | | | | | from RNA | tran- | function | |
| | | | | | polymerase II | scription, | | |
| | | | | | promoter | protein | | |
| | | | | | | translation | | |
| Cpsf1 | 94230 | CPSF1 | 29894 | −2.58 | mRNA splicing, via | RNA | CS score, | |
| | | | | | spliceosome | tran- | function | |
| | | | | | | scription, | | |
| | | | | | | protein | | |
| | | | | | | translation | | |
| Cpsf2 | 51786 | CPSF2 | 53981 | −2.55 | mRNA | RNA | CS score, | |
| | | | | | polyadenylation | tran- | function | |
| | | | | | | scription, | | |
| | | | | | | protein | | |
| | | | | | | translation | | |
| Cpsf3l | 71957 | CPSF3L | 54973 | −2.09 | snRNA processing | RNA | CS score, | |
| | | | | | | tran- | function | |
| | | | | | | scription, | | |
| | | | | | | protein | | |
| | | | | | | translation | | |
| Dars | 226414 | DARS | 1615 | −2.90 | translation | RNA | CS score, | |
| | | | | | | tran- | function | |
| | | | | | | scription, | | |
| | | | | | | protein | | |
| | | | | | | translation | | |
| Dbr1 | 83703 | DBR1 | 51163 | −3.75 | RNA splicing, via | RNA | CS score, | |
| | | | | | transesterification | tran- | function | |
| | | | | | reactions | scription, | | |
| | | | | | | protein | | |
| | | | | | | translation | | |
| Ddx18 | 66942 | DDX18 | 8886 | −2.33 | RNA secondary | RNA | CS score, | |
| | | | | | structure unwinding | tran- | function | |
| | | | | | | scription, | | |
| | | | | | | protein | | |
| | | | | | | translation | | |
| Ddx23 | 74351 | DDX23 | 9416 | −3.01 | RNA secondary | RNA | CS score, | |
| | | | | | structure unwinding | tran- | function | |
| | | | | | | scription, | | |
| | | | | | | protein | | |
| | | | | | | translation | | |
| Ddx24 | 27225 | DDX24 | 57062 | −1.40 | RNA secondary | RNA | CS score, | |
| | | | | | structure unwinding | tran- | function | |
| | | | | | | scription, | | |
| | | | | | | protein | | |
| | | | | | | translation | | |
| Ddx41 | 72935 | DDX41 | 51428 | −1.74 | RNA secondary | RNA | CS score, | |
| | | | | | structure unwinding | tran- | function | |
| | | | | | | scription, | | |
| | | | | | | protein | | |
| | | | | | | translation | | |
| Ddx46 | 212880 | DDX46 | 9879 | −2.79 | mRNA splicing, via | RNA | CS score, | |
| | | | | | spliceosome | tran- | function | |
| | | | | | | scription, | | |
| | | | | | | protein | | |
| | | | | | | translation | | |
| Ddx47 | 67755 | DDX47 | 51202 | −2.20 | RNA secondary | RNA | CS score, | |
| | | | | | structure unwinding | tran- | function | |
| | | | | | | scription, | | |
| | | | | | | protein | | |
| | | | | | | translation | | |
| Ddx49 | 234374 | DDX49 | 54555 | −3.20 | RNA secondary | RNA | CS score, | |
| | | | | | structure unwinding | tran- | function | |
| | | | | | | scription, | | |
| | | | | | | protein | | |
| | | | | | | translation | | |
| Ddx54 | 71990 | DDX54 | 79039 | −2.94 | RNA secondary | RNA | CS score, | |
| | | | | | structure unwinding | tran- | function | |
| | | | | | | scription, | | |
| | | | | | | protein | | |
| | | | | | | translation | | |
| Ddx56 | 52513 | DDX56 | 54606 | −2.85 | rRNA processing | RNA | CS score, | |
| | | | | | | tran- | function | |
| | | | | | | scription, | | |
| | | | | | | protein | | |
| | | | | | | translation | | |
| Dgcr14 | 27886 | DGCR14 | 8220 | −1.76 | mRNA splicing, via | RNA | CS score, | |
| | | | | | spliceosome | tran- | function | |
| | | | | | | scription, | | |
| | | | | | | protein | | |
| | | | | | | translation | | |
| Dhx15 | 13204 | DHX15 | 1665 | −2.58 | mRNA processing | RNA | CS score, | |
| | | | | | | tran- | function | |
| | | | | | | scription, | | |
| | | | | | | protein | | |
| | | | | | | translation | | |
| Dhx16 | 69192 | DHX16 | 8449 | −1.35 | mRNA processing | RNA | CS score, | |
| | | | | | | tran- | function | |
| | | | | | | scription, | | |
| | | | | | | protein | | |
| | | | | | | translation | | |
| Dhx38 | 64340 | DHX38 | 9785 | −1.76 | mRNA splicing, via | RNA | CS score, | |
| | | | | | spliceosome | tran- | function | |
| | | | | | | scription, | | |
| | | | | | | protein | | |
| | | | | | | translation | | |
| Diexf | 215193 | DIEXF | 27042 | −2.03 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Dimt1 | 66254 | DIMT1 | 27292 | −1.87 | rRNA methylation | RNA | CS score, | |
| | | | | | | tran- | function | |
| | | | | | | scription, | | |
| | | | | | | protein | | |
| | | | | | | translation | | |
| Dis3 | 72662 | DIS3 | 22894 | −1.77 | mRNA catabolic | RNA | CS score, | |
| | | | | | process | tran- | function | |
| | | | | | | scription, | | |
| | | | | | | protein | | |
| | | | | | | translation | | |
| Dkc1 | 245474 | DKC1 | 1736 | −2.37 | box H/ACA snoRNA | RNA | CS score, | He J, et al. |
| | | | | | 3′-end processing | tran- | mouse | Oncogene. 2002 |
| | | | | | | scription, | K.O., | Oct. 31; 21(50): 7740- |
| | | | | | | protein | function | 4 |
| | | | | | | translation | | |
| Dnajc17 | 69408 | DNAJC17 | 55192 | −2.25 | negative regulation of | RNA | CS score, | Amendola E, et al. |
| | | | | | transcription from | tran- | mouse | Endocrinology. |
| | | | | | RNA polymerase II | scription, | K.O., | 2010 |
| | | | | | promoter | protein | function | April; 151(4): 1948-58 |
| | | | | | | translation | | |
| Ears2 | 67417 | EARS2 | 124454 | −1.91 | tRNA aminoacylation | RNA | CS score, | |
| | | | | | for protein translation | tran- | function | |
| | | | | | | scription, | | |
| | | | | | | protein | | |
| | | | | | | translation | | |
| Ebna1bp2 | 69072 | EBNA1BP2 | 10969 | −1.52 | ribosome biogenesis | RNA | CS score, | |
| | | | | | | tran- | function | |
| | | | | | | scription, | | |
| | | | | | | protein | | |
| | | | | | | translation | | |
| Eef1a1 | 13627 | EEF1A1 | 1915 | −3.11 | translational | RNA | CS score, | |
| | | | | | elongation | tran- | function | |
| | | | | | | scription, | | |
| | | | | | | protein | | |
| | | | | | | translation | | |
| Eef1g | 67160 | EEF1G | 1937 | −1.42 | translation | RNA | CS score, | |
| | | | | | | tran- | function | |
| | | | | | | scription, | | |
| | | | | | | protein | | |
| | | | | | | translation | | |
| Eef2 | 13629 | EEF2 | 1938 | −3.53 | translation | RNA | CS score, | |
| | | | | | | tran- | function | |
| | | | | | | scription, | | |
| | | | | | | protein | | |
| | | | | | | translation | | |
| Eftud2 | 20624 | EFTUD2 | 9343 | −3.79 | mRNA splicing, via | RNA | CS score, | |
| | | | | | spliceosome | tran- | function | |
| | | | | | | scription, | | |
| | | | | | | protein | | |
| | | | | | | translation | | |
| Eif1ad | 69860 | EIF1AD | 84285 | −2.26 | translational initiation | RNA | CS score, | |
| | | | | | | tran- | function | |
| | | | | | | scription, | | |
| | | | | | | protein | | |
| | | | | | | translation | | |
| Eif2b1 | 209354 | EIF2B1 | 1967 | −2.23 | regulation of | RNA | CS score, | |
| | | | | | translational initiation | tran- | function | |
| | | | | | | scription, | | |
| | | | | | | protein | | |
| | | | | | | translation | | |
| Eif2b3 | 108067 | EIF2B3 | 8891 | −3.00 | translational initiation | RNA | CS score, | |
| | | | | | | tran- | function | |
| | | | | | | scription, | | |
| | | | | | | protein | | |
| | | | | | | translation | | |
| Eif2s1 | 13665 | EIF2S1 | 1965 | −3.93 | translation | RNA | CS score, | |
| | | | | | | tran- | function | |
| | | | | | | scription, | | |
| | | | | | | protein | | |
| | | | | | | translation | | |
| Eif3c | 56347 | EIF3C | 8663 | −2.59 | formation of | RNA | CS score, | |
| | | | | | translation | tran- | function | |
| | | | | | preinitiation complex | scription, | | |
| | | | | | | protein | | |
| | | | | | | translation | | |
| n/a | n/a | EIF3CL | 728689 | −2.71 | formation of | RNA | CS score, | |
| | | | | | translation | tran- | function | |
| | | | | | preinitiation complex | scription, | | |
| | | | | | | protein | | |
| | | | | | | translation | | |
| Eif3d | 55944 | EIF3D | 8664 | −3.23 | formation of | RNA | CS score, | |
| | | | | | translation | tran- | function | |
| | | | | | preinitiation complex | scription, | | |
| | | | | | | protein | | |
| | | | | | | translation | | |
| Eif3f | 66085 | EIF3F | 8665 | −1.44 | formation of | RNA | CS score, | |
| | | | | | translation | tran- | function | |
| | | | | | preinitiation complex | scription, | | |
| | | | | | | protein | | |
| | | | | | | translation | | |
| Eif3g | 53356 | EIF3G | 8666 | −3.10 | translational initiation | RNA | CS score, | |
| | | | | | | tran- | function | |
| | | | | | | scription, | | |
| | | | | | | protein | | |
| | | | | | | translation | | |
| Eif3i | 54709 | EIFI | 8668 | −2.24 | formation of | RNA | CS score, | |
| | | | | | translation | tran- | function | |
| | | | | | preinitiation complex | scription, | | |
| | | | | | | protein | | |
| | | | | | | translation | | |
| Eif3l | 223691 | EIF3L | 51386 | −1.28 | translational initiation | RNA | CS score, | |
| | | | | | | tran- | function | |
| | | | | | | scription, | | |
| | | | | | | protein | | |
| | | | | | | translation | | |
| Eif4a1 | 13681 | EIF4A1 | 1973 | −1.97 | translational initiation | RNA | CS score, | |
| | | | | | | tran- | function | |
| | | | | | | scription, | | |
| | | | | | | protein | | |
| | | | | | | translation | | |
| Eif4a3 | 192170 | EIF4A3 | 9775 | −4.32 | RNA splicing | RNA | CS score, | |
| | | | | | | tran- | function | |
| | | | | | | scription, | | |
| | | | | | | protein | | |
| | | | | | | translation | | |
| Eif4g1 | 208643 | EIF4G1 | 1981 | −1.79 | nuclear- | RNA | CS score, | |
| | | | | | transcribed | tran- | function | |
| | | | | | mRNA catabolic | scription, | | |
| | | | | | process, nonsense- | protein | | |
| | | | | | mediated decay | translation | | |
| Eif5b | 226982 | EIF5B | 9669 | −2.93 | translational initiation | RNA | CS score, | |
| | | | | | | tran- | function | |
| | | | | | | scription, | | |
| | | | | | | protein | | |
| | | | | | | translation | | |
| Eif6 | 16418 | EIF6 | 3692 | −2.75 | mature ribosome | RNA | CS score, | Gandin V, et al. |
| | | | | | assembly | tran- | mouse | Nature, 2008 Oct. |
| | | | | | | scription, | K.O., | 2; 455(7213): 684-8 |
| | | | | | | protein | function | |
| | | | | | | translation | | |
| Elac2 | 68626 | ELAC2 | 60528 | −2.06 | tRNA 3′-trailer | RNA | CS score, | |
| | | | | | cleavage, | tran- | function | |
| | | | | | endonucleolytic | scription, | | |
| | | | | | | protein | | |
| | | | | | | translation | | |
| Ell | 13716 | ELL | 8178 | −2.23 | transcription | RNA | CS score, | Mitani K, et al. |
| | | | | | elongation from RNA | tran- | mouse | Biochem Biophys |
| | | | | | polymerase II | scription, | K.O., | Res Commun. 2000 |
| | | | | | promoter | protein | function | Dec. 20; 279(2): 563- |
| | | | | | | translation | | 7 |
| Etf1 | 225363 | ETF1 | 2107 | −2.44 | translational | RNA | CS score, | |
| | | | | | termination | tran- | function | |
| | | | | | | scription, | | |
| | | | | | | protein | | |
| | | | | | | translation | | |
| Exosc2 | 227715 | EXOSC2 | 23404 | −1.66 | exonucleolytic | RNA | CS score, | |
| | | | | | trimming to generate | tran- | function | |
| | | | | | mature 3′-end of 5.8S | scription, | | |
| | | | | | rRNA from tricistronic | protein | | |
| | | | | | rRNA transcript | translation | | |
| | | | | | (SSU-rRNA, 5.8S | | | |
| | | | | | rRNA, LSU-rRNA) | | | |
| Exosc4 | 109075 | EXOSC4 | 54512 | −3.21 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Exosc5 | 27998 | EXOSC5 | 56915 | −2.09 | rRNA catabolic | RNA | CS score, | |
| | | | | | process | tran- | function | |
| | | | | | | scription, | | |
| | | | | | | protein | | |
| | | | | | | translation | | |
| n/a | n/a | EXOSC6 | 118460 | −3.20 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Exosc7 | 66446 | EXOSC7 | 23016 | −2.17 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Exosc8 | 69639 | EXOSC8 | 11340 | −2.08 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Fars2 | 69955 | FARS2 | 10667 | −1.90 | tRNA aminoacylation | RNA | CS score, | |
| | | | | | for protein translation | tran- | function | |
| | | | | | | scription, | | |
| | | | | | | protein | | |
| | | | | | | translation | | |
| Farsa | 66590 | FARSA | 2193 | −3.30 | phenylalanyl-tRNA | RNA | CS score, | |
| | | | | | aminoacylation | tran- | function | |
| | | | | | | scription, | | |
| | | | | | | protein | | |
| | | | | | | translation | | |
| Farsb | 23874 | FARSB | 10056 | −2.49 | phenylalanyl-tRNA | RNA | CS score, | |
| | | | | | aminoacylation | tran- | function | |
| | | | | | | scription, | | |
| | | | | | | protein | | |
| | | | | | | translation | | |
| Fau | 14109 | FAU | 2197 | −2.64 | translation | RNA | CS score, | |
| | | | | | | tran- | function | |
| | | | | | | scription, | | |
| | | | | | | protein | | |
| | | | | | | translation | | |
| Fip1l1 | 66899 | FIP1L1 | 81608 | −1.93 | mRNA processing | RNA | CS score, | |
| | | | | | | tran- | function | |
| | | | | | | scription, | | |
| | | | | | | protein | | |
| | | | | | | translation | | |
| Ftsj3 | 56095 | FTSJ3 | 117246 | −1.50 | rRNA methylation | RNA | CS score, | |
| | | | | | | tran- | function | |
| | | | | | | scription, | | |
| | | | | | | protein | | |
| | | | | | | translation | | |
| Gle1 | 74412 | GLE1 | 2733 | −1.89 | mRNA export from | RNA | CS score, | |
| | | | | | nucleus | tran- | function | |
| | | | | | | scription, | | |
| | | | | | | protein | | |
| | | | | | | translation | | |
| Gnl3l | 237107 | GNL3L | 54552 | −1.35 | ribosome biogenesis | RNA | CS score, | |
| | | | | | | tran- | function | |
| | | | | | | scription, | | |
| | | | | | | protein | | |
| | | | | | | translation | | |
| Gtf2e1 | 74197 | GTF2E1 | 2960 | −1.22 | transcriptional open | RNA | CS score, | |
| | | | | | complex formation at | tran- | function | |
| | | | | | RNA polymerase II | scription, | | |
| | | | | | promoter | protein | | |
| | | | | | | translation | | |
| Gtpbp4 | 69237 | GTPBP4 | 23560 | −2.25 | ribosome biogenesis | RNA | CS score, | |
| | | | | | | tran- | function | |
| | | | | | | scription, | | |
| | | | | | | protein | | |
| | | | | | | translation | | |
| Hars | 15115 | HARS | 3035 | −3.49 | histidyl-tRNA | RNA | CS score, | |
| | | | | | aminoacylation | tran- | function | |
| | | | | | | scription, | | |
| | | | | | | protein | | |
| | | | | | | translation | | |
| Hars2 | 70791 | HARS2 | 23438 | −1.92 | histidyl-tRNA | RNA | CS score, | |
| | | | | | aminoacylation | tran- | function | |
| | | | | | | scription, | | |
| | | | | | | protein | | |
| | | | | | | translation | | |
| Heatr1 | 217995 | HEATR1 | 55127 | −2.58 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Hnrnpc | 15381 | HNRNPC | 3183 | −1.95 | mRNA splicing, via | RNA | CS score, | Williamson D J, et |
| | | | | | spliceosome | tran- | mouse | al. Mol Cell Biol. |
| | | | | | | scription, | K.O., | 2000 |
| | | | | | | protein | function | June; 20(11): 4094- |
| | | | | | | translation | | 105 |
| Hnrnpk | 15387 | HNRNPK | 3190 | −2.39 | mRNA splicing, via | RNA | CS score, | |
| | | | | | spliceosome | tran- | function | |
| | | | | | | scription, | | |
| | | | | | | protein | | |
| | | | | | | translation | | |
| Hnrnpl | 15388 | HNRNPL | 3191 | −1.88 | mRNA processing | RNA | CS score, | Gaudreau M C, et al. |
| | | | | | | tran- | mouse | J Immunol. 2012 |
| | | | | | | scription, | K.O., | Jun. 1; 188(11): 5377- |
| | | | | | | protein | function | 88 |
| | | | | | | translation | | |
| Hnrnpu | 51810 | HNRNPU | 3192 | −2.44 | mRNA splicing, via | RNA | CS score, | Roshon M J, et al. |
| | | | | | spliceosome | tran- | mouse | Transgenic Res. |
| | | | | | | scription, | K.O., | 2005 April; 14(2): 179- |
| | | | | | | protein | function | 92 |
| | | | | | | translation | | |
| Iars | 105148 | IARS | 3376 | −3.87 | isoleucyl-tRNA | RNA | CS score, | |
| | | | | | aminoacylation | tran- | function | |
| | | | | | | scription, | | |
| | | | | | | protein | | |
| | | | | | | translation | | |
| Iars2 | 381314 | IARS2 | 55699 | −2.83 | tRNA aminoacylation | RNA | CS score, | |
| | | | | | for protein translation | tran- | function | |
| | | | | | | scription, | | |
| | | | | | | protein | | |
| | | | | | | translation | | |
| Imp3 | 102462 | IMP3 | 55272 | −3.46 | rRNA processing | RNA | CS score, | |
| | | | | | | tran- | function | |
| | | | | | | scription, | | |
| | | | | | | protein | | |
| | | | | | | translation | | |
| Imp4 | 27993 | IMP4 | 92856 | −2.01 | rRNA processing | RNA | CS score, | |
| | | | | | | tran- | function | |
| | | | | | | scription, | | |
| | | | | | | protein | | |
| | | | | | | translation | | |
| Ints1 | 68510 | INTS1 | 26173 | −1.93 | snRNA processing | RNA | CS score, | Nakayama M, et al. |
| | | | | | | tran- | mouse | FASEB J. 2006 |
| | | | | | | scription, | K.O., | August; 20(10): 1718-20 |
| | | | | | | protein | function | |
| | | | | | | translation | | |
| Ints4 | 101861 | INTS4 | 92105 | −1.75 | snRNA processing | RNA | CS score, | |
| | | | | | | tran- | function | |
| | | | | | | scription, | | |
| | | | | | | protein | | |
| | | | | | | translation | | |
| Ints5 | 109077 | INTS5 | 80789 | −2.10 | snRNA processing | RNA | CS score, | |
| | | | | | | tran- | function | |
| | | | | | | scription, | | |
| | | | | | | protein | | |
| | | | | | | translation | | |
| Ints8 | 72656 | INTS8 | 55656 | −1.35 | snRNA processing | RNA | CS score, | |
| | | | | | | tran- | function | |
| | | | | | | scription, | | |
| | | | | | | protein | | |
| | | | | | | translation | | |
| Ints9 | 210925 | INTS9 | 55756 | −2.26 | snRNA processing | RNA | CS score, | |
| | | | | | | tran- | function | |
| | | | | | | scription, | | |
| | | | | | | protein | | |
| | | | | | | translation | | |
| Isg20l2 | 229504 | ISG20L2 | 81875 | −2.27 | ribosome biogenesis | RNA | CS score, | |
| | | | | | | tran- | function | |
| | | | | | | scription, | | |
| | | | | | | protein | | |
| | | | | | | translation | | |
| Kars | 85305 | KARS | 3735 | −2.76 | tRNA aminoacylation | RNA | CS score, | |
| | | | | | for protein translation | tran- | function | |
| | | | | | | scription, | | |
| | | | | | | protein | | |
| | | | | | | translation | | |
| n/a | n/a | KIAA0391 | 9692 | −1.56 | tRNA processing | RNA | CS score, | |
| | | | | | | tran- | function | |
| | | | | | | scription, | | |
| | | | | | | protein | | |
| | | | | | | translation | | |
| Lars | 107045 | LARS | 51520 | −1.83 | tRNA aminoacylation | RNA | CS score, | |
| | | | | | for protein translation | tran- | function | |
| | | | | | | scription, | | |
| | | | | | | protein | | |
| | | | | | | translation | | |
| Lars2 | 102436 | LARS2 | 23395 | −1.60 | tRNA aminoacylation | RNA | CS score, | |
| | | | | | for protein translation | tran- | function | |
| | | | | | | scription, | | |
| | | | | | | protein | | |
| | | | | | | translation | | |
| Las1l | 76130 | LAS1L | 81887 | −2.12 | rRNA processing | RNA | CS score, | |
| | | | | | | tran- | function | |
| | | | | | | scription, | | |
| | | | | | | protein | | |
| | | | | | | translation | | |
| Lrpprc | 72416 | LRPPRC | 10128 | −1.39 | negative regulation | RNA | CS score, | Ruzzenente B, et al. |
| | | | | | of mitochondrial RNA | tran- | mouse | EMBO J. 2012 Jan. |
| | | | | | catabolic process | scription, | K.O., | 18; 31(2): 143-56 |
| | | | | | | protein | function | |
| | | | | | | translation | | |
| Lsm2 | 27756 | LSM2 | 57819 | −2.96 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Lsm3 | 67678 | LSM3 | 27258 | −1.66 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Lsm7 | 66094 | LSM7 | 51690 | −1.96 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Magoh | 17149 | MAGOH | 4116 | −1.78 | nuclear- | RNA | CS score, | Silver D L, et al. Nat |
| | | | | | transcribed | tran- | mouse | Neurosci. 2010 |
| | | | | | mRNA catabolic | scription, | K.O., | May; 13(5): 551-8 |
| | | | | | process, nonsence- | protein | function | |
| | | | | | dependent decay | translation | | |
| Mars | 216443 | MARS | 4141 | −3.24 | methionyl-tRNA | RNA | CS score, | |
| | | | | | aminoacylation | tran- | function | |
| | | | | | | scription, | | |
| | | | | | | protein | | |
| | | | | | | translation | | |
| Mars2 | 212679 | MARS2 | 92935 | −2.31 | tRNA aminoacylation | RNA | CS score, | |
| | | | | | for protein translation | tran- | function | |
| | | | | | | scription, | | |
| | | | | | | protein | | |
| | | | | | | translation | | |
| Med17 | 234959 | MED17 | 9440 | −1.78 | regulation of | RNA | CS score, | |
| | | | | | transcription from | tran- | function | |
| | | | | | RNA polymerase II | scription, | | |
| | | | | | promoter | protein | | |
| | | | | | | translation | | |
| Med20 | 56771 | MED20 | 9477 | −2.00 | regulation of | RNA | CS score, | |
| | | | | | transcription from | tran- | function | |
| | | | | | RNA polymerase II | scription, | | |
| | | | | | promoter | protein | | |
| | | | | | | translation | | |
| Med22 | 20933 | MED22 | 6837 | −1.86 | regulation of | RNA | CS score, | |
| | | | | | transcription from | tran- | function | |
| | | | | | RNA polymerase II | scription, | | |
| | | | | | promoter | protein | | |
| | | | | | | translation | | |
| Med27 | 68975 | MED27 | 9442 | −1.48 | regulation of | RNA | CS score, | |
| | | | | | transcription from | tran- | function | |
| | | | | | RNA polymerase II | scription, | | |
| | | | | | promoter | protein | | |
| | | | | | | translation | | |
| Med30 | 69790 | MED30 | 90390 | −2.21 | regulation of | RNA | CS score, | |
| | | | | | transcription from | tran- | function | |
| | | | | | RNA polymerase II | scription, | | |
| | | | | | promoter | protein | | |
| Med8 | 80509 | MED8 | 112950 | −1.64 | regulation of | RNA | CS score, | |
| | | | | | transcription from | tran- | function | |
| | | | | | RNA polymerase II | scription, | | |
| | | | | | promoter | protein | | |
| | | | | | | translation | | |
| Mepce | 231803 | MEPCE | 56257 | −2.08 | negative regulation of | RNA | CS score, | |
| | | | | | transcription from | tran- | function | |
| | | | | | RNA polymerase II | scription, | | |
| | | | | | promoter | protein | | |
| | | | | | | translation | | |
| Mettl16 | 67493 | METTL16 | 79066 | −2.10 | rRNA base | RNA | CS score, | |
| | | | | | methylation | tran- | function | |
| | | | | | | scription, | | |
| | | | | | | protein | | |
| | | | | | | translation | | |
| Mphosph10 | 67973 | MPHOSPH10 | 10199 | −1.85 | RNA splicing, via | RNA | CS score, | |
| | | | | | transesterification | tran- | function | |
| | | | | | reactions | scription, | | |
| | | | | | | protein | | |
| | | | | | | translation | | |
| Mrpl10 | 107732 | MRPL10 | 124995 | −1.38 | translation | RNA | CS score, | |
| | | | | | | tran- | function | |
| | | | | | | scription, | | |
| | | | | | | protein | | |
| | | | | | | translation | | |
| Mrpl12 | 56282 | MRPL12 | 6182 | −1.56 | translation | RNA | CS score, | |
| | | | | | | tran- | function | |
| | | | | | | scription, | | |
| | | | | | | protein | | |
| | | | | | | translation | | |
| Mrpl21 | 353242 | MRPL21 | 219927 | −1.91 | translation | RNA | CS score, | |
| | | | | | | tran- | function | |
| | | | | | | scription, | | |
| | | | | | | protein | | |
| | | | | | | translation | | |
| Mrpl28 | 68611 | MRPL28 | 10573 | −1.50 | translation | RNA | CS score, | |
| | | | | | | tran- | function | |
| | | | | | | scription, | | |
| | | | | | | protein | | |
| | | | | | | translation | | |
| Mrpl3 | 94062 | MRPL3 | 11222 | −1.58 | translation | RNA | CS score, | |
| | | | | | | tran- | function | |
| | | | | | | scription, | | |
| | | | | | | protein | | |
| | | | | | | translation | | |
| Mrpl34 | 94065 | MRPL34 | 64981 | −1.66 | translation | RNA | CS score, | |
| | | | | | | tran- | function | |
| | | | | | | scription, | | |
| | | | | | | protein | | |
| | | | | | | translation | | |
| Mrpl4 | 66163 | MRPL4 | 51073 | −2.41 | translation | RNA | CS score, | |
| | | | | | | tran- | function | |
| | | | | | | scription, | | |
| | | | | | | protein | | |
| | | | | | | translation | | |
| Mrpl41 | 107733 | MRPL41 | 64975 | −2.15 | translation | RNA | CS score, | |
| | | | | | | tran- | function | |
| | | | | | | scription, | | |
| | | | | | | protein | | |
| | | | | | | translation | | |
| Mrpl51 | 66493 | MRPL51 | 51258 | −1.40 | translation | RNA | CS score, | |
| | | | | | | tran- | function | |
| | | | | | | scription, | | |
| | | | | | | protein | | |
| | | | | | | translation | | |
| Mrps14 | 64659 | MRPS14 | 63931 | −1.82 | translation | RNA | CS score, | |
| | | | | | | tran- | function | |
| | | | | | | scription, | | |
| | | | | | | protein | | |
| | | | | | | translation | | |
| Mrps15 | 66407 | MRPS15 | 64960 | −1.28 | translation | RNA | CS score, | |
| | | | | | | tran- | function | |
| | | | | | | scription, | | |
| | | | | | | protein | | |
| | | | | | | translation | | |
| Mrps16 | 66242 | MRPS16 | 51021 | −2.29 | translation | RNA | CS score, | |
| | | | | | | tran- | function | |
| | | | | | | scription, | | |
| | | | | | | protein | | |
| | | | | | | translation | | |
| Mrps18a | 68565 | MRPS18A | 55168 | −1.55 | translation | RNA | CS score, | |
| | | | | | | tran- | function | |
| | | | | | | scription, | | |
| | | | | | | protein | | |
| | | | | | | translation | | |
| Mrps2 | 118451 | MRPS2 | 51116 | −1.59 | translation | RNA | CS score, | |
| | | | | | | tran- | function | |
| | | | | | | scription, | | |
| | | | | | | protein | | |
| | | | | | | translation | | |
| Mrps21 | 66292 | MRPS21 | 54460 | −1.51 | translation | RNA | CS score, | |
| | | | | | | tran- | function | |
| | | | | | | scription, | | |
| | | | | | | protein | | |
| | | | | | | translation | | |
| Mrps24 | 64660 | MRPS24 | 64951 | −1.71 | translation | RNA | CS score, | |
| | | | | | | tran- | function | |
| | | | | | | scription, | | |
| | | | | | | protein | | |
| | | | | | | translation | | |
| Mrps6 | 121022 | MRPS6 | 64968 | −1.65 | translation | RNA | CS score, | |
| | | | | | | tran- | function | |
| | | | | | | scription, | | |
| | | | | | | protein | | |
| | | | | | | translation | | |
| Nars | 70223 | NARS | 4677 | −3.31 | tRNA aminoacylation | RNA | CS score, | |
| | | | | | for protein translation | tran- | function | |
| | | | | | | scription, | | |
| | | | | | | protein | | |
| | | | | | | translation | | |
| Nars2 | 244141 | NARS2 | 79731 | −1.32 | tRNA aminoacylation | RNA | CS score, | |
| | | | | | for protein translation | tran- | function | |
| | | | | | | scription, | | |
| | | | | | | protein | | |
| | | | | | | translation | | |
| Ncbp2 | 68092 | NCBP2 | 22916 | −3.00 | mRNA cis splicing, | RNA | CS score, | |
| | | | | | via spliceosome | tran- | function | |
| | | | | | | scription, | | |
| | | | | | | protein | | |
| | | | | | | translation | | |
| Nedd8 | 18002 | NEDD8 | 4738 | −2.45 | regulation of | RNA | CS score, | |
| | | | | | transcription form | tran- | function | |
| | | | | | RNA polymerase II | scription, | | |
| | | | | | promoter | protein | | |
| | | | | | | translation | | |
| Ngdn | 68966 | NGDN | 25983 | −2.35 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Nhp2 | 52530 | NHP2 | 55651 | −1.74 | rRNA pseudouridine | RNA | CS score, | |
| | | | | | synthesis | tran- | function | |
| | | | | | | scription, | | |
| | | | | | | protein | | |
| | | | | | | translation | | |
| Nip7 | 66164 | NIP7 | 51388 | −2.03 | ribosome assembly | RNA | CS score, | |
| | | | | | | tran- | function | |
| | | | | | | scription, | | |
| | | | | | | protein | | |
| | | | | | | translation | | |
| Noc2l | 57741 | NOC2L | 26155 | −2.34 | negative regulation of | RNA | CS score, | |
| | | | | | transcription from | tran- | function | |
| | | | | | RNA polymerase II | scription, | | |
| | | | | | promoter | protein | | |
| | | | | | | translation | | |
| Noc4l | 100608 | NOC4L | 79050 | −2.11 | ribosome biogenesis | RNA | CS score, | |
| | | | | | | tran- | function | |
| | | | | | | scription, | | |
| | | | | | | protein | | |
| | | | | | | translation | | |
| Nol6 | 230082 | NOL6 | 65083 | −2.28 | rRNA processing | RNA | CS score, | |
| | | | | | | tran- | function | |
| | | | | | | scription, | | |
| | | | | | | protein | | |
| | | | | | | translation | | |
| Nol9 | 74035 | NOL9 | 79707 | −2.20 | cleavage in ITS2 | RNA | CS score, | |
| | | | | | between 5.8S rRNA | tran- | function | |
| | | | | | and LSU-rRNA of | scription, | | |
| | | | | | tricistronic rRNA | protein | | |
| | | | | | transcript (SSU- | translation | | |
| | | | | | rRNA, 5.8S rRNA, | | | |
| | | | | | LSU-rRNA) | | | |
| Nop16 | 28126 | NOP16 | 51491 | −2.10 | ribosomal large | RNA | CS score, | |
| | | | | | subunit biogenesis | tran- | function | |
| | | | | | | scription, | | |
| | | | | | | protein | | |
| | | | | | | translation | | |
| Nop2 | 110109 | NOP2 | 4839 | −2.14 | rRNA processing | RNA | CS score, | |
| | | | | | | tran- | function | |
| | | | | | | scription, | | |
| | | | | | | protein | | |
| | | | | | | translation | | |
| Nop58 | 55989 | NOP58 | 51602 | −2.54 | rRNA modification | RNA | CS score, | |
| | | | | | | tran- | function | |
| | | | | | | scription, | | |
| | | | | | | protein | | |
| | | | | | | translation | | |
| Nsa2 | 59050 | NSA2 | 10412 | −1.78 | rRNA processing | RNA | CS score, | |
| | | | | | | tran- | function | |
| | | | | | | scription, | | |
| | | | | | | protein | | |
| | | | | | | translation | | |
| Nudt21 | 68219 | NUDT21 | 11051 | −2.36 | mRNA | RNA | CS score, | |
| | | | | | polyadenylation | tran- | function | |
| | | | | | | scription, | | |
| | | | | | | protein | | |
| | | | | | | translation | | |
| Osgep | 66246 | OSGEP | 55644 | −1.98 | tRNA processing | RNA | CS score, | |
| | | | | | | tran- | function | |
| | | | | | | scription, | | |
| | | | | | | protein | | |
| | | | | | | translation | | |
| Pabpn1 | 54196 | PABPN1 | 8106 | −1.92 | mRNA splicing, via | RNA | CS score, | |
| | | | | | spliceosome | tran- | function | |
| | | | | | | scription, | | |
| | | | | | | protein | | |
| | | | | | | translation | | |
| Pdcd11 | 18572 | PDCD11 | 22984 | −1.47 | rRNA processing | RNA | CS score, | |
| | | | | | | tran- | function | |
| | | | | | | scription, | | |
| | | | | | | protein | | |
| | | | | | | translation | | |
| Pes1 | 64934 | PES1 | 23481 | −2.92 | maturation of LSU- | RNA | CS score, | Lerch-Gaggl A, et |
| | | | | | rRNA from tricistronic | tran- | mouse | al. J Biol Chem. |
| | | | | | rRNA transcript | scription, | K.O., | 2002 Nov. |
| | | | | | (SSU-rRNA, 5.8S | protein | function | 22; 277(47): 45347- |
| | | | | | rRNA, LSU-rRNA) | translation | | 55 |
| Phb | 18673 | PHB | 5245 | −2.26 | regulation of | RNA | CS score, | He B, et al. |
| | | | | | transcription from | tran- | mouse | Endocrinology. |
| | | | | | RNA polymerase II | scription, | K.O., | 2011 |
| | | | | | promoter | protein | function | March; 152(3): 1047-56 |
| | | | | | | translation | | |
| Phf5a | 68479 | PHF5A | 84844 | −3.52 | mRNA splicing, via | RNA | CS score, | |
| | | | | | spliceosome | tran- | function | |
| | | | | | | scription, | | |
| | | | | | | protein | | |
| | | | | | | translation | | |
| Pnn | 18949 | PNN | 5411 | −1.34 | mRNA splicing, via | RNA | CS score, | Joo J H, et al. Dev |
| | | | | | spliceosome | tran- | mouse | Dyn. 2007 |
| | | | | | | scription, | K.O., | August; 236(8): 2147-58 |
| | | | | | | protein | function | |
| | | | | | | translation | | |
| Polr1b | 20017 | POLR1B | 84172 | −3.23 | transcription from | RNA | CS score, | Chen H, et al. |
| | | | | | RNA polymerase I | tran- | mouse | Biochem Biophys |
| | | | | | promoter | scription, | K.O., | Res Commun. 2008 |
| | | | | | | protein | function | Jan. 25; 365(4): 636- |
| | | | | | | translation | | 42 |
| Polr1c | 20016 | POLR1C | 9533 | −2.79 | transcription from | RNA | CS score, | |
| | | | | | RNA polymerase I | tran- | function | |
| | | | | | promoter | scription, | | |
| | | | | | | protein | | |
| | | | | | | translation | | |
| Polr2a | 20020 | POLR2A | 5430 | −3.15 | transcription from | RNA | CS score, | |
| | | | | | RNA polymerase II | tran- | function | |
| | | | | | promoter | scription, | | |
| | | | | | | protein | | |
| | | | | | | translation | | |
| Polr2b | 231329 | POLR2B | 5431 | −3.09 | transcription from | RNA | CS score, | |
| | | | | | RNA polymerase II | tran- | function | |
| | | | | | promoter | scription, | | |
| | | | | | | protein | | |
| | | | | | | translation | | |
| Polr2c | 20021 | POLR2C | 5432 | −3.15 | mRNA splicing, via | RNA | CS score, | |
| | | | | | spliceosome | tran- | function | |
| | | | | | | scription, | | |
| | | | | | | protein | | |
| | | | | | | translation | | |
| Polr2d | 69241 | POLR2D | 5433 | −2.23 | nuclear- | RNA | CS score, | |
| | | | | | transcribed mRNA | tran- | function | |
| | | | | | catabolic process, | scription, | | |
| | | | | | deadenylation- | protein | | |
| | | | | | dependent decay | translation | | |
| Polr2f | 69833 | POLR2F | 5435 | −2.31 | transcription from | RNA | CS score, | |
| | | | | | RNA polymerase I | tran- | function | |
| | | | | | promoter | scription, | | |
| | | | | | | protein | | |
| | | | | | | translation | | |
| Polr2g | 67710 | POLR2G | 5436 | −2.78 | nuclear- | RNA | CS score, | |
| | | | | | transcribed mRNA | tran- | function | |
| | | | | | catabolic process, | scription, | | |
| | | | | | exonucleolytic | protein | | |
| | | | | | | translation | | |
| Polr2h | 245841 | POLR2H | 5437 | −1.83 | transcription from | RNA | CS score, | |
| | | | | | RNA polymerase I | tran- | function | |
| | | | | | promoter | scription, | | |
| | | | | | | protein | | |
| | | | | | | translation | | |
| Polr2i | 69920 | POLR2I | 5438 | −2.92 | maintenance of | RNA | CS score, | |
| | | | | | transcriptional fidelity | tran- | function | |
| | | | | | during DNA- | scription, | | |
| | | | | | templated | protein | | |
| | | | | | transcription | translation | | |
| | | | | | elongation from RNA | | | |
| | | | | | polymerase II | | | |
| | | | | | promoter | | | |
| Polr2j | 20022 | POLR2J | 5439 | −3.31 | mRNA splicing, via | RNA | CS score, | |
| | | | | | spliceosome | tran- | function | |
| | | | | | | scription, | | |
| | | | | | | protein | | |
| | | | | | | translation | | |
| Polr21 | 66491 | POLR2L | 5441 | −3.55 | mRNA splicing, via | RNA | CS score, | |
| | | | | | spliceosome | tran- | function | |
| | | | | | | scription, | | |
| | | | | | | protein | | |
| | | | | | | translation | | |
| Polr3e | 26939 | POLR3E | 55718 | −2.33 | transcription from | RNA | CS score, | |
| | | | | | RNA polymerase III | tran- | function | |
| | | | | | promoter | scription, | | |
| | | | | | | protein | | |
| | | | | | | translation | | |
| Pop1 | 67724 | POP1 | 10940 | −1.79 | tRNA 5′-leader | RNA | CS score, | |
| | | | | | removal | tran- | function | |
| | | | | | | scription, | | |
| | | | | | | protein | | |
| | | | | | | translation | | |
| Pop4 | 66161 | POP4 | 10775 | −1.87 | RNA phosphodiester | RNA | CS score, | |
| | | | | | bond hydrolysis | tran- | function | |
| | | | | | | scription, | | |
| | | | | | | protein | | |
| | | | | | | translation | | |
| Ppa1 | 67895 | PPA1 | 5464 | −1.63 | tRNA aminoacylation | RNA | CS score, | |
| | | | | | for protein translation | tran- | function | |
| | | | | | | scription, | | |
| | | | | | | protein | | |
| | | | | | | translation | | |
| Ppan | 235036 | PPAN | 56342 | −1.62 | ribosomal large | RNA | CS score, | |
| | | | | | subunit assembly | tran- | function | |
| | | | | | | scription, | | |
| | | | | | | protein | | |
| | | | | | | translation | | |
| Ppp2ca | 19052 | PPP2CA | 5515 | −3.01 | nuclear- | RNA | CS score, | Gu P, et al. |
| | | | | | transcribed mRNA | tran- | mouse | Genesis. 2012 |
| | | | | | catabolic process, | scription, | K.O., | May; 50(5): 429-36 |
| | | | | | nonsense- | protein | function | |
| | | | | | mediated decay | translation | | |
| Prim1 | 19075 | PRIM1 | 5557 | −2.07 | DNA replication, | RNA | CS score, | |
| | | | | | synthesis of RNA | tran- | function | |
| | | | | | primer | scription, | | |
| | | | | | | protein | | |
| | | | | | | translation | | |
| Prpf38b | 66921 | PRPF38B | 55119 | −2.68 | mRNA processing | RNA | CS score, | |
| | | | | | | tran- | function | |
| | | | | | | scription, | | |
| | | | | | | protein | | |
| | | | | | | translation | | |
| Prpf4 | 70052 | PRPF4 | 9128 | −2.24 | RNA splicing | RNA | CS score, | |
| | | | | | | tran- | function | |
| | | | | | | scription, | | |
| | | | | | | protein | | |
| | | | | | | translation | | |
| Prpf8 | 192159 | PRPF8 | 10594 | −3.43 | mRNA splicing, via | RNA | CS score, | |
| | | | | | spliceosome | tran- | function | |
| | | | | | | scription, | | |
| | | | | | | protein | | |
| | | | | | | translation | | |
| Ptcd1 | 71799 | PTCD1 | 26024 | −1.77 | tRNA 3′-end | RNA | CS score, | |
| | | | | | processing | tran- | function | |
| | | | | | | scription, | | |
| | | | | | | protein | | |
| | | | | | | translation | | |
| Pwp2 | 110816 | PWP2 | 5822 | −2.52 | ribosomal small | RNA | CS score, | |
| | | | | | subunit assembly | tran- | function | |
| | | | | | | scription, | | |
| | | | | | | protein | | |
| | | | | | | translation | | |
| Qars | 97541 | QARS | 5859 | −3.35 | tRNA aminoacylation | RNA | CS score, | |
| | | | | | for protein translation | tran- | function | |
| | | | | | | scription, | | |
| | | | | | | protein | | |
| | | | | | | translation | | |
| Ran | 19384 | RAN | 5901 | −3.09 | ribosomal large | RNA | CS score, | |
| | | | | | subunit export from | tran- | function | |
| | | | | | nucleus | scription, | | |
| | | | | | | protein | | |
| | | | | | | translation | | |
| Rars | 104458 | RARS | 5917 | −2.30 | tRNA aminoacylation | RNA | CS score, | |
| | | | | | for protein translation | tran- | function | |
| | | | | | | scription, | | |
| | | | | | | protein | | |
| | | | | | | translation | | |
| Rars2 | 109093 | RARS2 | 57038 | −1.93 | arginyl-tRNA | RNA | CS score, | |
| | | | | | aminoacylation | tran- | function | |
| | | | | | | scription, | | |
| | | | | | | protein | | |
| | | | | | | translation | | |
| Rbm25 | 67039 | RBM25 | 58517 | −2.15 | regulation of | RNA | CS score, | |
| | | | | | alternative mRNA | tran- | function | |
| | | | | | splicing, via | scription, | | |
| | | | | | spliceosome | protein | | |
| | | | | | | translation | | |
| Rbm8a | 60365 | RBM8A | 9939 | −2.97 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rbmx | 19655 | RBMX | 27316 | −1.95 | regulation of | RNA | CS score, | |
| | | | | | alternative mRNA | tran- | function | |
| | | | | | splicing, via | scription, | | |
| | | | | | spliceosome | protein | | |
| | | | | | | translation | | |
| Rcl1 | 59028 | RCL1 | 10171 | −2.08 | endonucleolytic | RNA | CS score, | |
| | | | | | cleavage of | tran- | function | |
| | | | | | tricistronic rRNA | scription, | | |
| | | | | | transcript (SSU- | protein | | |
| | | | | | rRNA, 5.8S rRNA, | translation | | |
| | | | | | LSU-rRNA) | | | |
| Rngtt | 24018 | RNGTT | 8732 | −2.90 | transcription from | RNA | CS score, | |
| | | | | | RNA polymerase II | tran- | function | |
| | | | | | promoter | scription, | | |
| | | | | | | protein | | |
| | | | | | | translation | | |
| Rnmt | 67897 | RNMT | 8731 | −1.45 | 7-methylguanosine | RNA | CS score, | |
| | | | | | mRNA capping | tran- | function | |
| | | | | | | scription, | | |
| | | | | | | protein | | |
| | | | | | | translation | | |
| Rnpc3 | 67225 | RNPC3 | 55599 | −1.95 | mRNA splicing, via | RNA | CS score, | |
| | | | | | spliceosome | tran- | function | |
| | | | | | | scription, | | |
| | | | | | | protein | | |
| | | | | | | translation | | |
| Rpap1 | 68925 | RPAP1 | 26015 | −2.58 | transcription from | RNA | CS score, | |
| | | | | | RNA polymerase II | tran- | function | |
| | | | | | promoter | scription, | | |
| | | | | | | protein | | |
| | | | | | | translation | | |
| Rpl10 | 110954 | RPL10 | 6134 | −3.76 | translation | RNA | CS score, | |
| | | | | | | tran- | function | |
| | | | | | | scription, | | |
| | | | | | | protein | | |
| | | | | | | translation | | |
| Rpl10a | 19896 | RPL10A | 4736 | −2.15 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl11 | 67025 | RPL11 | 6135 | −2.99 | translation | RNA | CS score, | |
| | | | | | | tran- | function | |
| | | | | | | scription, | | |
| | | | | | | protein | | |
| | | | | | | translation | | |
| Rpl12 | 269261 | RPL12 | 6136 | −2.64 | ribosomal large | RNA | CS score, | |
| | | | | | subunit assembly | tran- | function | |
| | | | | | | scription, | | |
| | | | | | | protein | | |
| | | | | | | translation | | |
| Rpl13 | 270106 | RPL13 | 6137 | −3.28 | translation | RNA | CS score, | |
| | | | | | | tran- | function | |
| | | | | | | scription, | | |
| | | | | | | protein | | |
| | | | | | | translation | | |
| Rpl14 | 67115 | RPL14 | 9045 | −2.92 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl15 | 66480 | RPL15 | 6138 | −3.50 | translation | RNA | CS score, | |
| | | | | | | tran- | function | |
| | | | | | | scription, | | |
| | | | | | | protein | | |
| | | | | | | translation | | |
| Rpl18 | 19899 | RPL18 | 6141 | −3.72 | translation | RNA | CS score, | |
| | | | | | | tran- | function | |
| | | | | | | scription, | | |
| | | | | | | protein | | |
| | | | | | | translation | | |
| Rpl18a | 76808 | RPL18A | 6142 | −3.37 | translation | RNA | CS score, | |
| | | | | | | tran- | function | |
| | | | | | | scription, | | |
| | | | | | | protein | | |
| | | | | | | translation | | |
| Rpl23 | 65019 | RPL23 | 9349 | −3.02 | translation | RNA | CS score, | |
| | | | | | | tran- | function | |
| | | | | | | scription, | | |
| | | | | | | protein | | |
| | | | | | | translation | | |
| n/a | n/a | RPL23A | 6147 | −4.25 | translation | RNA | CS score, | |
| | | | | | | tran- | function | |
| | | | | | | scription, | | |
| | | | | | | protein | | |
| | | | | | | translation | | |
| Rpl24 | 68193 | RPL24 | 6152 | −2.55 | ribosomal large | RNA | CS score, | Oliver E R, et al. |
| | | | | | subunit assembly | tran- | mouse | Development. 2004 |
| | | | | | | scription, | K.O., | August; 131(16): 3907- |
| | | | | | | protein | function | 20 |
| | | | | | | translation | | |
| Rpl26 | 19941 | RPL26 | 6154 | −2.88 | translation | RNA | CS score, | |
| | | | | | | tran- | function | |
| | | | | | | scription, | | |
| | | | | | | protein | | |
| | | | | | | translation | | |
| Rpl27 | 19942 | RPL27 | 6155 | −2.25 | translation | RNA | CS score, | |
| | | | | | | tran- | function | |
| | | | | | | scription, | | |
| | | | | | | protein | | |
| | | | | | | translation | | |
| Rpl27a | 26451 | RPL27A | 6157 | −2.87 | translation | RNA | CS score, | Terzian T, et al. J |
| | | | | | | tran- | mouse | Pathol. 2011 |
| | | | | | | scription, | K.O., | August; 224(4): 540-52 |
| | | | | | | protein | function | |
| | | | | | | translation | | |
| Rpl3 | 27367 | RPL3 | 6122 | −3.27 | ribosomal large | RNA | CS score, | |
| | | | | | subunit assembly | tran- | function | |
| | | | | | | scription, | | |
| | | | | | | protein | | |
| | | | | | | translation | | |
| Rpl30 | 19946 | RPL30 | 6156 | −2.53 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl31 | 114641 | RPL31 | 6160 | −1.92 | translation | RNA | CS score, | |
| | | | | | | tran- | function | |
| | | | | | | scription, | | |
| | | | | | | protein | | |
| | | | | | | translation | | |
| Rpl32 | 19951 | RPL32 | 6161 | −3.70 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| n/a | n/a | RPL34 | 6164 | −2.37 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl35 | 66489 | RPL35 | 11224 | −2.25 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl35a | 57808 | RPL35A | 6165 | −3.20 | translation | RNA | CS score, | |
| | | | | | | tran- | function | |
| | | | | | | scription, | | |
| | | | | | | protein | | |
| | | | | | | translation | | |
| Rpl36 | 54217 | RPL36 | 25873 | −3.44 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl37 | 67281 | RPL37 | 6167 | −3.02 | translation | RNA | CS score, | |
| | | | | | | tran- | function | |
| | | | | | | scription, | | |
| | | | | | | protein | | |
| | | | | | | translation | | |
| Rpl37a | 19981 | RPL37A | 6168 | −2.62 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl38 | 67671 | RPL38 | 6169 | −2.57 | translation | RNA | CS score, | MORGAN W C, et |
| | | | | | | tran- | mouse | al. J Hered. 1950 |
| | | | | | | scription, | K.O., | August; 41(8): 208-15 |
| | | | | | | protein | function | |
| | | | | | | translation | | |
| Rpl4 | 67891 | RPL4 | 6124 | −2.67 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl5 | 100503670 | RPL5 | 6125 | −3.20 | translation | RNA | CS score, | |
| | | | | | | tran- | function | |
| | | | | | | scription, | | |
| | | | | | | protein | | |
| | | | | | | translation | | |
| Rpl6 | 19988 | RPL6 | 6128 | −3.07 | translation | RNA | CS score, | |
| | | | | | | tran- | function | |
| | | | | | | scription, | | |
| | | | | | | protein | | |
| | | | | | | translation | | |
| Rpl7 | 19989 | RPL7 | 6129 | −2.15 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpl7a | 27176 | RPL7A | 6130 | −3.45 | ribosome biogenesis | RNA | CS score, | |
| | | | | | | tran- | function | |
| | | | | | | scription, | | |
| | | | | | | protein | | |
| | | | | | | translation | | |
| Rpl7l1 | 66229 | RPL7L1 | 285855 | −1.86 | maturation of LSU- | RNA | CS score, | |
| | | | | | rRNA from tricistronic | tran- | function | |
| | | | | | rRNA, transcript | scription, | | |
| | | | | | (SSU-rRNA, 5.8S | protein | | |
| | | | | | rRNA, LSU-rRNA) | translation | | |
| Rpl8 | 26961 | RPL8 | 6132 | −4.00 | translation | RNA | CS score, | |
| | | | | | | tran- | function | |
| | | | | | | scription, | | |
| | | | | | | protein | | |
| | | | | | | translation | | |
| Rpl9 | 20005 | RPL9 | 6133 | −3.57 | translation | RNA | CS score, | |
| | | | | | | tran- | function | |
| | | | | | | scription, | | |
| | | | | | | protein | | |
| | | | | | | translation | | |
| Rplp0 | 11837 | RPLP0 | 6175 | −2.61 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rpp21 | 67676 | RPP21 | 79897 | −2.96 | tRNA processing | RNA | CS score, | |
| | | | | | | tran- | function | |
| | | | | | | scription, | | |
| | | | | | | protein | | |
| | | | | | | translation | | |
| Rpp30 | 54364 | RPP30 | 10556 | −1.79 | tRNA processing | RNA | CS score, | |
| | | | | | | tran- | function | |
| | | | | | | scription, | | |
| | | | | | | protein | | |
| | | | | | | translation | | |
| Rps10 | 67097 | RPS10 | 6204 | −2.88 | ribosomal small | RNA | CS score, | |
| | | | | | subunit assembly | tran- | function | |
| | | | | | | scription, | | |
| | | | | | | protein | | |
| | | | | | | translation | | |
| Rps11 | 27207 | RPS11 | 6205 | −2.93 | translation | RNA | CS score, | |
| | | | | | | tran- | function | |
| | | | | | | scription, | | |
| | | | | | | protein | | |
| | | | | | | translation | | |
| Rps12 | 20042 | RPS12 | 6206 | −3.33 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rps13 | 68052 | RPS13 | 6207 | −3.13 | translation | RNA | CS score, | |
| | | | | | | tran- | function | |
| | | | | | | scription, | | |
| | | | | | | protein | | |
| | | | | | | translation | | |
| n/a | n/a | RPS14 | 6208 | −3.18 | translation | RNA | CS score, | |
| | | | | | | tran- | function | |
| | | | | | | scription, | | |
| | | | | | | protein | | |
| | | | | | | translation | | |
| Rps15 | 20054 | RPS15 | 6209 | −3.20 | ribosomal small | RNA | CS score, | |
| | | | | | subunit assembly | tran- | function | |
| | | | | | | scription, | | |
| | | | | | | protein | | |
| | | | | | | translation | | |
| Rps15a | 267019 | RPS15A | 6210 | −3.18 | translation | RNA | CS score, | |
| | | | | | | tran- | function | |
| | | | | | | scription, | | |
| | | | | | | protein | | |
| | | | | | | translation | | |
| Rps16 | 20055 | RPS16 | 6217 | −2.35 | translation | RNA | CS score, | |
| | | | | | | tran- | function | |
| | | | | | | scription, | | |
| | | | | | | protein | | |
| | | | | | | translation | | |
| Rps17 | 20068 | RPS17 | 6218 | −2.69 | ribosomal small | RNA | CS score, | |
| | | | | | subunit assembly | tran- | function | |
| | | | | | | scription, | | |
| | | | | | | protein | | |
| | | | | | | translation | | |
| Rps19 | 20085 | RPS19 | 6223 | −3.49 | translation | RNA | CS score, | Matsson H, et al. |
| | | | | | | tran- | mouse | Mol Cell Biol. 2004 |
| | | | | | | scription, | K.O., | May; 24(9): 4032-7 |
| | | | | | | protein | function | |
| | | | | | | translation | | |
| Rps2 | 16898 | RPS2 | 6187 | −2.50 | translation | RNA | CS score, | |
| | | | | | | tran- | function | |
| | | | | | | scription, | | |
| | | | | | | protein | | |
| | | | | | | translation | | |
| Rps21 | 66481 | RPS21 | 6227 | −1.84 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rps23 | 66475 | RP523 | 6228 | −2.86 | translation | RNA | CS score, | |
| | | | | | | tran- | function | |
| | | | | | | scription, | | |
| | | | | | | protein | | |
| | | | | | | translation | | |
| Rps25 | 75617 | RPS25 | 6230 | −2.38 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| n/a | n/a | RPS3A | 6189 | −3.72 | translation | RNA | CS score, | |
| | | | | | | tran- | function | |
| | | | | | | scription, | | |
| | | | | | | protein | | |
| | | | | | | translation | | |
| Rps4x | 20102 | RPS4X | 6191 | −3.04 | translation | RNA | CS score, | |
| | | | | | | tran- | function | |
| | | | | | | scription, | | |
| | | | | | | protein | | |
| | | | | | | translation | | |
| Rps5 | 20103 | RPS5 | 6193 | −2.61 | translation | RNA | CS score, | |
| | | | | | | tran- | function | |
| | | | | | | scription, | | |
| | | | | | | protein | | |
| | | | | | | translation | | |
| Rps6 | 20104 | RPS6 | 6194 | −3.31 | translation | RNA | CS score, | |
| | | | | | | tran- | function | |
| | | | | | | scription, | | |
| | | | | | | protein | | |
| | | | | | | translation | | |
| Rps7 | 20115 | RPS7 | 6201 | −2.97 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rps8 | 20116 | RPS8 | 6202 | −3.44 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Rps9 | 76846 | RPS9 | 6203 | −3.16 | translation | RNA | CS score, | |
| | | | | | | tran- | function | |
| | | | | | | scription, | | |
| | | | | | | protein | | |
| | | | | | | translation | | |
| Rpsa | 16785 | RPSA | 3921 | −3.06 | ribosomal small | RNA | CS score, | Han J, et al. MGI |
| | | | | | subunit assembly | tran- | mouse | Direct Data |
| | | | | | | scription, | K.O., | Submission. 2008 |
| | | | | | | protein | function | |
| | | | | | | translation | | |
| Rsl24d1 | 225215 | RSL24D1 | 51187 | −2.76 | translation | RNA | CS score, | |
| | | | | | | tran- | function | |
| | | | | | | scription, | | |
| | | | | | | protein | | |
| | | | | | | translation | | |
| Sars | 20226 | SARS | 6301 | −2.67 | tRNA aminoacylation | RNA | CS score, | |
| | | | | | for protein translation | tran- | function | |
| | | | | | | scription, | | |
| | | | | | | protein | | |
| | | | | | | translation | | |
| Sars2 | 71984 | SARS2 | 54938 | −2.25 | seryl-tRNA | RNA | CS score, | |
| | | | | | aminoacylation | tran- | function | |
| | | | | | | scription, | | |
| | | | | | | protein | | |
| | | | | | | translation | | |
| Sart1 | 20227 | SART1 | 9092 | −2.13 | maturation of 5S | RNA | CS score, | |
| | | | | | rRNA | tran- | function | |
| | | | | | | scription, | | |
| | | | | | | protein | | |
| | | | | | | translation | | |
| Sart3 | 53890 | SART3 | 9733 | −1.88 | RNA processing | RNA | CS score, | |
| | | | | | | tran- | function | |
| | | | | | | scription, | | |
| | | | | | | protein | | |
| | | | | | | translation | | |
| Sdad1 | 231452 | SDAD1 | 55153 | −1.96 | ribosomal large | RNA | CS score, | |
| | | | | | subunit export from | tran- | function | |
| | | | | | nucleus | scription, | | |
| | | | | | | protein | | |
| | | | | | | translation | | |
| Sf1 | 22668 | SF1 | 7536 | −3.04 | mRNA splicing, via | RNA | CS score, | Shitashige M, et al. |
| | | | | | spliceosome | tran- | mouse | Cancer Sci. 2007 |
| | | | | | | scription, | K.O., | December; 98(12): 1862-7 |
| | | | | | | protein | function | |
| | | | | | | translation | | |
| Sf3a1 | 67465 | SF3A1 | 10291 | −3.18 | mRNA 3′-splice site | RNA | CS score, | |
| | | | | | recognition | tran- | function | |
| | | | | | | scription, | | |
| | | | | | | protein | | |
| | | | | | | translation | | |
| Sf3a2 | 20222 | SF3A2 | 8175 | −2.66 | mRNA 3′-splice site | RNA | CS score, | |
| | | | | | recognition | tran- | function | |
| | | | | | | scription, | | |
| | | | | | | protein | | |
| | | | | | | translation | | |
| Sf3a3 | 75062 | SF3A3 | 10946 | −2.26 | mRNA splicing, via | RNA | CS score, | |
| | | | | | transesterification | tran- | function | |
| | | | | | reactions | scription, | | |
| | | | | | | protein | | |
| | | | | | | translation | | |
| Sf3b2 | 319322 | SF3B2 | 10992 | −2.51 | mRNA splicing, via | RNA | CS score, | |
| | | | | | spliceosome | tran- | function | |
| | | | | | | scription, | | |
| | | | | | | protein | | |
| | | | | | | translation | | |
| Sf3b3 | 101943 | SF3B3 | 23450 | −4.13 | RNA splicing, via | RNA | CS score, | |
| | | | | | transesterification | tran- | function | |
| | | | | | reactions | scription, | | |
| | | | | | | protein | | |
| | | | | | | translation | | |
| Sf3b4 | 107701 | SF3B4 | 10262 | −2.60 | RNA splicing, via | RNA | CS score, | |
| | | | | | transesterification | tran- | function | |
| | | | | | reactions | scription, | | |
| | | | | | | protein | | |
| | | | | | | translation | | |
| Sfpq | 71514 | SFPQ | 6421 | −2.27 | negative regulation of | RNA | CS score, | |
| | | | | | transcription from | tran- | function | |
| | | | | | RNA polymerase II | scription, | | |
| | | | | | promoter | protein | | |
| | | | | | | translation | | |
| Sin3a | 20466 | SIN3A | 25942 | −1.74 | negative regulation of | RNA | CS score, | Dannenberg J H, et |
| | | | | | transcription from | tran- | mouse | al. Genes Dev. |
| | | | | | RNA polymerase II | scription, | K.O., | 2005 Jul. |
| | | | | | promoter | protein | function | 1; 19(13): 581-95 |
| | | | | | | translation | | |
| Smg5 | 229512 | SMG5 | 23381 | −2.35 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Smg6 | 103677 | SMG6 | 23293 | −1.18 | nuclear- | RNA | CS score, | |
| | | | | | transcribed mRNA | tran- | function | |
| | | | | | catabolic process, | scription, | | |
| | | | | | nonsense- | protein | | |
| | | | | | mediated decay | translation | | |
| Snrnp25 | 78372 | SNRNP25 | 79622 | −2.43 | mRNA processing | RNA | CS score, | |
| | | | | | | tran- | function | |
| | | | | | | scription, | | |
| | | | | | | protein | | |
| | | | | | | translation | | |
| Snrnp27 | 66618 | SNRNP27 | 11017 | −1.36 | mRNA processing | RNA | CS score, | |
| | | | | | | tran- | function | |
| | | | | | | scription, | | |
| | | | | | | protein | | |
| | | | | | | translation | | |
| Snrpd2 | 107686 | SNRPD2 | 6633 | −2.47 | RNA splicing | RNA | CS score, | |
| | | | | | | tran- | function | |
| | | | | | | scription, | | |
| | | | | | | protein | | |
| | | | | | | translation | | |
| Snrpf | 69878 | SNRPF | 6636 | −3.58 | mRNA splicing, via | RNA | CS score, | |
| | | | | | spliceosome | tran- | function | |
| | | | | | | scription, | | |
| | | | | | | protein | | |
| | | | | | | translation | | |
| Srrm1 | 51796 | SRRM1 | 10250 | −1.81 | mRNA processing | RNA | CS score, | |
| | | | | | | tran- | function | |
| | | | | | | scription, | | |
| | | | | | | protein | | |
| | | | | | | translation | | |
| Srsf1 | 110809 | SRSF1 | 6426 | −2.75 | mRNA 5′-splice site | RNA | CS score, | Xu X, et al. Cell. |
| | | | | | recognition | tran- | mouse | 2005 Jan. |
| | | | | | | scription, | K.O., | 14; 120(1): 59-72 |
| | | | | | | protein | function | |
| | | | | | | translation | | |
| Srsf2 | 20382 | SRSF2 | 6427 | −3.66 | regulation of | RNA | CS score, | Ding J H, et al. |
| | | | | | alternative mRNA | tran- | mouse | EMBO J. 2004 Feb. |
| | | | | | splicing, via | scription, | K.O., | 25; 23(4): 885-96 |
| | | | | | spliceosome | protein | function | |
| | | | | | | translation | | |
| Srsf3 | 20383 | SRSF3 | 6428 | −2.28 | mRNA splicing, via | RNA | CS score, | Jumaa H et al. Curr |
| | | | | | spliceosome | tran- | mouse | Biol. 1999 Aug. |
| | | | | | | scription, | K.O., | 26; 9(16): 399-902 |
| | | | | | | protein | function | |
| | | | | | | translation | | |
| Srsf7 | 225027 | SRSF7 | 6432 | −2.06 | mRNA splicing, via | RNA | CS score, | |
| | | | | | spliceosome | tran- | function | |
| | | | | | | scription, | | |
| | | | | | | protein | | |
| | | | | | | translation | | |
| Ssu72 | 68991 | SSU72 | 29101 | −2.57 | mRNA | RNA | CS score, | |
| | | | | | polyadenylation | tran- | function | |
| | | | | | | scription, | | |
| | | | | | | protein | | |
| | | | | | | translation | | |
| Sugp1 | 70616 | SUGP1 | 57794 | −1.36 | RNA processing | RNA | CS score, | |
| | | | | | | tran- | function | |
| | | | | | | scription, | | |
| | | | | | | protein | | |
| | | | | | | translation | | |
| Tars | 110960 | TARS | 6897 | −2.53 | tRNA aminoacylation | RNA | CS score, | |
| | | | | | for protein translation | tran- | function | |
| | | | | | | scription, | | |
| | | | | | | protein | | |
| | | | | | | translation | | |
| Tars2 | 71807 | TARS2 | 80222 | −1.91 | threonyl-tRNA | RNA | CS score, | |
| | | | | | aminoacylation | tran- | function | |
| | | | | | | scription, | | |
| | | | | | | protein | | |
| | | | | | | translation | | |
| Tbl3 | 213773 | TBL3 | 10607 | −2.41 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Thoc2 | 331401 | THOC2 | 57187 | −2.52 | mRNA processing | RNA | CS score, | |
| | | | | | | tran- | function | |
| | | | | | | scription, | | |
| | | | | | | protein | | |
| | | | | | | translation | | |
| Thoc5 | 107829 | THOC5 | 8563 | −1.57 | mRNA processing | RNA | CS score, | Mancini A, et al. |
| | | | | | | tran- | mouse | BMC Biol 2010; 8:1 |
| | | | | | | scription, | K.O., | |
| | | | | | | protein | function | |
| | | | | | | translation | | |
| Thoc7 | 66231 | THOC7 | 80145 | −2.23 | mRNA processing | RNA | CS score, | |
| | | | | | | tran- | function | |
| | | | | | | scription, | | |
| | | | | | | protein | | |
| | | | | | | translation | | |
| Timeless | 21853 | TIMELESS | 8914 | −2.27 | negative regulation | RNA | CS score, | Gotter A L, et al. Nat |
| | | | | | of transcription from | tran- | mouse | Neurosci. 2000 |
| | | | | | RNA polymerase II | scription, | K.O., | August; 3(8): 755-6 |
| | | | | | promoter | protein | function | |
| | | | | | | translation | | |
| Tsen2 | 381802 | TSEN2 | 80746 | −1.41 | tRNA-type intron | RNA | CS score, | |
| | | | | | splice site recognition | tran- | function | |
| | | | | | and cleavage | scription, | | |
| | | | | | | protein | | |
| | | | | | | translation | | |
| Tsr1 | 104662 | TSR1 | 55720 | −1.76 | ribosome biogenesis | RNA | CS score, | |
| | | | | | | tran- | function | |
| | | | | | | scription, | | |
| | | | | | | protein | | |
| | | | | | | translation | | |
| Tsr2 | 69499 | TSR2 | 90121 | −2.82 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Tufm | 233870 | TUFM | 7284 | −1.92 | translational | RNA | CS score, | |
| | | | | | elongation | tran- | function | |
| | | | | | | scription, | | |
| | | | | | | protein | | |
| | | | | | | translation | | |
| Tut1 | 70044 | TUT1 | 64852 | −2.65 | mRNA | RNA | CS score, | |
| | | | | | polyadenylation | tran- | function | |
| | | | | | | scription, | | |
| | | | | | | protein | | |
| | | | | | | translation | | |
| Twistnb | 28071 | TWISTNB | 221830 | −2.17 | transcription from | RNA | CS score, | |
| | | | | | RNA polymerase I | tran- | function | |
| | | | | | promoter | scription, | | |
| | | | | | | protein | | |
| | | | | | | translation | | |
| U2af1 | 108121 | U2AF1 | 7307 | −2.41 | mRNA splicing, via | RNA | CS score, | |
| | | | | | spliceosome | tran- | function | |
| | | | | | | scription, | | |
| | | | | | | protein | | |
| | | | | | | translation | | |
| U2af2 | 22185 | U2AF2 | 11338 | −2.80 | mRNA processing | RNA | CS score, | |
| | | | | | | tran- | function | |
| | | | | | | scription, | | |
| | | | | | | protein | | |
| | | | | | | translation | | |
| Uba52 | 22186 | UBA52 | 7311 | −2.54 | translation | RNA | CS score, | |
| | | | | | | tran- | function | |
| | | | | | | scription, | | |
| | | | | | | protein | | |
| | | | | | | translation | | |
| Ubl5 | 66177 | UBL5 | 59286 | −2.56 | mRNA splicing, via | RNA | CS score, | |
| | | | | | spliceosome | tran- | function | |
| | | | | | | scription, | | |
| | | | | | | protein | | |
| | | | | | | translation | | |
| Upf1 | 19704 | UPF1 | 5976 | −2.63 | nuclear- | RNA | CS score, | Medghalchi S M, et |
| | | | | | transcribed mRNA | tran- | mouse | al. Hum Mol Genet. |
| | | | | | catabolic process, | scription, | K.O., | 2001 Jan. |
| | | | | | nonsense- | protein | function | 15; 10(2): 99-105 |
| | | | | | mediated decay | translation | | |
| Upf2 | 326622 | UPF2 | 26019 | −2.16 | nuclear- | RNA | CS score, | Weischenfeldt J, et |
| | | | | | transcribed mRNA | tran- | mouse | al. Genes Dev. |
| | | | | | catabolic process, | scription, | K.O., | 2008 May |
| | | | | | nonsense- | protein | function | 15; 22(10): 1381-96 |
| | | | | | mediated decay | translation | | |
| Utp15 | 105372 | UTP15 | 84135 | −1.65 | maturation of SSU- | RNA | CS score, | |
| | | | | | RNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Utp20 | 70683 | UTP20 | 27340 | −2.28 | endonucleolytic | RNA | CS score, | |
| | | | | | cleavage in ITS1 to | tran- | function | |
| | | | | | separate SSU-rRNA | scription, | | |
| | | | | | from 5.8S rRNA and | protein | | |
| | | | | | LSU-rRNA from | translation | | |
| | | | | | tricistronic rRNA | | | |
| | | | | | transcript (SSU- | | | |
| | | | | | rRNA, 5.8S rRNA, | | | |
| | | | | | LSU-rRNA) | | | |
| Utp23 | 78581 | U1P23 | 84294 | −2.54 | rRNA processing | RNA | CS score, | |
| | | | | | | tran- | function | |
| | | | | | | scription, | | |
| | | | | | | protein | | |
| | | | | | | translation | | |
| Utp3 | 65961 | UTP3 | 57050 | −1.58 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Utp6 | 216987 | UTP6 | 55813 | −1.99 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Vars | 22321 | VARS | 7407 | −3.35 | tRNA aminoacylation | RNA | CS score, | |
| | | | | | for protein translation | tran- | function | |
| | | | | | | scription, | | |
| | | | | | | protein | | |
| | | | | | | translation | | |
| Wars | 22375 | WARS | 7453 | −2.22 | tryptophanyl-tRNA | RNA | CS score, | |
| | | | | | aminoacylation | tran- | function | |
| | | | | | | scription, | | |
| | | | | | | protein | | |
| | | | | | | translation | | |
| Wdr12 | 57750 | WDR12 | 55759 | −2.16 | maturation of LSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Wdr3 | 269470 | WDR3 | 10885 | −2.65 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Wdr33 | 74320 | WDR33 | 55339 | −2.63 | mRNA | RNA | CS score, | |
| | | | | | polyadenylation | tran- | function | |
| | | | | | | scription, | | |
| | | | | | | protein | | |
| | | | | | | translation | | |
| Wdr36 | 225348 | WDR36 | 134430 | −2.04 | rRNA processing | RNA | CS score, | Gallenberger M, et |
| | | | | | | tran- | mouse | al. Hum Mol Genet. |
| | | | | | | scription, | K.O., | 2011 Feb. |
| | | | | | | protein | function | 1; 20(3): 422-35 |
| | | | | | | translation | | |
| Wdr46 | 57315 | WDR46 | 9277 | −2.41 | maturation of SSU- | RNA | CS score, | |
| | | | | | rRNA from tricistronic | tran- | function | |
| | | | | | rRNA transcript | scription, | | |
| | | | | | (SSU-rRNA, 5.8S | protein | | |
| | | | | | rRNA, LSU-rRNA) | translation | | |
| Wdr61 | 66317 | WDR61 | 80349 | −2.63 | nuclear- | RNA | CS score, | |
| | | | | | transcribed mRNA | tran- | function | |
| | | | | | catabolic process, | scription, | | |
| | | | | | exonucleolytic, | protein | | |
| | | | | | 3′-5′ | translation | | |
| Wdr75 | 73674 | WDR75 | 84128 | −2.12 | regulation of | RNA | CS score, | |
| | | | | | transcription from | tran- | function | |
| | | | | | RNA polymerase II | scription, | | |
| | | | | | promoter | protein | | |
| | | | | | | translation | | |
| Xpo1 | 103573 | XPO1 | 7514 | −3.50 | ribosomal large | RNA | CS score, | |
| | | | | | subunit export from | tran- | function | |
| | | | | | nucleus | scription, | | |
| | | | | | | protein | | |
| | | | | | | translation | | |
| Yars | 107271 | YARS | 8565 | −2.78 | tRNA aminoacylation | RNA | CS score, | |
| | | | | | for protein translation | tran- | function | |
| | | | | | | scription, | | |
| | | | | | | protein | | |
| | | | | | | translation | | |
| Yars2 | 70120 | YARS2 | 51067 | −2.40 | translation | RNA | CS score, | |
| | | | | | | tran- | function | |
| | | | | | | scription, | | |
| | | | | | | protein | | |
| | | | | | | translation | | |
| Ythdc1 | 231386 | YTHDC1 | 91746 | −2.35 | mRNA splice site | RNA | CS score, | |
| | | | | | selection | tran- | function | |
| | | | | | | scription, | | |
| | | | | | | protein | | |
| | | | | | | translation | | |
| Zbtb8os | 67106 | ZBTB8OS | 339487 | −2.54 | tRNA splicing, via | RNA | CS score, | |
| | | | | | endonucleolytic | tran- | function | |
| | | | | | cleavage and ligation | scription, | | |
| | | | | | | protein | | |
| | | | | | | translation | | |
| Zc3h3 | 223642 | ZC3H3 | 23144 | −1.22 | mRNA | RNA | CS score, | |
| | | | | | polyadenylation | tran- | function | |
| | | | | | | scription, | | |
| | | | | | | protein | | |
| | | | | | | translation | | |

### DOCUMENTS CITED

Rossant J, Nagy A. Nature Biotechnology, January, pp. 23. (1999)
Thomson J A et al. Science. 282(5391):1145. (1998)
Takahashi K, Yamanaka S. Cell. 126(4):663. (2006)
Takahashi K et al. Cell. 131(5):861. (2007)
Hussein S M et al. Nature. 471(7336):58. (2011)
Laurent L C et al. Stem Cell. 8(1):106. (2011)
Lister R et al. Nature. 471(7336):68. (2011)
Yoshida Y, Yamanaka S. Circulation. 122(1):80. (2010)
Choo A B et al. STEM CELLS. 26(6):1454. (2008)
Tan H L et al. STEM CELLS. 27(8):1792. (2009)
Ben-David U, Nudel N, Benvenisty N. Nature Communications. 4: (2013a)
Fong C Y, Peh G S L, Gauthaman K, Bongso A. Stem Cell Rev and Rep. 5(1):72. (2009)
Tang C et al. Nature Biotechnology. 29(9):829. (2011)
Blum B, Bar-Nur O, Golan-Lev T, Benvenisty N. Nature Biotechnology. 27(3):281. (2009)
Menendez S et al. Aging Cell. 11(1):41. (2012)
Chung S et al. J Neurochem. 97(5):1467. (2006)
Eiges R et al. Current Biology. 11(7):514. (2001)
Huber I et al. The FASEB Journal. 21(10):2551. (2007)
Rong Z, Fu X, Wang M, Xu Y. Journal of Biological Chemistry. 287(39):32338. (2012)
Schuldiner M, Itskovitz-Eldor J, Benvenisty N. STEM CELLS. 21(3):257. (2003)
Ben-David U et al. Cell Stem Cell. 12(2):167. (2013b)
Dabir D V et al. Developmental Cell. 25(1):81. (2013)
Tohyama S et al. Stem Cell. 12(1):127. (2013)
Ghosh Z et al. Cancer Research. 71(14):5030. (2011)
Ben-David U, Benvenisty N. Nat Protoc. 9(3):729. (2014)
Lim T-T et al. PLoS ONE. 8(7):e70543. (2013)
Halloran P J, Fenton R G. Cancer Research. 58(17):3855. (1998)
Tomicic M T, Thust R, Kaina B. Oncogene. 21(14):2141. (2002)
Di Stasi A et al. N Engl J Med. 365(18):1673. (2011)
Santamaría D et al. Nature. 448(7155):811. (2007)
Diril M K et al. Proceedings of the National Academy of Sciences. 109(10):3826. (2012)
Scognamiglio R et al. Cell. 164(4):668. (2016)
Fernandez-Miranda G et al. Development. 138(13):2661. (2011)
Pellettieri J, Sánchez Alvarado A. Annu. Rev. Genet. 41:83. (2007)
Bianconi E et al. Ann. Hum. Biol. 40(6):463. (2013)
Wang T et al. Science. 350(6264):1096. (2015)
Morgan D O. (2007)
FUNG T, POON R. Seminars in Cell and Developmental Biology. 16(3):335. (2005)
Peters J-M. Mol Cell. 9(5):931. (2002)
Fukagawa T. Front Biosci. 13(13):2705. (2007)
Cassimeris L. Current Opinion in Cell Biology. 11(1):134. (1999)
Satyanarayana A et al. Development. 135(20):3389. (2008)
Kittler R et al. Nature. 432(7020):1036. (2004)
Harborth J et al. J. Cell. Sci. 114(Pt 24):4557. (2001)
Silk A D, Holland A J, Cleveland D W. The Journal of Cell Biology. 184(5):677. (2009)
Oda H et al. Molecular and Cellular Biology. 29(8):2278. (2009)
Liu Q et al. Genes & Development. 14(12):1448. (2000)
Martin-McCaffrey L et al. Developmental Cell. 7(5):763. (2004)
Uren A G et al. Curr. Biol. 10(21):1319. (2000)
Leung C G et al. J Exp Med. 204(7):1603. (2007)
Cutts S M et al. Human Molecular Genetics. 8(7):1145. (1999)
Howman E V et al. Proceedings of the National Academy of Sciences. 97(3):1148. (2000)
Chauvière M, Kress C, Kress M. Biochem Biophys Res Commun. 372(4):513. (2008)
Miura K et al. Biochem Biophys Res Commun. 341(1):132. (2006)
Magnaghi P et al. Nat Chem Biol. 9(9):548. (2013)
Smith E D et al. Molecular and Cellular Biology. 24(3):1168. (2004)
Morham S G, Kluckman K D, Voulomanos N, Smithies O. Molecular and Cellular Biology. 16(12):6804. (1996)
Akimitsu N et al. Genes Cells. 8(4):393. (2003)
Jeganathan K et al. The Journal of Cell Biology. 179(2):255. (2007)
Ono T et al. Cell. 115(1):109. (2003)
Bharadwaj R, Qi W, Yu H. J. Biol. Chem. 279(13):13076. (2004)
Parisi G, Fornasari M S, Echave J. FEBS Lett. 562(1-3):1. (2004)
Harborth J, Weber K, Osborn M. J. Biol. Chem. 275(41):31979. (2000)
Krull S et al. Mol. Biol. Cell. 15(9):4261. (2004)
Topper L M et al. Cell Cycle. 1(4):282. (2002)
Zhang Y-W et al. J. Biol. Chem. 284(27):18085. (2009)
Simmons A D et al. Human Molecular Genetics. 8(12):2155. (1999)
Plate M et al. Mol Cells. 29(4):355. (2010)
Franco M et al. Proceedings of the National Academy of Sciences. 95(17):9926. (1998)
Ohno M, Shimura Y. Genes & Development. 10(8):997. (1996)
Achsel T et al. EMBO J. 18(20):5789. (1999)
Shichijo S et al. J Exp Med. 187(3):277. (1998)
Jurica M S et al. RNA. 8(4):426. (2002)
Chari A et al. Cell. 135(3):497. (2008)
Xu C et al. J. Biol. Chem. 281(23):15900. (2006)
Yeh H et al. Genomics. 23(2):443. (1994)
Kokkola T et al. FEBS Lett. 585(17):2665. (2011)
Chan C C et al. RNA. 10(2):200. (2004)
Zhou M et al. Proceedings of the National Academy of Sciences. 105(47):18139. (2008)
Takatsu H et al. J. Biol. Chem. 273(38):24693. (1998)
Chen Y et al. Cell Research. 22(2):333. (2012)
Mansfeld J et al. Nat. Cell Biol. 13(10):1234. (2011)
He F, Wang C-T, Gou L-T. Genet. Mol. Res. 8(4):1466. (2009)
Lieu A-S et al. Int J Oncol. 37(2):429. (2010)
Preuß E et al. Human Gene Therapy. 21(8):929. (2010)
Neschadim A et al. Molecular Therapy. 20(5):1002. (2012)
Hedley D, Ogilvie L, Springer C. Nat Rev Cancer. 7(11):870. (2007)
Nawa A et al. Anticancer Agents Med Chem. 8(2):232. (2008)
Danks M K et al. Cancer Research. 67(1):22. (2007)
Shah K. Adv. Drug Deliv. Rev. 64(8):739. (2012)
Ramnaraine M et al. Cancer Research. 63(20):6847. (2003)
Denny W A. J. Biomed. Biotechnol. 2003(1):48. (2003)
Ireton G C, McDermott G, Black M E, Stoddard B L. J. Mol. Biol. 315(4):687. (2002)
Humerickhouse R et al. Cancer Research. 60(5):1189. (2000)
Wierdl M et al. Cancer Research. 61(13):5078. (2001)
Kojima A, Hackett N R, Ohwada A, Crystal R G. J. Clin. Invest. 101(8):1789. (1998)
Straathof K C et al. Blood. 105(11):4247. (2005)
Soucek L et al. Cancer Research. 62(12):3507. (2002)
Oricchio E et al. Cell Reports, pp. 1. (2014)
Olive M et al. J. Biol. Chem. 272(30):18586. (1997)
Urlinger S et al. Proceedings of the National Academy of Sciences. 97(14):7963. (2000)
Agha-Mohammadi S et al. J Gene Med. 6(7):817. (2004)
Gossen M et al. Science. 268(5218):1766. (1995)
Banaszynski L A et al. Cell. 126(5):995. (2006)
Maeder M L et al. Nature Methods. 10(3):243. (2013)
Pollock R, Clackson T. Current Opinion in Biotechnology. 13(5):459. (2002)
Mullick A et al. BMC Biotechnology. 6(1):43. (2006)
No D, Yao T P, Evans R M. Proceedings of the National Academy of Sciences. 93(8):3346. (1996)
Stanley S A et al. Nature Medicine. 21(1):92. (2015)
Rollins C T et al. Proceedings of the National Academy of Sciences. 97(13):7096. (2000)
Cong L et al. Science. 339(6121):819. (2013)
Hsu P D et al. Nature Biotechnology. 31(9):827. (2013)
Ran F A et al. Nat Protoc. 8(11):2281. (2013)
Gertsenstein M et al. PLoS ONE. 5(6):e11260. (2010)
Adewumi O et al. Nature Biotechnology. 25(7):803. (2007)
Guy C T, Cardiff R D, Muller W J. Molecular and Cellular Biology. 12(3):954. (1992)
Szymczak A L et al. Nature Biotechnology. 22(5):589. (2004)
Nagy A. et al. Manipulating the Mouse Embryo. (2003)

1. (canceled)

2. An isolated, genetically modified mammalian cell comprising an exogenous polynucleotide encoding herpes simplex virus-thymidine kinase (HSV-TK) operably linked to a promoter of an endogenous cyclin dependent kinase 1 (CDK1) gene encoding CDK1, wherein the cell co-expresses the HSV-TK and the CDK1 under control of the CDK1 gene promoter.

3. The mammalian cell of claim 2, wherein the mammalian cell is selected from the group consisting of a human, mouse, rat, and non-human primate cell.

4. The mammalian cell of claim 2, wherein the mammalian cell is a human cell.

5. The mammalian cell of claim 2, wherein the cell further comprises an exogenous polynucleotide encoding HSV-TK operably linked to a promoter of an endogenous DNA topoisomerase II alpha (TOP2A) gene encoding TOP2A, wherein the cell co-expresses the HSV-TK and the TOP2A under control of the TOP2A gene promoter.

6. A population comprising a plurality of the mammalian cell of claim 2.

7. A method of making the mammalian cell of claim 2, comprising operably linking an exogenous polynucleotide encoding HSV-TK to a promoter of an endogenous CDK1 gene encoding CDK1, whereby the HSV-TK and the CDK1 are co-expressed under control of the CDK1 gene promoter.

8. The method of claim 7, wherein the mammalian cell is selected from the group consisting of a human, mouse, rat, and non-human primate cell.

9. The method of claim 7, wherein the mammalian cell is a human cell.

10. The method of claim 7, wherein the method further comprises operably linking an exogenous polynucleotide encoding HSV-TK to a promoter of an endogenous TOP2A gene encoding TOP2A, whereby the HSV-TK and the TOP2A are co-expressed under control of the TOP2A gene promoter.

11. A method of promoting death of the mammalian cell of claim 2 comprising contacting the mammalian cell with ganciclovir, thereby promoting the death thereof.

12. The method of claim 11, wherein the mammalian cell is selected from the group consisting of a human, mouse, rat, and non-human primate cell.

13. The method of claim 11, wherein the mammalian cell is a human cell.

14. The method of claim 11, wherein the mammalian cell is a human cell and wherein the human cell is in a human subject that has been administered the human cell.

15. The method of claim 11, wherein, prior to the contacting, the method comprises administering the mammalian cell to a subject.

16. The method of claim 15, wherein the mammalian cell is a human cell, and wherein the subject is a human subject.

17. A method of performing cell therapy comprising administering to a subject the mammalian cell of claim 2.

18. The method of claim 17, wherein the mammalian cell is selected from the group consisting of a human, mouse, rat, and non-human primate cell.

19. The method of claim 17, wherein the mammalian cell is a human cell.

20. The method of claim 17, wherein the mammalian cell is a human cell, and wherein the subject is a human subject.

21. The method of claim 17, wherein the method further comprises administering ganciclovir to the subject, thereby promoting death of the mammalian cell.


SEQUENCE LISTING (155 sequences)

SEQ ID NO: 1 - DNA, 562 bp, Artificial Sequence
  Synthetic DNA
gtcgacggttctggcgtgaaacagactttgaattttgaccttctcaagttggcgggagacgtggagtccaacccagggcccatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtggcggccgcattaccgcgg

SEQ ID NO: 2 - DNA, 2031 bp, Artificial Sequence
  Synthetic DNA
gtcgacattaccgcgggttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgagatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggctagcaagctt

SEQ ID NO: 3 - DNA, 1848 bp, Artificial Sequence
  Plasmid DNA
gcggccgcatccaggcggccgagatccgaagttcctatactatttgaagaataggaacttcagatcccccgggctgcaggatctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatcgagaaattgatgatctattaaacaataaagatgtccacatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatccggtcgacgaagttcctatactatttgaagaataggaacttcggatctggccggcctagggataacagggtaatggatcccccgggctgcaggaattcgatatcaagctt

SEQ ID NO: 4 - DNA, 8003 bp, Artificial Sequence
  synthetic DNA
ggtctaccttaagccaaggtagaaatacttggttgaaaaatgtttctgctgccaggcagtggcagcacacaccagcacttggggggacagaggcaggcagatctcttgagtttaaggccagcctagtctatagagcaagtcctggaacacccagggctacacagagaaaccttgtctcagaaaaccaaacaaaaagttctgttctggtagtgttggctaactgcttttttattgccacttcactcttgtttgtgtggtctgtgaaaaggaagacagaccctttggcatctttgtcaggggagattgatggcctggggggacctccaggaaggggcttattgtagttttaggggttctcgtttctctgtgttgaacttccgcagagctgtgcttcctcctgattggacctgctgtgctcattcagatcaattaaaaggcaggttttgctgtgtgagggagattattgtgaaagatttaggagccagcctactctagctgggggagggagcactagggattgaaccccagtctttggcccctgttgcttgggtgtcctgtcactgagctgtgcccttggccctgtcatcaggctttgtcagtggattcccatgtggttgtctgttttattattttcctctgcaatggggcttgaaccatgggccacacacacacactcacacactctgctccttgttttgagaacgtctcactgacttgccctggttggccctgaggtcactggcgtagatagactttggacctgggactcctacagcggccttcccagtagtaggtctacagcaccagactaggttcctctggtatttctgaaagggcattggcttgtataatacaggaacattggcatcttgggaggtgtgagcctggagtctacagtgtcggtcccctgtgtgtcatttggattttgaggcagggtctcctgtagccctgactggcctcattctctgggtgctaagattacacagatgtgtgccctgctggggccctggtcctgctcgttttgcctgatagggaagctagccacccactgagcagccttctcagtcctgtcttgtttattcttttttattttttcacagtttatgatatgactcgtctttaatggctgtaactgctgctgacctcattgcattgttcattgaggtcattcactattttgataaataaactgtagttctaaatcactaatcatttgaatcccctcctaccgctttggcttagatttcttttcatttttcttttttttctttttttttggtttttttgagacagggtttctctgtgtagctctggctgtcctggaactcactttgtagaccaggctggcctcaaactctgcctccagagtgctggtattaaagttgtatgccagccaggtatatgccacccaggtatatgccagccaggatatggctgctttggtcctttgactttagagatctgaaaagcaagaggtctatggtacttacccactaatgagcagaaatcagaaataaaataggttcaacttagtaatttgcatatagctaattgtctgaagttcttggtgacacctgcacagcttcctaaaagacaaccgtgtgtggtctgtcctgtgccaggtagtgacgctgtggtaccgatctccagaagtgttgctgggctcggctcgttactccactccggttgacatctggagtatagggaccatatttgcagaactggccaccaagaagccgcttttccacggcgactcagagattgaccagctcttcaggatcttcaggtaattgtgttttatacctaagctgttaaaagcctgtatggttaaaataataaaggcaccgtgtatgtgataggacttttgcttcctttcagagctctgggcactcctaacaacgaagtgtggccagaagtcgagtccctgcaggactacaagaacacctttcccaagtggaagccggggagcctcgcatcccacgtcaagaacctggacgagaacggcttggatttgctctcagtaagtgagctgttctttatggttcgagcgtggtgcccttgggtgtcagtagtgacaggtaagggtgactggatgtactcagtcctggacttgtcagcacgaagacacagcagctgagtcggatgcccggtaatcctgtagtgacagcgagctgcctttgaggtgtgaagtgcctggtggtctgaggagcctctggtgttggactttgtcatttacttctcatccggtaggagaggctggactcacacaccttcttgcttgagagtatgttgatgagttacaatgactcactcctctctgtttgctactgagccctcagttcatcggtttacactgttgactgcttaggatactgagctgttcaggcttttgacagcataactccattgctcaggtctggccttgtttcctcagaaaagctagcaaagactaattaattctgattttcttcagttgtcttggtgcacaatattagcaatgcaaagaacaaaattactgttcaggattaataaaggtctcctttaggaaatttgctgtaggtagagcagggtttaaaatctgagtttgtagaaggaatcaagtagacgattcagttggtctttccgctcccttgcaatctataaacgatgccaccaaatggggctcagatttgtccagacccctacagctgtcacatagtttgttacatattggtattggctaacttagaagccttcccgtagcctgggtccaatctgctggtattatctaaagttattagctctgtgtgtgtgtgtgtgtgtgtgtgtgtgtgtgtgtgtgtgtgtgtgttgctgggggctatggacagaaggcagagctttcttgaggtggtgtaggaaggttggtcattgactgttgaggcgcagccctacttctcagtctttaaacaaaggacttcacttaacagacatcagttctgggcaccaatttcctctgttgagccaacaccctgcaatagctaagcttgccgtggccctgctgcctgctgagcactgtggctatcactgccctgtcccccactccagcggcagactgctgagttagcagggcctgtgtctatcgcaggaccccagaagcaaactgccccagttggagaccactggattattgatgagacttggcttcagtttattttctcccacattgatggattcgggcaagtttcttgctgtccttgaaagacttggtgtcttaagccaaacagtgtggcacatctttctcatatgtctgtgcggactgacagacatatcctcaaggccttcatatttatttgtttgcctttttaaaaatttacttatttattttacatatgcaaatacactgttgctgtcctcagacacagtagaagagggcatcggatcccattacagatggttgtgagccaccatgtggttgctgggagttgaactcaggacttctggaagagtagtcatcgctcttaattgctgagccatctctccacctccttaatctattttctctgaagataatatgcatgatcagatttaagatctggagtatttaaattagtttacaataattagtccatctattataactgtgtttataaaaatgctatggggtttttcctttgttggcttgacgaaggggttgtgtggacactatcatgaaacactataactgattgctactttttcagttgtgaataacagatggacatttaatttctagggtagatataattagctagataatgtgaagatttatgttttggttttaagctgtctgggtgcttatcttataatttataacaggttaccaaggtgtctgtgtgtgatttaagtatcttggagaggtaaatagagggcaggcttctaaatttatttccctttttctctctagaaaatgctagtctatgatcctgccaaacgaatctctggcaaaatggccctgaagcacccgtactttgatgacttggacaatcagattaagaagatgtagccctctggatggatgtccctgtctgctggtcgtaggggaagatcgtgttgtttaccgttggctctcttcctgtcttgtatagttttctttgtttgtaaactgtcatctggacttttcttaatttcctacgtataacttaattaacatgtaaatattattccatatgaatttaaatataattctgtatatgtgcagatgtcactgtggtggctgttaattactataacacaagtgttaattactacaacataagacttgagtctccctagacttcccagcagccattcctgcagctcggagcacagttgaaggagctgagctcaggcctcgtgatgctttcaagtgcctccgtgttctggatatatatgattcctggtcagtttcttgccatttggaaactacaacccacctacggacagtgtttttctacttgtgcttaagcagttgggatgagaaggccaaagacccgaggatgtctagagtaatgacccccagatggaagtgcaccaaagctggctgggtttcacagctagagatcaggggctgtccagagcaggacagcttagaacatttatgaagactccctatttttaggtttgttgtaaagctgttgtctagttggattcctgtgctctgcatggtcagaggtaggttagaggatttgccttggcttctaaatccaatttgaaaactgcttaaaatctcctgtcctctcttagcagtgtctaaaaatgtccttgtccaaatatttagctgagattcctcactttggaaaaggagccgtatcgctgtgctgcttagtgtaattcttagaagcagcctgacttatctgctagcagtcaaagggatgcctgagacgactgctcctcttagaactaaaggctgggatgcttaagtttgtctactgtttggaggatctcggtaagactgagcccctgttcctgtggccacctcagtttaccagtacctcagcctcagcctcctgcatttgctggagtcagggaaggtccccagccctgagccctgacgctcctgattgtagagactgtcagttggaggtaaaacgttcattgaagtagtcagcagccacatgcatttagggcactccagtgtcagagaccatcctggaggtttctaaccctgccgctggcagtctactcccaagacagatcagttagagtggtcagcaaacaccaactgctgcagaaacctgtgttggtgtggttccctgctgctgctgggaactgggcccaagactagagagcttggggctgcggttgatcatgggttctgttcctgcattacaccttgaaatccaagccttctaatatctcccttcggatcataagttgtgaatttggtcctccgcccccgccaggttttctatacttgggtttgtcttgctgacattttcaagagtcctgactaagacggtgattagtgtgacatgacttgagaactaccgatttgaagcacacttgaagttaacaaattctctcatgattatacttttaactttttataagattgcttgagctcacccagatctctgttgggaagtaactgggtaacaaaagccgttgcactggtttgacagctaacaactgttggtactttgtattcagaaggaatgaggtagcgattgaatggctggggtgttgtttccacagtttatacactaaaaatttgggtagccaggaggtagtggcgcgcaatctttaatctcagcagaggcagtcctatctcttgagttggaggccagcctgcctgagttccagaccagccagggctacaggaagaagtcttaaaaaaattttttttccctgtggatgtaaacccatgagaatgactgctgtatctatcactttctgtataatagaggtttcttccgccgcagcgctccattggtctcagcccctcatgttgggggcgggaaggaggggcaagcgtccccaagcacaggaaggcctgctgaagagttggcggctttgagggttcaaatcctgcttgggtctgaggagtcccccaaacgatcttggactgtaaaaacctgccaggaacaagcgctaagggatggttctcaacctgtgggtcgcgaccccctggggagttggacagccctttcctgaggatcagaacaaacatcctgcatatcagatattaacgtggcatttcataactgcaaacttacagttaggaagtagcagtgacaaagaattttatgggtggggtggggtgggggctcaccaccatattaaagggttacagcattaggagggttgacaacagaaccatcgcactgagggaaagtgagttagcacgaagagatgagcatttcccagaacacagaataagacacgtttaaccaacatcagtctgtaaccccagcggactgtgacaagctctcacccatgccgtggagctcagaactagcattgcacgcttttaccccatctttggttgcctcactccctttgagtagtaagtccctcttcaaagtaatagcctctaaacagcctgctgcgcatgctctagtcctttgctttcctccacgggtaggccacaggcttaagcagcagttggaaatggaacttgggctttctggctcctggtttagaaaactcttgaaatggacaagatttagtcctaggcaagttctgtgtagtcagcaatggaagcaaaggggggtggggcaaacattccagtcaggtggaatctgagcccttgagagtgtgaggcaggaggatcactaggcaggagttctcaaccagcctagacgacagcttggaacccatcgcactccacaatgaggacccctcagcttctgaagagaggagagctgctagctctgagaagggtgtataaaatgggcttttggttcccagacatcagacttgaagaggcaaccgagtaagccagctatagactaagtaagattgtcgtacctagcctactccttgggagctcattcttcagagtatgttgatggatgacctaacgccatctcagatgctggagcattttgctggaaaggctctcagcactagcgaggctgcctcaatgtaatcctgcctcagcctgagaactgattatgagttaccgtttggtactttatgtatttttctctcttcctagtaaccaaggacaaacctgcttccttccagccacgtggcagacactagctgtactaaagtattattacacaccggtcccctgctgccagaacaaagaactcagcctcgagaaatttctcctattatatttgggatccagcatgtcccaagacagggacgactcaataatttaatgtgcctaatggcacggaataaaaggattattcacaaacagaaaaccggacttaagcagatgtttatttgaacacaaataccataaccctccttctcatgatgttcccaattacacgttgcaacattttcacgaagaagctctttctaaaacggccttctagggagcgaataaacaggactatatttggctaacagctgcttctgaaaactgagatgtcagcaaagcaagctgttgtttaaagaaaactaattacccagctaccttgggcttggatgggggtaaataacgccctcccgtttcttggcagtgatttgtctgggattttaatactgttaagattttatttctcttcctccacttctcctttggatcaaatggtttttaaaggattccttgtgttcctgctgccaatggcgagtcctttgcgtttgccgcccctgcttcgtcaccaactgctattttgtttagtgcaaagcagtgtgtttaaggacacactaaactttagatgacccagtgacacagtgatccgtatttgcaagcgtctggttatgcagatgtcattgtgttgtttatcagcaatgtcagcaatgtctttttttttttttttttatgcacaggctttgaaacttattttggaaattcattcattttgaggggaggcagtgagctgggagttatacaatctctcttt

SEQ ID NO: 5 - DNA, 50 bp, Artificial Sequence
  Synthetic DNA
ccctgaagcacccgtactttgatgacttggacaatcagattaagaagatg

SEQ ID NO: 6 - DNA, 50 bp, Artificial Sequence
  Synthetic DNA
tagccctctggatggatgtccctgtctgctggtcgtaggggaagatcgtg

SEQ ID NO: 7 - DNA, 755 bp, Artificial Sequence
  synthetic DNA
tgtggcacatctttctcatatgtctgtgcggactgacagacatatcctcaaggccttcatatttatttgtttgcctttttaaaaatttacttatttattttacatatgcaaatacactgttgctgtcctcagacacagtagaagagggcatcggatcccattacagatggttgtgagccaccatgtggttgctgggagttgaactcaggacttctggaagagtagtcatcgctcttaattgctgagccatctctccacctccttaatctattttctctgaagataatatgcatgatcagatttaagatctggagtatttaaattagtttacaataattagtccatctattataactgtgtttataaaaatgctatggggtttttcctttgttggcttgacgaaggggttgtgtggacactatcatgaaacactataactgattgctactttttcagttgtgaataacagatggacatttaatttctagggtagatataattagctagataatgtgaagatttatgttttggttttaagctgtctgggtgcttatcttataatttataacaggttaccaaggtgtctgtgtgtgatttaagtatcttggagaggtaaatagagggcaggcttctaaatttatttccctttttctctctagaaaatgctagtctatgatcctgccaaacgaatctctggcaaaatggccctgaagcacccgtactttgatgacttggacaatcagattaagaagatg

SEQ ID NO: 8 - DNA, 842 bp, Artificial Sequence
  synthetic DNA
ccctctggatggatgtccctgtctgctggtcgtaggggaagatcgtgttgtttaccgttggctctcttcctgtcttgtatagttttctttgtttgtaaactgtcatctggacttttcttaatttcctacgtataacttaattaacatgtaaatattattccatatgaatttaaatataattctgtatatgtgcagatgtcactgtggtggctgttaattactataacacaagtgttaattactacaacataagacttgagtctccctagacttcccagcagccattcctgcagctcggagcacagttgaaggagctgagctcaggcctcgtgatgctttcaagtgcctccgtgttctggatatatatgattcctggtcagtttcttgccatttggaaactacaacccacctacggacagtgtttttctacttgtgcttaagcagttgggatgagaaggccaaagacccgaggatgtctagagtaatgacccccagatggaagtgcaccaaagctggctgggtttcacagctagagatcaggggctgtccagagcaggacagcttagaacatttatgaagactccctatttttaggtttgttgtaaagctgttgtctagttggattcctgtgctctgcatggtcagaggtaggttagaggatttgccttggcttctaaatccaatttgaaaactgcttaaaatctcctgtcctctcttagcagtgtctaaaaatgtccttgtccaaatatttagctgagattcctcactttggaaaaggagccgtatcgctgtgctgcttagtgtaattcttagaagcagcctgacttatctgctagcagtca

SEQ ID NO: 9 - DNA, 30 bp, Artificial Sequence
  oligonucleotide
tgtggcacatctttctcatatgtctgtgcg

SEQ ID NO: 10 - DNA, 30 bp, Artificial Sequence
  oligonucleotide
gaagcagcctgacttatctgctagcagtca

SEQ ID NO: 11 - DNA, 2034 bp, Artificial Sequence
  synthetic DNA
ggttctggcgtgaaacagactttgaattttgaccttctcaagttggcgggagacgtggagtccaacccagggcccataacttcgtataatgtatgctatacgaagttattaggtctgaagaggagtttacgtccagccaagctagcttggctgcaggtcgtcgacggtatcgataagcttgatatcgaatttcgagggcccctgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagagcttggcgcgcc

SEQ ID NO: 12 - DNA, 1971 bp, Artificial Sequence
  synthetic DNA
ggcgcgccataacttcgtataatgtatgctatacgaagttatctatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgagatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagatctgaattc

SEQ ID NO: 13 - DNA, 1726 bp, Artificial Sequence
  synthetic DNA
gcggccgcatctagagtcgaccagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcatcccggggatctgatatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgaggtcgacgaattcggatcc

SEQ ID NO: 14 - DNA, 847 bp, Artificial Sequence
  synthetic DNA
gttacttcccttcgagcctcaatgtcctcatttgtaaaatgacattaatacctacttttagctgtgggaattgagtaccatgatttatacaaagcagtttgtatggtgctggttacatgagagttcagatggtaactagttagtaaaaaatctctagtgtgcttgttgattttattttattttagtatttcttaaagatcaaatttaacatcaatcctaaactttatttagctttttctggcgcgtaaactaacatactaagttgtgtgactataattcatttagtgactcatttttagctatttttataacacattgtgctatggggggttttggaacttgctggaagctacatcagaaactgccatagttaattgccatttcaagaatgttgtaaataactcaggtggccgtttaattctcaatgtaaatataattaactagacgtctttcctatatttgtgtctcagttttaaagctatttctggatgcttgagtcttaccgtaattgataacaaaaagaggttattgagaatatctatgatttacagagtaagttattctagacctcaagagtgaaatgtaggggaggagacatttgtgtgttaaactaatggaaatgctcatttaatagatattcactgaaagtattagttttggtttattgctagaaaagttgaggttttatggagatttttgtaaaaaatggtttatttcctaaataaatatctctttttcttttttctcccagaaaatgttaatctatgatccagccaaacgaatttctggcaaaatggcactgaatcatccatattttaatgatttggacaatcagattaagaagatg

SEQ ID NO: 15 - DNA, 4382 bp, Artificial Sequence
  synthetic DNA
ggttctggcgtgaaacagactttgaattttgaccttctcaagttggcgggagacgtggagtccaacccagggcccatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtggcggccgcagatccgaagttcctatactatttgaagaataggaacttcagatcccccgggctgcaggatctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatcgagaaattgatgatctattaaacaataaagatgtccacatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatccggtcgacgaagttcctattcttcaaatagtataggaacttcggatctggccggcctagggataacagggtaatggatcccccgggctgcaggaattcgatatcaagctgcgggttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgagatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 16 - DNA, 931 bp, Artificial Sequence
  synthetic DNA
ctttctgacaaaaagtttccatatgttatatcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagtttaaaactacacattttgacatagtgtttattagcagccatctaaaaaggctctaatgtatatttaactaaaattactagctttgggaattaaactgtttaacaaataatgctgctcattgtgattcttacctataagcagcctaatttgaattatttgctgc

SEQ ID NO: 17 - DNA, 1623 bp, Artificial Sequence
  synthetic DNA
accggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatatcagatccccgggatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctggtcgactctagatgcggcgaagttcctattcttcaaatagtataggaacttcggccggcc

SEQ ID NO: 18 - DNA, 3990 bp, Artificial Sequence
  synthetic DNA
ggatccggttctggcgtgaaacagactttgaattttgaccttctcaagttggcgggagacgtggagtccaacccagggcccataacttcgtataatgtatgctatacgaagttattaggtctgaagaggagtttacgtccagccaagctagcttggctgcaggtcgtcgacggtatcgataagcttgatatcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctcccgaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagagcttggcgcgccataacttcgtataatgtatgctatacgaagttatctatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgagatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 19 - DNA, 3860 bp, Artificial Sequence
  synthetic DNA
ggatccggttctggcgtgaaacagactttgaattttgaccttctcaagttggcgggagacgtggagtccaacccagggcccataacttcgtataatgtatgctatacgaagttattaggtctgaagaggagtttacgtccagccaagctagcttggctgcaggtcgtcgacggtatcggccgcatctagagtcgaccagcttctgatggaattagaacttggcaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcatcccggggatctgatatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtaagcttcagctgctcgagatctagatggatgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgaggtcgacgaattcggatcgcgccataacttcgtataatgtatgctatacgaagttatctatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgagatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 20 - DNA, 4848 bp, Artificial Sequence
  synthetic DNA
tccaccggcggcatggacgagctgtacaagtagctttctgacaaaaagtttccatatgttatgtcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggaggtgcttatcttggctttcgagtctgagtttaaaactacacattttgacatagtgtttattagcagccatctaaaaaggctctaatgtatatttaactaaaattactagctttgggaattaaactgtttaacaaataatgctgctcattgtgattcttacctataagcagcctaatttgaattatttgctgcaatcgaattcccgcggccgccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcggccgcgaattcactagtgattgttacttcccttcgagcctcaatgtcctcatttgtaaaatgacattaatacctacttttagctgtgggaattgagtaccatgatttatacaaagcagtttgtatggtgctggttacatgagagttcagatggtaactagttagtaaaaaatctctagtgtgcttgttgattttattttattttagtatttcttaaagatcaaatttaacatcaatcctaaactttatttagctttttctggcgtgtaaactaacatactaagttgtgtgactataattcatttagtgactcatttttagctatttttataacacattgtgctatggggggttttggaacttgctggaagctacatcagaaactgccatagttaattgccatttcaagaatgttgtaaataactcaggtggccgtttaattctcaatgtaaatataattaactagacatctttcctatatttgtgtctcagttttaaagctatttctggatgcttgagtcttaccgtaattgataacaaaaagaggttattgagaatatctatgatttacagagtaagttattctagacctcaagagtgaaatgtaggggaggagacatttgtgtgttaaactaatggaaatgctcatttaatagatattcactgaaagtattagttttggtttattgctagaaaagttgaggttttatggagatttttgtaaaaaatggtttatttcctaaataaatatctctttttcttttttctcccagaaaatgttaatctatgatccagccaaacgaatttctggcaaaatggcactgaatcatccatattttaatgatttggacaatcagattaagaagatgggatccggttctggcgtgaa

SEQ ID NO: 21 - DNA, 8507 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagaagatgtagccctcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 8507 bp, Artificial Sequence
  synthetic DNA
catatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacagggacatccatccaggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgagggcctatttcccatgattccttcatatttg

SEQ ID NO: 23 - DNA, 35 bp, Mus musculus
acagggacatccatccagagggctacatcttctta

SEQ ID NO: 24 - DNA, 8509 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctatctgttgacataacatagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 9177 bp, Artificial Sequence
  synthetic DNA
ctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctatctgttgacataacatagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttt

SEQ ID NO: 26 - DNA, 21 bp, Homo sapiens
ccatatgttatatcaacagat

SEQ ID NO: 27 - DNA, 22 bp, Artificial Sequence
  oligonucleotide
ggttggtcattgactgttgagg

SEQ ID NO: 28 - DNA, 20 bp, Artificial Sequence
  oligonucleotide
gtgctggtgacaggggtagc

SEQ ID NO: 29 - DNA, 24 bp, Artificial Sequence
  oligonucleotide
atggccacaaccatggtgagcaag

SEQ ID NO: 30 - DNA, 25 bp, Artificial Sequence
  oligonucleotide
cctcctgcatttgctggagtcaggg

SEQ ID NO: 31 - DNA, 22 bp, Artificial Sequence
  oligonucleotide
tgatggattcgggcaagtttct

SEQ ID NO: 32 - DNA, 20 bp, Artificial Sequence
  oligonucleotide
acatcggcaaggtgtgggtc

SEQ ID NO: 33 - DNA, 25 bp, Artificial Sequence
  oligonucleotide
accgagatcctccaaacagtagaca

SEQ ID NO: 34 - DNA, 14437 bp, Artificial Sequence
  synthetic DNA
  misc_feature (8819)..(8821): n is a, c, g, or t
gtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggatcgatcctctagagtcgactagtaacggccgccagtgtgctggaattcggcttacaagtttgtacaaaaaagcaggctagctcactcattaggcaccccaggctttacactttatgcttccggctcgtatgttgtgtggaattgtgagcggataacaatttcacacaggaaacagctatgaccatgattacgccaagcttgcatgcctgcaggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccttctagaggatccccgggtaccgagctcgacccaacttttctatactctatacaaagttggtcagtcagaaacaactttggcacatatcaatattatgctctcgttaattaactcgaggtaaaacttaagcttggtaccgagctcggatccnnnatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgcggccgcactagtctagagcgatcgcccgggtttaaacgctagcaacaagctcgtcatcgctttgcagaagagcacaactttgtataataaagttgagctaaaacgcggcctcgaatcgaattccgcccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaatacccagctttcttgtacaaagtggtaagccgaattctgcagattcgactagcttggcacgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggccagtggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgatccggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaacc

SEQ ID NO: 35 - DNA, 7602 bp, Artificial Sequence
  synthetic DNA
actcttcctttttcaatgaccgctttggaaaaacaaagactgtatttcctggaaattaatgtttattcaataaactgtgtattcagctatatcacatagtggtgaggctgaaatgaggcgggaagaggcggttggggcttaattatatcaatttgggtggccccacagcgcctccaaggcgccagtcctgttttgacaagttgcctctggaagcctctacaatgcctctcttctttttctccagagtaagcggaggccaggggcccccggcctctgcttaatactaaaaaaaacagctgttgtcatagtaatgattgggtggaaacattccaggcctgggtggagaggctttttgcttcctcttgcaaaaccacactgacattccaggcctgggtggagaggctttttgcttcctcttgcaaaaccacactgccctctggagggcagttgcctagcaactaactaaaagaggatgtcgcacggccagctgcggtcagttagtcacttcctgcttaactgacttgacattttctattttaagagtcgggaggaaaattactgtgttggaggccctccgccatcttctgaagctgaatcgaattaacttgtttattgcagcttataatggttacaaataaagcaatagcatcacaaatttcacaaataaagcatttttttcactgcattctagttgtggtttgtccaaactcatcaatgtatcttatcatgtctggatctgatatcatcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatttcgacctgcagcccaagctgtaccccctcgaaactgacaggagaaccaccatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgcgaccagcttgatatcgaattaattcactcctcaggtgcaggctgcctatcagaaggtggtggctggtgtggccaatgccctggctcacaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatggatcgatagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatcttctgaggcggaaagaacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 36 - DNA, 20 bp, Artificial Sequence
  oligonucleotide
gtatctggcggcagggtagc

SEQ ID NO: 37 - DNA, 21 bp, Artificial Sequence
  oligonucleotide
tttgttgctggcattgttctg

SEQ ID NO: 38 - DNA, 24 bp, Artificial Sequence
  oligonucleotide
tggacgtaaactcctcttcagacc

SEQ ID NO: 39 - DNA, 24 bp, Artificial Sequence
  oligonucleotide
gaggactacaccatcgtggaacag

SEQ ID NO: 40 - DNA, 21 bp, Artificial Sequence
  oligonucleotide
ggcttccaccaaagaccacag

SEQ ID NO: 41 - DNA, 2041 bp, Artificial Sequence
  synthetic DNA
taccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagctt

SEQ ID NO: 42 - DNA, 413 bp, Artificial Sequence
  synthetic DNA
ccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcgg

SEQ ID NO: 43 - DNA, 1224 bp, Artificial Sequence
  synthetic DNA
ggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatcc

SEQ ID NO: 44 - DNA, 1044 bp, Artificial Sequence
  synthetic DNA
actagttaattaagaaactgacctaaagacttgcctccagccccatcctatgggagcattttcttaactgaggttctctcctctcaagatgacttcagcttgtgacaatgtgacctggcctaaagctagccagcacagcacaacaccttccattctttcttctaaggtgcttatggacatttctttctctttgagtaccttgagtgggattgtctgtggctgcttttgggaagcattcaattgaatgatttttgttttttgtttatttcccccgagacagggtttctctgtgtagccctggctgtcctggaactcactttgtagacctcgaactcagaaatccgcctgcctctgcctcccaagtgctgggattaatggcatgtgccaccactgcctggagaattgaatgcttataaggtaaacagatgcttttattaaaaacaacaacaacaactgttattcctagtggttggactttacagttgtactttgtttccttttgaaacagtgttgcagcgaacgaggccacaggttggccttgaacttgttcttccctgtgctaggattacatcgtgcacaacttccttctcgcagactcttattattttaagaaaaactaaaacagtacaccattttagtacggtagtagtgtgctgaaattcagccctggggatcagggccttttgctttttttggtaaattttgagacatagtgtcactaagttgctttagctagctgtgtatcagtctccacagtggccggcttgacaggcctgcttgctttgtgcatgtgaaggggctaagcccttggctgtgtagaatatacaaacgcagtgtctgagaaggctcagaatggctcacttgcaagaacagctaaaccctaacaagaccatctgaccatctgggatgactgacgtttgccggctacgcgggaggccctggaaaagatgtcttccagatgatgcttagactgtttttgagttagtagcacggaatcacagcactgtttcccctcattttagggtccgtcgtaacctgactagt

SEQ ID NO: 45 - DNA, 36 bp, Artificial Sequence
  oligonucleotide
actagttaattaagaaactgacctaaagacttgcct

SEQ ID NO: 46 - DNA, 35 bp, Artificial Sequence
  oligonucleotide
actagtcaggttacgacggaccctaaaatgagggg

SEQ ID NO: 47 - DNA, 682 bp, Artificial Sequence
  synthetic DNA
ccatggtaccgttcgtatagcatacattatacgaagttatttgagtaactatggaagactatatcaaaatagagaaaattggagaaggtaagtggttttagcagaatgttttttgaaaagaagataacaatgctactgtggaacacagaaacctttactgagtgcagtcgtagatgcatctgtacgcgtgcgcatgagtgtgctgtgagagttggtgtttgtggcctcctagacaggatctgggatcactctgagcacacctgtaggggattgtctttgactctattaattgacgtgggcaggaacctcccccagtgtgggtgccttccttgcctgagatcctggactgtgtgactggagaagggtgtcaagccacagtgaacctcactctcccactgcctgtgctcttgctgctgctgctgctggactctaaccccgatcctgagtgcaaacaaacccttccttgggctacttttgtctgggtgtttctcacagcaccagcaaaaggaaggcacgcagctgggcgtggtggcacacacctttaatcccagcacttgggaggcagaggcaggcggatttctgagttcgaggccagcctggtctacaaagttccaggatagccagggctatacagagaaaccctgtctcgaaaaacagaaaagaaaaaaaaaagaaagcatgc

SEQ ID NO: 48 - DNA, 51 bp, Artificial Sequence
  oligonucleotide
gcatgctttcttttttttttcttttctgtttttcgagacagggtttctctg

SEQ ID NO: 49 - DNA, 72 bp, Artificial Sequence
  oligonucleotide
ccatggtaccgttcgtatagcatacattatacgaagttatttgagtaactatggaagactatatcaaaatag

SEQ ID NO: 50 - DNA, 24 bp, Artificial Sequence
  oligonucleotide
caccgacgacggaccctaaaatga

SEQ ID NO: 51 - DNA, 24 bp, Artificial Sequence
  oligonucleotide
aaactcattttagggtccgtcgtc

SEQ ID NO: 52 - DNA, 24 bp, Artificial Sequence
  oligonucleotide
caccgatatcaaaatagagaaaat

SEQ ID NO: 53 - DNA, 24 bp, Artificial Sequence
  oligonucleotide
aaacattttctctattttgatatc

SEQ ID NO: 54 - DNA, 23 bp, Artificial Sequence
  oligonucleotide
ccaggccacatatgattagttcc

SEQ ID NO: 55 - DNA, 23 bp, Artificial Sequence
  oligonucleotide
gtgagccaagaaattgtttctcc

SEQ ID NO: 56 - DNA, 22 bp, Artificial Sequence
  oligonucleotide
ttgacctccatagaagacaccg

SEQ ID NO: 57 - DNA, 19 bp, Artificial Sequence
  oligonucleotide
gctaagtcccctgccttgg

SEQ ID NO: 58 - DNA, 1978 bp, Artificial Sequence
  synthetic DNA
ggatccggttctggcgtgaaacagactttgaattttgaccttctcaagttggcgggagacgtggagtccaacccagggcccatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgagatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcg

SEQ ID NO: 59 - DNA, 42 bp, Artificial Sequence
  oligonucleotide
gctttctgacaaaaagtttcaatatgttatgtcaacagatag

SEQ ID NO: 60 - DNA, 42 bp, Artificial Sequence
  oligonucleotide
ctatctgttgacataacatattgaaactttttgtcagaaagc

SEQ ID NO: 61 - DNA, 758 bp, Artificial Sequence
  synthetic DNA
ggagaatcctggcccactcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagctttctgacaaaaagtttc

SEQ ID NO: 62 - DNA, 6080 bp, Artificial Sequence
  synthetic DNA
tagctttctgacaaaaagtttcaatatgttatgtcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggaggtgcttatcttggctttcgagtctgagtttaaaactacacattttgacatagtgtttattagcagccatctaaaaaggctctaatgtatatttaactaaaattactagctttgggaattaaactgtttaacaaataatgctgctcattgtgattcttacctataagcagcctaatttgaattatttgctgcaatcgaattcccgcggccgccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcggccgcgaattcactagtgattgttacttcccttcgagcctcaatgtcctcatttgtaaaatgacattaatacctacttttagctgtgggaattgagtaccatgatttatacaaagcagtttgtatggtgctggttacatgagagttcagatggtaactagttagtaaaaaatctctagtgtgcttgttgattttattttattttagtatttcttaaagatcaaatttaacatcaatcctaaactttatttagctttttctggcgtgtaaactaacatactaagttgtgtgactataattcatttagtgactcatttttagctatttttataacacattgtgctatggggggttttggaacttgctggaagctacatcagaaactgccatagttaattgccatttcaagaatgttgtaaataactcaggtggccgtttaattctcaatgtaaatataattaactagacatctttcctatatttgtgtctcagttttaaagctatttctggatgcttgagtcttaccgtaattgataacaaaaagaggttattgagaatatctatgatttacagagtaagttattctagacctcaagagtgaaatgtaggggaggagacatttgtgtgttaaactaatggaaatgctcatttaatagatattcactgaaagtattagttttggtttattgctagaaaagttgaggttttatggagatttttgtaaaaaatggtttatttcctaaataaatatctctttttcttttttctcccagaaaatgttaatctatgatccagccaaacgaatttctggcaaaatggcactgaatcatccatattttaatgatttggacaatcagattaagaagatgggatccggttctggcgtgaaacagactttgaattttgaccttctcaagttggcgggagacgtggagtccaacccagggcccatggccagctacccctgtcaccagcacgccagcgccttcgaccaggccgccagaagcaggggccacagcaaccggcggaccgccctgagacccaggcggcagcaggaagccaccgaagtccggctggaacagaagatgcccaccctgttaagggtgtacatcgacggcccccacggcatgggcaagaccaccaccacccagctgctggtggccctgggcagccgggacgacatcgtgtacgtgcccgagcccatgacctactggcaggtgctgggcgccagcgagaccatcgccaacatctacaccacacagcacaggctggaccagggcgagatctctgccggcgacgccgccgtggtgatgaccagcgcccagatcacaatgggcatgccctacgccgtgaccgacgccgtgctggcccctcacgtgggcggcgaggccggctctagccacgcccctccccctgccctgaccctgatcttcgaccggcaccccatcgcccacctgctgtgctaccctgccgccagatacctgatgggcagcatgaccccccaggccgtgctggccttcgtggccctgatcccccccaccctgcccggcaccaacatcgtgctgggagccctgcccgaggaccggcacatcgaccggctggccaagcggcagagacccggcgagcggctggacctggccatgctggccgccatccggcgggtgtacggcctgctggccaacaccgtgagatacctgcagggcggagggtcttggtgggaggactggggccagctgtccggcaccgccgtgccacctcagggcgccgagccccagagcaatgccggccctcggccccacatcggcgacaccctgtttaccctgttcagagcccccgagctgctggcccccaacggcgacctgtacaacgtgttcgcctgggccctggacgtgctggccaagaggctgcggcccatgcacgtgttcatcctggactacgaccagagccctgccggctgcagggacgccctgctgcagctgaccagcggcatggtgcagacccacgtgaccacccccggcagcatccccaccatctgcgacctggcccggaccttcgcccgggagatgggcgaggccaacggctccggagagggcagaggaagtctgctaacatgcggtgacgtcgaggagaatcctggcccactcgaggtga

SEQ ID NO: 63 - DNA, 58 bp, Artificial Sequence
  oligonucleotide
taccgttcgtatagcatacattatacgaagttataccatgtctagactggacaagagc

SEQ ID NO: 64 - DNA, 31 bp, Artificial Sequence
  oligonucleotide
gcagagggaaaaagatctgcgactctagagg

SEQ ID NO: 65 - DNA, 23 bp, Artificial Sequence
  oligonucleotide
acgagtgtacacacacgaggtag

SEQ ID NO: 66 - DNA, 22 bp, Artificial Sequence
  oligonucleotide
tgtcaaccggagtggagtaacg

SEQ ID NO: 67 - DNA, 19 bp, Artificial Sequence
  oligonucleotide
atggaggggaatacagccc

SEQ ID NO: 68 - DNA, 20 bp, Artificial Sequence
  oligonucleotide
ttctttgcagctccttcgtt

SEQ ID NO: 69 - DNA, 982 bp, Artificial Sequence
  synthetic DNA
aagatacatctctgcttctttctggattgatttttctttgaagatgaatgtgagaaatagaacttaaaggtctattctgagtgtgctttataaatgattttatcatcagtgatgggtgttagaaaaatctaggtaattatgcctggagttctgacgtttcattggcaaacttcagaggaagtcttgaacttctggttgtcagcttttttttttaacccattcattgtgattcaagtataccgtttttctgcttagtccttgacattgtttaggttataattagaattttatctgtggaattgcacttttcattcttttgttttacggtgcttaagtgatatttcctactcttggaaaaagtaccttgaaagtcttgagcatgtttgtctgtgacctcagcaagcagaggaaaaagattaaaattgtcacgtgggcaactgtaaacacttaaaatgtttttgtcgttttaaacaaaattttcctaatgaacagtctgaacacattttagttctgtcctcataactttaaaaaataatgttttgaataaaattaggagtatcataaagcggtataaatttgttgaaatatttgggtataactttggcacacgcaggtactactttaaagtgtcacaaaaagggaacataattctggtttttgtttggaaagaatagacaagttgatacctgaatttgagggttattttgattggaagaagctaaatactacactagtatagaatatacacgtttccaatgtctcaggagtcaagaaggatggaaagttattgtaggagtccttacccttgtaaatattgtttgaatgtaattaatatttatggaatacttatgctgcagtaggcactgtggaaaatgctttacatatagtgtttcattaatgcttaaaactactcgagttagtggcatgtaattaagtcagtttttccttcactttaggatctaccatacccattgactaactc

SEQ ID NO: 70 - DNA, 41 bp, Artificial Sequence
  oligonucleotide
aaatcctgcaggaagatacatctctgcttctttctggattg

SEQ ID NO: 71 - DNA, 45 bp, Artificial Sequence
  oligonucleotide
aaatcctgcaggagttagtcaatgggtatggtagatcctaaagtg

SEQ ID NO: 72 - DNA, 943 bp, Artificial Sequence
  synthetic DNA
aagattataccaaaatagagaaaattggagaaggtgagtggttttagtaaaataaattttatggaatgatttaacaatgctacaacttctgtaatatgaacacatttaaatatttattaaaattcaaccattaagttgtcttgtagtaccagtgtgcattaacatatgttctttacttaataaacgcaaatttgctcacatttcaagagaattgaaattaccgaatccctaatgaccgttcgcctcacccccatagtcttacagtcttctgatactaaacacttatagtagtacttgtattttgaagacactattgataacttgcatgtgtgttgaggggcagaggttagggctgcagaattatgctaacttcataatatatttttagttttagtctctttcaattgtttttaggatgataaatatttttatccagactttatagactggttacaataattgacttaatctaatttaaataaaatttctatttagttaccctaagtataaatgacctgtcatatagacatgtttaagctattctaaaattataacttatgtatataatccttccctttctgtggtacttgaaggaatgtttagatctatatatggcacaaatgaggggtatatcccttcgcctgttcctcgactccagcagaacattctcgattctccaggtacaaaataatttgctaatagaatccagtttatataggttctgccagaagaaaattcccaactatatttttaatgtgatcaaagaccccacttgacaagaaatagtgaaaaacaagggggaatactggagggaatttagtggaataggagtgacaagttagtgtggtgttcgattggaggtgtgtgtgtgtatacatatatatatatattttttttttaaatcacttaaatacctcatacccattctactaaacaaattttg

SEQ ID NO: 73 - DNA, 38 bp, Artificial Sequence
  oligonucleotide
ttaaccatggaagattataccaaaatagagaaaattgg

SEQ ID NO: 74 - DNA, 40 bp, Artificial Sequence
  oligonucleotide
tttagcatgccaaaatttgtttagtagaatgggtatgagg

SEQ ID NO: 75 - DNA, 8604 bp, Artificial Sequence
  synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggaagatacatctctgcttctttctggattgatttttctttgaagatgaatgtgagaaatagaacttaaaggtctattctgagtgtgctttataaatgattttatcatcagtgatgggtgttagaaaaatctaggtaattatgcctggagttctgacgtttcattggcaaacttcagaggaagtcttgaacttctggttgtcagcttttttttttaacccattcattgtgattcaagtataccgtttttctgcttagtccttgacattgtttaggttataattagaattttatctgtggaattgcacttttcattcttttgttttacggtgcttaagtgatatttcctactcttggaaaaagtaccttgaaagtcttgagcatgtttgtctgtgacctcagcaagcagaggaaaaagattaaaattgtcacgtgggcaactgtaaacacttaaaatgtttttgtcgttttaaacaaaattttcctaatgaacagtctgaacacattttagttctgtcctcataactttaaaaaataatgttttgaataaaattaggagtatcataaagcggtataaatttgttgaaatatttgggtataactttggcacacgcaggtactactttaaagtgtcacaaaaagggaacataattctggtttttgtttggaaagaatagacaagttgatacctgaatttgagggttattttgattggaagaagctaaatactacactagtatagaatatacacgtttccaatgtctcaggagtcaagaaggatggaaagttattgtaggagtccttacccttgtaaatattgtttgaatgtaattaatatttatggaatacttatgctgcagtaggcactgtggaaaatgctttacatatagtgtttcattaatgcttaaaactactcgagttagtggcatgtaattaagtcagtttttccttcactttaggatctaccatacccattgactaactcctgcaggcggccgcgaattcactagtgattaccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttatcgaattcccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggccgccatggaagattataccaaaatagagaaaattggagaaggtgagtggttttagtaaaataaattttatggaatgatttaacaatgctacaacttctgtaatatgaacacatttaaatatttattaaaattcaaccattaagttgtcttgtagtaccagtgtgcattaacatatgttctttacttaataaacgcaaatttgctcacatttcaagagaattgaaattaccgaatccctaatgaccgttcgcctcacccccatagtcttacagtcttctgatactaaacacttatagtagtacttgtattttgaagacactattgataacttgcatgtgtgttgaggggcagaggttagggctgcagaattatgctaacttcataatatatttttagttttagtctctttcaattgtttttaggatgataaatatttttatccagactttatagactggttacaataattgacttaatctaatttaaataaaatttctatttagttaccctaagtataaatgacctgtcatatagacatgtttaagctattctaaaattataacttatgtatataatccttccctttctgtggtacttgaaggaatgtttagatctatatatggcacaaatgaggggtatatcccttcgcctgttcctcgactccagcagaacattctcgattctccaggtacaaaataatttgctaatagaatccagtttatataggttctgccagaagaaaattcccaactatatttttaatgtgatcaaagaccccacttgacaagaaatagtgaaaaacaagggggaatactggagggaatttagtggaataggagtgacaagttagtgtggtgttcgattggaggtgtgtgtgtgtatacatatatatatatattttttttttaaatcacttaaatacctcatacccattctactaaacaaattttggcatgcgacgtcgggcccaattcgccc

SEQ ID NO: 76 - DNA, 24 bp, Artificial Sequence
  oligonucleotide
caccgataccaaaatagagaaaat

SEQ ID NO: 77 - DNA, 23 bp, Artificial Sequence
  oligonucleotide
aaacattttctctattttggtac

SEQ ID NO: 78 - DNA, 24 bp, Artificial Sequence
  oligonucleotide
caccgcatagttagtcaatgggta

SEQ ID NO: 79 - DNA, 24 bp, Artificial Sequence
  oligonucleotide
aaactacccattgactaactatgc

SEQ ID NO: 80 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgataccaaaatagagaaaatgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 81 - DNA, 8440 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccaccgcatagttagtcaatgggtagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 82 - DNA, 8509 bp, Artificial Sequence
  synthetic DNA
cgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatagttagtcaatgggtagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaa

SEQ ID NO: 83 - DNA, 20 bp, Artificial Sequence
  oligonucleotide
ggcccacctggagctgttaa

SEQ ID NO: 84 - DNA, 22 bp, Artificial Sequence
  oligonucleotide
cgccgtttatgactttgctctt

SEQ ID NO: 85 - DNA, 20 bp, Artificial Sequence
  oligonucleotide
agacgccatccacgctgttt

SEQ ID NO: 86 - DNA, 20 bp, Artificial Sequence
  oligonucleotide
gccaaaactcataggccaaa

SEQ ID NO: 87 - DNA, 870 bp, Artificial Sequence
  oligonucleotide
ccacagggagtgttagtgctgctcgcgattttgctggcatctctatactcttcgtgtgtggcagcctaagaccgcaagcagaagaacttgccagaactttgccagcattaactcagcgcttctcaacttcctaaggttgcgacccttcaacatagcgtctcatgttgtgatgccctgctaccataattattccattgctacttcataactgtaatttttttgctgctgttatgagttgtaaggtaaactatctgatatgcgacccccaagagtcacgacccacaggctgagaaccgctgccttggccagccttgagggagggatggcgttgtttcctcggtcaggaaaaaaaaaggaaaactcatcgcaggattaagactctgtaatctcaacagatctgaaataaagaatgagcatccgttttctagttatattcagaaggacggcttagtcaaccactttgcacttccaaaatgccttccctctcttcagagtgtctctcagagcacatggaccttcctcattggtcagattttctgtcagtaccaccaacaattgctattggtccattttgaagattctcccgccttcttttacctaattggtttattcgaaaaggcggtcttttgttcgaataaaagacgatctacgattggttactgcaaacagagccaatgagagcgagtcggtgattggttcctctgatttgggcgggctagataaaggttcaaatgaaactagggaagctctcctaaccgacaagcgtctgtgaggggagcgtccaggtcccgcgtcgcgcgtcgcgggtcgcctagcgtttcccgctggttttgtcgctttccggattctcgagcctcttcaggaccgtca

SEQ ID NO: 88 - DNA, 37 bp, Artificial Sequence
  synthetic DNA
aattcctgcaggccacagggagtgttagtgctgctcg

SEQ ID NO: 89 - DNA, 36 bp, Artificial Sequence
  synthetic DNA
ttactagttgacggtcctgaagaggctcgagaatcc

SEQ ID NO: 90 - DNA, 818 bp, Artificial Sequence
  synthetic DNA
agttgtcaccgctgcaggtacagctcgcggatggggccgggtagtagagcctttacgtggcctctgcgttcatggctgacggcgagagttgaggttccagggccgagcgctggccggcgaccggctggatccggtcagccccatcttctgggccttgaggggtcgtggactgggtgaagctctttccgaaatgatatctataatctaatctgggttcttgagtgttgtgtttttattgtttgtgggagcaggttcaactagtcctgcgatcggtctgtagtctaatggcttctgtgtgacttccagccggcttatactcgaattcagtggccctcttcccttccctgggtgatttgaataactcatcacggaaggtgcagagcatggcagttcccattgaggagctggccgaccgagtggctcaaaaaaagtccagtttcgggatgtgaatgtagccaagggtttgcattgctagcatcccaactgctacgggtggataccatctgtaatcggcacgagcctatgtcggcactcactaaatattttcccattagatggaagaagttgagtgtagtttatcgatgacataaaaaatattactcaaggcagaaatgtccatacttgcagaactaaaatacctatttaaacgggcatctgtagccagacctagtggggcaggcctgaaatggcagctgctccagagactgaggcaagaggatgccttaagctatttaatgggactctcaaaaacagcaacagggggtgaaaaaaatggtctgggaattttgctcagtggtcagagcatttgcctagcatgt

SEQ ID NO: 91 - DNA, 27 bp, Artificial Sequence
  oligonucleotide
ccgtcaccatggagttgtcaccgctgc

SEQ ID NO: 92 - DNA, 30 bp, Artificial Sequence
  oligonucleotide
cccttgcatgcacatgctaggcaaatgctc

SEQ ID NO: 93 - DNA, 8347 bp, Artificial Sequence
  synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggccacagggagtgttagtgctgctcgcgattttgctggcatctctatactcttcgtgtgtggcagcctaagaccgcaagcagaagaacttgccagaactttgccagcattaactcagcgcttctcaacttcctaaggttgcgacccttcaacatagcgtctcatgttgtgatgccctgctaccataattattccattgctacttcataactgtaatttttttgctgctgttatgagttgtaaggtaaactatctgatatgcgacccccaagagtcacgacccacaggctgagaaccgctgccttggccagccttgagggagggatggcgttgtttcctcggtcaggaaaaaaaaaggaaaactcatcgcaggattaagactctgtaatctcaacagatctgaaataaagaatgagcatccgttttctagttatattcagaaggacggcttagtcaaccactttgcacttccaaaatgccttccctctcttcagagtgtctctcagagcacatggaccttcctcattggtcagattttctgtcagtaccaccaacaattgctattggtccattttgaagattctcccgccttcttttacctaattggtttattcgaaaaggcggtcttttgttcgaataaaagacgatctacgattggttactgcaaacagagccaatgagagcgagtcggtgattggttcctctgatttgggcgggctagataaaggttcaaatgaaactagggaagctctcctaaccgacaagcgtctgtgaggggagcgtccaggtcccgcgtcgcgcgtcgcgggtcgcctagcgtttcccgctggttttgtcgctttccggattctcgagcctcttcaggaccgtcaactagtgattaccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttatcgaattcccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggccgccatggagttgtcaccgctgcaggtacagctcgcggatggggccgggtagtagagcctttacgtggcctctgcgttcatggctgacggcgagagttgaggttccagggccgagcgctggccggcgaccggctggatccggtcagccccatcttctgggccttgaggggtcgtggactgggtgaagctctttccgaaatgatatctataatctaatctgggttcttgagtgttgtgtttttattgtttgtgggagcaggttcaactagtcctgcgatcggtctgtagtctaatggcttctgtgtgacttccagccggcttatactcgaattcagtggccctcttcccttccctgggtgatttgaataactcatcacggaaggtgcagagcatggcagttcccattgaggagctggccgaccgagtggctcaaaaaaagtccagtttcgggatgtgaatgtagccaagggtttgcattgctagcatcccaactgctacgggtggataccatctgtaatcggcacgagcctatgtcggcactcactaaatattttcccattagatggaagaagttgagtgtagtttatcgatgacataaaaaatattactcaaggcagaaatgtccatacttgcagaactaaaatacctatttaaacgggcatctgtagccagacctagtggggcaggcctgaaatggcagctgctccagagactgaggcaagaggatgccttaagctatttaatgggactctcaaaaacagcaacagggggtgaaaaaaatggtctgggaattttgctcagtggtcagagcatttgcctagcatgtgcatgcgacgtcgggcccaattcgccc

SEQ ID NO: 94 - DNA, 24 bp, Artificial Sequence
  oligonucleotide
caccgcatggtgacggtcctgaag

SEQ ID NO: 95 - DNA, 24 bp, Artificial Sequence
  oligonucleotide
aaaccttcaggaccgtcaccatgc

SEQ ID NO: 96 - DNA, 24 bp, Artificial Sequence
  oligonucleotide
caccgccgctgcaggtacagctcg

SEQ ID NO: 97 - DNA, 24 bp, Artificial Sequence
  oligonucleotide
aaaccgagctgtacctgcagcggc

SEQ ID NO: 98 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccgctgcaggtacagctcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 99 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcatggtgacggtcctgaaggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 100 - DNA, 874 bp, Artificial Sequence
  synthetic DNA
catttcagattcgaaccatgacacatttccatagcagctgtgccattttacaccacccaccccaatgttccaccaggcttctatgaaaccactgattatataagtattttcctgattagagtacacgttcatgatagatcatttagaaaactcaaaaaagcaaaatgacgaaattctctttattctaattacggagccatgacctgaattcatattactcccgggagaaaaggaaaagccggtttttttctcaggcaattctgtcattgtgtttgatagcatggtccaataatagtgctggcgcctgccctgcctggaccttgtcctcagattcctccgtcctggctgtccccacgaccctatcttctgaaaaggtcatgcagttaaccaggcttctgaatcatatttgctggttgctttcaggagtgtcacgggtgctgtgtggagctgtcacattcttgggcatctgtttccgtatttttaaagccacctcaacgtttgatagaaaagcataaaacttttgaggaagtgagctctacaaacccaccacccacgtaagaactgagccgtcctgcccagagggcatctgaaaaagagatcaacaggaaaagggtggggaaatcctgccttccttcccgtcttcaagtacccattggcttcagacctttattctcattggtttatctggatgtcgctccacgctggctttccagaaccagccactgagagtcacaaagttgggatttaaaagatgcggtgggaggggcgggtgattttttgaactgctggcgaccgagttctggcggtcctgacgcgcaccttgggcttgctcgcgttcggtttcggcagcaggaccaagaaggcttagggaagg

SEQ ID NO: 101 - DNA, 42 bp, Artificial Sequence
  oligonucleotide
aattcctgcaggcatttcagattcgaaccatgacacatttcc

SEQ ID NO: 102 - DNA, 37 bp, Artificial Sequence
  oligonucleotide
ttactagtccttccctaagccttcttggtcctgctgc

SEQ ID NO: 103 - DNA, 825 bp, Artificial Sequence
  synthetic DNA
gcccgcgtcgcaaaccgcagaccccaaggaggagaccctccagcccggcgcctggaccctcgcgacagagctccagtgtaggtaagcgagcctgccctgcacctccgaggccggtgcggtcatcctgtggggccgggcctgtagagggacggttcctggtcacggcttgccgctaacctcgcgtaggggtaatggtttttttgtttatttgtttgtttgtttgcttgtttgttcctgaccggtttacagcagggagaaaaacgaaatgcagacaagacaaggagttggagcaaagacaaactcattctgacggacacggggtctttttttgcatttatttactttgcatgtattttgcatttattttgtgttcgtgctctggcgacggccagaagagagcgtcggaaccccggggccggagttacagttgtgagctgcccttttttttagagtgctaggaagagaactcggcccccctggaagagcagcctgggctattaacagctgaaccagctttctaactcctactctttattatttatttgcagcactagagatggaacccaggactccgtgtatgttaggcaagggttctactacagaactatatccccaaccctctccccgacccttttttttttaagacgggaactcggtatgtatcggtgtgtagccttggcttgctgacctgaaacgtgctatgtagactaggctagcctagagctcacagatctctgcctgtgttggggatggttgtatgcagtgtgtattcaggaactatttctgagatctggttacaatccagacactgcctcaatgtgatgt

SEQ ID NO: 104 - DNA, 23 bp, Artificial Sequence
  oligonucleotide
agccatgggcccgcgtcgcaaac

SEQ ID NO: 105 - DNA, 34 bp, Artificial Sequence
  oligonucleotide
aattgcatgcacatcacattgaggcagtgtctgg

SEQ ID NO: 106 - DNA, 8358 bp, Artificial Sequence
  synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcatttcagattcgaaccatgacacatttccatagcagctgtgccattttacaccacccaccccaatgttccaccaggcttctatgaaaccactgattatataagtattttcctgattagagtacacgttcatgatagatcatttagaaaactcaaaaaagcaaaatgacgaaattctctttattctaattacggagccatgacctgaattcatattactcccgggagaaaaggaaaagccggtttttttctcaggcaattctgtcattgtgtttgatagcatggtccaataatagtgctggcgcctgccctgcctggaccttgtcctcagattcctccgtcctggctgtccccacgaccctatcttctgaaaaggtcatgcagttaaccaggcttctgaatcatatttgctggttgctttcaggagtgtcacgggtgctgtgtggagctgtcacattcttgggcatctgtttccgtatttttaaagccacctcaacgtttgatagaaaagcataaaacttttgaggaagtgagctctacaaacccaccacccacgtaagaactgagccgtcctgcccagagggcatctgaaaaagagatcaacaggaaaagggtggggaaatcctgccttccttcccgtcttcaagtacccattggcttcagacctttattctcattggtttatctggatgtcgctccacgctggctttccagaaccagccactgagagtcacaaagttgggatttaaaagatgcggtgggaggggcgggtgattttttgaactgctggcgaccgagttctggcggtcctgacgcgcaccttgggcttgctcgcgttcggtttcggcagcaggaccaagaaggcttagggaaggactagtgattaccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttatcgaattcccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggccgccatgggcccgcgtcgcaaaccgcagaccccaaggaggagaccctccagcccggcgcctggaccctcgcgacagagctccagtgtaggtaagcgagcctgccctgcacctccgaggccggtgcggtcatcctgtggggccgggcctgtagagggacggttcctggtcacggcttgccgctaacctcgcgtaggggtaatggtttttttgtttatttgtttgtttgtttgcttgtttgttcctgaccggtttacagcagggagaaaaacgaaatgcagacaagacaaggagttggagcaaagacaaactcattctgacggacacggggtctttttttgcatttatttactttgcatgtattttgcatttattttgtgttcgtgctctggcgacggccagaagagagcgtcggaaccccggggccggagttacagttgtgagctgcccttttttttagagtgctaggaagagaactcggcccccctggaagagcagcctgggctattaacagctgaaccagctttctaactcctactctttattatttatttgcagcactagagatggaacccaggactccgtgtatgttaggcaagggttctactacagaactatatccccaaccctctccccgacccttttttttttaagacgggaactcggtatgtatcggtgtgtagccttggcttgctgacctgaaacgtgctatgtagactaggctagcctagagctcacagatctctgcctgtgttggggatggttgtatgcagtgtgtattcaggaactatttctgagatctggttacaatccagacactgcctcaatgtgatgtgcatgcgacgtcgggcccaattcgccc

SEQ ID NO: 107 - DNA, 24 bp, Artificial Sequence
  oligonucleotide
caccgtcgcaaaccgcagacccca

SEQ ID NO: 108 - DNA, 24 bp, Artificial Sequence
  oligonucleotide
aaactggggtctgcggtttgcgac

SEQ ID NO: 109 - DNA, 24 bp, Artificial Sequence
  oligonucleotide
caccgtggctggcctcgaggggcc

SEQ ID NO: 110 - DNA, 24 bp, Artificial Sequence
  oligonucleotide
aaacggcccctcgaggccagccac

SEQ ID NO: 111 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcgcaaaccgcagaccccagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 112 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggctggcctcgaggggccgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 113 - DNA, 29 bp, Artificial Sequence
  oligonucleotide
tctggaactcactatgtaggtctactggc

SEQ ID NO: 114 - DNA, 22 bp, Artificial Sequence
  oligonucleotide
ttgacctccatagaagacaccg

SEQ ID NO: 115 - DNA, 30 bp, Artificial Sequence
  oligonucleotide
ctcttcttctcacctggtctcttcaggacc

SEQ ID NO: 116 - DNA, 24 bp, Artificial Sequence
  oligonucleotide
acctcctctccttacatgctggtc

SEQ ID NO: 117 - DNA, 23 bp, Artificial Sequence
  oligonucleotide
tacattccactggcagcttaggg

SEQ ID NO: 118 - DNA, 617 bp, Artificial Sequence
  synthetic DNA
ggagctccggcgctgccccagatctggcagctgtacctcaagaactaccgcatcgccaccttcaagaactggcccttcctggaggactgcgcctgcaccccagagcgagtgagtcccagcttccagcgactccaccctgggcccgacggtgccttagttgtgtgacccgggatcagaacccgctatccccaccctcgcttcgtactgttagccaggctgctttgctctgctctgtcctccacgaggccaccccccccccccgctctccccaccctctgcctgaaccactgttagcctccgggcctcttcccactgtcctcagttctcggcccggaacgatctctgggaatcatgggataaggtcagactgttgatcccgctcacgagctgtcattttcagatggcggaggctggcttcatccactgccctaccgagaacgagcctgatttggcccagtgttttttctgctttaaggaattggaaggctgggaacccgatgacaacccgatgtaagtcccacaggctactctcggtgggtttcctggtgttccccttccattgagagccttttgggttgaactggagttgggacaaaccatttgtatttagtagaaga

SEQ ID NO: 119 - DNA, 31 bp, Artificial Sequence
  oligonucleotide
caccatgggagctccggcgctgccccagatc

SEQ ID NO: 120 - DNA, 34 bp, Artificial Sequence
  oligonucleotide
taactggaacacttgagcaagaagcatgctcttc

SEQ ID NO: 121 - DNA, 775 bp, Artificial Sequence
  synthetic DNA
cagtgtgaagctctttagaagccactgcaaccacaggccgcccgacaggaacagagacactgaaaacgggcccgcagcaaggcaggctcagcagccaacagtcacacccaggaagcagtatttttcttctgctcctggactctcttgcggtgtatggctgcttccctttggtctgagccaggccgatggtctcagaaatagacacccattgactttcttttccagcgctgggacatacagaccccgcctccatcccagggtgtctataggaaggatggcggctgctgcagggaggagggtctcctgtcttcctaagggcgcccctccaccagcctgtgggtgggtccgaggcacttccattccgatatctagctggccaaatcctgcaaaccttgaggcaggaagaacctgcagagcacatgggacttgcagcggacatgctttaaagaggtgccccaggcccgtccaccgccctcggccaccctccgtgtcctctggggagcagctgcggaagattcgagtcagaatagcaagaaggaaccgcagcagaaggtacaactcccagcatgccctgcgcccgccacgcccacaaggccaggcgcagatgggcgtggggcgggactttcccggctcgcctcgcgccgtccactcccagaaggcagcgggcgagggcgtggggccggggctctcccggcatgctctgcggcgcgcctccgcccgcgcgatttgaatcctgcgtttgagtcgtcttggcggaggttgtggtgacgccatc

SEQ ID NO: 122 - DNA, 39 bp, Artificial Sequence
  oligonucleotide
aattcctgcaggcagtgtgaagctctttagaagccactg

SEQ ID NO: 123 - DNA, 35 bp, Artificial Sequence
  oligonucleotide
ttactagtgatggcgtcaccacaacctccgccaag

SEQ ID NO: 124 - DNA, 8050 bp, Artificial Sequence
  synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcagtgtgaagctctttagaagccactgcaaccacaggccgcccgacaggaacagagacactgaaaacgggcccgcagcaaggcaggctcagcagccaacagtcacacccaggaagcagtatttttcttctgctcctggactctcttgcggtgtatggctgcttccctttggtctgagccaggccgatggtctcagaaatagacacccattgactttcttttccagcgctgggacatacagaccccgcctccatcccagggtgtctataggaaggatggcggctgctgcagggaggagggtctcctgtcttcctaagggcgcccctccaccagcctgtgggtgggtccgaggcacttccattccgatatctagctggccaaatcctgcaaaccttgaggcaggaagaacctgcagagcacatgggacttgcagcggacatgctttaaagaggtgccccaggcccgtccaccgccctcggccaccctccgtgtcctctggggagcagctgcggaagattcgagtcagaatagcaagaaggaaccgcagcagaaggtacaactcccagcatgccctgcgcccgccacgcccacaaggccaggcgcagatgggcgtggggcgggactttcccggctcgcctcgcgccgtccactcccagaaggcagcgggcgagggcgtggggccggggctctcccggcatgctctgcggcgcgcctccgcccgcgcgatttgaatcctgcgtttgagtcgtcttggcggaggttgtggtgacgccatcactagtgattaccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttatcgaattcccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggccgccatgggagctccggcgctgccccagatctggcagctgtacctcaagaactaccgcatcgccaccttcaagaactggcccttcctggaggactgcgcctgcaccccagagcgagtgagtcccagcttccagcgactccaccctgggcccgacggtgccttagttgtgtgacccgggatcagaacccgctatccccaccctcgcttcgtactgttagccaggctgctttgctctgctctgtcctccacgaggccaccccccccccccgctctccccaccctctgcctgaaccactgttagcctccgggcctcttcccactgtcctcagttctcggcccggaacgatctctgggaatcatgggataaggtcagactgttgatcccgctcacgagctgtcattttcagatggcggaggctggcttcatccactgccctaccgagaacgagcctgatttggcccagtgttttttctgctttaaggaattggaaggctgggaacccgatgacaacccgatgtaagtcccacaggctactctcggtgggtttcctggtgttccccttccattgagagccttttgggttgaactggagttgggacaaaccatttgtatttagtagaagagcatgcgacgtcgggcccaattcgccc

SEQ ID NO: 125 - DNA, 24 bp, Artificial Sequence
  oligonucleotide
caccggacgccatcatgggagctc

SEQ ID NO: 126 - DNA, 24 bp, Artificial Sequence
  oligonucleotide
aaacgagctcccatgatggcgtcc

SEQ ID NO: 127 - DNA, 24 bp, Artificial Sequence
  oligonucleotide
caccggccaagacgactcaaacgc

SEQ ID NO: 128 - DNA, 24 bp, Artificial Sequence
  oligonucleotide
aaacgcgtttgagtcgtcttggcc

SEQ ID NO: 129 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacgccatcatgggagctcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 130 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccaagacgactcaaacgcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 131 - DNA, 24 bp, Artificial Sequence
  oligonucleotide
cactcgtttgcaacgccaacctgg

SEQ ID NO: 132 - DNA, 22 bp, Artificial Sequence
  oligonucleotide
gaacccgatgacaacccgatag

SEQ ID NO: 133 - DNA, 24 bp, Artificial Sequence
  oligonucleotide
tccatctgcttcttgacagtgagg

SEQ ID NO: 134 - DNA, 817 bp, Artificial Sequence
  synthetic DNA
gattaaaggcgtgcaccactactgtctgccttttgttttggttttatcataattgttgcatttttgcccgcaggaggatcaggaattcaaggacaacctctacagccaacccagttgagggccagcctgttctatcgagatcctgtctccggacaaaaaaagtttcatgagagcgtggtgtgttggcacagtcttttagtccgagcactgacgcagtagattaaggcagatcttcatgatttggtgcggtcctgggctgcatccaaagaccttgtttccaaaacaaacaaacgaaaaagtgtaataaaaatacaagcaaggttttgcagaaataaatctgtggcctcaagggtccctttctccagcttttcccagaggtgtcaaattacataacagtctcaaacccaggaaattgaactttggcacaacccactgaccggtccgaatttcaaagtctttttcctattgacctacaaggttttcaagaatcatgttgtaagcaactgtgttctgaggaatctatgtttaaaaacccatccgtggatcttggcccagggtccagagactgagctagccacgccccggccgcgccgcagccactcccacggcagttcaagtgttaagtcccaaagaccgcgctctgtgcatgcgcagacccgtccacagctggctcctagccaacccggccggacgagcacccggcgccgtcacgtgacgcacccaaccggcgtcgacctataaaaggccgggcgttgacgtcagcggtctcttccgccgcagccgccgccatcgtcggcgcgcttccctgttcacctct

SEQ ID NO: 135 - DNA, 37 bp, Artificial Sequence
  oligonucleotide
tttaacctgcagggattaaaggcgtgcaccactactg

SEQ ID NO: 136 - DNA, 36 bp, Artificial Sequence
  oligonucleotide
tatataactagtagaggtgaacagggaagcgcgccg

SEQ ID NO: 137 - DNA, 826 bp, Artificial Sequence
  synthetic DNA
gactctgagaatccgtcgccatccgccaccatggtgagtctcctcggctccgctagactcggggaccgagacgaatctctggggcagcgggacgtggctgtagcgggacgctgagagggacgggaggaagagacatggctgccctggcccgggcggcaggacgtggtcgggccgcggcgccatatctgcgcgtccctgagggccttgggagtgtcaactgccgaggtcggggtgttttcttgaagtccttcaactccccgcggccgccggggtgactgcgggaggggttgtgcttggtgatgtggcagcgggcaaagcgccgtccccgcgcccctggtgacgggcggagggtgtcctcgggaggtgacagcctgtagggctggcttccttggacacctccagtgggctgaacgccttccgggccctttccggtagcccccgtctctgttttctatctgagttcacacgtgagcaccggtccccataatctaagaaagtggctcactgggcctagtggcgcattgtggcctttgatccgggctttgaccttggcgcacagcacccagtggtttggggaagaggtgtgtgtagcagaggaggttttttcgtgctttggtcccaatcaatccggcatctttgcagtgccgaggtggccgtgcaccttggctttgaattcttgtgctgaggttatgtgacttgagcctcaagatagggtcttctagcacaggcttgctcttaagtgtcgcagttgtcggtttcggcgtttgtttagagctgtggacacatctgtgaacttttgatgcttatttcagaggtcctgggttgt

SEQ ID NO: 138 - DNA, 36 bp, Artificial Sequence
  oligonucleotide
ttatagcatgcgactctgagaatccgtcgccatccg

SEQ ID NO: 139 - DNA, 36 bp, Artificial Sequence
  oligonucleotide
ttatagcatgcacaacccaggacctctgaaataagc

SEQ ID NO: 140 - DNA, 8317 bp, Artificial Sequence
  synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcagggattaaaggcgtgcaccactactgtctgccttttgttttggttttatcataattgttgcatttttgcccgcaggaggatcaggaattcaaggacaacctctacagccaacccagttgagggccagcctgttctatcgagatcctgtctccggacaaaaaaagtttcatgagagcgtggtgtgttggcacagtcttttagtccgagcactgacgcagtagattaaggcagatcttcatgatttggtgcggtcctgggctgcatccaaagaccttgtttccaaaacaaacaaacgaaaaagtgtaataaaaatacaagcaaggttttgcagaaataaatctgtggcctcaagggtccctttctccagcttttcccagaggtgtcaaattacataacagtctcaaacccaggaaattgaactttggcacaacccactgaccggtccgaatttcaaagtctttttcctattgacctacaaggttttcaagaatcatgttgtaagcaactgtgttctgaggaatctatgtttaaaaacccatccgtggatcttggcccagggtccagagactgagctagccacgccccggccgcgccgcagccactcccacggcagttcaagtgttaagtcccaaagaccgcgctctgtgcatgcgcagacccgtccacagctggctcctagccaacccggccggacgagcacccggcgccgtcacgtgacgcacccaaccggcgtcgacctataaaaggccgggcgttgacgtcagcggtctcttccgccgcagccgccgccatcgtcggcgcgcttccctgttcacctctactagtgattaccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttatcgaattcccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggccgccatggcggccgggagcatgcgactctgagaatccgtcgccatccgccaccatggtgagtctcctcggctccgctagactcggggaccgagacgaatctctggggcagcgggacgtggctgtagcgggacgctgagagggacgggaggaagagacatggctgccctggcccgggcggcaggacgtggtcgggccgcggcgccatatctgcgcgtccctgagggccttgggagtgtcaactgccgaggtcggggtgttttcttgaagtccttcaactccccgcggccgccggggtgactgcgggaggggttgtgcttggtgatgtggcagcgggcaaagcgccgtccccgcgcccctggtgacgggcggagggtgtcctcgggaggtgacagcctgtagggctggcttccttggacacctccagtgggctgaacgccttccgggccctttccggtagcccccgtctctgttttctatctgagttcacacgtgagcaccggtccccataatctaagaaagtggctcactgggcctagtggcgcattgtggcctttgatccgggctttgaccttggcgcacagcacccagtggtttggggaagaggtgtgtgtagcagaggaggttttttcgtgctttggtcccaatcaatccggcatctttgcagtgccgaggtggccgtgcaccttggctttgaattcttgtgctgaggttatgtgacttgagcctcaagatagggtcttctagcacaggcttgctcttaagtgtcgcagttgtcggtttcggcgtttgtttagagctgtggacacatctgtgaacttttgatgcttatttcagaggtcctgggttgtgcatgcgacgtcgggcccaattcgccc

SEQ ID NO: 141 - DNA, 23 bp, Artificial Sequence
  oligonucleotide
caccgtcggctccgctagactcg

SEQ ID NO: 142 - DNA, 23 bp, Artificial Sequence
  oligonucleotide
aaaccgagtctagcggagccgac

SEQ ID NO: 143 - DNA, 23 bp, Artificial Sequence
  oligonucleotide
caccgactcaccatggtggcgga

SEQ ID NO: 144 - DNA, 23 bp, Artificial Sequence
  oligonucleotide
aaactccgccaccatggtgagtc

SEQ ID NO: 145 - DNA, 8435 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcggctccgctagactcggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 146 - DNA, 8435 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactcaccatggtggcggagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 147 - DNA, 30 bp, Artificial Sequence
  oligonucleotide
agaccaggttagccttgaacaaagatctcc

SEQ ID NO: 148 - DNA, 6681 bp, Artificial Sequence
  synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatggtcgacctgcaggcggccgcgaattcactagtgattaccgttcgtatagcatacattatacgaagttataccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatcccaaataccactgaattaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatcctctagagtcgcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttatcgaattcccgcggtggcggccgcacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctccgcggccgccatggcggccgggagcatgcgacgtcgggcccaattcgccc

SEQ ID NO: 149 - DNA, 8434 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 150 - DNA, 8506 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 151 - DNA, 8436 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggatcccgggagctttaccggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 152 - DNA, 8435 bp, Artificial Sequence
  synthetic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccctcgccgcggtaaagctccgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 153 - DNA, 32 bp, Mus musculus
cgaccagcagacagggacatccatccagaggg

SEQ ID NO: 154 - DNA, 32 bp, Mus musculus
gctggtcgtctgtccctgtaggtaggtctccc

SEQ ID NO: 155 - DNA, 18 bp, Artificial Sequence
  Synthetic Construct
ctcccgatgtagaagaat